(19) United States
(12) Patent Application Publication
(10) Pub. No.: US 2025/0145602 A1
(43) Pub. Date: May 8, 2025

(54) Organic Compound, and Organic Light Emitting Device Including the Same

(71) Applicant: LG Display Co., Ltd., Seoul (KR)

(72) Inventors: Min-Surk Hyung, Paju-si (KR); Seon-Keun Yoo, Paju-si (KR); Shin-Han Kim, Paju-si (KR); Sang-Beom Kim, Paju-si (KR)

(21) Appl. No.: 18/920,719

(22) Filed: Oct. 18, 2024

(30) Foreign Application Priority Data
Nov. 3, 2023 (KR) 10-2023-0150895

(51) Int. Cl. C07D 413/14; C07D 417/14; C09K 11/02; C09K 11/06; H10K 50/16; H10K 85/30; H10K 85/60

(52) U.S. Cl. CPC C07D 413/14; C07D 417/14; C09K 11/02; C09K 11/06; H10K 50/16; H10K 85/30; H10K 85/622; H10K 85/654; H10K 85/656; H10K 85/6572; H10K 85/6574; H10K 85/6576

(57) ABSTRACT

An organic compound, and an organic light emitting diode and an organic light emitting device including the same are disclosed. For example, an organic compound is represented by the following chemical formula. The organic light emitting diode and the organic light emitting device each includes the organic compound.

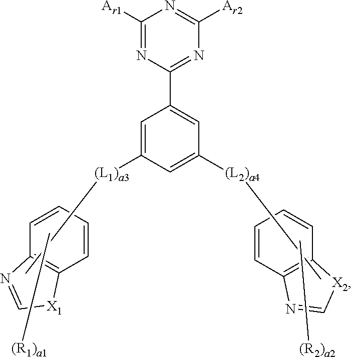

### CROSS-REFERENCE TO RELATED APPLICATIONS

The present application claims the benefit of and the priority to Republic of Korea Patent Application No. 10-2023-0150895 filed in the Republic of Korea on Nov. 3, 2023.

### TECHNICAL FIELD

The present disclosure relates to an organic compound, and more specifically, to an organic compound having improvements in the emitting efficiency and the lifespan and an organic light emitting device including the same.

### BACKGROUND

Recently, as demand for a flat panel display device having a small area have increased, an organic light emitting display device including an organic light emitting diode (OLED) has been the subject of recent research and development.

The OLED emits light by injecting electrons from a cathode as an electron injection electrode and holes from an anode as a hole injection electrode into an emitting material layer (EML), combining the electrons with the holes, generating an exciton, and transforming the exciton from an excited state to a ground state. A flexible substrate, for example, a plastic substrate, can be used as a base substrate where elements are formed. In addition, the organic light emitting display device can be operated at a voltage (e.g., 10V or below) lower than a voltage to operate other display devices. Moreover, the organic light emitting display device has improved power consumption and color.

The OLED includes a first electrode as an anode on a substrate, a second electrode as a cathode being spaced apart from and facing the first electrode and an organic light emitting layer between the first and second electrodes.

Although there have been many studies and developments on the materials of the organic light emitting layer, the OLED still has a limitation in the emitting efficiency and the lifespan.

### SUMMARY

The present disclosure is directed to an organic compound and an organic light emitting device that substantially obviate one or more of the problems associated with the limitations and disadvantages of the related art.

Additional features and advantages of the present disclosure are set forth in the description which follows, and will be apparent from the description, or evident by practice of the present disclosure. The objectives and other advantages of the present disclosure are realized and attained by the features described herein as well as in the appended drawings.

To achieve these and other advantages in accordance with the purpose of the embodiments of the present disclosure, as described herein, an aspect of the present disclosure is an organic compound represented by Formula 1:

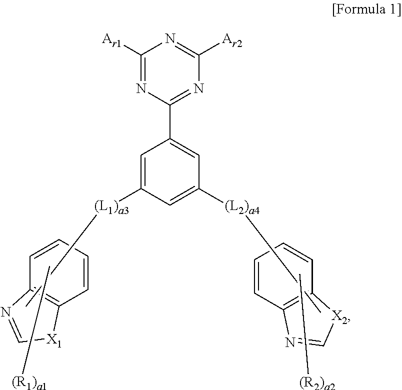

wherein each of a1 and a2 is independently an integer of 0 to 4, and each of a3 and a4 is independently 0 or 1, each of X1 and X2 is independently O or S, each of Ar₁ and Ar₂ is independently selected from the group consisting of a substituted or unsubstituted C6 to C60 aryl group and a substituted or unsubstituted C3 to C60 heteroaryl group containing one of N, O and S, each R₁ and R₂ is independently selected from the group consisting of a substituted or unsubstituted C1 to C10 alkyl group, a substituted or unsubstituted C3 to C30 cycloalkyl group, a substituted or unsubstituted C1 to C10 alkoxy group, a substituted or unsubstituted aryl group and a substituted or unsubstituted C3 to C60 heteroaryl group containing one of N, O and S, and each of L₁ and L₂ is independently selected from the group consisting of a substituted or unsubstituted C6 to C60 arylene group and a substituted or unsubstituted C3 to C60 heteroarylene group containing one of N, O and S. It will be understood that when a3 and/or a4 is 0, L1 and/or L2 is not present and the central phenyl ring and corresponding benzoxazole/benzothiazole moiety are directly linked by a single bond.

In preferred embodiments, Ar₁ and Ar₂ are different.

Another aspect of the present disclosure is an organic light emitting device comprising a substrate; and an organic light emitting diode positioned on the substrate and including a first electrode; a second electrode facing the first electrode; and a first emitting part between the first and second electrodes, the first emitting part including a first emitting material layer, a first electron transporting layer and a first hole blocking layer, wherein the first electron transporting layer is positioned between the first emitting material layer and the second electrode, and the first hole blocking layer is positioned between the first emitting material layer and the first electron transporting layer, and wherein at least one of the first electron transporting layer and the first hole blocking layer includes a first compound being an organic compound represented by Formula 1:

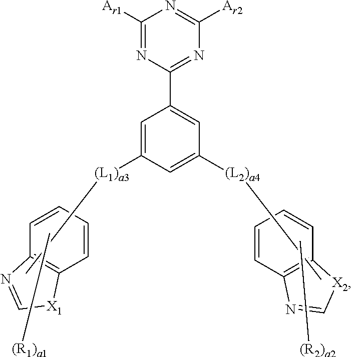

wherein each of a1 and a2 is independently an integer of 0 to 4, and each of a3 and a4 is independently 0 or 1, each of X1 and X2 is independently O or S, each of Ar and Ar₂ is independently selected from the group consisting of a substituted or unsubstituted C6 to C60 aryl group and a substituted or unsubstituted C3 to C60 heteroaryl group containing one of N, O and S, each R₁ and R₂ is independently selected from the group consisting of a substituted or unsubstituted C1 to C10 alkyl group, a substituted or unsubstituted C3 to C30 cycloalkyl group, a substituted or unsubstituted C1 to C10 alkoxy group, a substituted or unsubstituted aryl group and a substituted or unsubstituted C3 to C60 heteroaryl group containing one of N, O and S, and each of L₁ and L₂ is independently selected from the group consisting of a substituted or unsubstituted C6 to C60 arylene group and a substituted or unsubstituted C3 to C60 heteroarylene group containing one of N, O and S.

It is to be understood that both the foregoing general description and the following detailed description are exemplary and explanatory and are intended to further explain the present disclosure as claimed.

### BRIEF DESCRIPTION OF THE DRAWINGS

The accompanying drawings, which are included to provide a further understanding of the present disclosure and are incorporated in and constitute a part of this specification, illustrate embodiments of the present disclosure and together with the description serve to explain the principles of the present disclosure.

FIG. 1 illustrates a schematic circuit diagram for an organic light emitting display device according to an example embodiment of the present disclosure.

FIG. 2 illustrates a schematic cross-sectional view of an organic light emitting display device according to a first embodiment of the present disclosure.

FIG. 3 illustrates a schematic cross-sectional view of an OLED according to a second embodiment of the present disclosure.

FIG. 4 illustrates a schematic cross-sectional view of an OLED according to a third embodiment of the present disclosure.

FIG. 5 illustrates a schematic cross-sectional view of an organic light emitting display device according to a fourth embodiment of the present disclosure.

FIG. 6 illustrates a schematic cross-sectional view of an OLED according to a fifth embodiment of the present disclosure.

FIG. 7 illustrates a schematic cross-sectional view of an OLED according to a sixth embodiment of the present disclosure.

### DETAILED DESCRIPTION

Reference will now be made in detail to some of the examples and embodiments of the disclosure illustrated in the accompanying drawings. Wherever possible, the same reference numbers will be used throughout the drawings to refer to the same or like parts.

Advantages and features of the present disclosure, and implementation methods thereof will be clarified through following example embodiments described with reference to the accompanying drawings. The present disclosure may, however, be embodied in different forms and should not be construed as limited to the example embodiments set forth herein. Rather, these example embodiments are provided so that this disclosure may be sufficiently thorough and complete to assist those skilled in the art to fully understand the scope of the present disclosure. Further, the protected scope of the present disclosure is defined by claims and their equivalents.

The shapes, sizes, ratios, angles, numbers, and the like, which are illustrated in the drawings to describe various example embodiments of the present disclosure, are merely given by way of example. Therefore, the present disclosure is not limited to the illustrations in the drawings. The same or similar elements are designated by the same reference numerals throughout the specification unless otherwise specified.

In the following description, where the detailed description of the relevant known function or configuration may unnecessarily obscure an important point of the present disclosure, a detailed description of such known function of configuration may be omitted.

In the present specification, where the terms “comprise,” “have,” “include,” and the like are used, one or more other elements may be added unless the term, such as “only,” is used. An element described in the singular form is intended to include a plurality of elements, and vice versa, unless the context clearly indicates otherwise.

In construing an element, the element is to be construed as including an error or tolerance range even where no explicit description of such an error or tolerance range is provided.

In the description of the various embodiments of the present disclosure, where positional relationships are described, for example, where the positional relationship between two parts is described using “on,” “over,” “under,” “above,” “below,” “beside,” “next,” or the like, one or more other parts may be located between the two parts unless a more limiting term, such as “immediate(ly),” “direct(ly),” or “close(ly)” is used. For example, where an element or layer is disposed “on” another element or layer, a third layer or element may be interposed therebetween.

In describing a temporal relationship, when the temporal order is described as, for example, “after,” “subsequent,” “next,” or “before,” a case which is not continuous may be included unless a more limiting term, such as “just,” “immediate(ly),” or “direct(ly),” is used.

Although the terms “first,” “second,” and the like may be used herein to describe various elements, the elements should not be limited by these terms. These terms are used only to identify one element from another. For example, a first element could be termed a second element, and similarly, a second element could be termed a first element, without departing from the scope of the present disclosure.

Although the terms “first,” “second,” A, B, (a), (b), and the like may be used herein to describe various elements, the elements should not be interpreted to be limited by these terms as they are not used to define a particular order, precedence, or number of the corresponding elements. These terms are used only to identify one element from another.

The expression that an element or layer is “connected” to another element or layer means the element or layer can not only be directly connected to another element or layer, but also be indirectly connected or adhered to another element or layer with one or more intervening elements or layers “disposed,” or “interposed” between the elements or layers, unless otherwise specified.

The term “at least one” should be understood as including any and all combinations of one or more of the associated listed items. For example, the meaning of “at least one of a first element, a second element, and a third element” encompasses the combination of all three listed elements, combinations of any two of the three elements, as well as each individual element, the first element, the second element, and the third element.

Features of various embodiments of the present disclosure may be partially or overall coupled to or combined with each other, and may be variously inter-operated with each other and driven technically as those skilled in the art can sufficiently understand. Embodiments of the present disclosure may be carried out independently from each other, or may be carried out together in a co-dependent relationship.

Hereinafter, example embodiments of the present disclosure will be described in detail with reference to the accompanying drawings. In adding reference numerals to elements of each of the drawings, although the same elements are illustrated in other drawings, like reference numerals may refer to like elements. Also, for convenience of description, a scale in which each of the elements is illustrated in the accompanying drawings may differ from an actual scale. Thus, the illustrated elements are not limited to the specific scale in which they are illustrated in the drawings.

### <Organic Compound>

An organic compound of the present disclosure is represented by Formula 1.

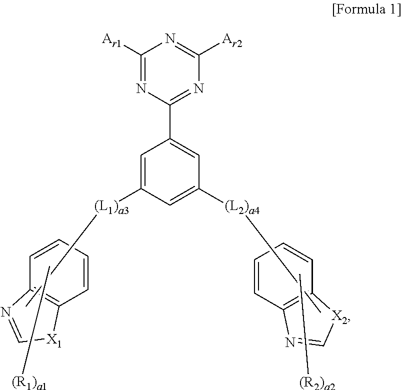

In Formula 1, each of a1 and a2 is independently an integer of 0 to 4, and each of a3 and a4 is independently 0 or 1,
each of X1 and X2 is independently O or S,
each of Ar₁ and Ar₂ is independently selected from the group consisting of a substituted or unsubstituted C6 to C60 aryl group and a substituted or unsubstituted C3 to C60 heteroaryl group containing one of N, O and S,
each R₁ and R₂ is independently selected from the group consisting of a substituted or unsubstituted C1 to C10 alkyl group, a substituted or unsubstituted C3 to C30 cycloalkyl group, a substituted or unsubstituted C1 to C10 alkoxy group, a substituted or unsubstituted aryl group and a substituted or unsubstituted C3 to C60 heteroaryl group containing one of N, O and S,
each of L₁ and L₂ is independently selected from the group consisting of a substituted or unsubstituted C6 to C60 arylene group and a substituted or unsubstituted C3 to C60 heteroarylene group containing one of N, O and S.

It will be appreciated that R-groups represent substitution as valency allows. Where said groups represent no-substitution, hydrogen atoms are present as required to satisfy the valency requirements of the compound.

In the present disclosure, without specific definition, a substituent of an alkyl group, a cycloalkyl group, an alkoxy group, an aryl group, a heteroaryl group, an arylene group, and a heteroarylene group may be selected from the group consisting of deuterium (D), halogen, cyano, a hydroxyl group, a C1 to C10 alkyl group, a C1 to C10 alkoxy group, a C3 to C30 cycloalkyl group, a C1 to C10 alkylsilyl group, a C1 to C10 alkylamino group, a C6 to C30 arylsilyl group, a C6 to C30 arylamino group, a C6 to C30 aryl group and a C3 to C30 heteroaryl group. For example, the substituent may be at least one selected from the group consisting of D, F, Br, CN, hydroxyl, methyl, ethyl, propyl, butyl (e.g., tert-butyl), methoxy, ethoxy, propoxy, butoxy (e.g., tert-butoxy), cyclopropyl, cyclobutyl, cryclopentyl, cyclohexyl, trimethylsilyl, trimethylamino, triphenylsiliyl, triphenylamino, phenyl, biphenyl, naphthyl, anthracenyl, pyridyl, carbazolyl, dibenzofuranyl and dibenzothiophenyl.

In the present disclosure, without specific definition, the term “alkyl” means a substituted or unsubstituted, saturated, linear or branched hydrocarbon chain radical. For example, the C1 to C10 alkyl group may be selected from the group consisting of methyl, ethyl, propyl and butyl, e.g., tert-butyl.

In the present disclosure, without specific definition, the term “aryl” means a monovalent monocyclic or polycyclic conjugated ring structure. For example, the C6 to C60 aryl group may be selected from the group consisting of phenyl, biphenyl, terphenyl, naphthyl, anthracenyl, pentanenyl, indenyl, indenoindenyl, heptalenyl, biphenylenyl, indacenyl, phenanthrenyl, benzophenanthrenyl, dibenzophenanthrenyl, azulenyl, pyrenyl, fluoranthenyl, triphenylenyl, chrysenyl, tetraphenyl, tetrasenyl, picenyl, pentaphenyl, pentacenyl, fluorenyl, indenofluorenyl, and spiro-fluorenyl.

In the present disclosure, without specific definition, the term “arylene” means a divalent monocyclic or polycyclic conjugated ring structure. For example, the C6 to C60 arylene group may be selected from the group consisting of phenylene, biphenylene, terphenylene, naphthylene, anthracenylene, pentanenylene, indenylene, indenoindenylene, heptalenylene, biphenylenylene, indacenylene, phenanthrenylene, benzophenanthrenylene, dibenzophenanthrenylene, azulenylene, pyrenylene, fluoranthenylene, triphenylenylene, chrysenylene, tetraphenylene, tetrasenylene, picenylene, pentaphenylene, pentacenylene, fluorenylene, indenofluorenylene, and spiro-fluorenylene.

In the present disclosure, without specific definition, the term “heteroaryl” refers to a 5- to 7-membered aromatic ring which includes 1, 2, 3 or 4 hetero atoms such as nitrogen, oxygen or sulfur and such rings fused to an aryl, cycloalkyl, heteroaryl or heterocycloalkyl ring. For example, the C3 to C60 heteroaryl group may be selected from the group consisting of pyrrolyl, pyridinyl, pyrimidinyl, pyrazinyl, pyridazinyl, triazinyl, tetrazinyl, imidazolyl, pyrazolyl, indolyl, isoindolyl, indazolyl, indolizinyl, pyrrolizinyl, carbazolyl, benzocarbazolyl, dibenzocarbazolyl, indolocarbazolyl, indenocarbazolyl, benzofurocarbazolyl, benzothienocarbazolyl, quinolinyl, isoquinolinyl, phthalazinyl, quinoxalinyl, cinnolinyl, quinazolinyl, quinozolinyl, purinyl, benzoquinolinyl, benzoisoquinolinyl, benzoquinazolinyl, benzoquinoxalinyl, acridinyl, phenanthrolinyl, perimidinyl, phenanthridinyl, pteridinyl, cinnolinyl, naphtharidinyl, furanyl, oxazinyl, oxazolyl, oxadiazolyl, triazolyl, dioxynyl, benzofuranyl, dibenzofuranyl, thiopyranyl, xanthenyl, chromanyl, isochromanyl, thioazinyl, thiophenyl, benzothiophenyl, dibenzothiophenyl, difuropyrazinyl, benzofurodibenzofuranyl, benzothienobenzothiophenyl, benzothienodibenzothiophenyl, benzothienobenzofuranyl, and benzothienodibenzofuranyl.

In the present disclosure, without specific definition, the term “heteroarylene” refers to the divalent counterpart of a heteroaryl group, as defined above. For example, the C3 to C60 heteroarylene group may be selected from the group consisting of pyrrolylene, pyridinylene, pyrimidinylene, pyrazinylene, pyridazinylene, triazinylene, tetrazinylene, imidazolylene, pyrazolylene, indolylene, isoindolylene, indazolylene, indolizinylene, pyrrolizinylene, carbazolylene, benzocarbazolylene, dibenzocarbazolylene, indolocarbazolylene, indenocarbazolylene, benzofurocarbazolylene, benzothienocarbazolylene, quinolinylene, isoquinolinylene, phthalazinylene, quinoxalinylene, cinnolinylene, quinazolinylene, quinozolinylene, purinylene, benzoquinolinylene, benzoisoquinolinylene, benzoquinazolinylene, benzoquinoxalinylene, acridinylene, phenanthrolinylene, perimidinylene, phenanthridinylene, pteridinylene, cinnolinylene, naphtharidinylene, furanylene, oxazinylene, oxazolylene, oxadiazolylene, triazolylene, dioxynylene, benzofuranylene, dibenzofuranylene, thiopyranylene, xanthenylene, chromanylene, isochromanylene, thioazinylene, thiophenylene, benzothiophenylene, dibenzothiophenylene, difuropyrazinylene, benzofurodibenzofuranylene, benzothienobenzothiophenylene, benzothienodibenzothiophenylene, benzothienobenzofuranylene, and benzothienodibenzofuranylene.

In an aspect of the present disclosure, each of a1, a2, a3 and a4 may be 0.

In an aspect of the present disclosure, each of Ar₁ and Ar₂ may be independently a substituted or unsubstituted C6 to C60 aryl group and may be the same or different.

In an aspect of the present disclosure, one of Ar₁ and Ar₂ may be a substituted or unsubstituted C6 to C60 aryl group, and the other one of Ar₁ and Ar₂ may be a substituted or unsubstituted C3 to C60 heteroaryl group containing one of N, O and S.

In an aspect of the present disclosure, each of Ar₁ and Ar₂ may be independently a substituted or unsubstituted C3 to C60 heteroaryl group containing one of N, O and S and may be the same or different.

In an aspect of the present disclosure, each of Ar₁ and Ar₂ may be independently selected from an aryl group represented by Formula 1a-1 and a heteroaryl group represented by Formula 1a-2. In each of Formulas 1a-1 and 1a-2, the mark “*” denotes a bonding site.

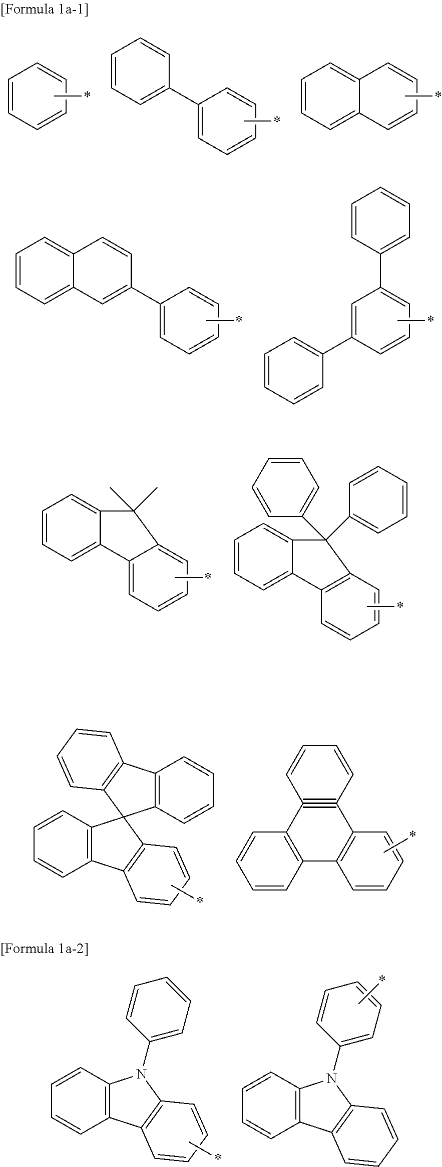

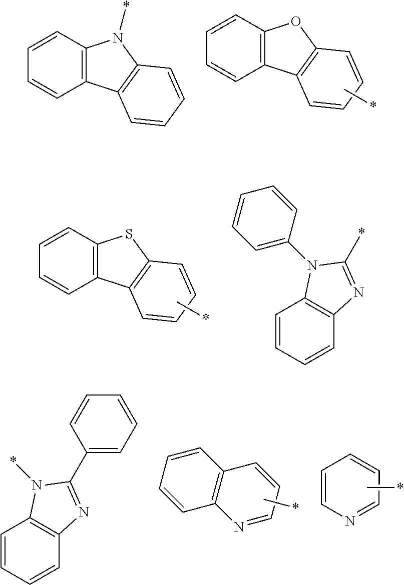

In an aspect of the present disclosure, a bonding position of each of a moiety including X₁ and a moiety of including X₂ may be specified. Namely, the organic compound of the present disclosure, which is represented by Formula 1, may be represented by Formula 1b-1.

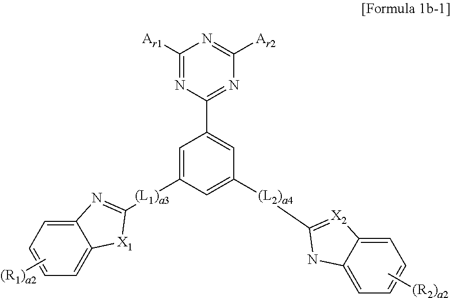

In Formula 1b-1, the definition of a1, a2, a3, a4, X1, X₂, Ar₁, Ar₂, R₁, R₂, L₁ and L₂ is the same as for Formula 1.

In an aspect of the present disclosure, each of a3 and a4 may be 0, and a bonding position of each of a moiety including X₁ and a moiety of including X₂ may be specified. Namely, the organic compound of the present disclosure, which is represented by Formula 1, may be represented by Formula 1b-2.

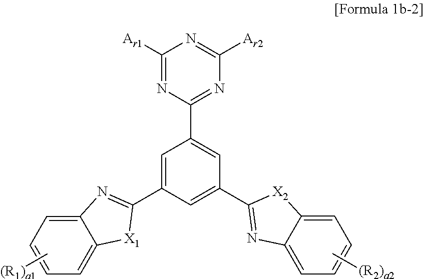

In Formula 1b-1, the definition of a1, a2, X₁, X₂, Ar₁, Ar₂, R₁ and R₂ is the same as for Formula 1.

For example, the organic compound of the present disclosure may be one of the compounds in Formula 2.

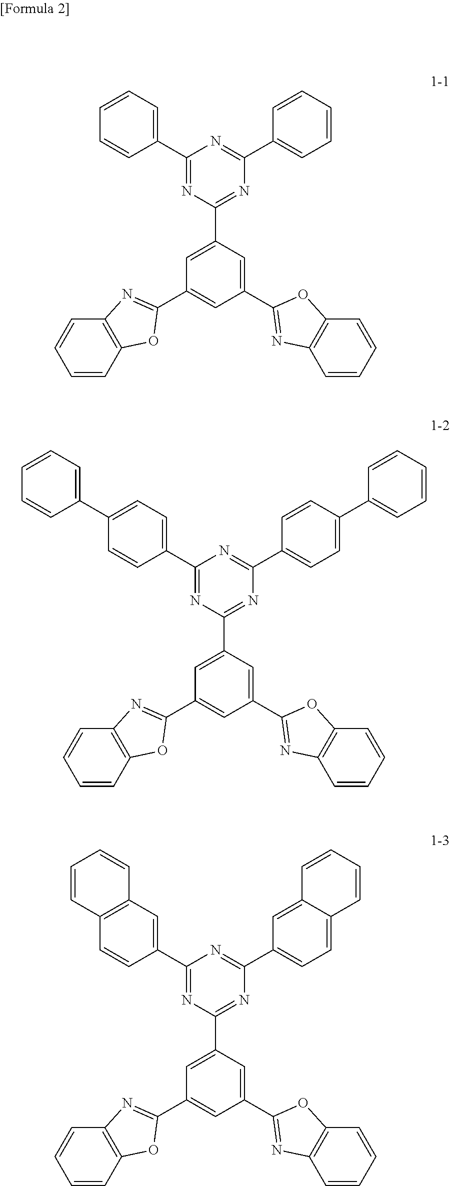 [SMILES: c1ccc(-c2nc(-c3ccccc3)nc(-c3cc(-c4nc5ccccc5o4)cc(-c4nc5ccccc5o4)c3)n2)cc1 ; c1ccc(-c2ccc(-c3nc(-c4ccc(-c5ccccc5)cc4)nc(-c4cc(-c5nc6ccccc6o5)cc(-c5nc6ccccc6o5)c4)n3)cc2)cc1 ; c1ccc2cc(-c3nc(-c4cc(-c5nc6ccccc6o5)cc(-c5nc6ccccc6o5)c4)nc(-c4ccc5ccccc5c4)n3)ccc2c1]

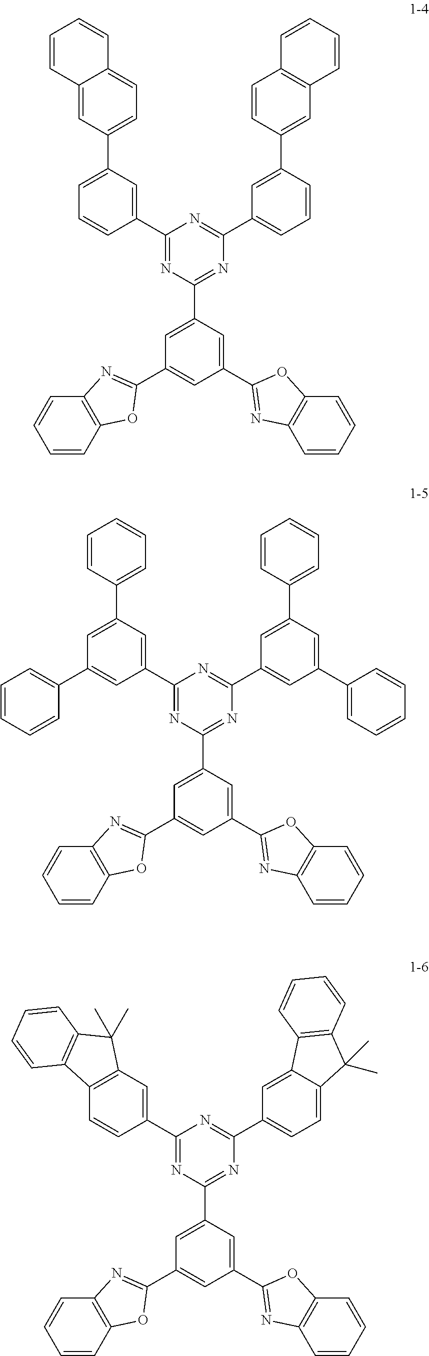 [SMILES: c1cc(-c2ccc3ccccc3c2)cc(-c2nc(-c3cccc(-c4ccc5ccccc5c4)c3)nc(-c3cc(-c4nc5ccccc5o4)cc(-c4nc5ccccc5o4)c3)n2)c1 ; c1ccc(-c2cc(-c3ccccc3)cc(-c3nc(-c4cc(-c5ccccc5)cc(-c5ccccc5)c4)nc(-c4cc(-c5nc6ccccc6o5)cc(-c5nc6ccccc6o5)c4)n3)c2)cc1 ; CC1(C)c2ccccc2-c2cc(-c3nc(-c4cc(-c5nc6ccccc6o5)cc(-c5nc6ccccc6o5)c4)nc(-c4ccc5c(c4)C(C)(C)c4ccccc4-5)n3)ccc21]

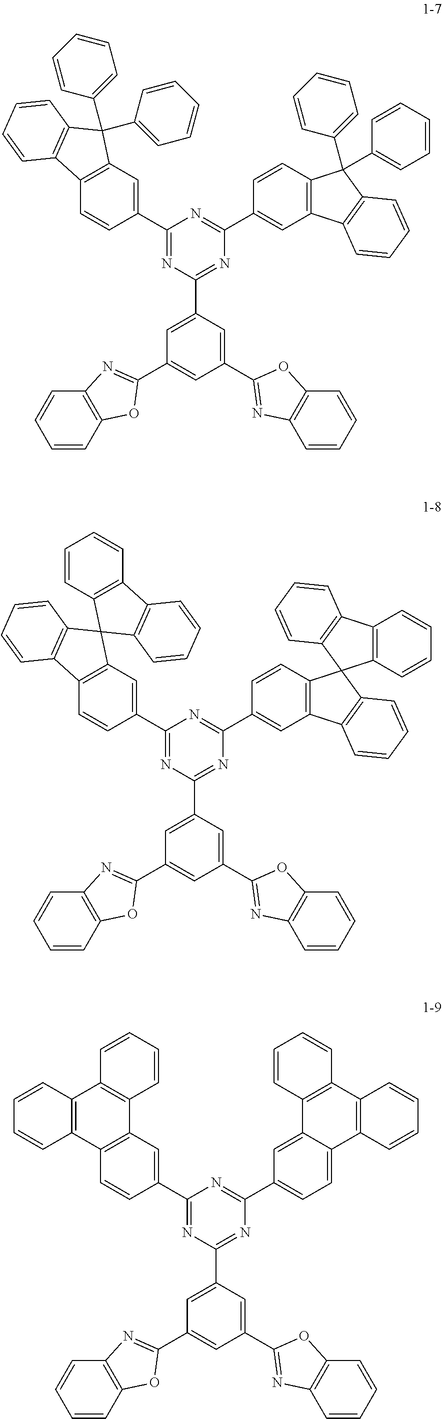 [SMILES: c1ccc(C2(c3ccccc3)c3ccccc3-c3cc(-c4nc(-c5cc(-c6nc7ccccc7o6)cc(-c6nc7ccccc7o6)c5)nc(-c5ccc6c(c5)C(c5ccccc5)(c5ccccc5)c5ccccc5-6)n4)ccc32)cc1 ; c1ccc2c(c1)-c1ccccc1C21c2ccccc2-c2cc(-c3nc(-c4cc(-c5nc6ccccc6o5)cc(-c5nc6ccccc6o5)c4)nc(-c4ccc5c(c4)C4(c6ccccc6-c6ccccc64)c4ccccc4-5)n3)ccc21 ; c1ccc2oc(-c3cc(-c4nc(-c5ccc6c7ccccc7c7ccccc7c6c5)nc(-c5ccc6c7ccccc7c7ccccc7c6c5)n4)cc(-c4nc5ccccc5o4)c3)nc2c1]

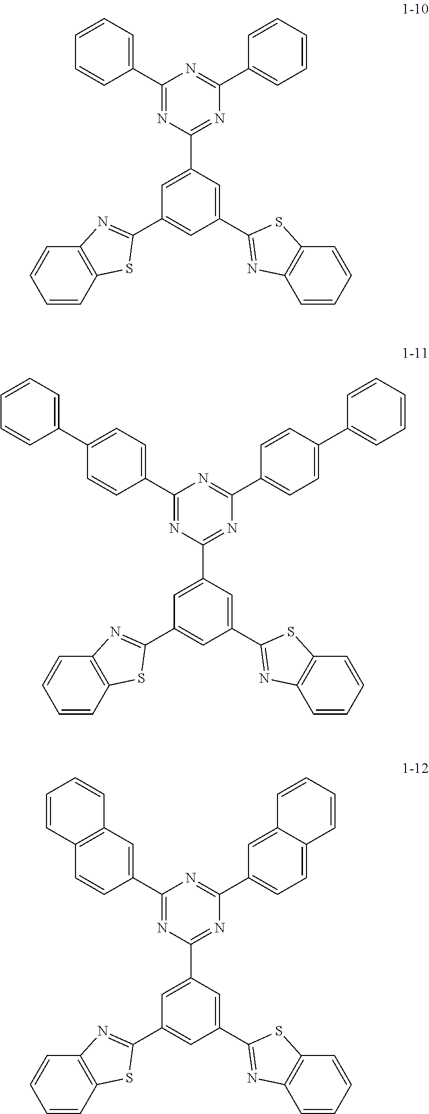 [SMILES: c1ccc(-c2nc(-c3ccccc3)nc(-c3cc(-c4nc5ccccc5s4)cc(-c4nc5ccccc5s4)c3)n2)cc1 ; c1ccc(-c2ccc(-c3nc(-c4ccc(-c5ccccc5)cc4)nc(-c4cc(-c5nc6ccccc6s5)cc(-c5nc6ccccc6s5)c4)n3)cc2)cc1 ; c1ccc2cc(-c3nc(-c4cc(-c5nc6ccccc6s5)cc(-c5nc6ccccc6s5)c4)nc(-c4ccc5ccccc5c4)n3)ccc2c1]

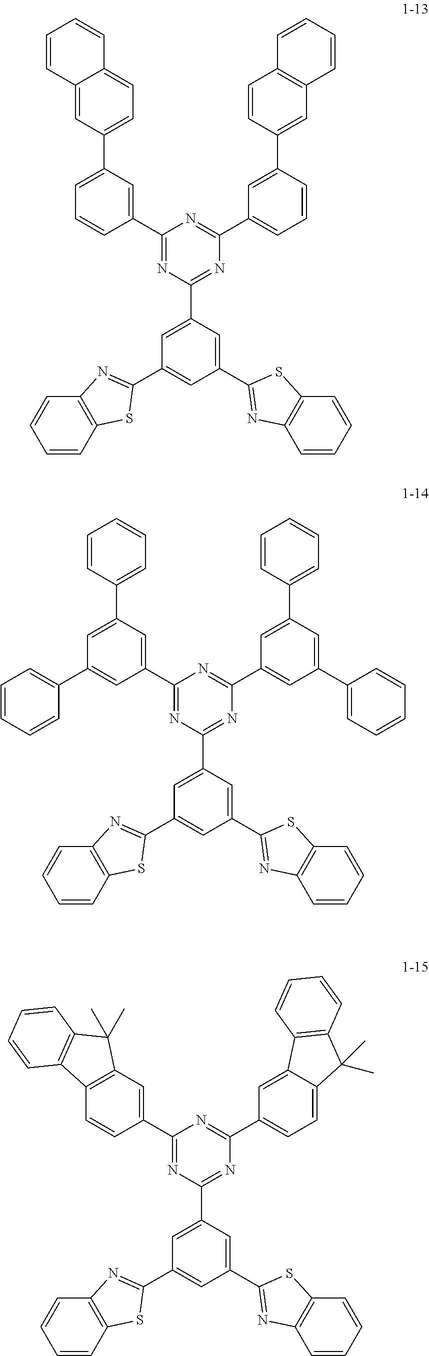 [SMILES: c1cc(-c2ccc3ccccc3c2)cc(-c2nc(-c3cccc(-c4ccc5ccccc5c4)c3)nc(-c3cc(-c4nc5ccccc5s4)cc(-c4nc5ccccc5s4)c3)n2)c1 ; c1ccc(-c2cc(-c3ccccc3)cc(-c3nc(-c4cc(-c5ccccc5)cc(-c5ccccc5)c4)nc(-c4cc(-c5nc6ccccc6s5)cc(-c5nc6ccccc6s5)c4)n3)c2)cc1 ; CC1(C)c2ccccc2-c2cc(-c3nc(-c4cc(-c5nc6ccccc6s5)cc(-c5nc6ccccc6s5)c4)nc(-c4ccc5c(c4)C(C)(C)c4ccccc4-5)n3)ccc21]

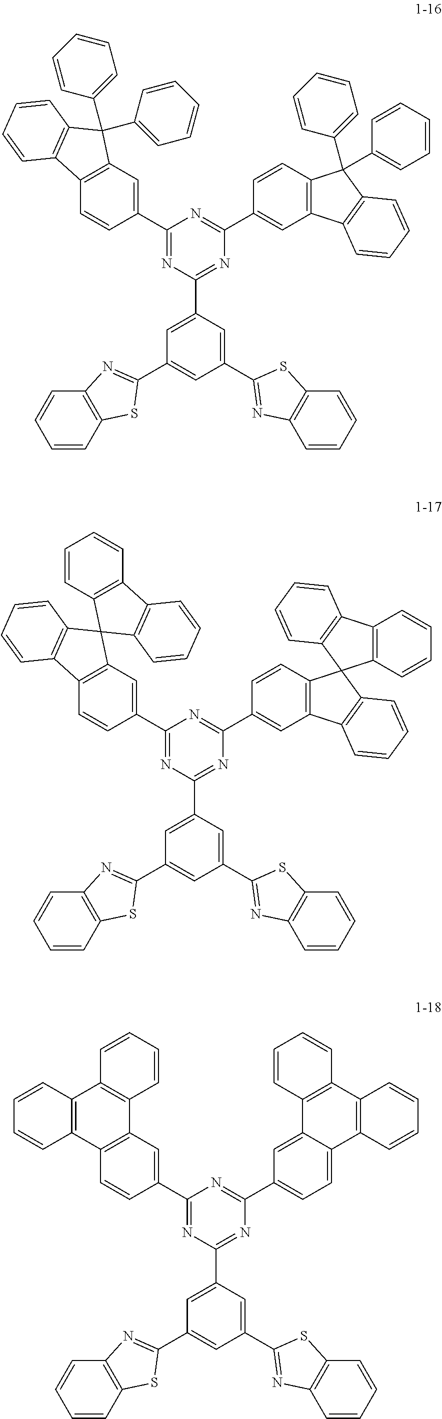 [SMILES: c1ccc(C2(c3ccccc3)c3ccccc3-c3cc(-c4nc(-c5cc(-c6nc7ccccc7s6)cc(-c6nc7ccccc7s6)c5)nc(-c5ccc6c(c5)C(c5ccccc5)(c5ccccc5)c5ccccc5-6)n4)ccc32)cc1 ; c1ccc2c(c1)-c1ccccc1C21c2ccccc2-c2cc(-c3nc(-c4cc(-c5nc6ccccc6s5)cc(-c5nc6ccccc6s5)c4)nc(-c4ccc5c(c4)C4(c6ccccc6-c6ccccc64)c4ccccc4-5)n3)ccc21 ; c1ccc2sc(-c3cc(-c4nc(-c5ccc6c7ccccc7c7ccccc7c6c5)nc(-c5ccc6c7ccccc7c7ccccc7c6c5)n4)cc(-c4nc5ccccc5s4)c3)nc2c1]

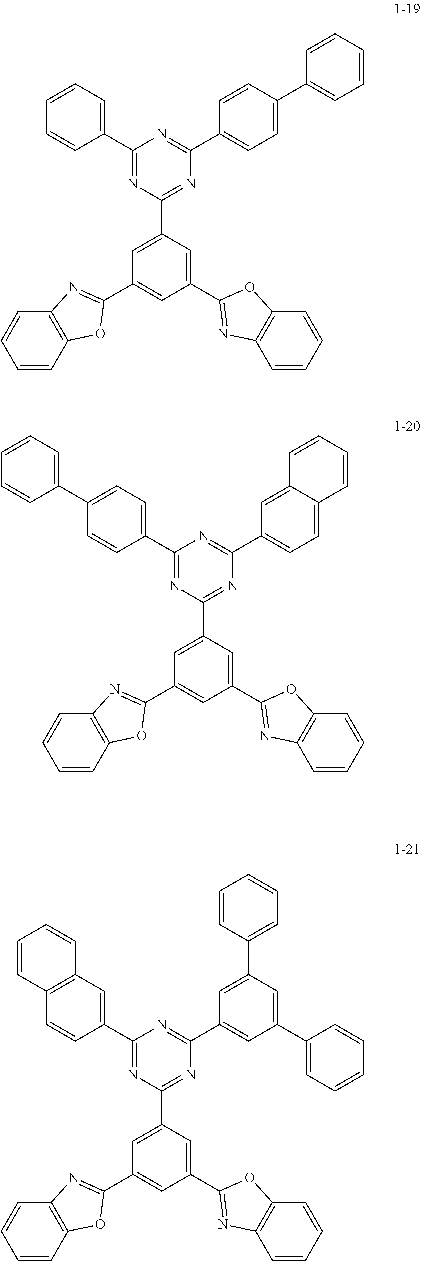 [SMILES: c1ccc(-c2ccc(-c3nc(-c4ccccc4)nc(-c4cc(-c5nc6ccccc6o5)cc(-c5nc6ccccc6o5)c4)n3)cc2)cc1 ; c1ccc(-c2ccc(-c3nc(-c4cc(-c5nc6ccccc6o5)cc(-c5nc6ccccc6o5)c4)nc(-c4ccc5ccccc5c4)n3)cc2)cc1 ; c1ccc(-c2cc(-c3ccccc3)cc(-c3nc(-c4cc(-c5nc6ccccc6o5)cc(-c5nc6ccccc6o5)c4)nc(-c4ccc5ccccc5c4)n3)c2)cc1]

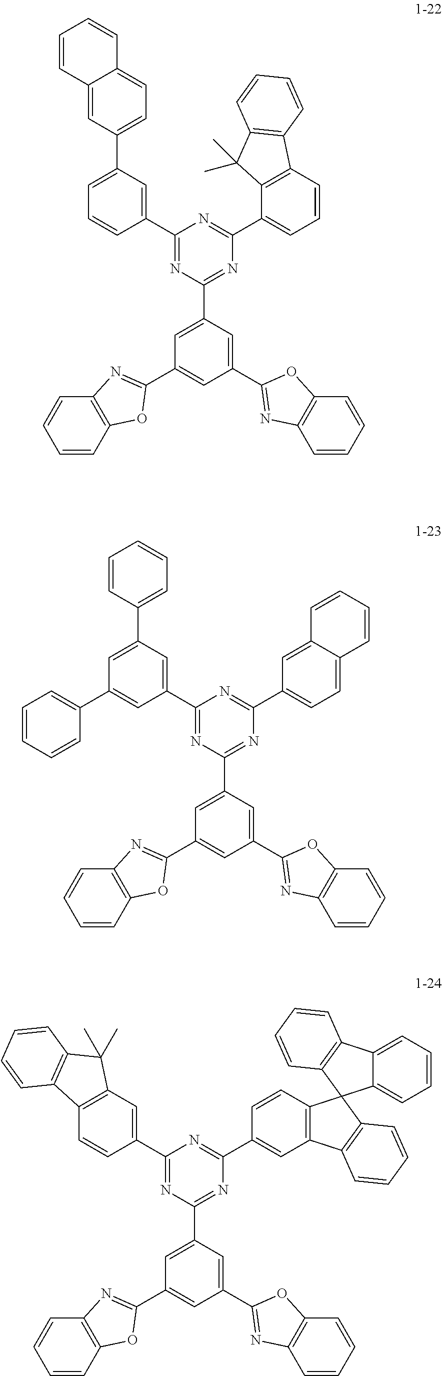 [SMILES: CC1(C)c2ccccc2-c2cccc(-c3nc(-c4cccc(-c5ccc6ccccc6c5)c4)nc(-c4cc(-c5nc6ccccc6o5)cc(-c5nc6ccccc6o5)c4)n3)c21 ; c1ccc(-c2cc(-c3ccccc3)cc(-c3nc(-c4cc(-c5nc6ccccc6o5)cc(-c5nc6ccccc6o5)c4)nc(-c4ccc5ccccc5c4)n3)c2)cc1 ; CC1(C)c2ccccc2-c2ccc(-c3nc(-c4cc(-c5nc6ccccc6o5)cc(-c5nc6ccccc6o5)c4)nc(-c4ccc5c(c4)-c4ccccc4C54c5ccccc5-c5ccccc54)n3)cc21]

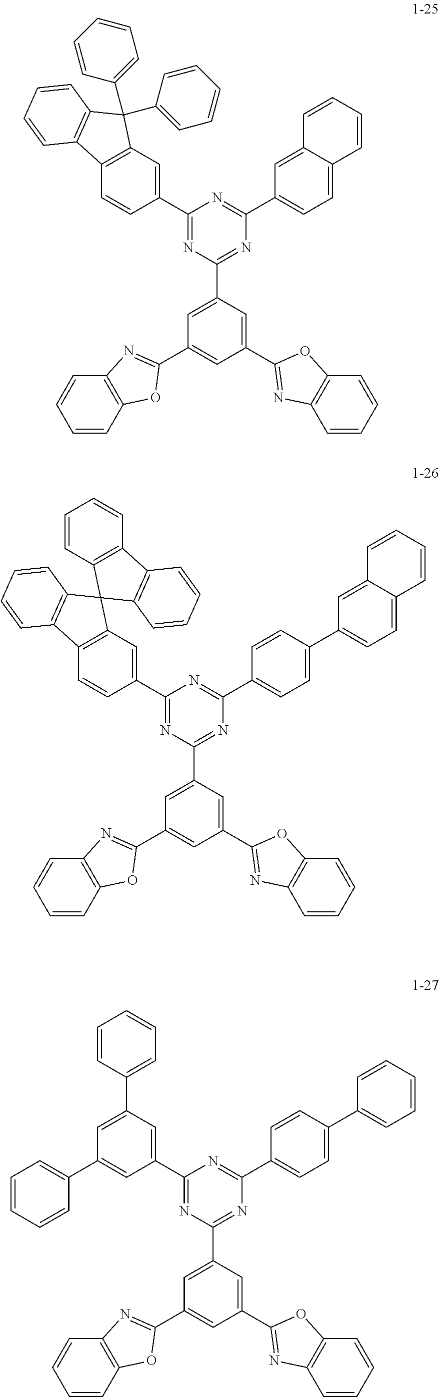 [SMILES: c1ccc(C2(c3ccccc3)c3ccccc3-c3ccc(-c4nc(-c5cc(-c6nc7ccccc7o6)cc(-c6nc7ccccc7o6)c5)nc(-c5ccc6ccccc6c5)n4)cc32)cc1 ; c1ccc2c(c1)-c1ccccc1C21c2ccccc2-c2ccc(-c3nc(-c4ccc(-c5ccc6ccccc6c5)cc4)nc(-c4cc(-c5nc6ccccc6o5)cc(-c5nc6ccccc6o5)c4)n3)cc21 ; c1ccc(-c2ccc(-c3nc(-c4cc(-c5ccccc5)cc(-c5ccccc5)c4)nc(-c4cc(-c5nc6ccccc6o5)cc(-c5nc6ccccc6o5)c4)n3)cc2)cc1]

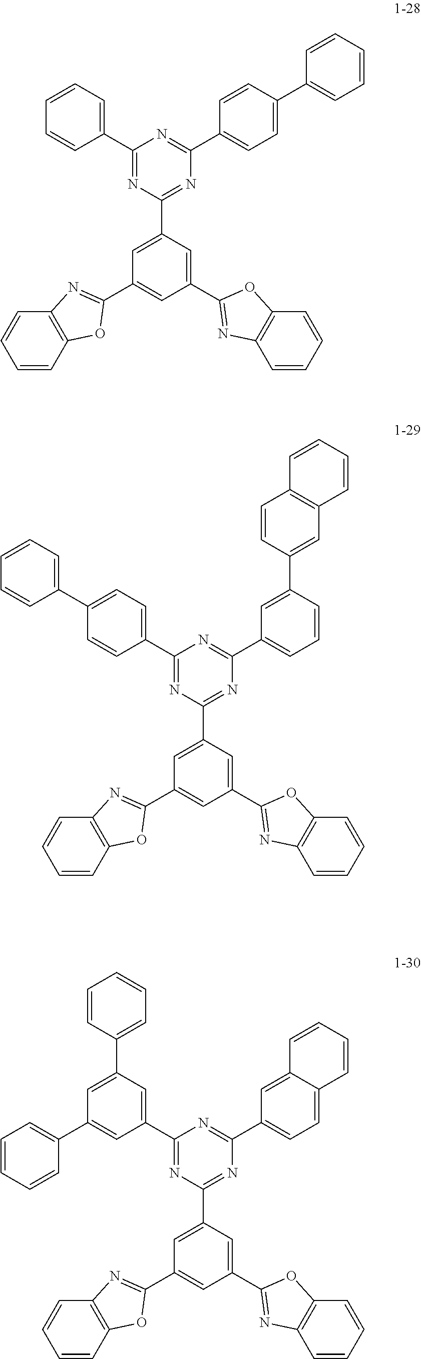 [SMILES: c1ccc(-c2ccc(-c3nc(-c4ccccc4)nc(-c4cc(-c5nc6ccccc6o5)cc(-c5nc6ccccc6o5)c4)n3)cc2)cc1 ; c1ccc(-c2ccc(-c3nc(-c4cccc(-c5ccc6ccccc6c5)c4)nc(-c4cc(-c5nc6ccccc6o5)cc(-c5nc6ccccc6o5)c4)n3)cc2)cc1 ; c1ccc(-c2cc(-c3ccccc3)cc(-c3nc(-c4cc(-c5nc6ccccc6o5)cc(-c5nc6ccccc6o5)c4)nc(-c4ccc5ccccc5c4)n3)c2)cc1]

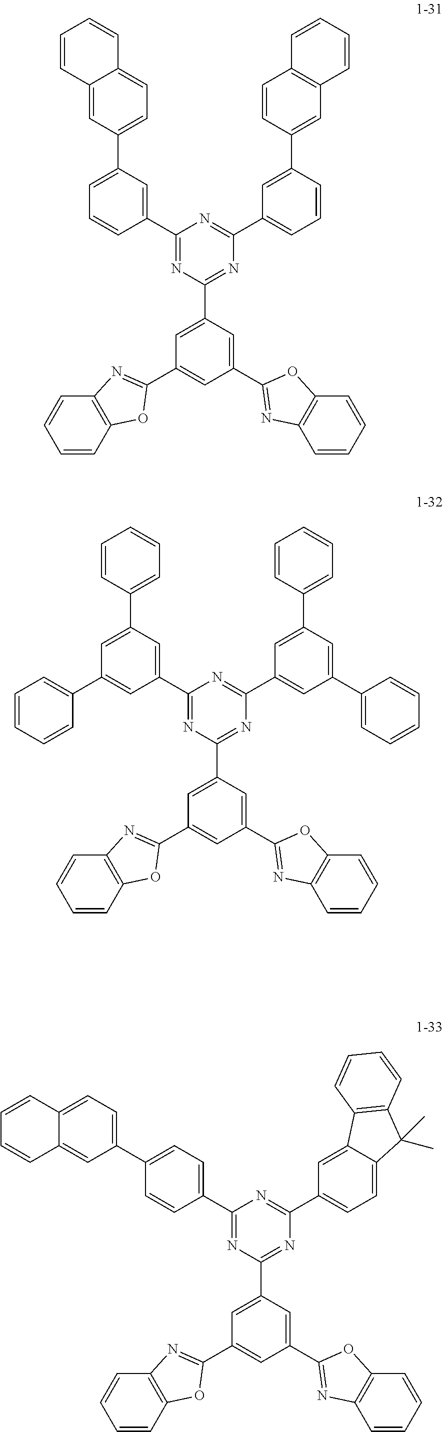 [SMILES: c1cc(-c2ccc3ccccc3c2)cc(-c2nc(-c3cccc(-c4ccc5ccccc5c4)c3)nc(-c3cc(-c4nc5ccccc5o4)cc(-c4nc5ccccc5o4)c3)n2)c1 ; c1ccc(-c2cc(-c3ccccc3)cc(-c3nc(-c4cc(-c5ccccc5)cc(-c5ccccc5)c4)nc(-c4cc(-c5nc6ccccc6o5)cc(-c5nc6ccccc6o5)c4)n3)c2)cc1 ; CC1(C)c2ccccc2-c2cc(-c3nc(-c4ccc(-c5ccc6ccccc6c5)cc4)nc(-c4cc(-c5nc6ccccc6o5)cc(-c5nc6ccccc6o5)c4)n3)ccc21]

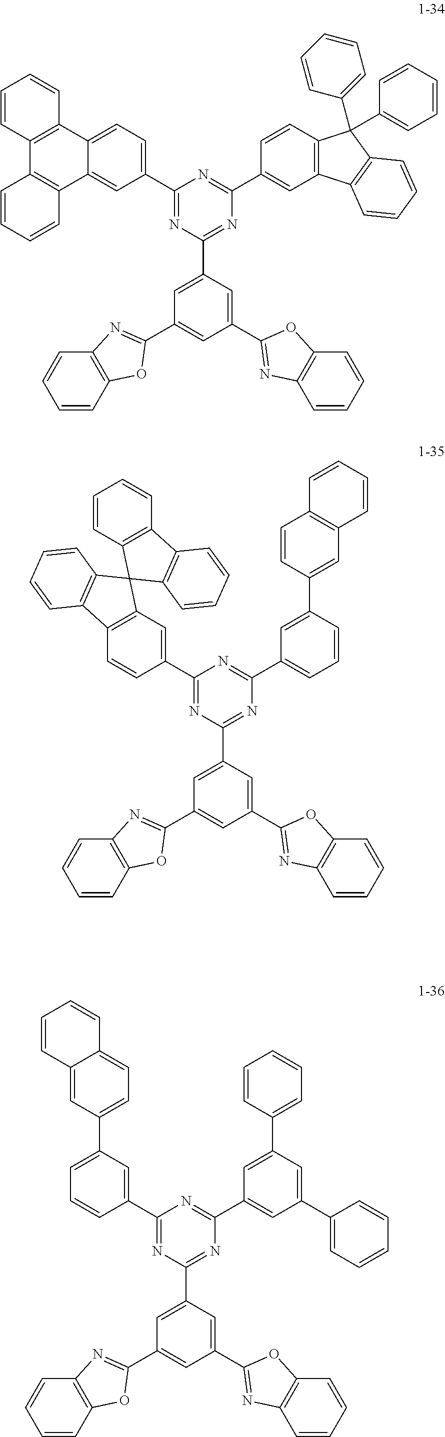 [SMILES: c1ccc(C2(c3ccccc3)c3ccccc3-c3cc(-c4nc(-c5cc(-c6nc7ccccc7o6)cc(-c6nc7ccccc7o6)c5)nc(-c5ccc6c7ccccc7c7ccccc7c6c5)n4)ccc32)cc1 ; c1cc(-c2ccc3ccccc3c2)cc(-c2nc(-c3cc(-c4nc5ccccc5o4)cc(-c4nc5ccccc5o4)c3)nc(-c3ccc4c(c3)C3(c5ccccc5-c5ccccc53)c3ccccc3-4)n2)c1 ; c1ccc(-c2cc(-c3ccccc3)cc(-c3nc(-c4cccc(-c5ccc6ccccc6c5)c4)nc(-c4cc(-c5nc6ccccc6o5)cc(-c5nc6ccccc6o5)c4)n3)c2)cc1]

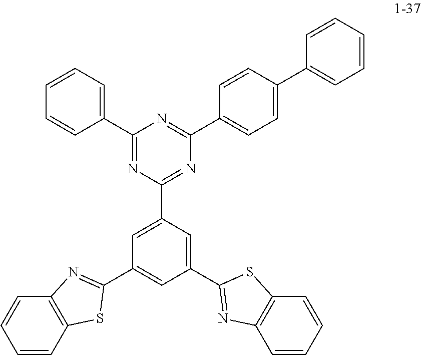 [SMILES: c1ccc(-c2ccc(-c3nc(-c4ccccc4)nc(-c4cc(-c5nc6ccccc6s5)cc(-c5nc6ccccc6s5)c4)n3)cc2)cc1]

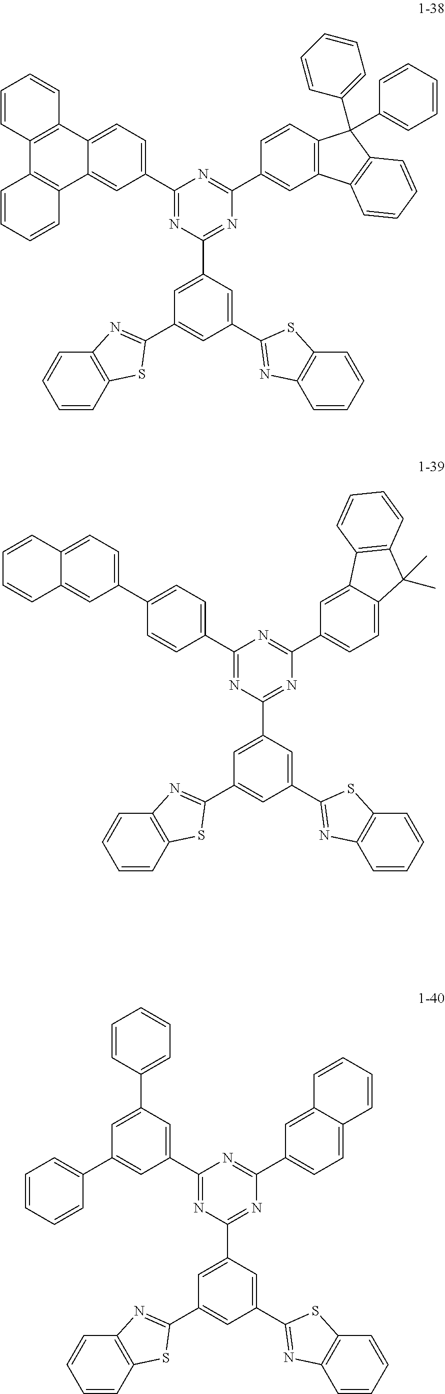 [SMILES: c1ccc(C2(c3ccccc3)c3ccccc3-c3cc(-c4nc(-c5cc(-c6nc7ccccc7s6)cc(-c6nc7ccccc7s6)c5)nc(-c5ccc6c7ccccc7c7ccccc7c6c5)n4)ccc32)cc1 ; CC1(C)c2ccccc2-c2cc(-c3nc(-c4ccc(-c5ccc6ccccc6c5)cc4)nc(-c4cc(-c5nc6ccccc6s5)cc(-c5nc6ccccc6s5)c4)n3)ccc21 ; c1ccc(-c2cc(-c3ccccc3)cc(-c3nc(-c4cc(-c5nc6ccccc6s5)cc(-c5nc6ccccc6s5)c4)nc(-c4ccc5ccccc5c4)n3)c2)cc1]

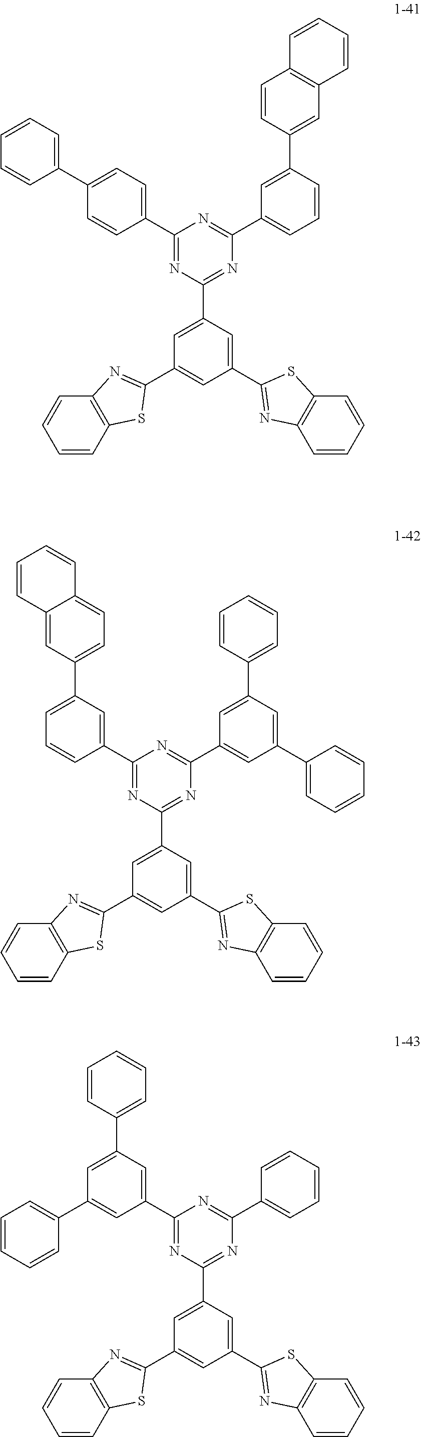 [SMILES: c1ccc(-c2ccc(-c3nc(-c4cccc(-c5ccc6ccccc6c5)c4)nc(-c4cc(-c5nc6ccccc6s5)cc(-c5nc6ccccc6s5)c4)n3)cc2)cc1 ; c1ccc(-c2cc(-c3ccccc3)cc(-c3nc(-c4cccc(-c5ccc6ccccc6c5)c4)nc(-c4cc(-c5nc6ccccc6s5)cc(-c5nc6ccccc6s5)c4)n3)c2)cc1 ; c1ccc(-c2cc(-c3ccccc3)cc(-c3nc(-c4ccccc4)nc(-c4cc(-c5nc6ccccc6s5)cc(-c5nc6ccccc6s5)c4)n3)c2)cc1]

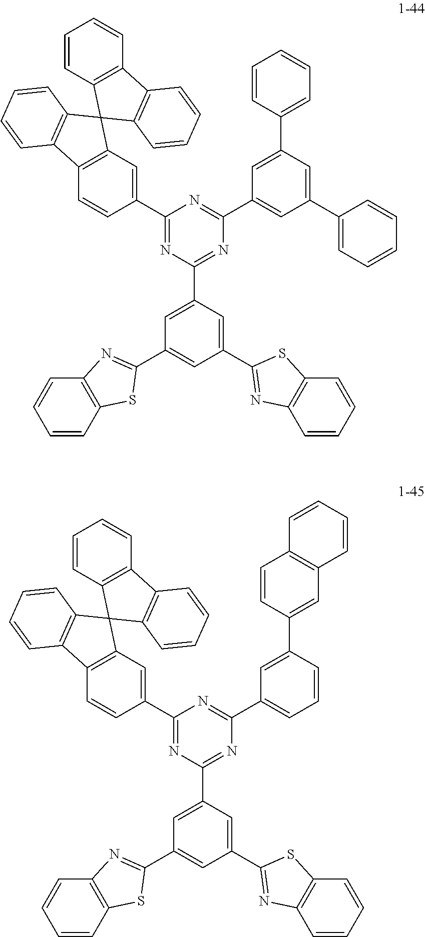 [SMILES: c1ccc(-c2cc(-c3ccccc3)cc(-c3nc(-c4cc(-c5nc6ccccc6s5)cc(-c5nc6ccccc6s5)c4)nc(-c4ccc5c(c4)C4(c6ccccc6-c6ccccc64)c4ccccc4-5)n3)c2)cc1 ; c1cc(-c2ccc3ccccc3c2)cc(-c2nc(-c3cc(-c4nc5ccccc5s4)cc(-c4nc5ccccc5s4)c3)nc(-c3ccc4c(c3)C3(c5ccccc5-c5ccccc53)c3ccccc3-4)n2)c1]

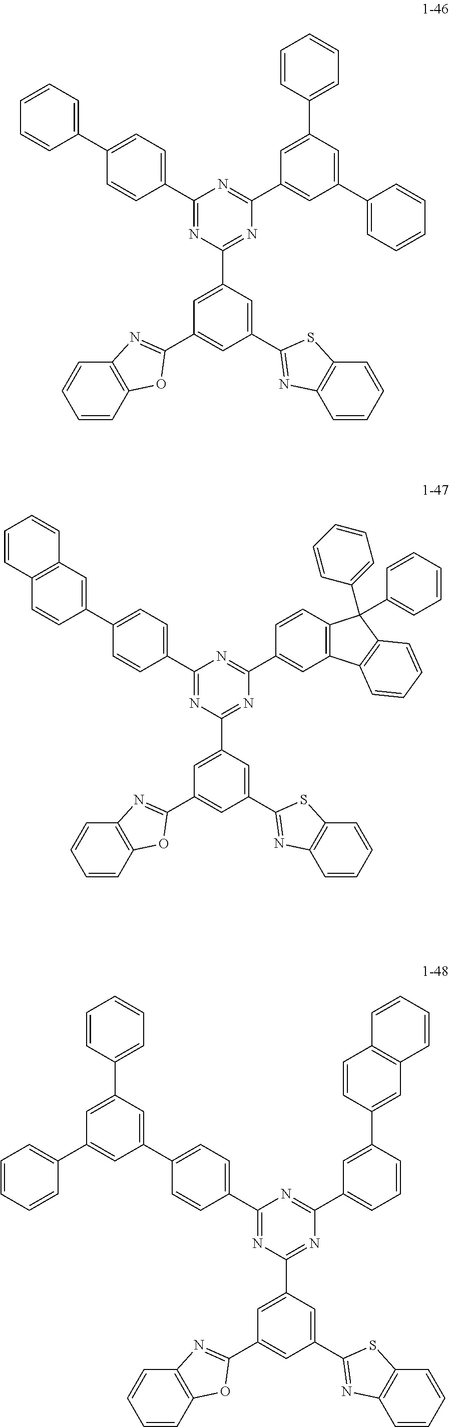 [SMILES: c1ccc(-c2ccc(-c3nc(-c4cc(-c5ccccc5)cc(-c5ccccc5)c4)nc(-c4cc(-c5nc6ccccc6o5)cc(-c5nc6ccccc6s5)c4)n3)cc2)cc1 ; c1ccc(C2(c3ccccc3)c3ccccc3-c3cc(-c4nc(-c5ccc(-c6ccc7ccccc7c6)cc5)nc(-c5cc(-c6nc7ccccc7o6)cc(-c6nc7ccccc7s6)c5)n4)ccc32)cc1 ; c1ccc(-c2cc(-c3ccccc3)cc(-c3ccc(-c4nc(-c5cccc(-c6ccc7ccccc7c6)c5)nc(-c5cc(-c6nc7ccccc7o6)cc(-c6nc7ccccc7s6)c5)n4)cc3)c2)cc1]

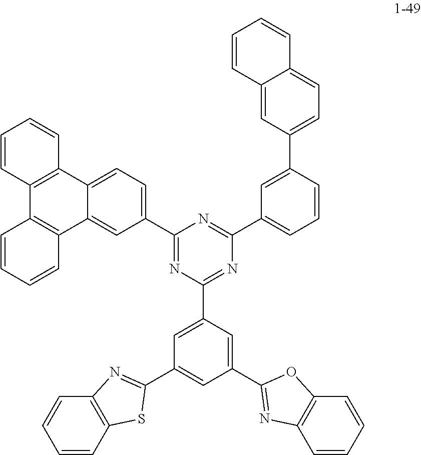 [SMILES: c1cc(-c2ccc3ccccc3c2)cc(-c2nc(-c3cc(-c4nc5ccccc5o4)cc(-c4nc5ccccc5s4)c3)nc(-c3ccc4c5ccccc5c5ccccc5c4c3)n2)c1]

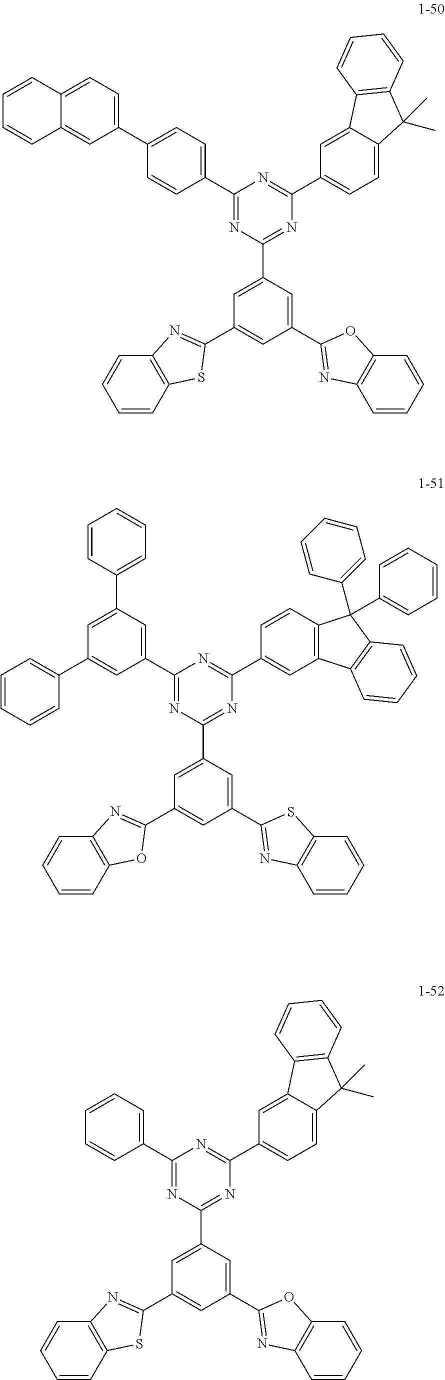 [SMILES: CC1(C)c2ccccc2-c2cc(-c3nc(-c4ccc(-c5ccc6ccccc6c5)cc4)nc(-c4cc(-c5nc6ccccc6o5)cc(-c5nc6ccccc6s5)c4)n3)ccc21 ; c1ccc(-c2cc(-c3ccccc3)cc(-c3nc(-c4cc(-c5nc6ccccc6o5)cc(-c5nc6ccccc6s5)c4)nc(-c4ccc5c(c4)-c4ccccc4C5(c4ccccc4)c4ccccc4)n3)c2)cc1 ; CC1(C)c2ccccc2-c2cc(-c3nc(-c4ccccc4)nc(-c4cc(-c5nc6ccccc6o5)cc(-c5nc6ccccc6s5)c4)n3)ccc21]

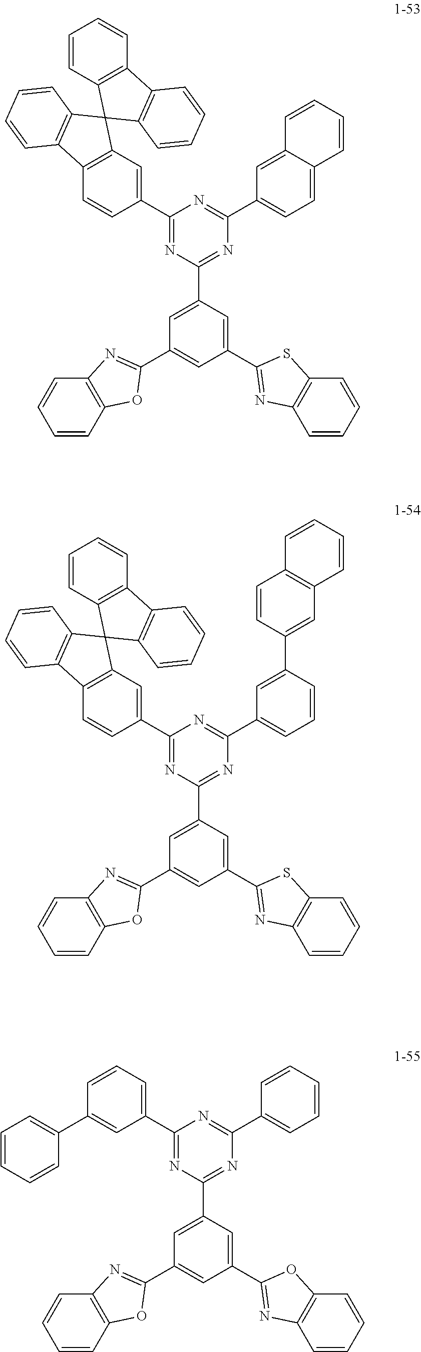 [SMILES: c1ccc2c(c1)-c1ccccc1C21c2ccccc2-c2ccc(-c3nc(-c4cc(-c5nc6ccccc6o5)cc(-c5nc6ccccc6s5)c4)nc(-c4ccc5ccccc5c4)n3)cc21 ; c1cc(-c2ccc3ccccc3c2)cc(-c2nc(-c3cc(-c4nc5ccccc5o4)cc(-c4nc5ccccc5s4)c3)nc(-c3ccc4c(c3)C3(c5ccccc5-c5ccccc53)c3ccccc3-4)n2)c1 ; c1ccc(-c2cccc(-c3nc(-c4ccccc4)nc(-c4cc(-c5nc6ccccc6o5)cc(-c5nc6ccccc6o5)c4)n3)c2)cc1]

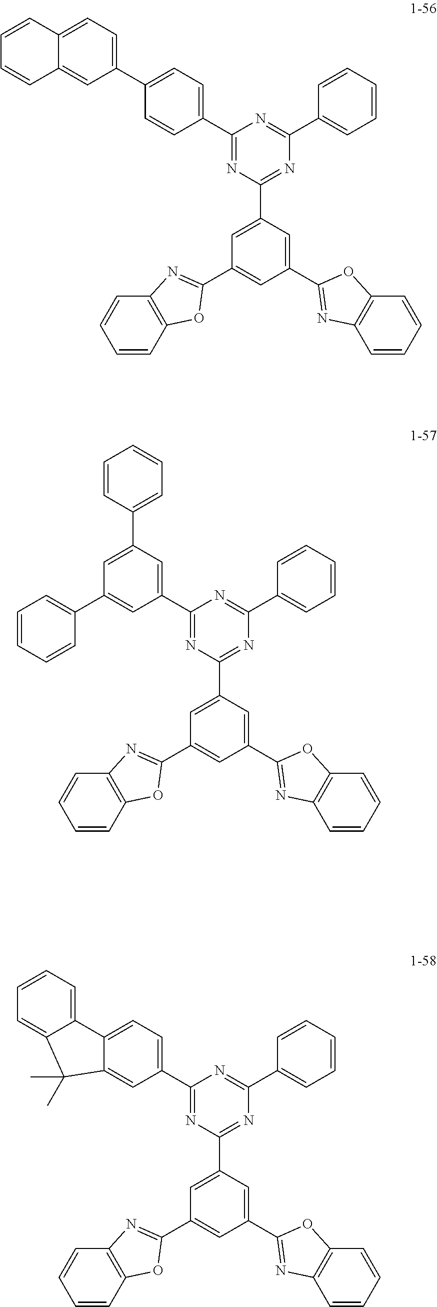 [SMILES: c1ccc(-c2nc(-c3ccc(-c4ccc5ccccc5c4)cc3)nc(-c3cc(-c4nc5ccccc5o4)cc(-c4nc5ccccc5o4)c3)n2)cc1 ; c1ccc(-c2cc(-c3ccccc3)cc(-c3nc(-c4ccccc4)nc(-c4cc(-c5nc6ccccc6o5)cc(-c5nc6ccccc6o5)c4)n3)c2)cc1 ; CC1(C)c2ccccc2-c2ccc(-c3nc(-c4ccccc4)nc(-c4cc(-c5nc6ccccc6o5)cc(-c5nc6ccccc6o5)c4)n3)cc21]

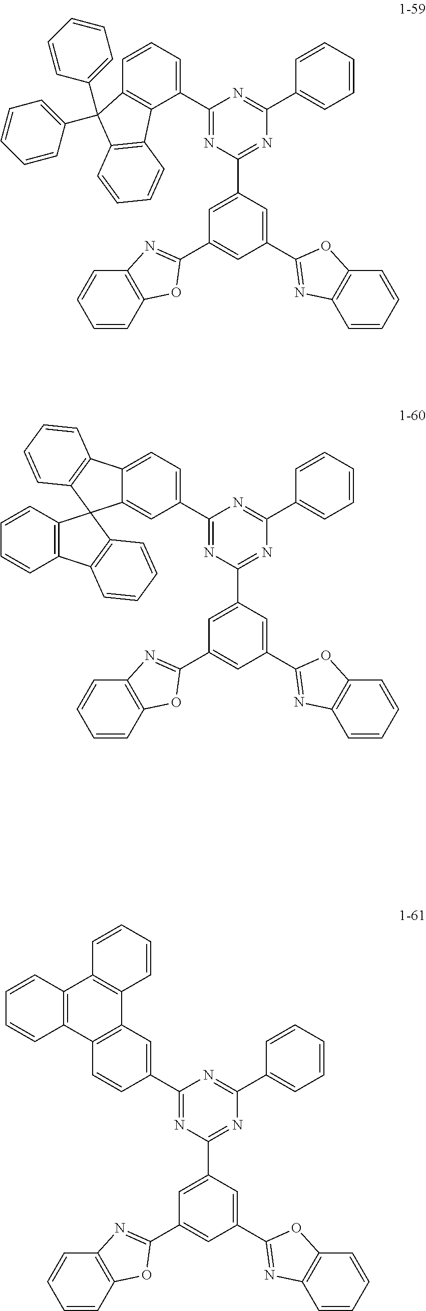 [SMILES: c1ccc(-c2nc(-c3cc(-c4nc5ccccc5o4)cc(-c4nc5ccccc5o4)c3)nc(-c3cccc4c3-c3ccccc3C4(c3ccccc3)c3ccccc3)n2)cc1 ; c1ccc(-c2nc(-c3cc(-c4nc5ccccc5o4)cc(-c4nc5ccccc5o4)c3)nc(-c3ccc4c(c3)C3(c5ccccc5-c5ccccc53)c3ccccc3-4)n2)cc1 ; c1ccc(-c2nc(-c3cc(-c4nc5ccccc5o4)cc(-c4nc5ccccc5o4)c3)nc(-c3ccc4c5ccccc5c5ccccc5c4c3)n2)cc1]

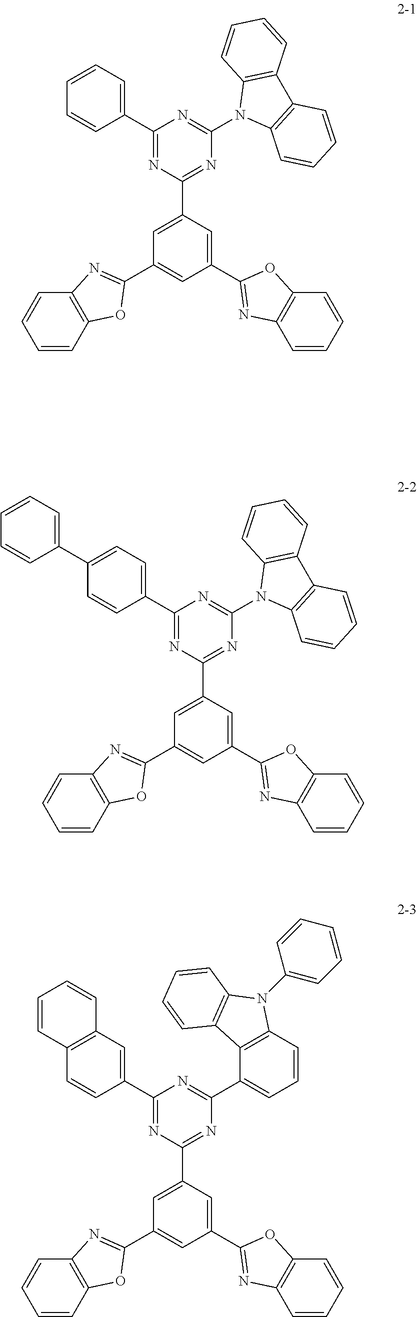 [SMILES: c1ccc(-c2nc(-c3cc(-c4nc5ccccc5o4)cc(-c4nc5ccccc5o4)c3)nc(-n3c4ccccc4c4ccccc43)n2)cc1 ; c1ccc(-c2ccc(-c3nc(-c4cc(-c5nc6ccccc6o5)cc(-c5nc6ccccc6o5)c4)nc(-n4c5ccccc5c5ccccc54)n3)cc2)cc1 ; c1ccc(-n2c3ccccc3c3c(-c4nc(-c5cc(-c6nc7ccccc7o6)cc(-c6nc7ccccc7o6)c5)nc(-c5ccc6ccccc6c5)n4)cccc32)cc1]

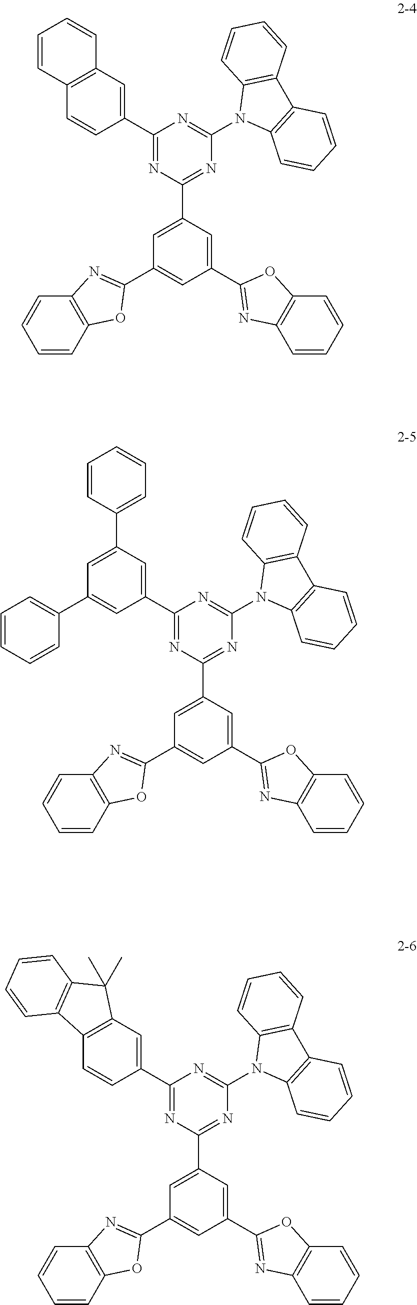 [SMILES: c1ccc2cc(-c3nc(-c4cc(-c5nc6ccccc6o5)cc(-c5nc6ccccc6o5)c4)nc(-n4c5ccccc5c5ccccc54)n3)ccc2c1 ; c1ccc(-c2cc(-c3ccccc3)cc(-c3nc(-c4cc(-c5nc6ccccc6o5)cc(-c5nc6ccccc6o5)c4)nc(-n4c5ccccc5c5ccccc54)n3)c2)cc1 ; CC1(C)c2ccccc2-c2ccc(-c3nc(-c4cc(-c5nc6ccccc6o5)cc(-c5nc6ccccc6o5)c4)nc(-n4c5ccccc5c5ccccc54)n3)cc21]

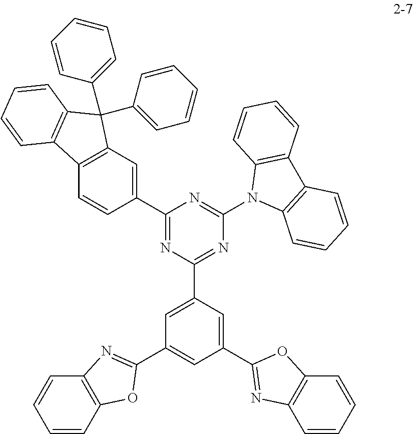 [SMILES: c1ccc(C2(c3ccccc3)c3ccccc3-c3ccc(-c4nc(-c5cc(-c6nc7ccccc7o6)cc(-c6nc7ccccc7o6)c5)nc(-n5c6ccccc6c6ccccc65)n4)cc32)cc1]

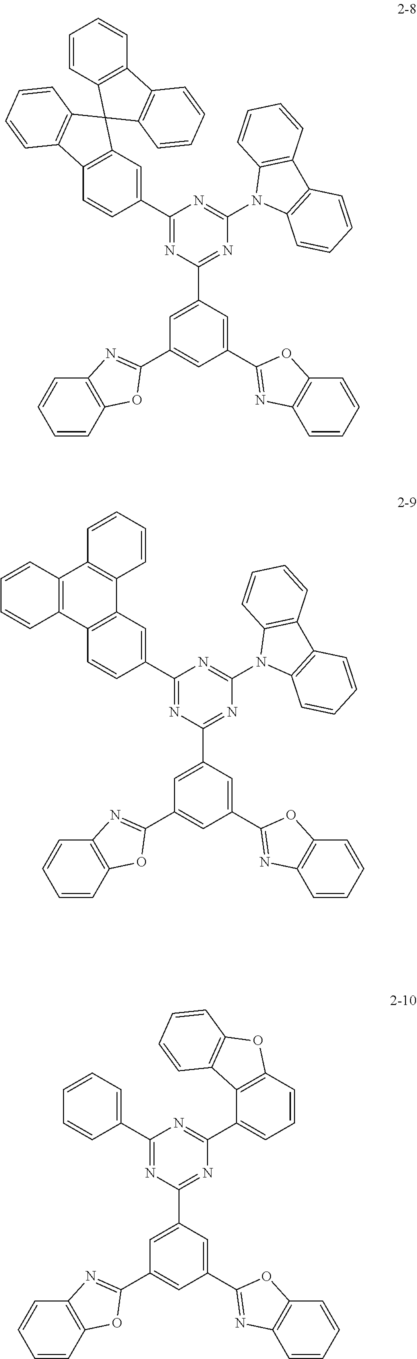 [SMILES: c1ccc2c(c1)-c1ccccc1C21c2ccccc2-c2ccc(-c3nc(-c4cc(-c5nc6ccccc6o5)cc(-c5nc6ccccc6o5)c4)nc(-n4c5ccccc5c5ccccc54)n3)cc21 ; c1ccc2oc(-c3cc(-c4nc(-c5ccc6c7ccccc7c7ccccc7c6c5)nc(-n5c6ccccc6c6ccccc65)n4)cc(-c4nc5ccccc5o4)c3)nc2c1 ; c1ccc(-c2nc(-c3cc(-c4nc5ccccc5o4)cc(-c4nc5ccccc5o4)c3)nc(-c3cccc4oc5ccccc5c34)n2)cc1]

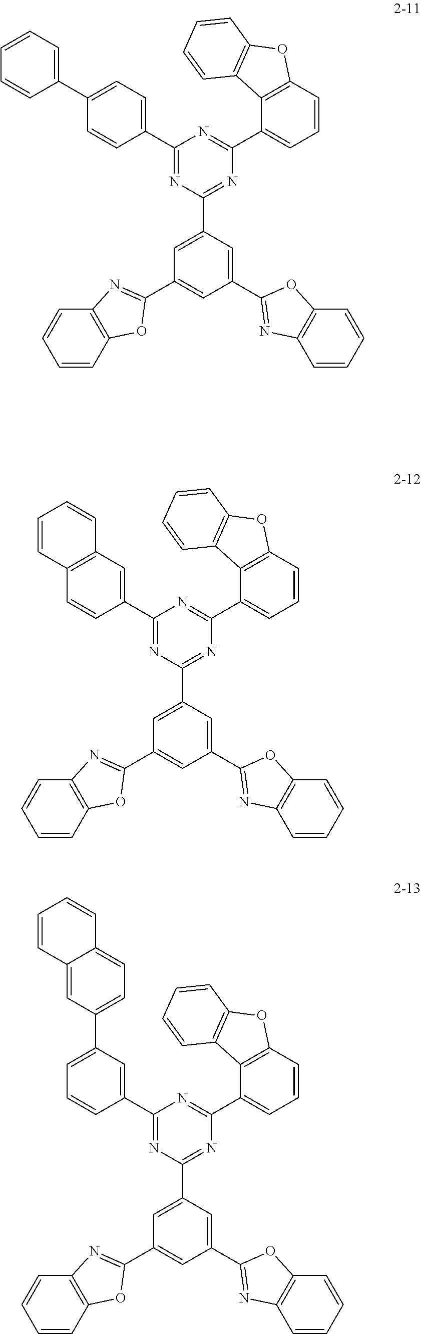 [SMILES: c1ccc(-c2ccc(-c3nc(-c4cc(-c5nc6ccccc6o5)cc(-c5nc6ccccc6o5)c4)nc(-c4cccc5oc6ccccc6c45)n3)cc2)cc1 ; c1ccc2cc(-c3nc(-c4cc(-c5nc6ccccc6o5)cc(-c5nc6ccccc6o5)c4)nc(-c4cccc5oc6ccccc6c45)n3)ccc2c1 ; c1cc(-c2ccc3ccccc3c2)cc(-c2nc(-c3cc(-c4nc5ccccc5o4)cc(-c4nc5ccccc5o4)c3)nc(-c3cccc4oc5ccccc5c34)n2)c1]

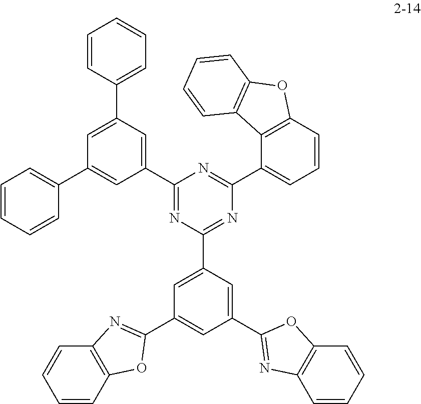 [SMILES: c1ccc(-c2cc(-c3ccccc3)cc(-c3nc(-c4cc(-c5nc6ccccc6o5)cc(-c5nc6ccccc6o5)c4)nc(-c4cccc5oc6ccccc6c45)n3)c2)cc1]

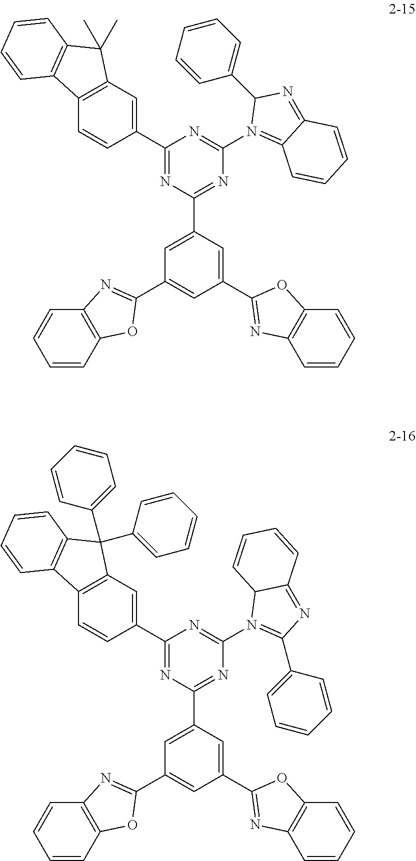

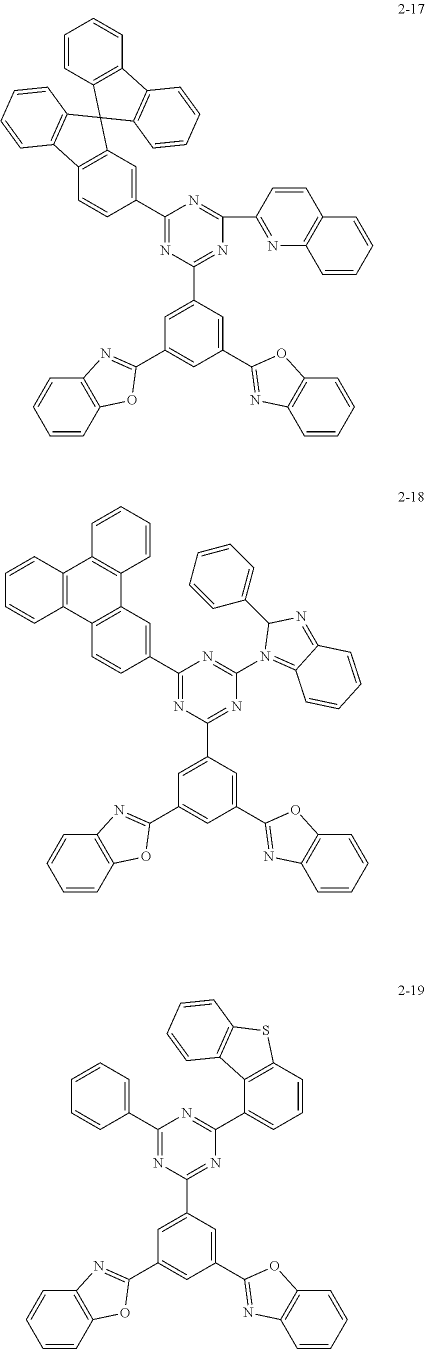

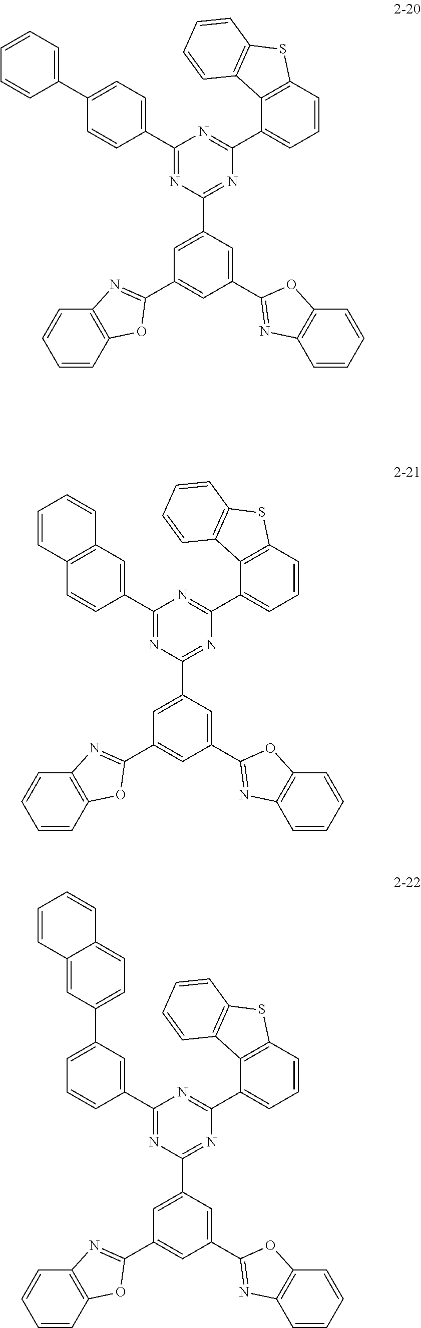 [SMILES: c1ccc(-c2ccc(-c3nc(-c4cc(-c5nc6ccccc6o5)cc(-c5nc6ccccc6o5)c4)nc(-c4cccc5sc6ccccc6c45)n3)cc2)cc1 ; c1ccc2cc(-c3nc(-c4cc(-c5nc6ccccc6o5)cc(-c5nc6ccccc6o5)c4)nc(-c4cccc5sc6ccccc6c45)n3)ccc2c1 ; c1cc(-c2ccc3ccccc3c2)cc(-c2nc(-c3cc(-c4nc5ccccc5o4)cc(-c4nc5ccccc5o4)c3)nc(-c3cccc4sc5ccccc5c34)n2)c1]

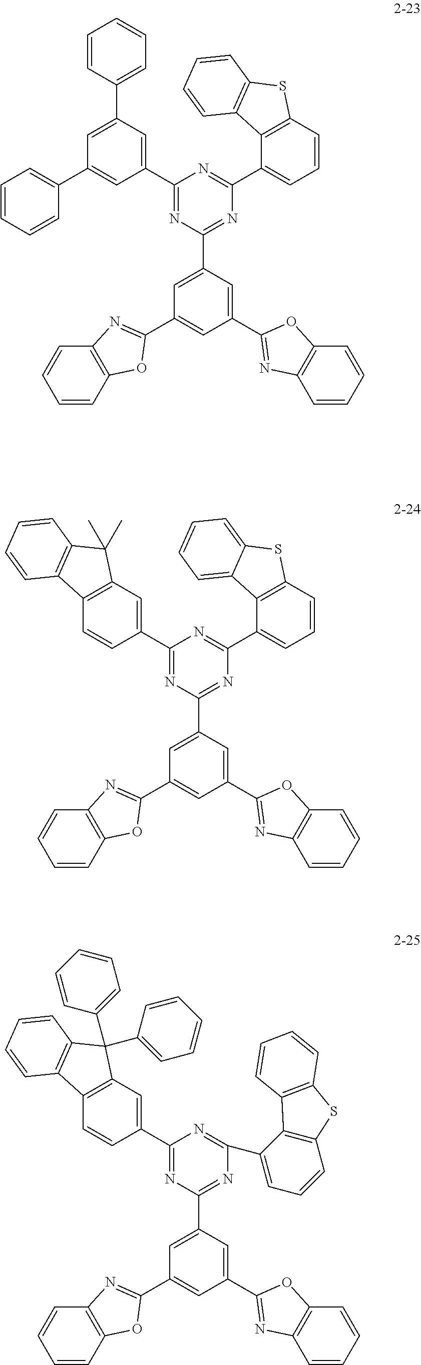 [SMILES: c1ccc(-c2cc(-c3ccccc3)cc(-c3nc(-c4cc(-c5nc6ccccc6o5)cc(-c5nc6ccccc6o5)c4)nc(-c4cccc5sc6ccccc6c45)n3)c2)cc1 ; CC1(C)c2ccccc2-c2ccc(-c3nc(-c4cc(-c5nc6ccccc6o5)cc(-c5nc6ccccc6o5)c4)nc(-c4cccc5sc6ccccc6c45)n3)cc21 ; c1ccc(C2(c3ccccc3)c3ccccc3-c3ccc(-c4nc(-c5cc(-c6nc7ccccc7o6)cc(-c6nc7ccccc7o6)c5)nc(-c5cccc6sc7ccccc7c56)n4)cc32)cc1]

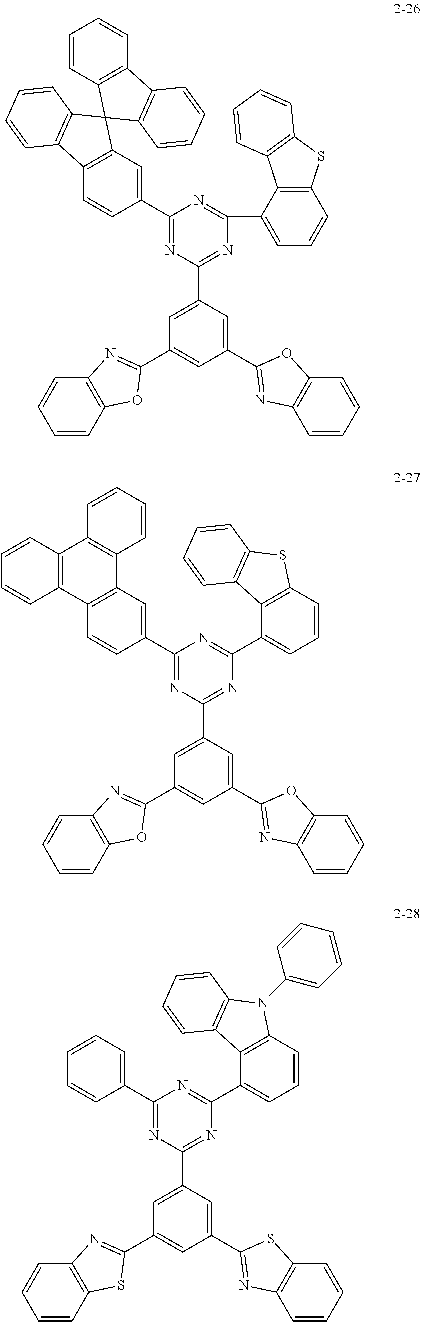 [SMILES: c1ccc2c(c1)-c1ccccc1C21c2ccccc2-c2ccc(-c3nc(-c4cc(-c5nc6ccccc6o5)cc(-c5nc6ccccc6o5)c4)nc(-c4cccc5sc6ccccc6c45)n3)cc21 ; c1ccc2oc(-c3cc(-c4nc(-c5ccc6c7ccccc7c7ccccc7c6c5)nc(-c5cccc6sc7ccccc7c56)n4)cc(-c4nc5ccccc5o4)c3)nc2c1 ; c1ccc(-c2nc(-c3cc(-c4nc5ccccc5s4)cc(-c4nc5ccccc5s4)c3)nc(-c3cccc4c3c3ccccc3n4-c3ccccc3)n2)cc1]

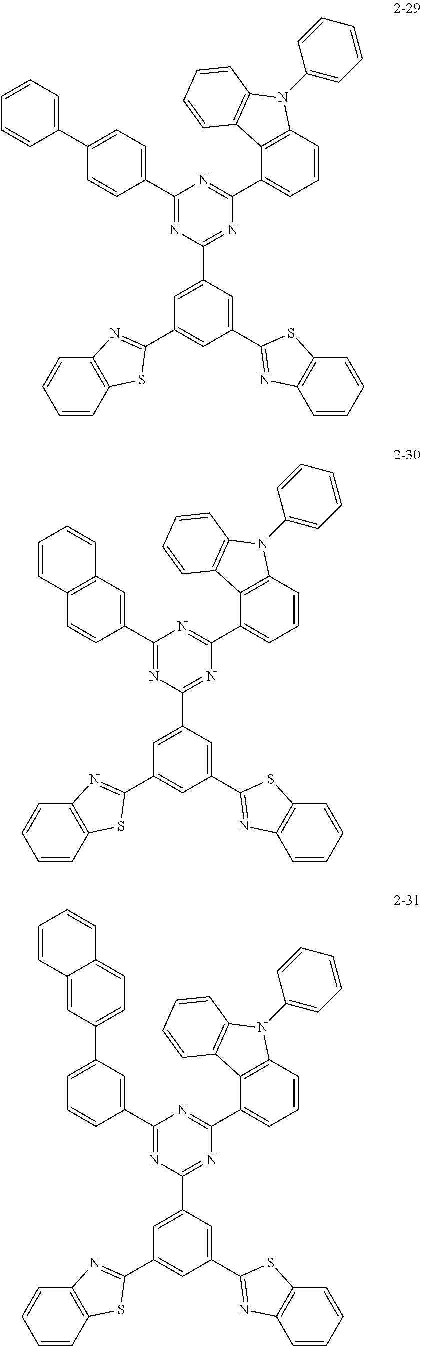 [SMILES: c1ccc(-c2ccc(-c3nc(-c4cc(-c5nc6ccccc6s5)cc(-c5nc6ccccc6s5)c4)nc(-c4cccc5c4c4ccccc4n5-c4ccccc4)n3)cc2)cc1 ; c1ccc(-n2c3ccccc3c3c(-c4nc(-c5cc(-c6nc7ccccc7s6)cc(-c6nc7ccccc7s6)c5)nc(-c5ccc6ccccc6c5)n4)cccc32)cc1 ; c1ccc(-n2c3ccccc3c3c(-c4nc(-c5cccc(-c6ccc7ccccc7c6)c5)nc(-c5cc(-c6nc7ccccc7s6)cc(-c6nc7ccccc7s6)c5)n4)cccc32)cc1]

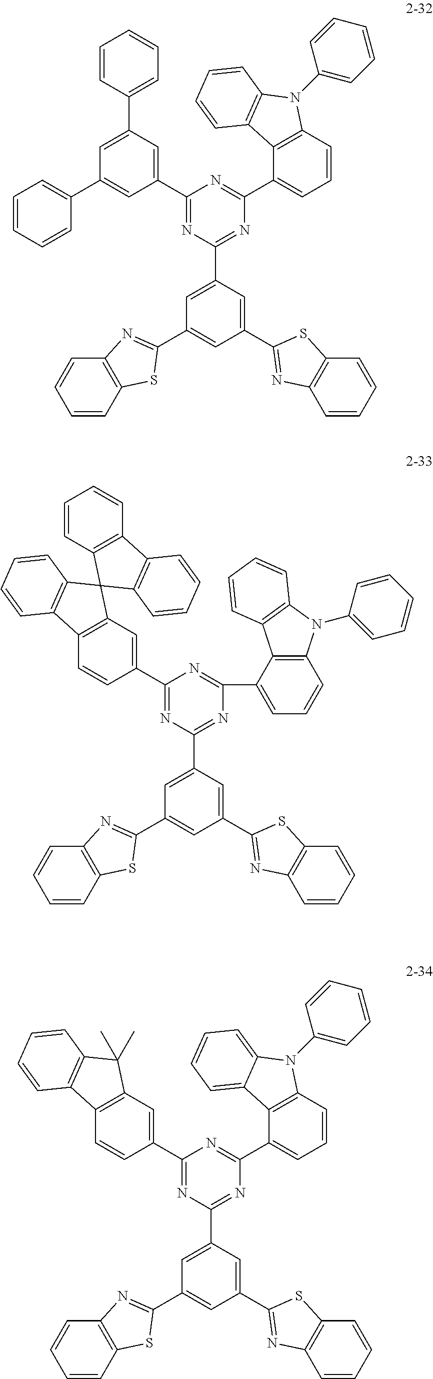 [SMILES: c1ccc(-c2cc(-c3ccccc3)cc(-c3nc(-c4cc(-c5nc6ccccc6s5)cc(-c5nc6ccccc6s5)c4)nc(-c4cccc5c4c4ccccc4n5-c4ccccc4)n3)c2)cc1 ; c1ccc(-n2c3ccccc3c3c(-c4nc(-c5cc(-c6nc7ccccc7s6)cc(-c6nc7ccccc7s6)c5)nc(-c5ccc6c(c5)C5(c7ccccc7-c7ccccc75)c5ccccc5-6)n4)cccc32)cc1 ; CC1(C)c2ccccc2-c2ccc(-c3nc(-c4cc(-c5nc6ccccc6s5)cc(-c5nc6ccccc6s5)c4)nc(-c4cccc5c4c4ccccc4n5-c4ccccc4)n3)cc21]

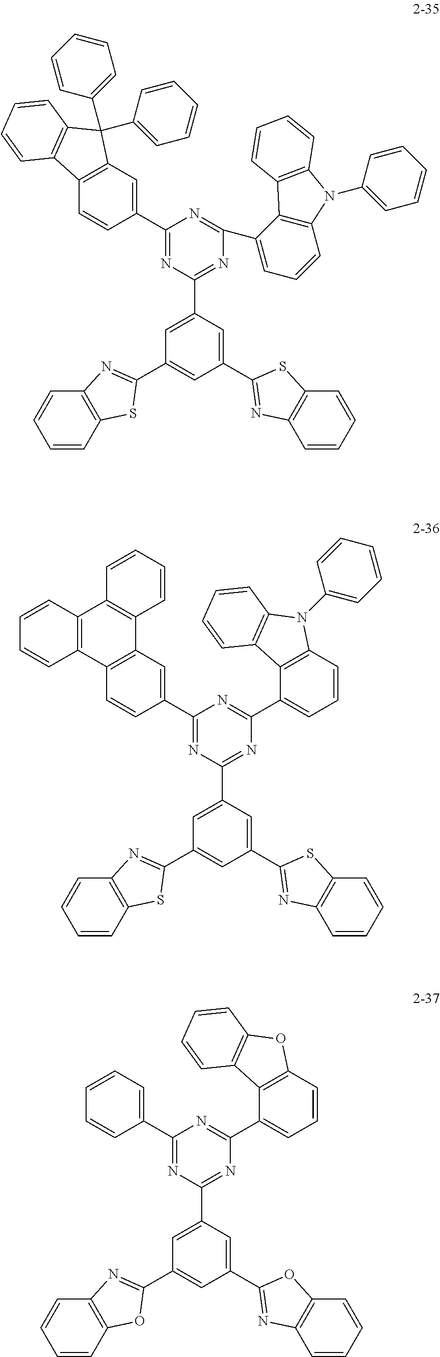 [SMILES: c1ccc(-n2c3ccccc3c3c(-c4nc(-c5cc(-c6nc7ccccc7s6)cc(-c6nc7ccccc7s6)c5)nc(-c5ccc6c(c5)C(c5ccccc5)(c5ccccc5)c5ccccc5-6)n4)cccc32)cc1 ; c1ccc(-n2c3ccccc3c3c(-c4nc(-c5cc(-c6nc7ccccc7s6)cc(-c6nc7ccccc7s6)c5)nc(-c5ccc6c7ccccc7c7ccccc7c6c5)n4)cccc32)cc1 ; c1ccc(-c2nc(-c3cc(-c4nc5ccccc5o4)cc(-c4nc5ccccc5o4)c3)nc(-c3cccc4oc5ccccc5c34)n2)cc1]

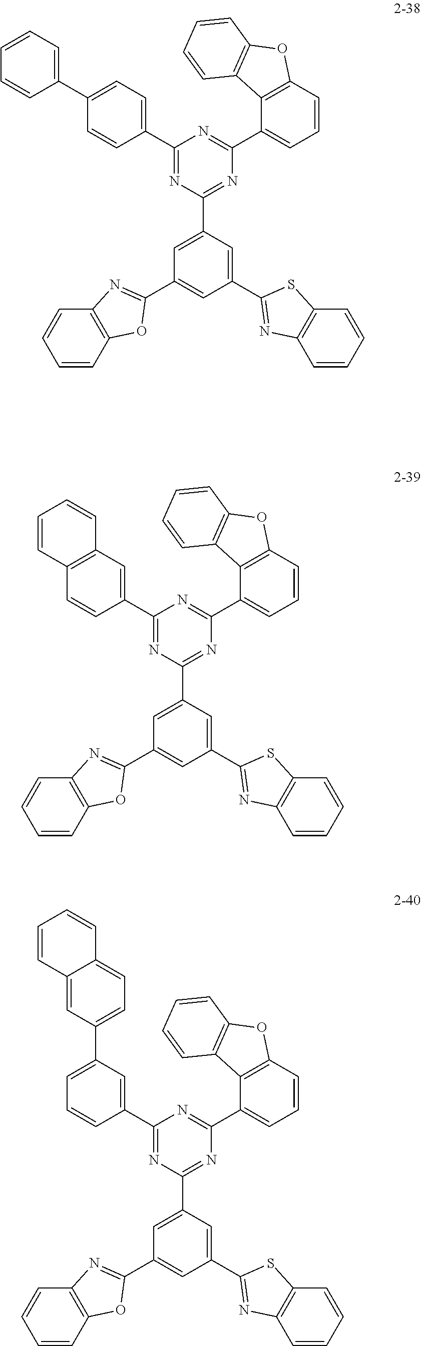 [SMILES: c1ccc(-c2ccc(-c3nc(-c4cc(-c5nc6ccccc6o5)cc(-c5nc6ccccc6s5)c4)nc(-c4cccc5oc6ccccc6c45)n3)cc2)cc1 ; c1ccc2cc(-c3nc(-c4cc(-c5nc6ccccc6o5)cc(-c5nc6ccccc6s5)c4)nc(-c4cccc5oc6ccccc6c45)n3)ccc2c1 ; c1cc(-c2ccc3ccccc3c2)cc(-c2nc(-c3cc(-c4nc5ccccc5o4)cc(-c4nc5ccccc5s4)c3)nc(-c3cccc4oc5ccccc5c34)n2)c1]

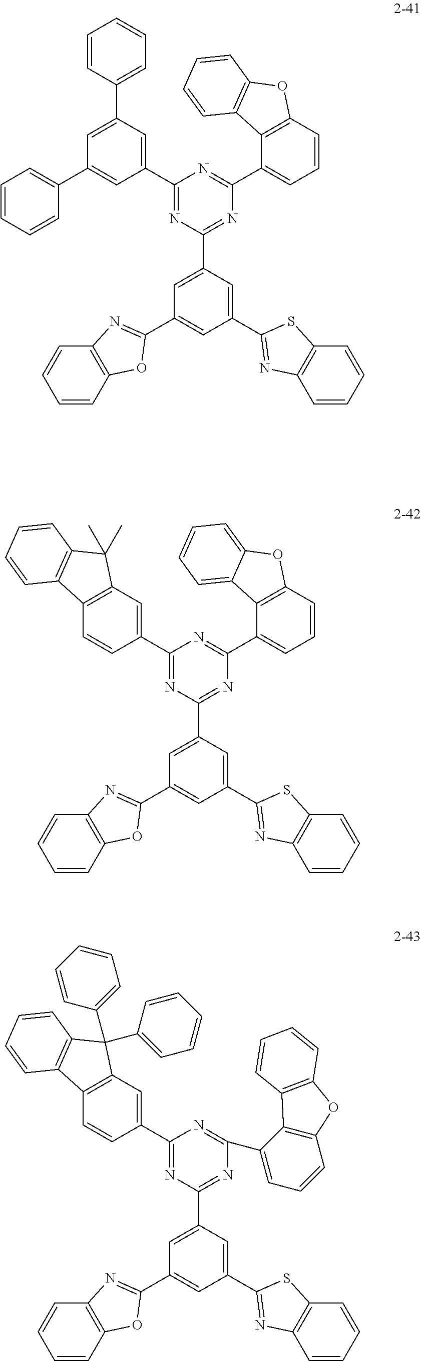 [SMILES: c1ccc(-c2cc(-c3ccccc3)cc(-c3nc(-c4cc(-c5nc6ccccc6o5)cc(-c5nc6ccccc6s5)c4)nc(-c4cccc5oc6ccccc6c45)n3)c2)cc1 ; CC1(C)c2ccccc2-c2ccc(-c3nc(-c4cc(-c5nc6ccccc6o5)cc(-c5nc6ccccc6s5)c4)nc(-c4cccc5oc6ccccc6c45)n3)cc21 ; c1ccc(C2(c3ccccc3)c3ccccc3-c3ccc(-c4nc(-c5cc(-c6nc7ccccc7o6)cc(-c6nc7ccccc7s6)c5)nc(-c5cccc6oc7ccccc7c56)n4)cc32)cc1]

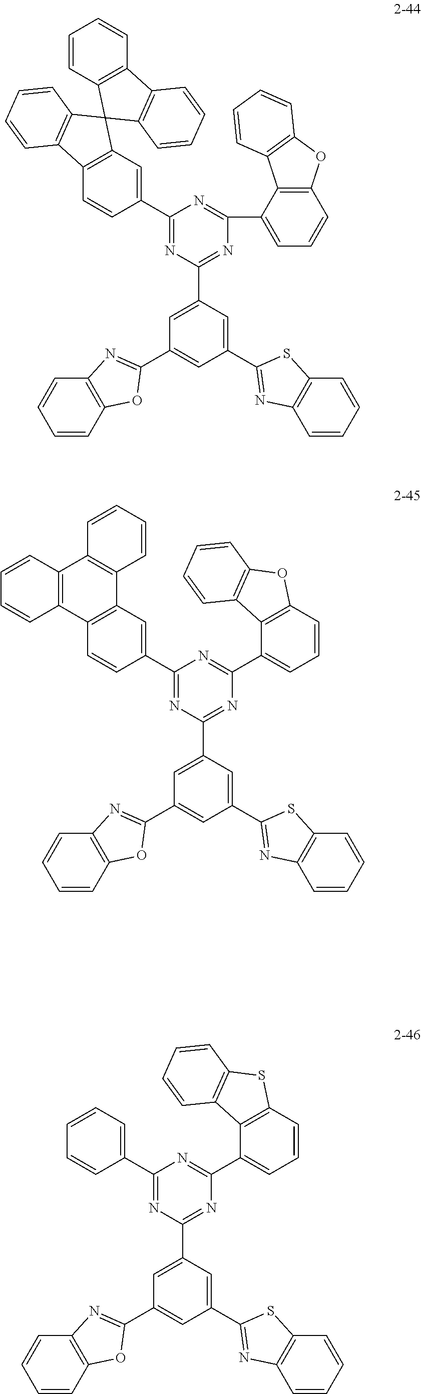 [SMILES: c1ccc2c(c1)-c1ccccc1C21c2ccccc2-c2ccc(-c3nc(-c4cc(-c5nc6ccccc6o5)cc(-c5nc6ccccc6s5)c4)nc(-c4cccc5oc6ccccc6c45)n3)cc21 ; c1ccc2oc(-c3cc(-c4nc(-c5ccc6c7ccccc7c7ccccc7c6c5)nc(-c5cccc6oc7ccccc7c56)n4)cc(-c4nc5ccccc5s4)c3)nc2c1 ; c1ccc(-c2nc(-c3cc(-c4nc5ccccc5o4)cc(-c4nc5ccccc5s4)c3)nc(-c3cccc4sc5ccccc5c34)n2)cc1]

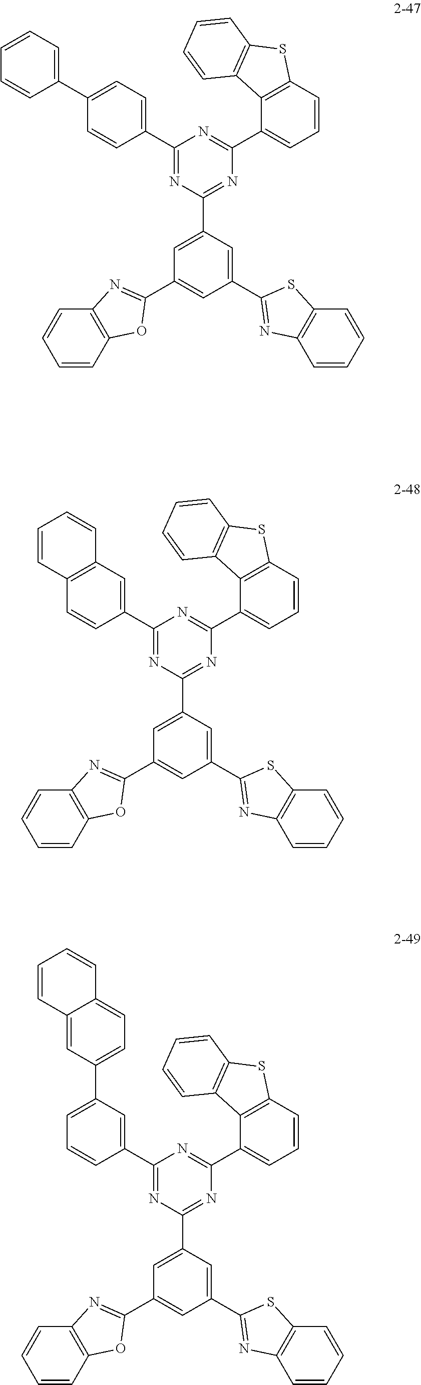 [SMILES: c1ccc(-c2ccc(-c3nc(-c4cc(-c5nc6ccccc6o5)cc(-c5nc6ccccc6s5)c4)nc(-c4cccc5sc6ccccc6c45)n3)cc2)cc1 ; c1ccc2cc(-c3nc(-c4cc(-c5nc6ccccc6o5)cc(-c5nc6ccccc6s5)c4)nc(-c4cccc5sc6ccccc6c45)n3)ccc2c1 ; c1cc(-c2ccc3ccccc3c2)cc(-c2nc(-c3cc(-c4nc5ccccc5o4)cc(-c4nc5ccccc5s4)c3)nc(-c3cccc4sc5ccccc5c34)n2)c1]

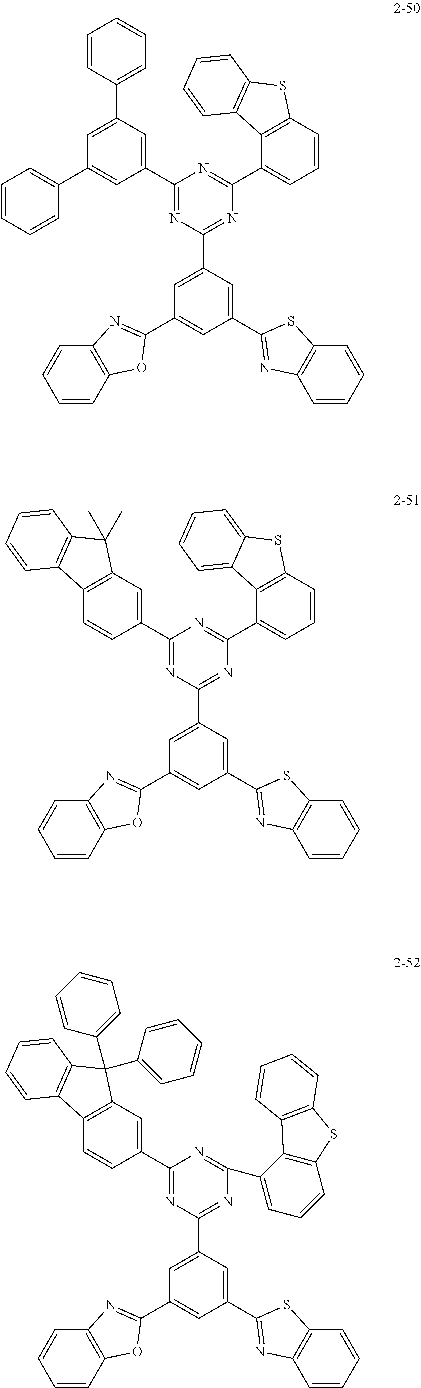 [SMILES: c1ccc(-c2cc(-c3ccccc3)cc(-c3nc(-c4cc(-c5nc6ccccc6o5)cc(-c5nc6ccccc6s5)c4)nc(-c4cccc5sc6ccccc6c45)n3)c2)cc1 ; CC1(C)c2ccccc2-c2ccc(-c3nc(-c4cc(-c5nc6ccccc6o5)cc(-c5nc6ccccc6s5)c4)nc(-c4cccc5sc6ccccc6c45)n3)cc21 ; c1ccc(C2(c3ccccc3)c3ccccc3-c3ccc(-c4nc(-c5cc(-c6nc7ccccc7o6)cc(-c6nc7ccccc7s6)c5)nc(-c5cccc6sc7ccccc7c56)n4)cc32)cc1]

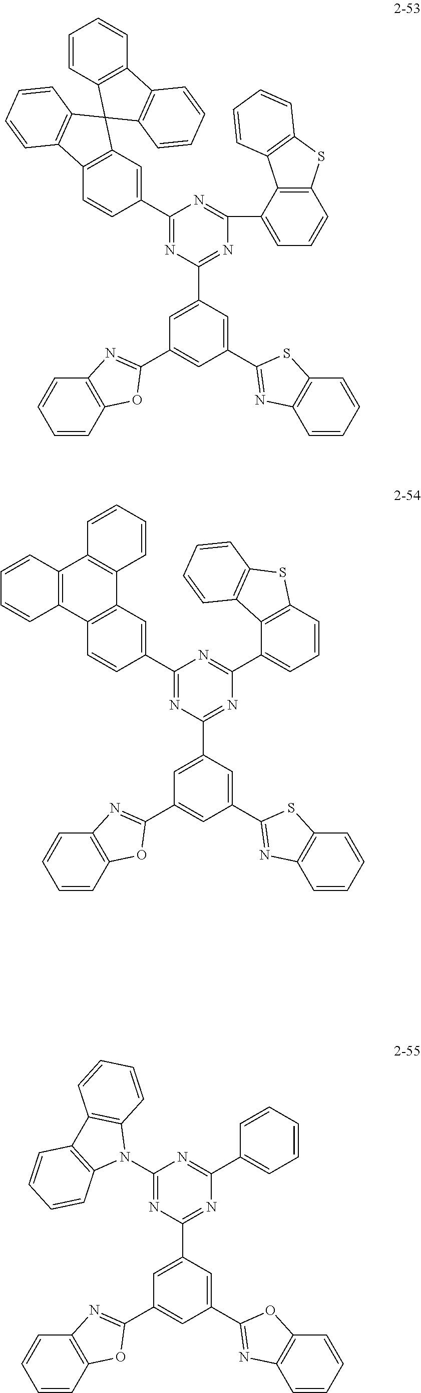 [SMILES: c1ccc2c(c1)-c1ccccc1C21c2ccccc2-c2ccc(-c3nc(-c4cc(-c5nc6ccccc6o5)cc(-c5nc6ccccc6s5)c4)nc(-c4cccc5sc6ccccc6c45)n3)cc21 ; c1ccc2oc(-c3cc(-c4nc(-c5ccc6c7ccccc7c7ccccc7c6c5)nc(-c5cccc6sc7ccccc7c56)n4)cc(-c4nc5ccccc5s4)c3)nc2c1 ; c1ccc(-c2nc(-c3cc(-c4nc5ccccc5o4)cc(-c4nc5ccccc5o4)c3)nc(-n3c4ccccc4c4ccccc43)n2)cc1]

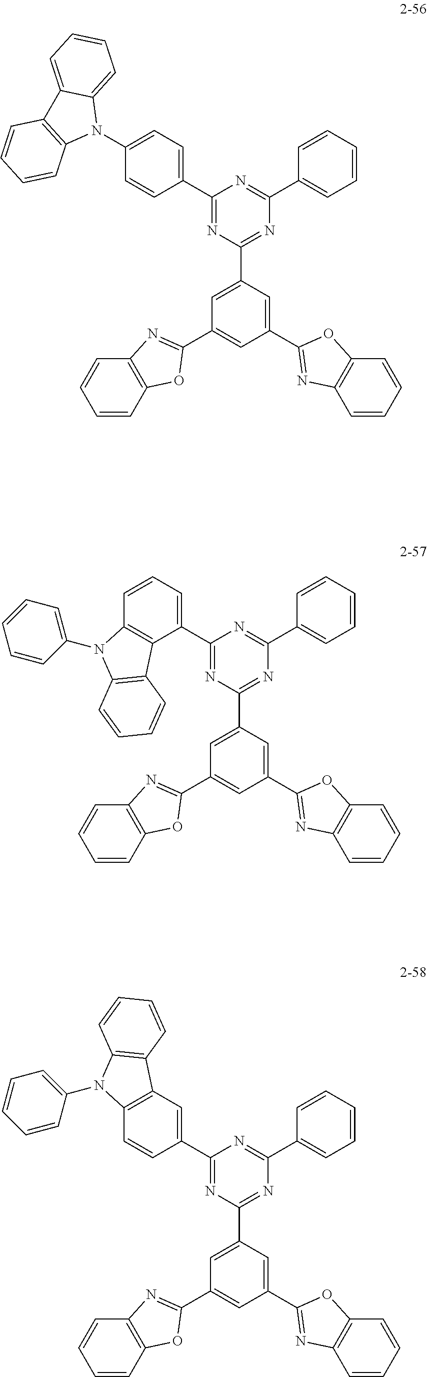 [SMILES: c1ccc(-c2nc(-c3ccc(-n4c5ccccc5c5ccccc54)cc3)nc(-c3cc(-c4nc5ccccc5o4)cc(-c4nc5ccccc5o4)c3)n2)cc1 ; c1ccc(-c2nc(-c3cc(-c4nc5ccccc5o4)cc(-c4nc5ccccc5o4)c3)nc(-c3cccc4c3c3ccccc3n4-c3ccccc3)n2)cc1 ; c1ccc(-c2nc(-c3cc(-c4nc5ccccc5o4)cc(-c4nc5ccccc5o4)c3)nc(-c3ccc4c(c3)c3ccccc3n4-c3ccccc3)n2)cc1]

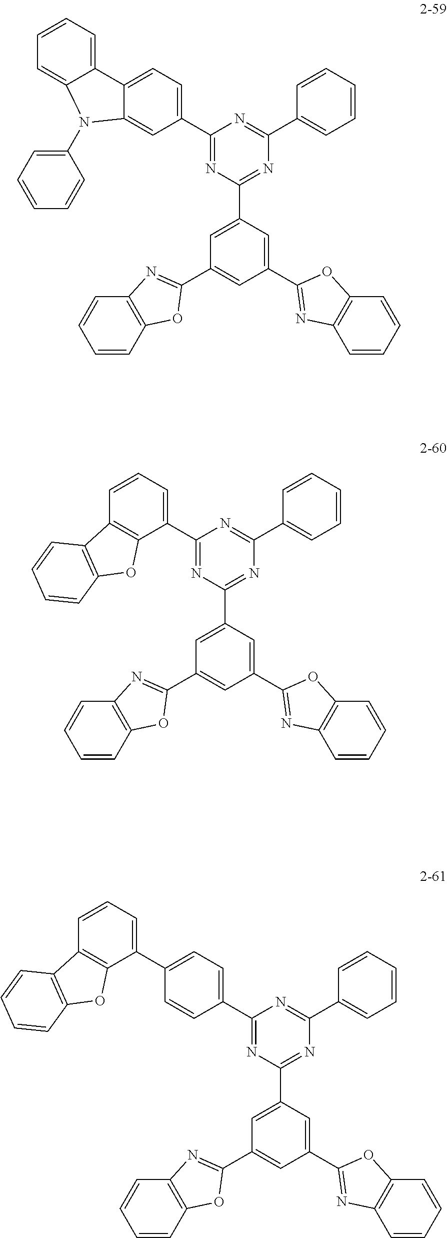 [SMILES: c1ccc(-c2nc(-c3cc(-c4nc5ccccc5o4)cc(-c4nc5ccccc5o4)c3)nc(-c3ccc4c5ccccc5n(-c5ccccc5)c4c3)n2)cc1 ; c1ccc(-c2nc(-c3cc(-c4nc5ccccc5o4)cc(-c4nc5ccccc5o4)c3)nc(-c3cccc4c3oc3ccccc34)n2)cc1 ; c1ccc(-c2nc(-c3ccc(-c4cccc5c4oc4ccccc45)cc3)nc(-c3cc(-c4nc5ccccc5o4)cc(-c4nc5ccccc5o4)c3)n2)cc1]

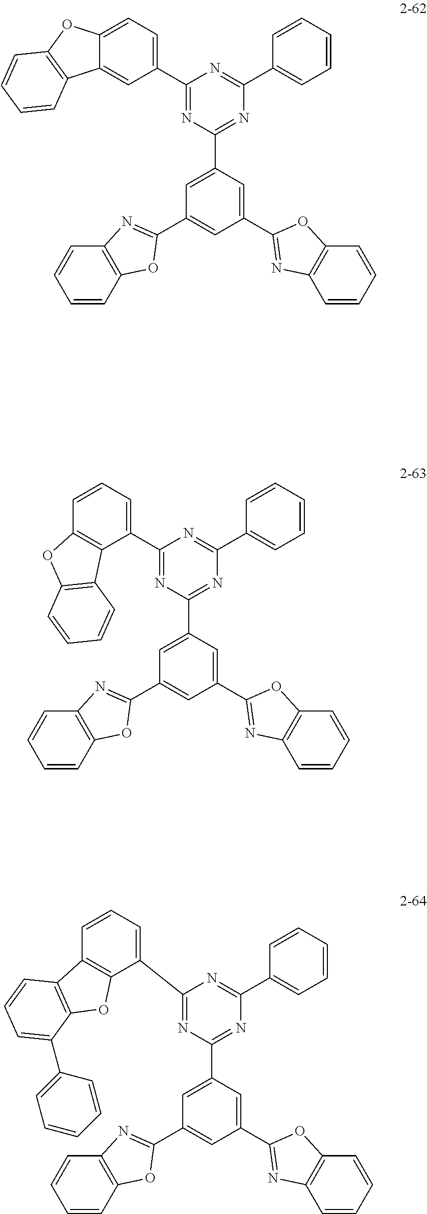 [SMILES: c1ccc(-c2nc(-c3cc(-c4nc5ccccc5o4)cc(-c4nc5ccccc5o4)c3)nc(-c3ccc4oc5ccccc5c4c3)n2)cc1 ; c1ccc(-c2nc(-c3cc(-c4nc5ccccc5o4)cc(-c4nc5ccccc5o4)c3)nc(-c3cccc4oc5ccccc5c34)n2)cc1 ; c1ccc(-c2nc(-c3cc(-c4nc5ccccc5o4)cc(-c4nc5ccccc5o4)c3)nc(-c3cccc4c3oc3c(-c5ccccc5)cccc34)n2)cc1]

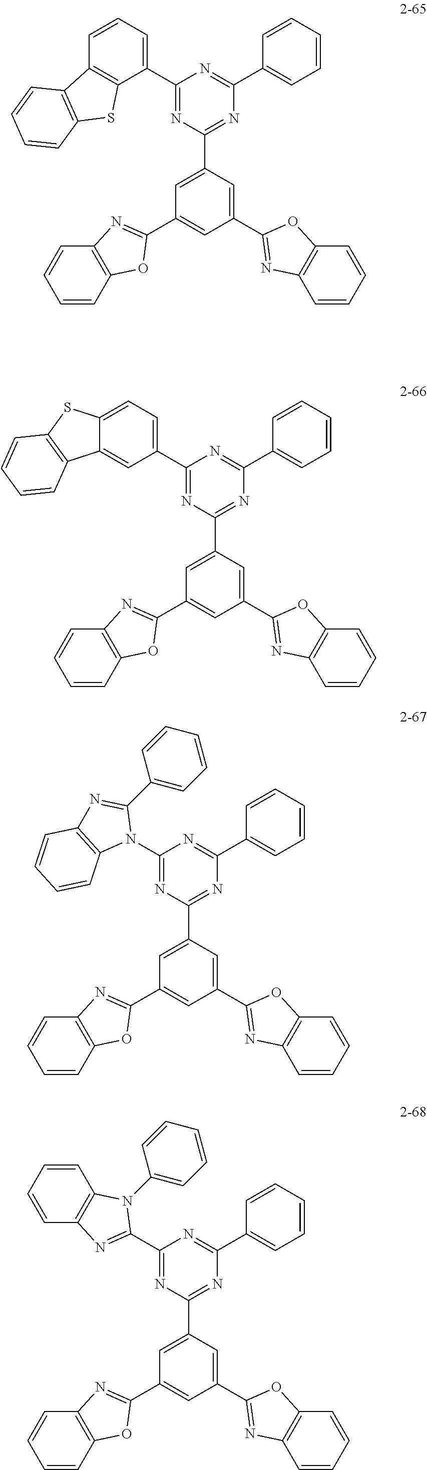 [SMILES: c1ccc(-c2nc(-c3cc(-c4nc5ccccc5o4)cc(-c4nc5ccccc5o4)c3)nc(-c3cccc4c3sc3ccccc34)n2)cc1 ; c1ccc(-c2nc(-c3cc(-c4nc5ccccc5o4)cc(-c4nc5ccccc5o4)c3)nc(-c3ccc4sc5ccccc5c4c3)n2)cc1 ; c1ccc(-c2nc(-c3cc(-c4nc5ccccc5o4)cc(-c4nc5ccccc5o4)c3)nc(-n3c(-c4ccccc4)nc4ccccc43)n2)cc1 ; c1ccc(-c2nc(-c3cc(-c4nc5ccccc5o4)cc(-c4nc5ccccc5o4)c3)nc(-c3nc4ccccc4n3-c3ccccc3)n2)cc1]

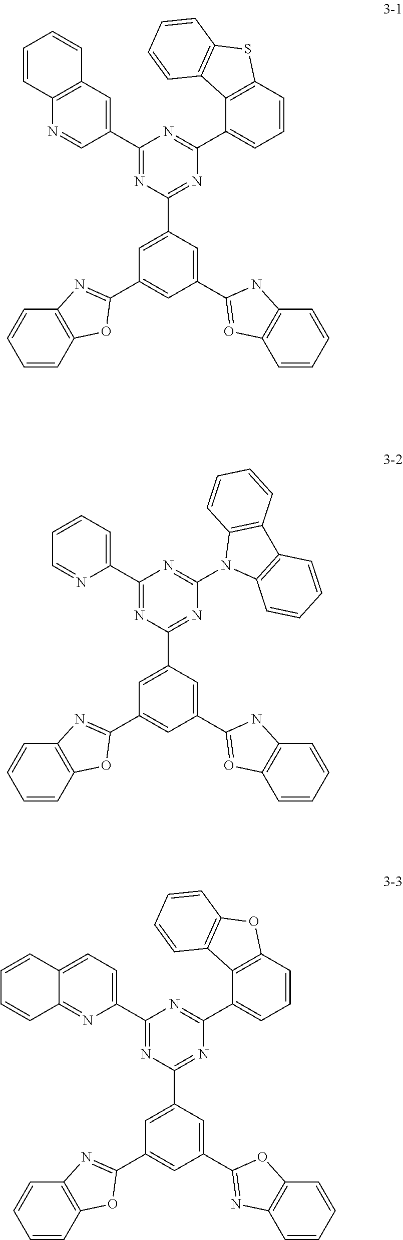

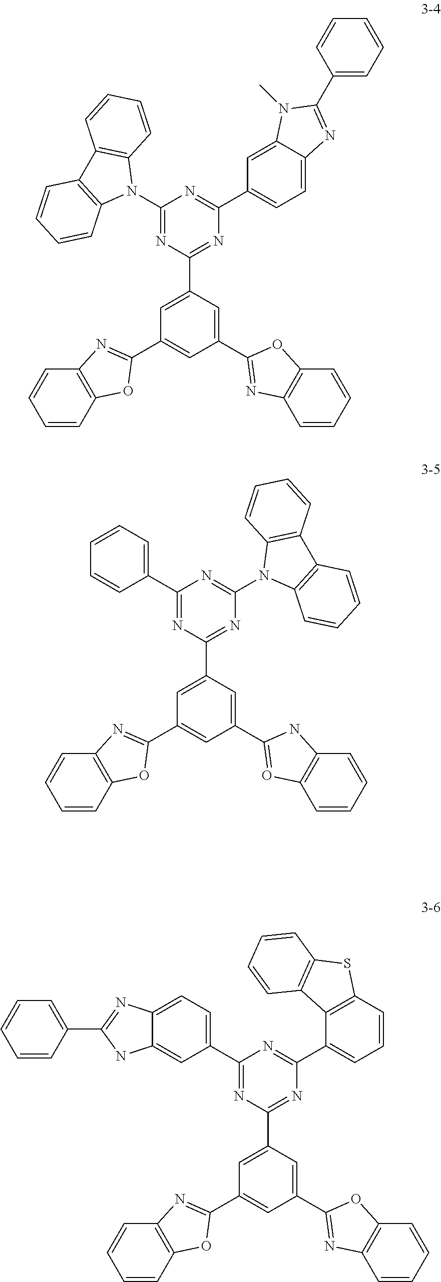

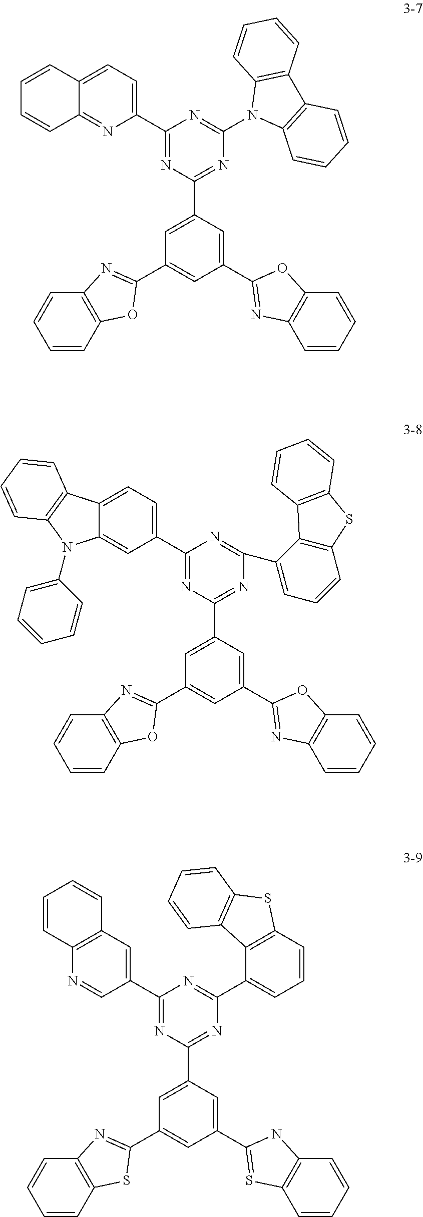

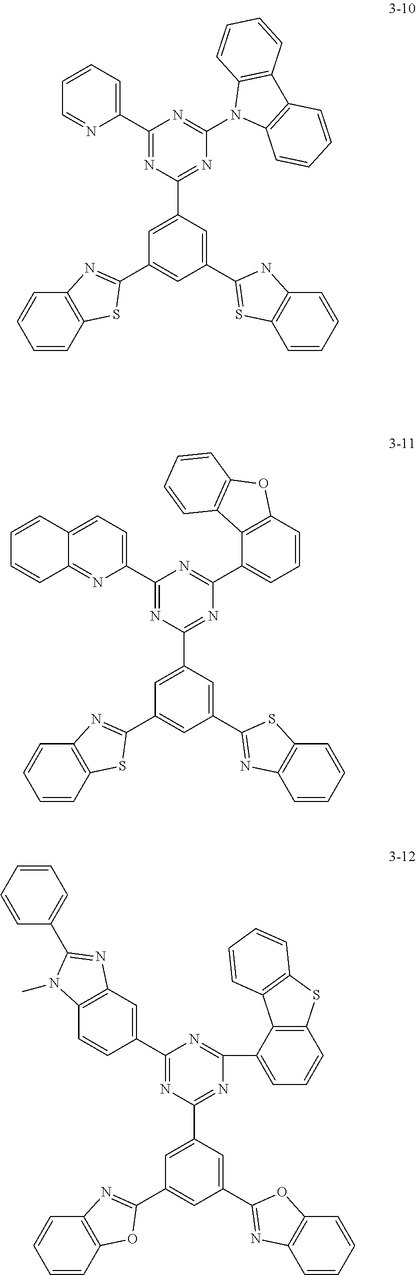

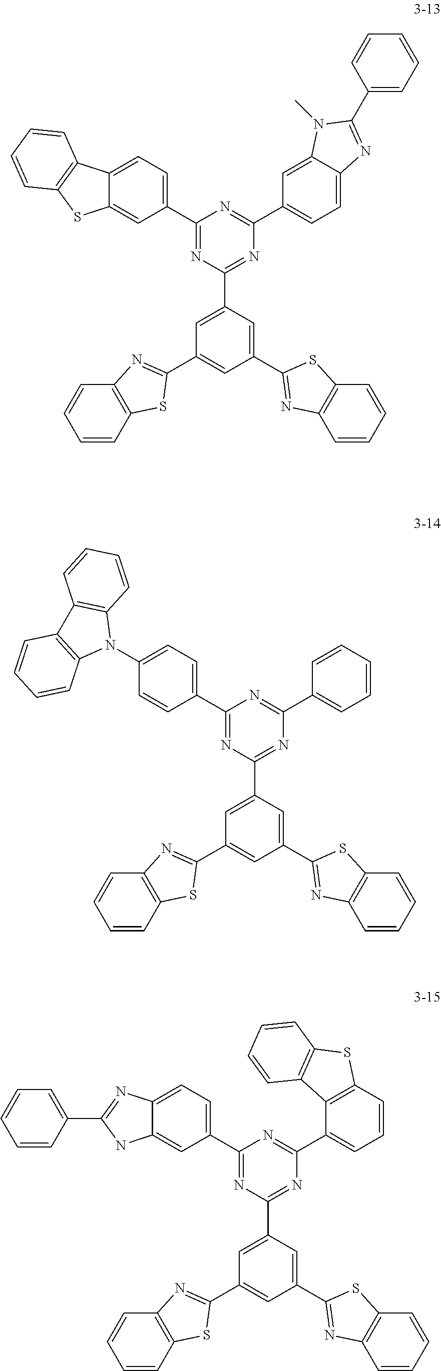 [SMILES: Cn1c(-c2ccccc2)nc2ccc(-c3nc(-c4cc(-c5nc6ccccc6s5)cc(-c5nc6ccccc6s5)c4)nc(-c4ccc5c(c4)sc4ccccc45)n3)cc21 ; c1ccc(-c2nc(-c3ccc(-n4c5ccccc5c5ccccc54)cc3)nc(-c3cc(-c4nc5ccccc5s4)cc(-c4nc5ccccc5s4)c3)n2)cc1 ; c1ccc(-c2nc3ccc(-c4nc(-c5cc(-c6nc7ccccc7s6)cc(-c6nc7ccccc7s6)c5)nc(-c5cccc6sc7ccccc7c56)n4)cc3[nH]2)cc1]

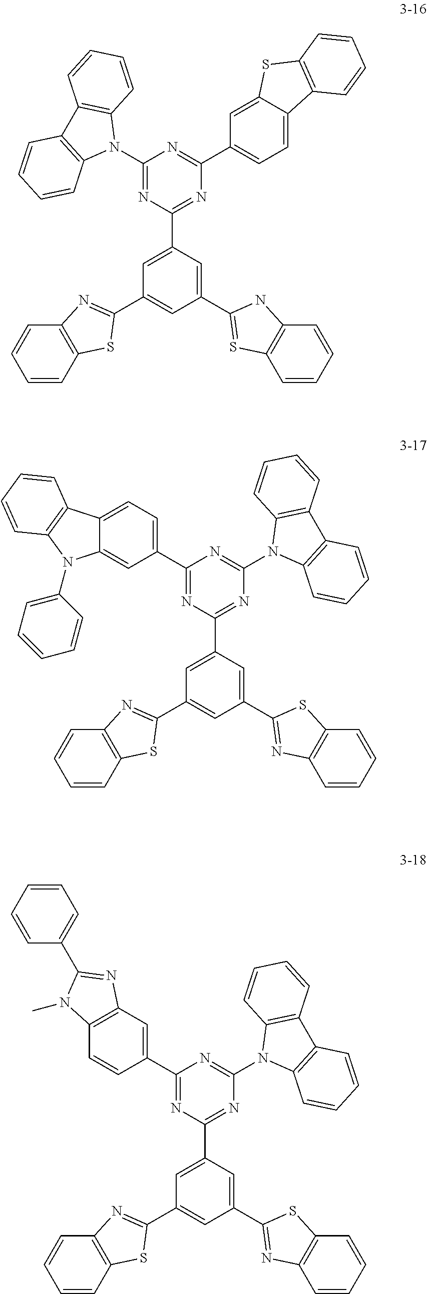

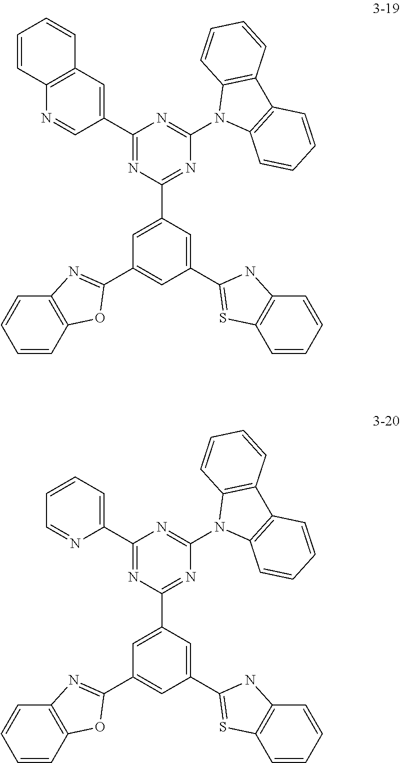

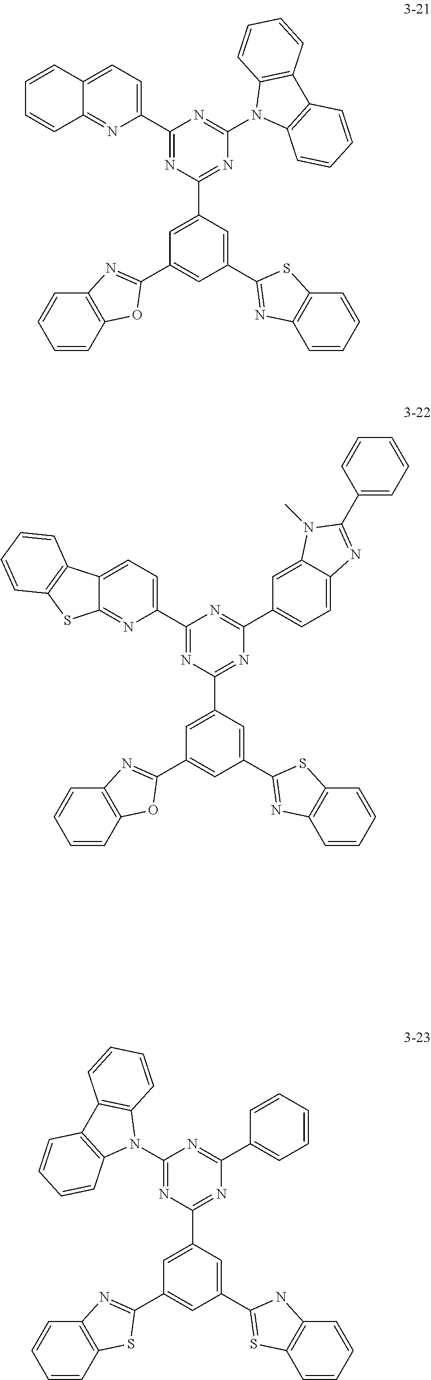

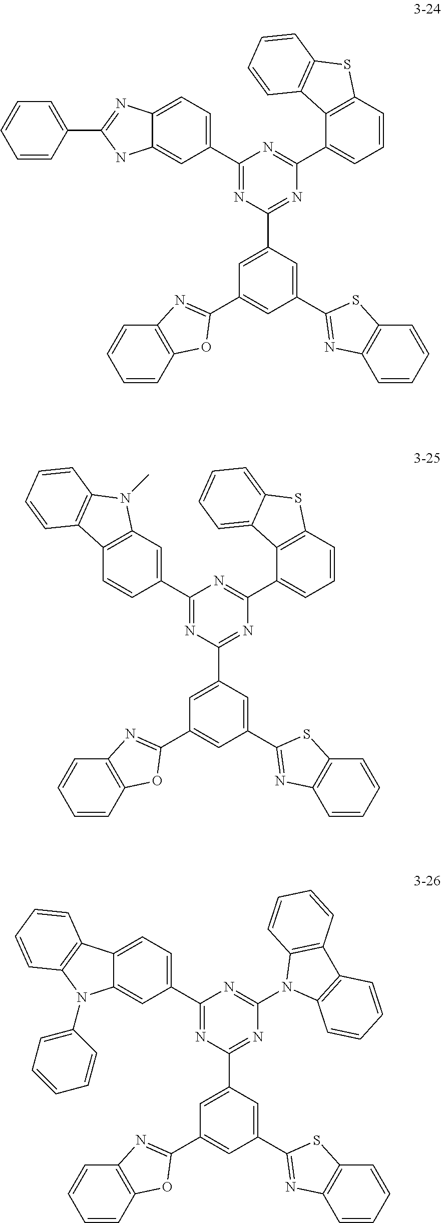 [SMILES: c1ccc(-c2nc3ccc(-c4nc(-c5cc(-c6nc7ccccc7o6)cc(-c6nc7ccccc7s6)c5)nc(-c5cccc6sc7ccccc7c56)n4)cc3[nH]2)cc1 ; Cn1c2ccccc2c2ccc(-c3nc(-c4cc(-c5nc6ccccc6o5)cc(-c5nc6ccccc6s5)c4)nc(-c4cccc5sc6ccccc6c45)n3)cc21 ; c1ccc(-n2c3ccccc3c3ccc(-c4nc(-c5cc(-c6nc7ccccc7o6)cc(-c6nc7ccccc7s6)c5)nc(-n5c6ccccc6c6ccccc65)n4)cc32)cc1]

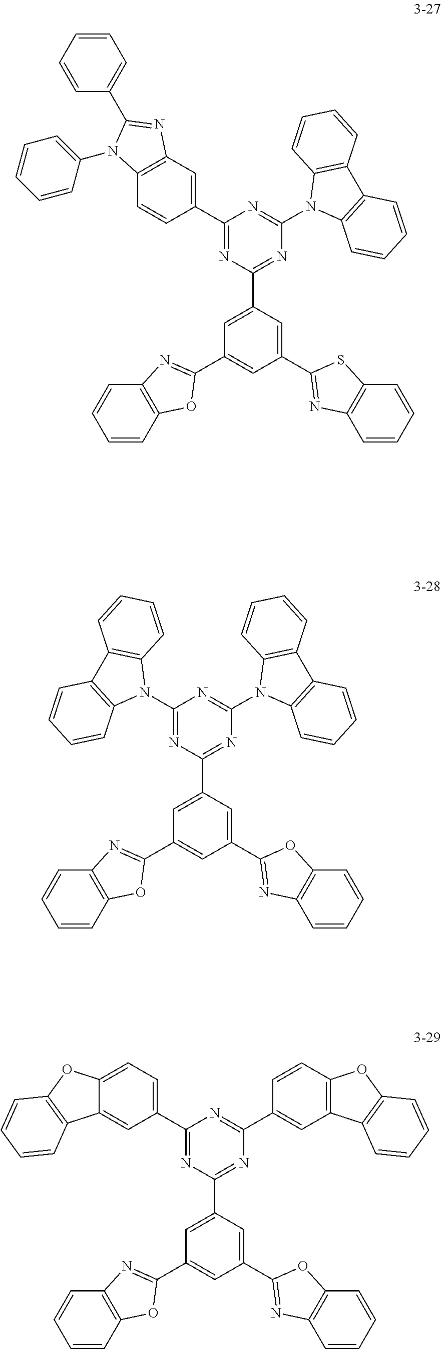 [SMILES: c1ccc(-c2nc3cc(-c4nc(-c5cc(-c6nc7ccccc7o6)cc(-c6nc7ccccc7s6)c5)nc(-n5c6ccccc6c6ccccc65)n4)ccc3n2-c2ccccc2)cc1 ; c1ccc2oc(-c3cc(-c4nc(-n5c6ccccc6c6ccccc65)nc(-n5c6ccccc6c6ccccc65)n4)cc(-c4nc5ccccc5o4)c3)nc2c1 ; c1ccc2oc(-c3cc(-c4nc(-c5ccc6oc7ccccc7c6c5)nc(-c5ccc6oc7ccccc7c6c5)n4)cc(-c4nc5ccccc5o4)c3)nc2c1]

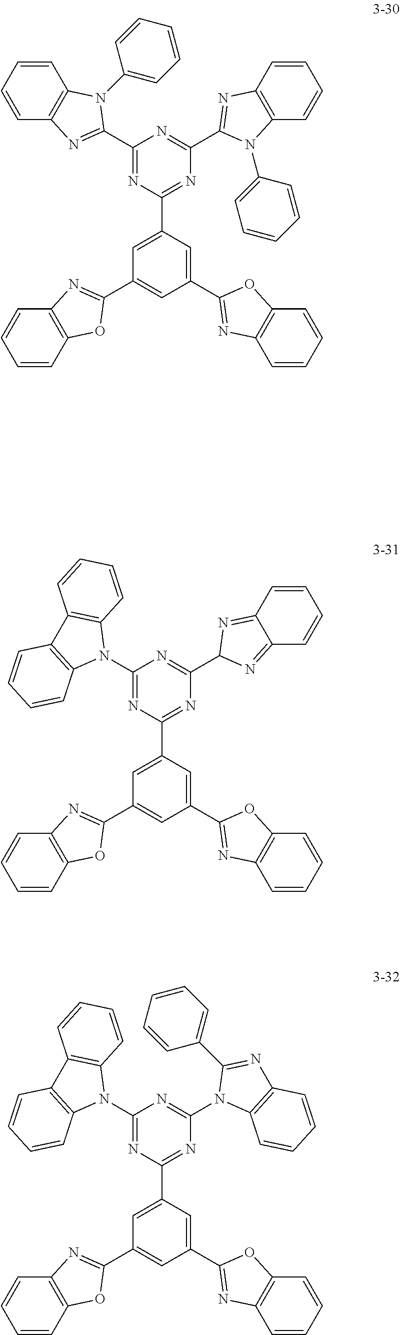 [SMILES: c1ccc(-n2c(-c3nc(-c4cc(-c5nc6ccccc6o5)cc(-c5nc6ccccc6o5)c4)nc(-c4nc5ccccc5n4-c4ccccc4)n3)nc3ccccc32)cc1 ; c1ccc2c(c1)=NC(c1nc(-c3cc(-c4nc5ccccc5o4)cc(-c4nc5ccccc5o4)c3)nc(-n3c4ccccc4c4ccccc43)n1)N=2 ; c1ccc(-c2nc3ccccc3n2-c2nc(-c3cc(-c4nc5ccccc5o4)cc(-c4nc5ccccc5o4)c3)nc(-n3c4ccccc4c4ccccc43)n2)cc1]

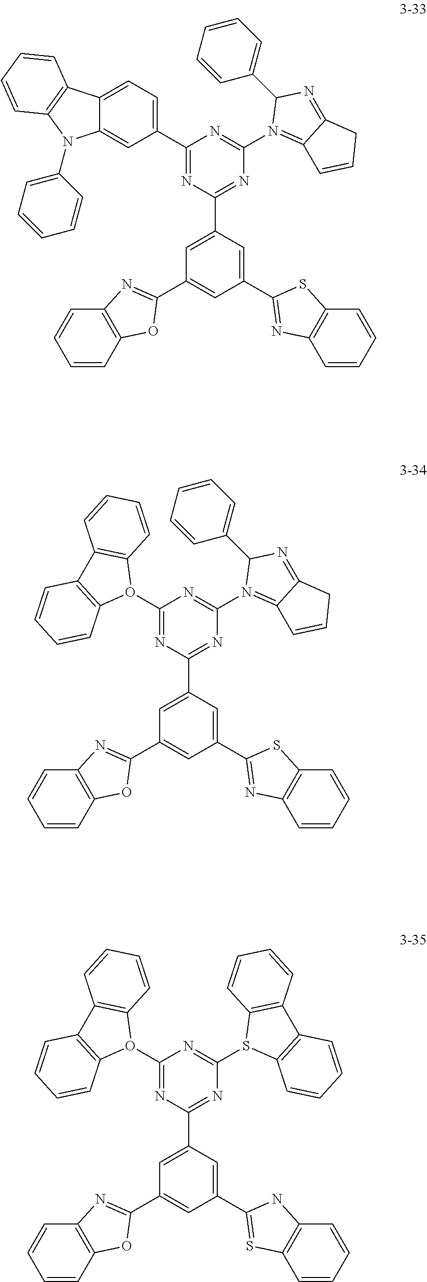

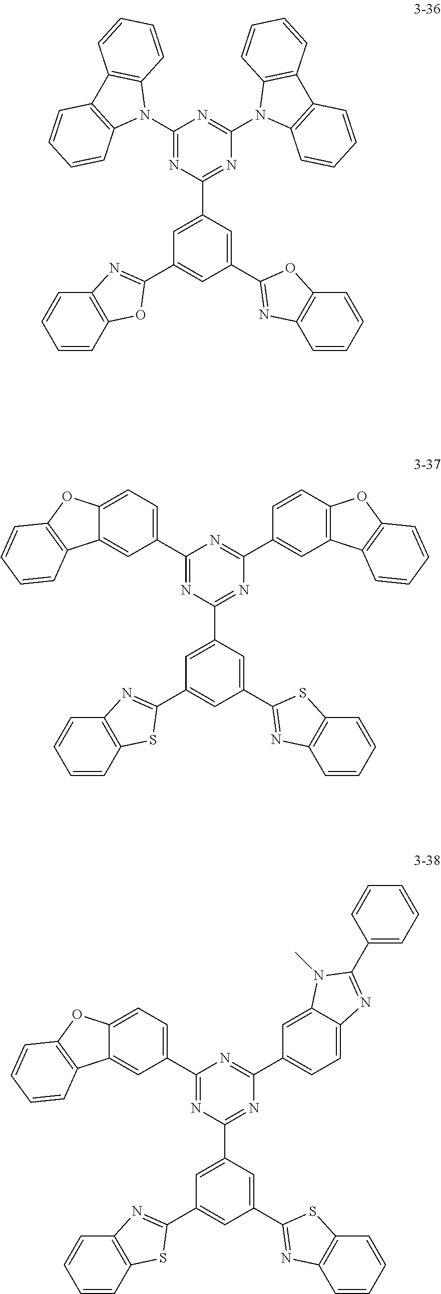 [SMILES: c1ccc2oc(-c3cc(-c4nc(-n5c6ccccc6c6ccccc65)nc(-n5c6ccccc6c6ccccc65)n4)cc(-c4nc5ccccc5o4)c3)nc2c1 ; c1ccc2sc(-c3cc(-c4nc(-c5ccc6oc7ccccc7c6c5)nc(-c5ccc6oc7ccccc7c6c5)n4)cc(-c4nc5ccccc5s4)c3)nc2c1 ; Cn1c(-c2ccccc2)nc2ccc(-c3nc(-c4cc(-c5nc6ccccc6s5)cc(-c5nc6ccccc6s5)c4)nc(-c4ccc5oc6ccccc6c5c4)n3)cc21]

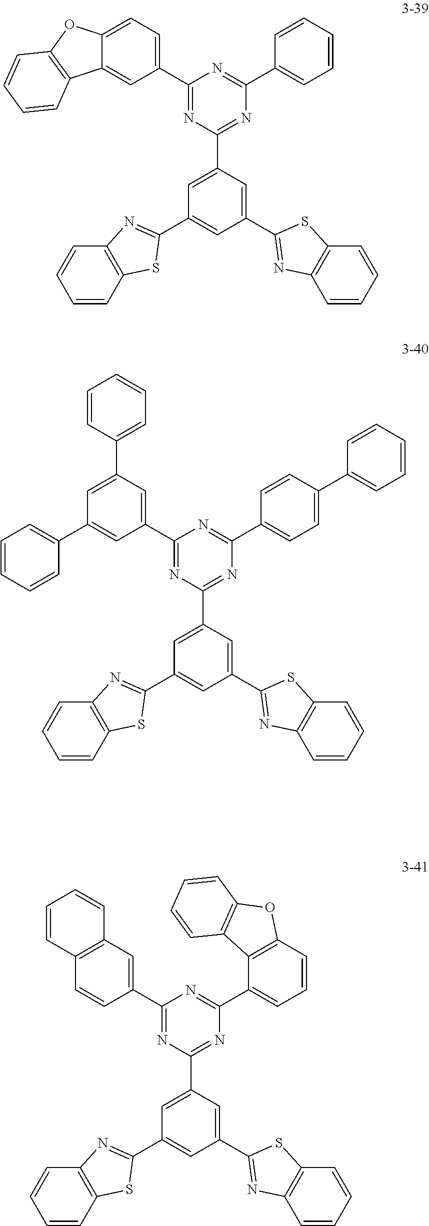 [SMILES: c1ccc(-c2nc(-c3cc(-c4nc5ccccc5s4)cc(-c4nc5ccccc5s4)c3)nc(-c3ccc4oc5ccccc5c4c3)n2)cc1 ; c1ccc(-c2ccc(-c3nc(-c4cc(-c5ccccc5)cc(-c5ccccc5)c4)nc(-c4cc(-c5nc6ccccc6s5)cc(-c5nc6ccccc6s5)c4)n3)cc2)cc1 ; c1ccc2cc(-c3nc(-c4cc(-c5nc6ccccc6s5)cc(-c5nc6ccccc6s5)c4)nc(-c4cccc5oc6ccccc6c45)n3)ccc2c1]

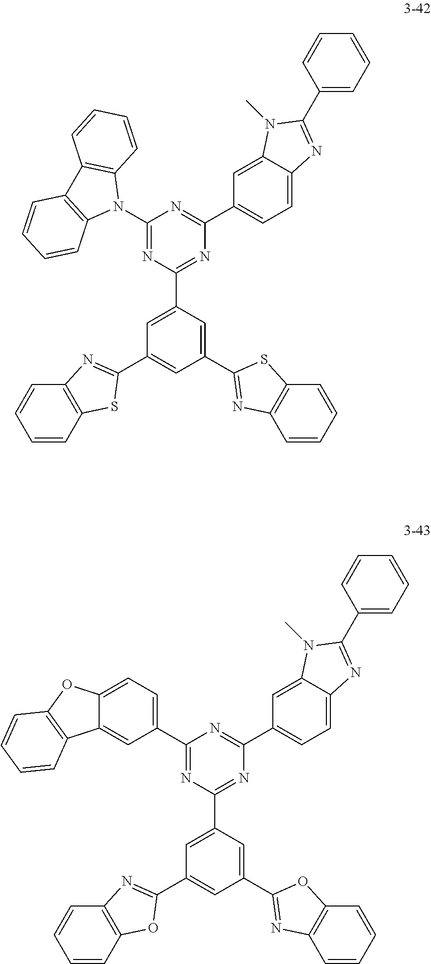 [SMILES: Cn1c(-c2ccccc2)nc2ccc(-c3nc(-c4cc(-c5nc6ccccc6s5)cc(-c5nc6ccccc6s5)c4)nc(-n4c5ccccc5c5ccccc54)n3)cc21 ; Cn1c(-c2ccccc2)nc2ccc(-c3nc(-c4cc(-c5nc6ccccc6o5)cc(-c5nc6ccccc6o5)c4)nc(-c4ccc5oc6ccccc6c5c4)n3)cc21]

The organic compound of the present disclosure, which is represented by Formula 1 and may be one of the compounds in Formula 2, has a wide bandgap, a low highest occupied molecular orbital (HOMO) and a high triplet energy (Ti). Accordingly, the stability and the carrier mobility of the organic compound are improved. The organic compound is used as at least one of a host of an emitting material layer, a hole blocking material of a hole blocking layer and an electron transporting material of an electron transporting layer so that at least one of the emitting efficiency and the lifespan of an OLED is improved.

### [Synthesis]

### 1. Synthesis of an Intermediate a

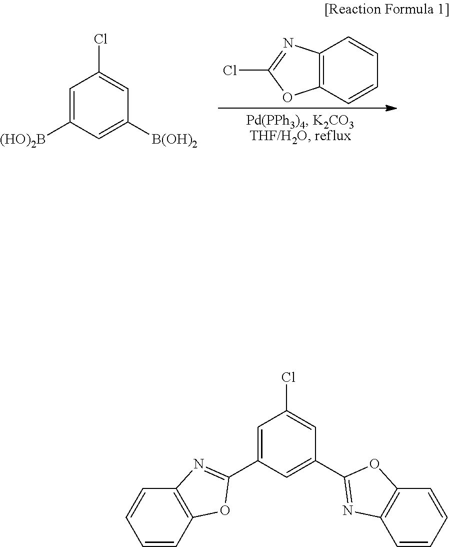

In a nitrogen atmosphere, 5-chlorophenyldiboronic acid 10 g (50 mmol) and 2-chlorobenzo[d]oxazole 16.9 g (110 mmol) were dissolved in tetrahydrofuran (THF) 200 mL. Potassium carbonate 17.3 g was dissolved in distilled water 50 mL, added to the THF solution, and stirred. Tetrakis(triphenylphosphine)palladium(0) 1.2 g (0.2 mmol) was added and refluxed for 12 hours. After completion of the reaction, it was cooled to room temperature, the organic layer and the water layer were separated, and the organic layer was distilled under reduced pressure. The reduced-pressure distilled filtrate was extracted with chloroform and water. The residual moisture was removed from the organic layer using magnesium sulfate and was distilled under reduced pressure. The target material was separated from the filtrate through silica gel column chromatography to obtain the intermediate A 9.5 g (27 mmol). (yield 55%)

### 2. Synthesis of an Intermediate B

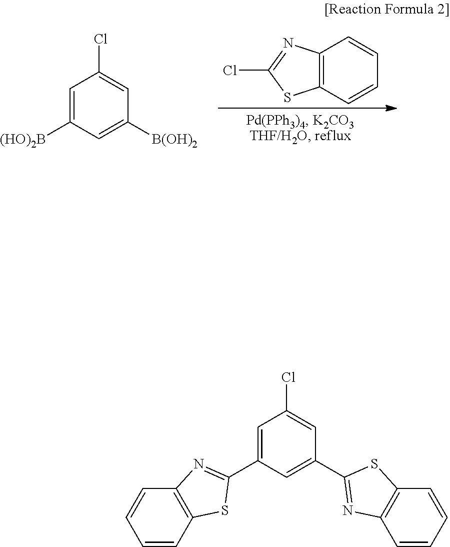

In a nitrogen atmosphere, 5-chlorophenyldiboronic acid 10 g (50 mmol) and 2-chlorobenzo[d]thiazole 18.6 g (110 mmol) were dissolved in THF 200 mL. Potassium carbonate 17.3 g was dissolved in distilled water 50 mL, added to the THF solution, and stirred. Tetrakis(triphenylphosphine)palladium(0) 1.2 g (0.2 mmol) was added and refluxed for 12 hours. After completion of the reaction, it was cooled to room temperature, the organic layer and the water layer were separated, and the organic layer was distilled under reduced pressure. The reduced-pressure distilled filtrate was extracted with chloroform and water. The residual moisture was removed from the organic layer using magnesium sulfate and was distilled under reduced pressure. The target material was separated from the filtrate through silica gel column chromatography to obtain the intermediate B 10.4 g (27 mmol). (yield 55%)

### 3. Synthesis of an Intermediate C

### (1) An Intermediate C-a

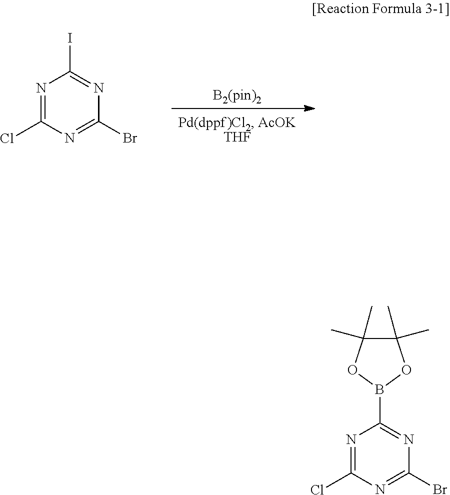 [SMILES: Clc1nc(Br)nc(I)n1 ; "B2(pin)2" ; "Pd(dppf)Cl2, AcOKTHF" ; CC1(C)OB(c2nc(Cl)nc(Br)n2)OC1(C)C]

In a nitrogen atmosphere, 2-bromo-4-chloro-6-iodo-1,3,5-triazine 10 g (31 mmol), bis(pinacolate)diboron (B₂(pin)₂) 8.7 g (34 mmol), dichloropalladium (Pd(dppf)Cl₂) 0.5 g (0.5 mmol) and potassium acetate (AcOK) 6.1 g (62 mmol) were dissolved in 200 mL of THF and stirred under reflux for 3 hours. The reaction solution was cooled to room temperature and filtered under reduced pressure with washing by methylene chloride (MC). The filtrate was concentrated under reduced pressure and then filtered through a silica gel filter. The solid was precipitated using methanol to obtain the intermediate C-a 8.5 g (28 mmol). (yield 90%)

### (2) An Intermediate C-b

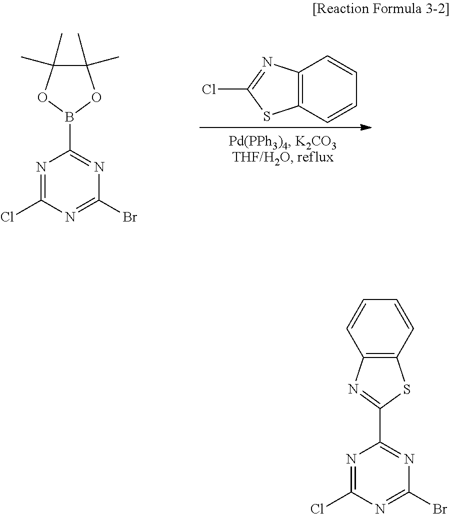

In a nitrogen atmosphere, the intermediate C-a 8.2 g (27 mmol) and 2-chlorobenzo[d]thiazole 5.1 g (30 mmol) were dissolved in THF 150 mL. Potassium carbonate 9.4 g was dissolved in 30 mL of distilled water, added to the THF solution, and stirred. Pd(PPh₃)₄(tetrakis(triphenylphosphine)palladium(0)) 0.6 g (1 mmol) was added and refluxed for 3 hours. After completion of the reaction, it was cooled to room temperature, the organic layer and the water layer were separated, and the organic layer was distilled under reduced pressure. The reduced-pressure distilled filtrate was extracted with chloroform and water. The residual moisture was removed from the organic layer using magnesium sulfate and was distilled under reduced pressure. The target material was separated from the filtrate through silica gel column chromatography to obtain the intermediate C-b 6.7 g (20 mmol). (yield 75%)

### (3) An Intermediate C-c

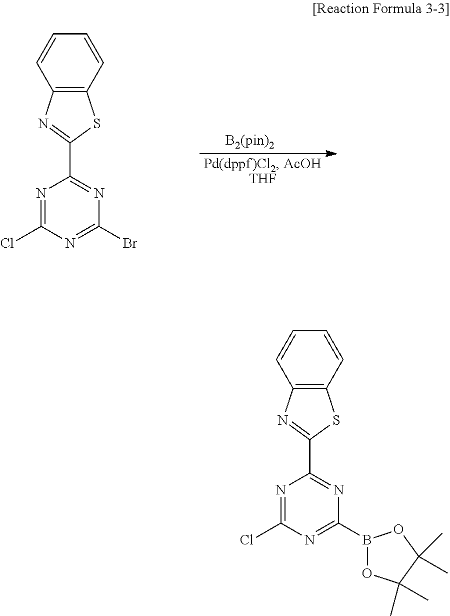 [SMILES: Clc1nc(Br)nc(-c2nc3ccccc3s2)n1 ; "Pd(dppf)Cl2, AcOHTHF" ; CC1(C)OB(c2nc(Cl)nc(-c3nc4ccccc4s3)n2)OC1(C)C]

In a nitrogen atmosphere, after the intermediate C-b 6.5 g (20 mmol), (B₂(pin)₂) 5.5 g (22 mmol), Pd(dppf)Cl₂ 0.3 g (0.4 mmol), and potassium acetate (AcOK) 3.9 g (40 mmol) were dissolved with 1,4-dioxane 200 mL, and the mixture was refluxed and stirred for 3 hours. The reaction solution was cooled to room temperature and filtered under reduced pressure with washing by MC. The filtrate was concentrated under reduced pressure and filtered through a silica gel filter. The solid was precipitated using methanol to obtain the intermediate C-c 6.7 g (18 mmol). (yield 90%)

### (4) An Intermediate C

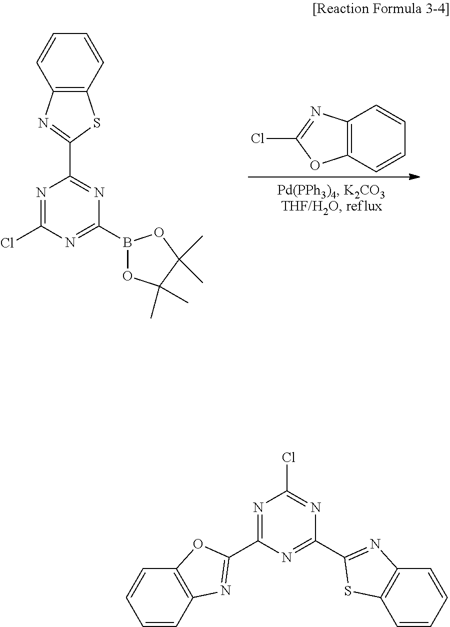

In a nitrogen atmosphere, the intermediate C-c 6.5 g (17 mmol) and 2-chlorobenzo[d]oxazole 2.9 g (19 mmol) were dissolved in THF 150 mL. Potassium carbonate 6.0 g was dissolved in distilled water 30 mL, added to the THF solution, and stirred. Pd(PPh₃)₄ (tetrakis(triphenylphosphine)palladium(0)) 0.4 g (0.3 mmol) was added and refluxed for 3 hours. After completion of the reaction, it was cooled to room temperature, the organic layer and the water layer were separated, and the organic layer was distilled under reduced pressure. The reduced-pressure distilled filtrate was extracted with chloroform and water. The residual moisture was removed from the organic layer using magnesium sulfate and was distilled under reduced pressure. The target material was separated from the filtrate through silica gel column chromatography to obtain the intermediate C 4.8 g (13 mmol). (yield 75%)

### 4. Synthesis of an Intermediate 1

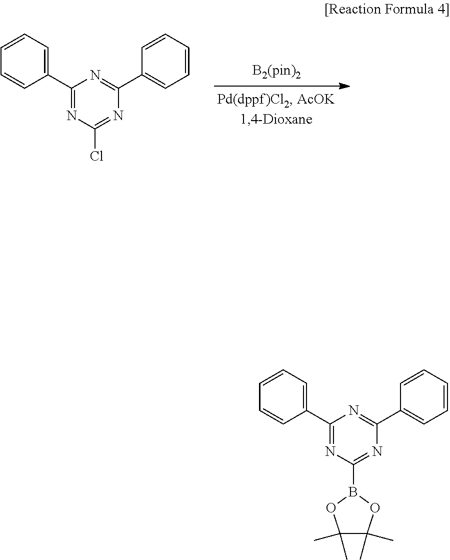 [SMILES: Clc1nc(-c2ccccc2)nc(-c2ccccc2)n1 ; CC1(C)OB(c2nc(-c3ccccc3)nc(-c3ccccc3)n2)OC1(C)C]

In a nitrogen atmosphere, 2-chloro-4,6-diphenyl-1,3,5-triazine 12 g (45 mmol), B₂(pin)₂ 12.5 g (49 mmol), Pd(dppf)Cl₂ 0.7 g (1 mmol), potassium acetate (AcOK) 8.8 g (90 mmol) were dissolved in 1,4-dioxane 150 mL and stirred under reflux for 3 hours. The reaction solution was cooled to room temperature and filtered under reduced pressure with washing by MC. The filtrate was concentrated under reduced pressure and then filtered through a silica gel filter. The solid was precipitated using methanol to obtain the intermediate 1 6.7 g (18 mmol). (yield 90%)

### 5. Synthesis of an Intermediate 2

(1) an intermediate 2-a

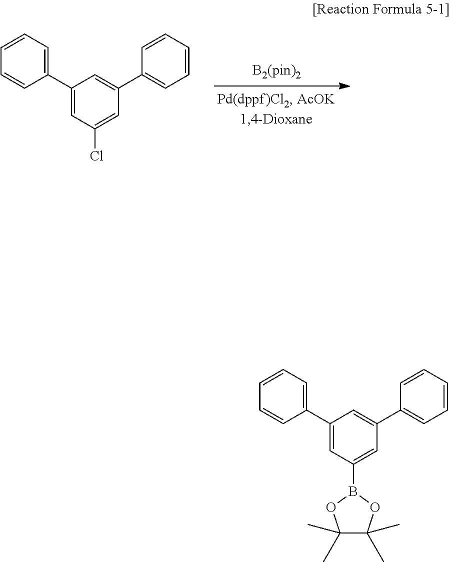 [SMILES: Clc1cc(-c2ccccc2)cc(-c2ccccc2)c1 ; CC1(C)OB(c2cc(-c3ccccc3)cc(-c3ccccc3)c2)OC1(C)C]

In a nitrogen atmosphere, 5′-chloro-(1,1′,3′,1″)terphenyl 10 g (38 mmol), B₂(pin)₂ 10.6 g (42 mmol), Pd(dppf)Cl₂ 0.6 g (0.8 mmol), potassium acetate (AcOK) 7.4 g (76 mmol) were dissolved in 1,4-dioxane 150 mL and stirred under reflux for 3 hours. The reaction solution was cooled to room temperature and filtered under reduced pressure with washing by MC. The filtrate was concentrated under reduced pressure and then filtered through a silica gel filter. The solid was precipitated using methanol to obtain the intermediate 2-a 12.1 g (34 mmol). (yield 90%)

(2) an intermediate 2-b

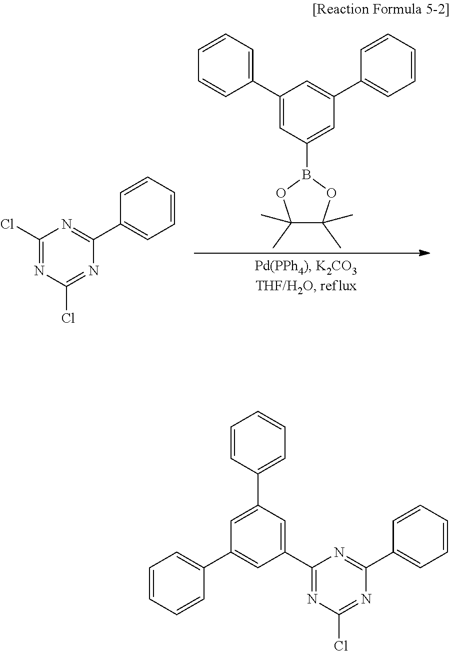 [SMILES: Clc1nc(Cl)nc(-c2ccccc2)n1 ; CC1(C)OB(c2cc(-c3ccccc3)cc(-c3ccccc3)c2)OC1(C)C ; Clc1nc(-c2ccccc2)nc(-c2cc(-c3ccccc3)cc(-c3ccccc3)c2)n1]

In a nitrogen atmosphere, 2,4-dichloro-6-phenyl-1,3,5-triazine 7.0 g (31 mmol) and the intermediate 2-a 12.1 g (34 mmol) were dissolved in THF 150 mL. Potassium carbonate 8.6 g (62 mmol) was dissolved in distilled water 30 mL, added to the THF solution, and stirred. Pd(PPh₃)₄(tetrakis(triphenylphosphine)palladium(0)) 0.7 g (1 mmol) was added and refluxed for 12 hours. After completion of the reaction, it was cooled to room temperature, the organic layer and the water layer were separated, and the organic layer was distilled under reduced pressure. The reduced-pressure distilled filtrate was extracted with chloroform and water. The residual moisture was removed from the organic layer using magnesium sulfate and was distilled under reduced pressure. The target material was separated from the filtrate through silica gel column chromatography to obtain the intermediate 2-b 8.5 g (20 mmol). (yield 65%)

(3) an intermediate 2

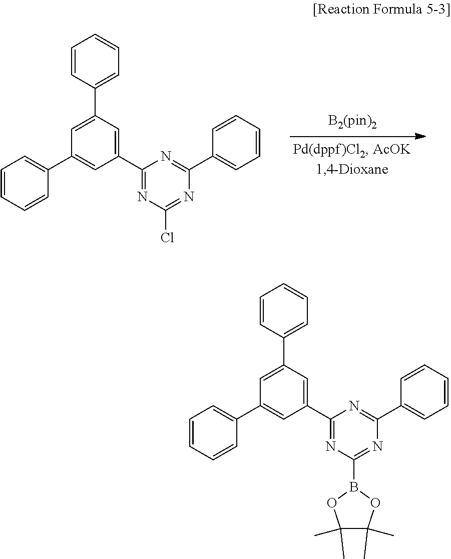 [SMILES: Clc1nc(-c2ccccc2)nc(-c2cc(-c3ccccc3)cc(-c3ccccc3)c2)n1 ; CC1(C)OB(c2nc(-c3ccccc3)nc(-c3cc(-c4ccccc4)cc(-c4ccccc4)c3)n2)OC1(C)C]

In a nitrogen atmosphere, the intermediate 2-b 11 g (26 mmol), B₂(pin)₂ 7.3 g (29 mmol), Pd(dppf)C120.4 g (1 mmol), and potassium acetate (AcOK) 5.1 g (52 mmol) were dissolved with 1,4-dioxane 150 mL, and the mixture was refluxed and stirred for 3 hours. The reaction solution was cooled to room temperature and filtered under reduced pressure with washing by MC. The filtrate was concentrated under reduced pressure and then filtered through a silica gel filter. The solid was precipitated using methanol to obtain the intermediate 2 11.4 g (22 mmol). (yield 85%)

### 6. Synthesis of an Intermediate 3

### (1) An Intermediate 3-a

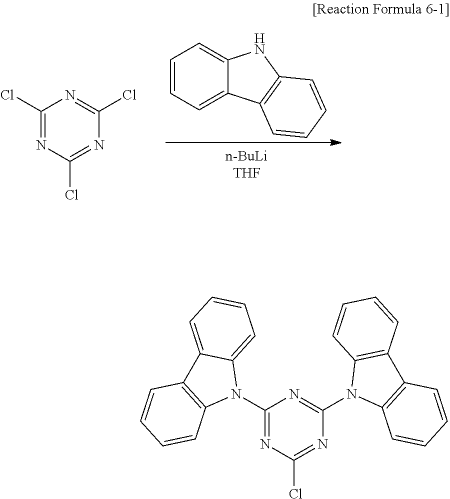

In a nitrogen atmosphere, carbazole 21.1 g (126 mmol) and THF were added to a two-neck round bottom flask. 2M n-BuLi (2M in Hexane solution) was slowly added and stirred for 30 minutes. 2,4,6-trichlorotriazine 9.3 g (50 mmol) dissolved in THF was slowly added and reacted at room temperature for 2 hours. After completion of the reaction, distilled water was added for quenching, and the resulting solid was filtered. The filtered material was washed sequentially with distilled water, methanol, and hexane under a reduced pressure filter and dried to obtain the intermediate 3-a 6.7 g (15 mmol). (yield 30%)

### (2) An Intermediate 3

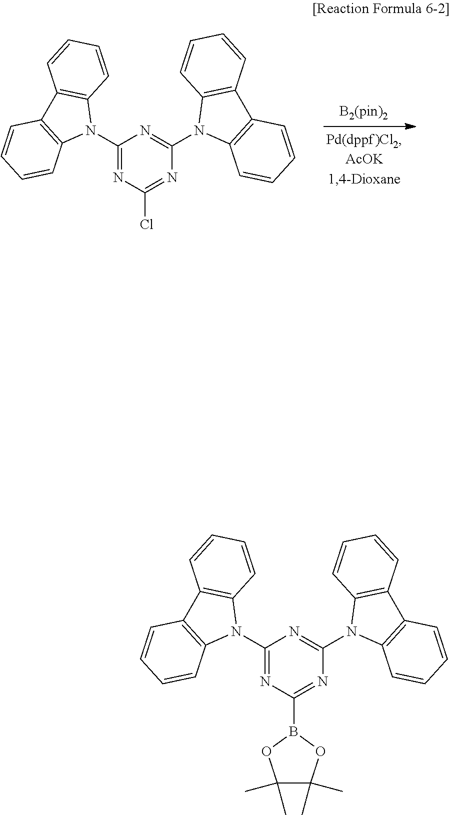 [SMILES: Clc1nc(-n2c3ccccc3c3ccccc32)nc(-n2c3ccccc3c3ccccc32)n1 ; CC1(C)OB(c2nc(-n3c4ccccc4c4ccccc43)nc(-n3c4ccccc4c4ccccc43)n2)OC1(C)C]

In a nitrogen atmosphere, the intermediate 3-a 6.5 g (15 mmol), B₂(pin)₂ 4.1 g (16 mmol), Pd(dppf)Cl₂ 0.2 g (0.3 mmol), and potassium acetate (AcOK) 2.9 g (52 mmol) were dissolved in 1,4-dioxane 60 mL. The mixture was stirred and refluxed for 5 hours. The reaction solution was cooled to room temperature and filtered under reduced pressure with washing by MC. The filtrate was concentrated under reduced pressure and then filtered through a silica gel filter. filter. The solid was precipitated using methanol to obtain the intermediate 3 6.7 g (12 mmol). (yield 85%)

### 7. Synthesis of an Intermediate 4

### (1) An Intermediate 4-a

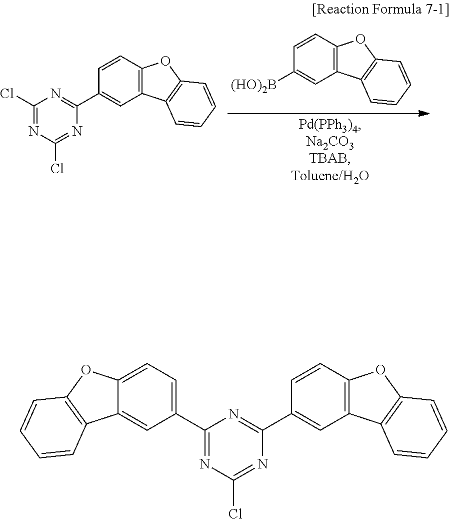 [SMILES: Clc1nc(Cl)nc(-c2ccc3oc4ccccc4c3c2)n1 ; OB(O)c1ccc2oc3ccccc3c2c1 ; Clc1nc(-c2ccc3oc4ccccc4c3c2)nc(-c2ccc3oc4ccccc4c3c2)n1]

In a nitrogen atmosphere, 2-4-dichloro-6-(dibenzo[b,d]furan-2-yl)-1,3,5-triazine 6.5 g (21 mmol) and dibenzofuran-2-ylboronic acid 4.8 g (23 mmol), and tetrabutylammonium bromide (TBAB) 0.7 g were dissolved in toluene 60 mL. A solution of sodium carbonate 5.4 g (51 mmol) dissolved in distilled water 10 mL was added to the toluene reaction solution and heated to 40° C. while maintaining the nitrogen atmosphere. Pd(PPh₃)₄ (tetrakis(triphenylphosphine)palladium(0)) 0.5 g (0.4 mmol) was added to the heated mixed solution and refluxed for 12 hours. After completion of the reaction, it was cooled to room temperature, the organic layer and the water layer were separated, and the organic layer was distilled under reduced pressure. The reduced-pressure distilled filtrate was extracted with chloroform and water. The residual moisture was removed from the organic layer using magnesium sulfate and was distilled under reduced pressure. The target material was separated from the filtrate through silica gel column chromatography to obtain the intermediate 4-a 5.1 g (11 mmol). (55% yield)

### (2) An Intermediate 4

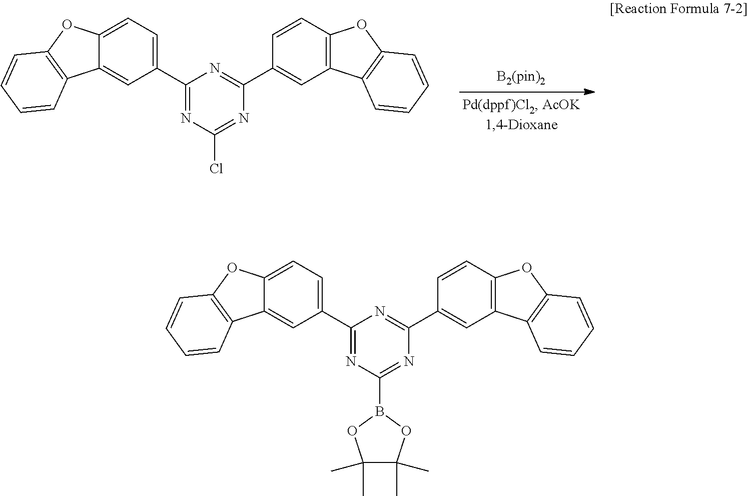 [SMILES: Clc1nc(-c2ccc3oc4ccccc4c3c2)nc(-c2ccc3oc4ccccc4c3c2)n1 ; CC1(C)OB(c2nc(-c3ccc4oc5ccccc5c4c3)nc(-c3ccc4oc5ccccc5c4c3)n2)OC1(C)C]

In a nitrogen atmosphere, the intermediate 4-a 5.0 g (11 mmol), B₂(pin)₂ 3.1 g (12 mmol), Pd(dppf)Cl₂ 0.2 g (0.2 mmol), and potassium acetate (AcOK) 2.2 g (22 mmol) were dissolved in 1,4-dioxane 50 mL. The mixture was stirred and refluxed for 3 hours. The reaction solution was cooled to room temperature and filtered under reduced pressure with washing by MC. The filtrate was concentrated under reduced pressure and then filtered through a silica gel filter. The solid was precipitated using methanol to obtain the intermediate 4 5.4 g (10 mmol). (yield 90%)

### 8. Synthesis of an Intermediate 5

### (1) An Intermediate 5-a

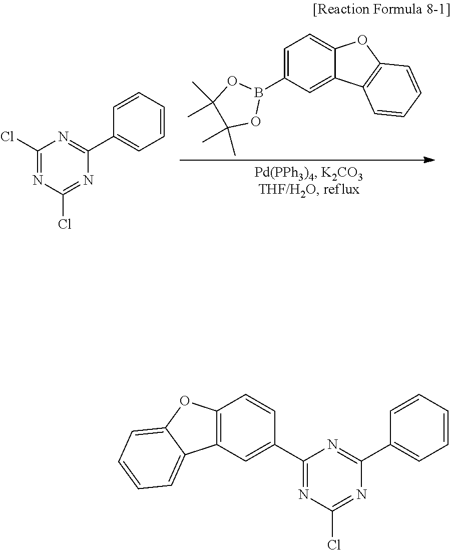

In a nitrogen atmosphere, 2,4-dichloro-6-phenyl-1,3,5-triazine 8.0 g (35 mmol) and 2-(dibenzo[b,d]furan-2-yl)-4,4,5,5-tetramethyl-1,3,2-dioxaborolane 11.5 g (39 mmol) were dissolved in THF 120 mL. Potassium carbonate 12.2 g (62 mmol) was dissolved in distilled water 30 mL, added to the THF solution, and stirred. Pd(PPh₃)₄ (tetrakis(triphenylphosphine)palladium(0)) 0.8 g (1 mmol) was added and refluxed for 12 hours. After completion of the reaction, the mixture was cooled to room temperature, the organic layer and the water layer were separated, and the organic layer was distilled under reduced pressure. The reduced-pressure distilled filtrate was extracted with chloroform and water. The residual moisture was removed from the organic layer using magnesium sulfate and was distilled under reduced pressure. The target material was separated from the filtrate through silica gel column chromatography to obtain the intermediate 5-a 9.5 g (27 mmol). (yield 75%)

### (2) An Intermediate 5

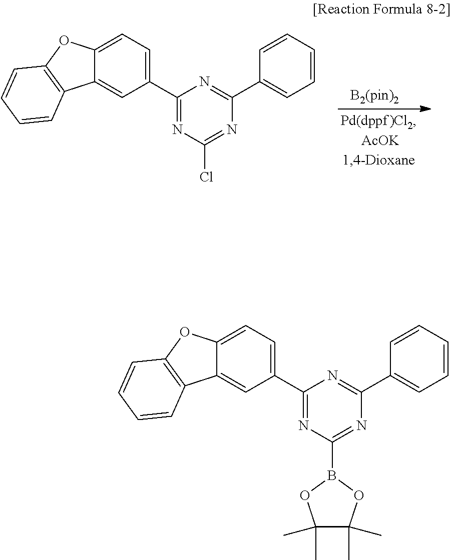 [SMILES: Clc1nc(-c2ccccc2)nc(-c2ccc3oc4ccccc4c3c2)n1 ; CC1(C)OB(c2nc(-c3ccccc3)nc(-c3ccc4oc5ccccc5c4c3)n2)OC1(C)C]

In a nitrogen atmosphere, the intermediate 5-a 11 g (31 mmol), B₂(pin)₂ 8.6 g (34 mmol), Pd(dppf)Cl₂ 0.4 g (0.6 mmol), and potassium acetate (AcOK) 6.0 g (61 mmol) were dissolved in 1,4-dioxane 100 mL. The mixture was stirred and refluxed for 3 hours. The reaction solution was cooled to room temperature and filtered under reduced pressure with washing by MC. The filtrate was concentrated under reduced pressure and then filtered through a silica gel filter. The solid was precipitated using methanol to obtain the intermediate 5 11.7 g (26 mmol). (yield 85%)

### 9. Synthesis of an Intermediate 6

### (1) An Intermediate 6-a

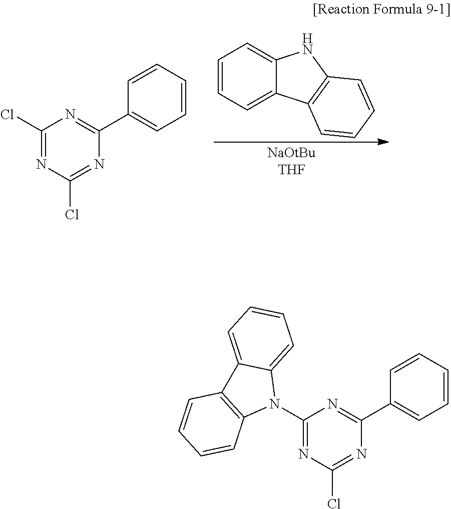

In a nitrogen atmosphere and under a temperature of 0° C., 2,4,6-trichloro-1,3,5-triazine 10.0 g (54 mmol), 9H-carbazole 11.5 g (39 mmol), and sodium tert-butoxide 5.7 g (60 mmol) were dissolved in THT 200 mL. The mixture was reacted at room temperature for 12 hours. Distilled water was added to the mixture for quenching, and the solid was filtered through a reduced pressure filter. The filtered solid was washed with distilled water and hexane, and then purified using a silica filter. The purified solution was concentrated and precipitated using acetone to obtain the intermediate 6-a 15.5 g (43 mmol). (yield 80%)

### (2) An Intermediate 6

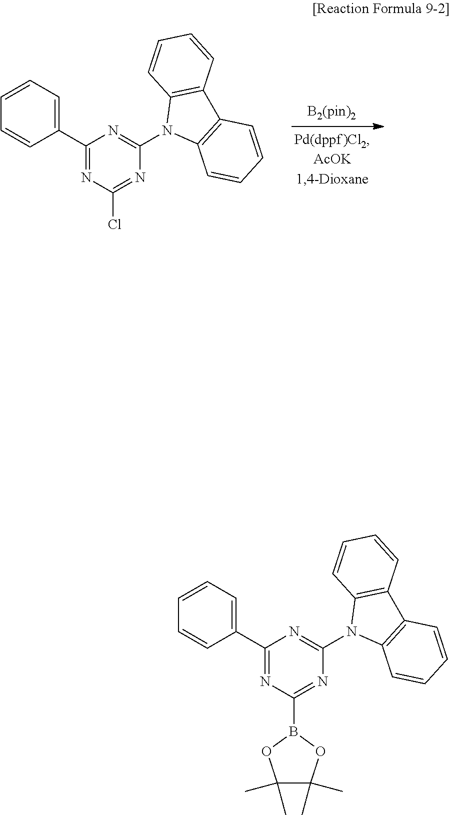 [SMILES: Clc1nc(-c2ccccc2)nc(-n2c3ccccc3c3ccccc32)n1 ; CC1(C)OB(c2nc(-c3ccccc3)nc(-n3c4ccccc4c4ccccc43)n2)OC1(C)C]

In a nitrogen atmosphere, the intermediate 6-a 11 g (31 mmol), B₂(pin)₂ 8.6 g (34 mmol), Pd(dppf)Cl₂ 0.5 g (0.6 mmol), and potassium acetate (AcOK) 6.1 g (62 mmol) were dissolved in 1,4-dioxane 100 mL. The mixture was stirred and refluxed for 3 hours. The reaction solution was cooled to room temperature and filtered under reduced pressure with washing by MC. The filtrate was concentrated under reduced pressure and then filtered through a silica gel filter. The solid was precipitated using methanol to obtain the intermediate 6 11.7 g (26 mmol). (yield 85%)

### 10. Synthesis of an Intermediate

### (1) An Intermediate 7-a

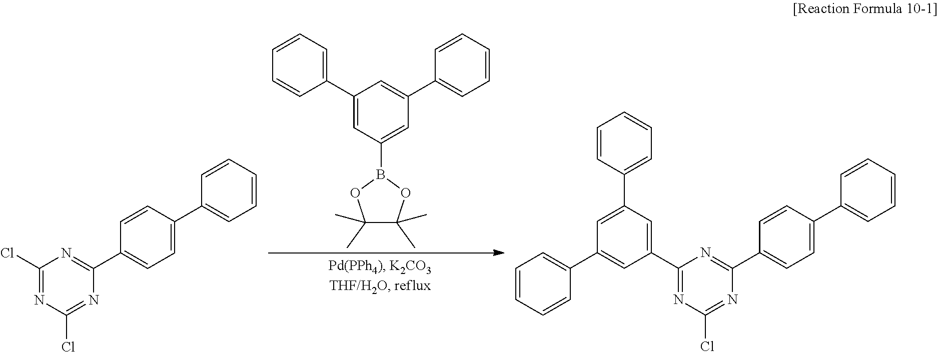 [SMILES: Clc1nc(Cl)nc(-c2ccc(-c3ccccc3)cc2)n1 ; CC1(C)OB(c2cc(-c3ccccc3)cc(-c3ccccc3)c2)OC1(C)C ; Clc1nc(-c2ccc(-c3ccccc3)cc2)nc(-c2cc(-c3ccccc3)cc(-c3ccccc3)c2)n1]

In a nitrogen atmosphere, 2-(4-biphenylyl)-4, 6-dichloro-1,3,5-triazine 10.0 g (33 mmol) and the intermediate 2-a 13.0 g (36 mmol) were dissolved in THF 150 mL. Potassium carbonate 11.4 g (83 mmol) was dissolved in 50 mL of distilled water, added to the THF solution, and stirred. Pd(PPh₃)₄(tetrakis(triphenylphosphine)palladium(0)) 0.8 g (1 mmol) was added and refluxed for 12 hours. After completion of the reaction, the mixture was cooled to room temperature, the organic layer and the water layer were separated, and the organic layer was distilled under reduced pressure. The reduced-pressure distilled filtrate was extracted with chloroform and water. The residual moisture was removed from the organic layer using magnesium sulfate and was distilled under reduced pressure. The target material was separated from the filtrate through silica gel column chromatography to obtain the intermediate 7-a 12.3 g (25 mmol). (yield 75%)

### (2) An Intermediate 7

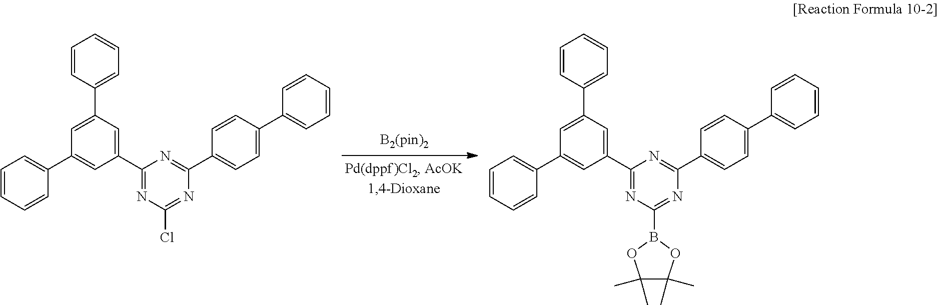 [SMILES: Clc1nc(-c2ccc(-c3ccccc3)cc2)nc(-c2cc(-c3ccccc3)cc(-c3ccccc3)c2)n1 ; CC1(C)OB(c2nc(-c3ccc(-c4ccccc4)cc3)nc(-c3cc(-c4ccccc4)cc(-c4ccccc4)c3)n2)OC1(C)C]

In a nitrogen atmosphere, the intermediate 7-a 12 g (24 mmol), B₂(pin)₂ 6.8 g (27 mmol), Pd(dppf)Cl₂ 0.4 g (0.5 mmol), and potassium acetate (AcOK) 4.7 g (48 mmol) were dissolved in 1,4-dioxane 100 mL. The mixture was stirred and refluxed for 3 hours. The reaction solution was cooled to room temperature and filtered under reduced pressure with washing by MC. The filtrate was concentrated under reduced pressure and then filtered through a silica gel filter. The solid was precipitated using methanol to obtain the intermediate 7 12.1 g (21 mmol). (yield 85%)

### 11. Synthesis of an Intermediate 8

### (1) An Intermediate 8-a

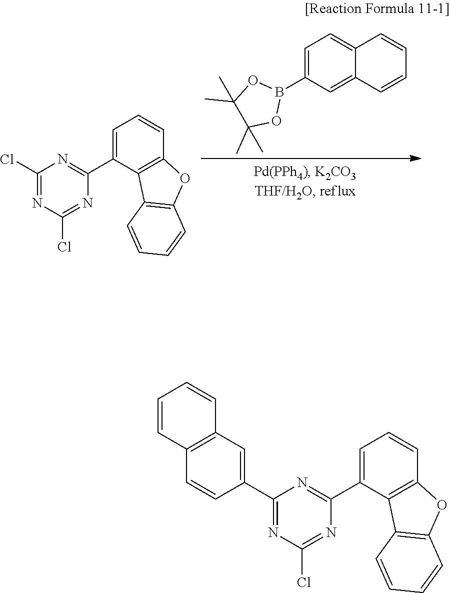 [SMILES: Clc1nc(Cl)nc(-c2cccc3oc4ccccc4c23)n1 ; CC1(C)OB(c2ccc3ccccc3c2)OC1(C)C ; Clc1nc(-c2ccc3ccccc3c2)nc(-c2cccc3oc4ccccc4c23)n1]

In a nitrogen atmosphere, 2,4-dichloro-6-(dibenzo[b,d]furan-1-yl)-1,3,5-triazine 10.0 g (32 mmol) and 4,4,5,5-tetramethyl-2-(naphthalen-2-yl)-1,3,2-dioxaborolane 8.8 g (35 mmol) were dissolved in THT 150 mL. Potassium carbonate 11.4 g (83 mmol) was dissolved in distilled water 50 mL, added to the THF solution, and stirred. Pd(PPh₃)₄ (tetrakis(triphenylphosphine)palladium(0)) 0.8 g (1 mmol) was added and refluxed for 12 hours. After completion of the reaction, the mixture was cooled to room temperature, the organic layer and the water layer were separated, and the organic layer was distilled under reduced pressure. The reduced-pressure distilled filtrate was extracted with chloroform and water. The residual moisture was removed from the organic layer using magnesium sulfate and was distilled under reduced pressure. The target material was separated from the filtrate through silica gel column chromatography to obtain the intermediate 8-a 12.1 g (24 mmol). (yield 75%)

### (2) An Intermediate 8

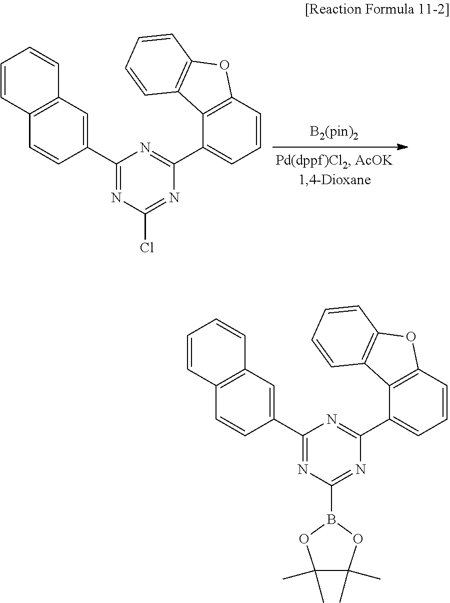 [SMILES: Clc1nc(-c2ccc3ccccc3c2)nc(-c2cccc3oc4ccccc4c23)n1 ; CC1(C)OB(c2nc(-c3ccc4ccccc4c3)nc(-c3cccc4oc5ccccc5c34)n2)OC1(C)C]

In a nitrogen atmosphere, the intermediate 8-a 9.5 g (23 mmol), B₂(pin)₂ 6.5 g (26 mmol), Pd(dppf)Cl₂ 0.3 g (0.5 mmol), and potassium acetate (AcOK) 4.6 g (47 mmol) were dissolved in 1,4-dioxane 100 mL. The mixture was stirred and refluxed for 3 hours. The reaction solution was cooled to room temperature and filtered under reduced pressure with washing by MC. The filtrate was concentrated under reduced pressure and then filtered through a silica gel filter. The solid was precipitated using methanol to obtain the intermediate 8 9.9 g (20 mmol). (yield 85%)

### 12. Synthesis of an Intermediate 9

### (1) An Intermediate 9-a

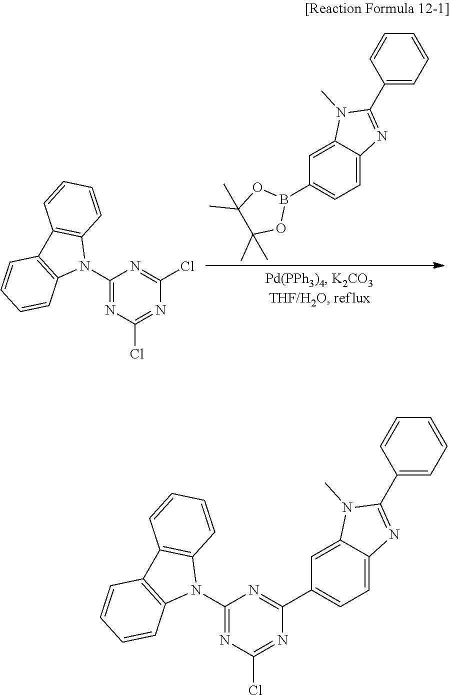 [SMILES: Clc1nc(Cl)nc(-n2c3ccccc3c3ccccc32)n1 ; Cn1c(-c2ccccc2)nc2ccc(B3OC(C)(C)C(C)(C)O3)cc21 ; Cn1c(-c2ccccc2)nc2ccc(-c3nc(Cl)nc(-n4c5ccccc5c5ccccc54)n3)cc21]

In a nitrogen atmosphere, 9-(4,6-dichloro-1,3,5-triazin-2-yl)-9H-carbazole 10.0 g (32 mmol) and 1-methyl-6-(4,4,5,5-tetramethyl)-1,3,2-dioxaborolan-2-yl)-2-phenyl-1H-benzo[d]imidazole 11.7 g (35 mmol) were dissolved in 150 mL of THF. Potassium carbonate 9.2 g (67 mmol) was dissolved in distilled water 50 mL, added to the THF solution, and stirred. Pd(PPh₃)₄(tetrakis(triphenylphosphine)palladium(0)) 0.7 g (0.6 mmol) was added and refluxed for 12 hours. After completion of the reaction, the mixture was cooled to room temperature, the organic layer and the water layer were separated, and the organic layer was distilled under reduced pressure. The reduced-pressure distilled filtrate was extracted with chloroform and water. The residual moisture was removed from the organic layer using magnesium sulfate and was distilled under reduced pressure. The target material was separated from the filtrate through silica gel column chromatography to obtain the intermediate 9-a 11.6 g (24 mmol). (yield 75%)

### (2) An Intermediate 9

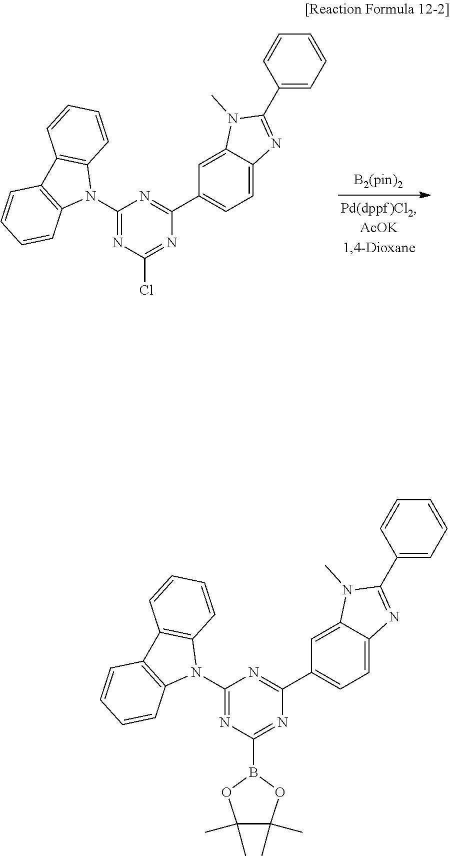 [SMILES: Cn1c(-c2ccccc2)nc2ccc(-c3nc(Cl)nc(-n4c5ccccc5c5ccccc54)n3)cc21 ; Cn1c(-c2ccccc2)nc2ccc(-c3nc(B4OC(C)(C)C(C)(C)O4)nc(-n4c5ccccc5c5ccccc54)n3)cc21]

In a nitrogen atmosphere, the intermediate 9-a 11.5 g (24 mmol), B₂(pin)₂ 6.6 g (26 mmol), Pd(dppf)Cl₂ 0.3 g (0.5 mmol), and potassium acetate (AcOK) 4.6 g (47 mmol) were dissolved in 1,4-dioxane 100 mL. The mixture was stirred and refluxed for 3 hours. The reaction solution was cooled to room temperature and filtered under reduced pressure with washing by MC. The filtrate was concentrated under reduced pressure and then filtered through a silica gel filter. The solid was precipitated using methanol to obtain the intermediate 9 11.6 g (20 mmol). (yield 85%)

### 13. Synthesis of an Intermediate 10

### (1) An Intermediate 10-a

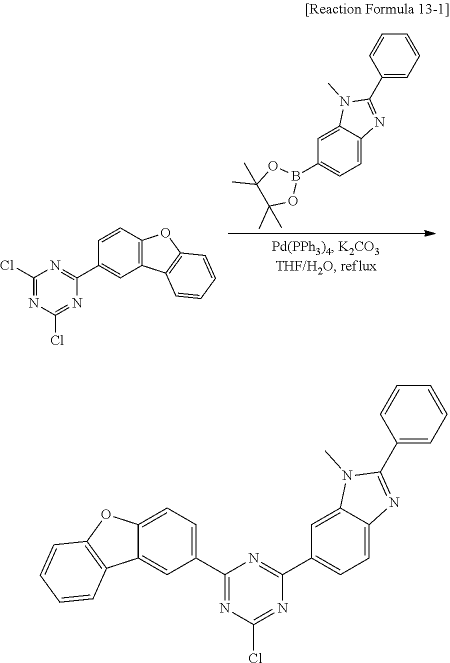 [SMILES: Clc1nc(Cl)nc(-c2ccc3oc4ccccc4c3c2)n1 ; Cn1c(-c2ccccc2)nc2ccc(B3OC(C)(C)C(C)(C)O3)cc21 ; Cn1c(-c2ccccc2)nc2ccc(-c3nc(Cl)nc(-c4ccc5oc6ccccc6c5c4)n3)cc21]

In a nitrogen atmosphere, 2-4-dichloro-6-(dibenzo[b,d]furan-2-yl)-1,3,5-triazine 10.0 g (32 mmol) and 1-methyl-6-(4,4,5,5-tetramethyl-1,3,2-dioxaborolan-2-yl)-2-phenyl-1H-benzo[d]imidazole 11.6 g (35 mmol) were dissolved in 150 mL of THF. Potassium carbonate 9.2 g (66 mmol) was dissolved in distilled water 50 mL, added to the THF solution, and stirred. Pd(PPh₃)₄(tetrakis(triphenylphosphine)palladium(0)) 0.7 g (0.6 mmol) was added and refluxed for 12 hours. After completion of the reaction, the mixture was cooled to room temperature, the organic layer and the water layer were separated, and the organic layer was distilled under reduced pressure. The reduced-pressure distilled filtrate was extracted with chloroform and water. The residual moisture was removed from the organic layer using magnesium sulfate and was distilled under reduced pressure. The target material was separated from the filtrate through silica gel column chromatography to obtain the intermediate 10-a 11.6 g (24 mmol). (yield 75%)

### (2) An Intermediate 10

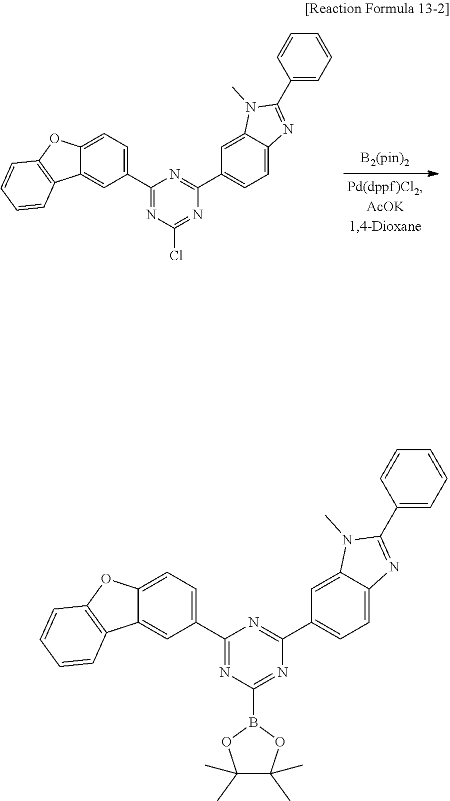 [SMILES: Cn1c(-c2ccccc2)nc2ccc(-c3nc(Cl)nc(-c4ccc5oc6ccccc6c5c4)n3)cc21 ; Cn1c(-c2ccccc2)nc2ccc(-c3nc(B4OC(C)(C)C(C)(C)O4)nc(-c4ccc5oc6ccccc6c5c4)n3)cc21]

In a nitrogen atmosphere, the intermediate 10-a 11.5 g (24 mmol), B₂(pin)₂ 6.6 g (26 mmol), Pd(dppf)Cl₂ 0.3 g (0.5 mmol), and potassium acetate (AcOK) 4.6 g (47 mmol) were dissolved in 1,4-dioxane 100 mL. The mixture was stirred and refluxed for 3 hours. The reaction solution was cooled to room temperature and filtered under reduced pressure with washing by MC. The filtrate was concentrated under reduced pressure and then filtered through a silica gel filter. The solid was precipitated using methanol to obtain the intermediate 10 11.6 g (20 mmol). (yield 85%)

### 14. Synthesis of the Compound 1-1

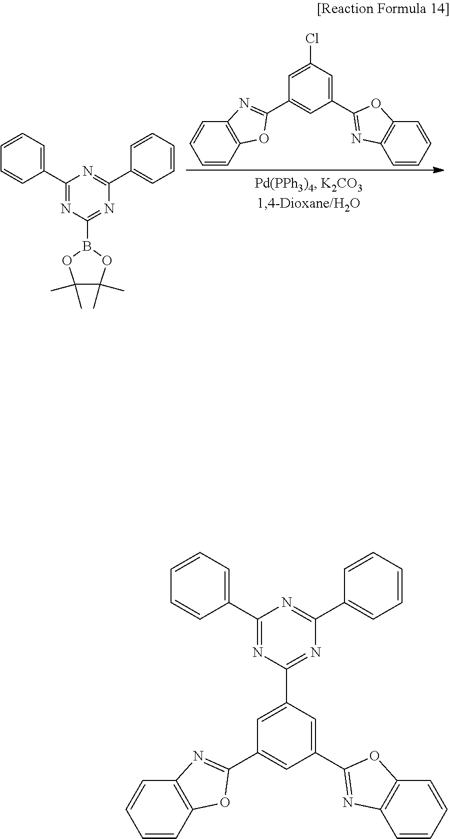

In a nitrogen atmosphere, the intermediate 1 3.6 g (10 mmol) and the intermediate A 3.8 g (11 mmol) were dissolved in 1,4-dioxane 100 mL. Potassium carbonate 2.9 g (21 mmol) was dissolved in distilled water 15 mL, added to the THF solution, and stirred. Pd(PPh₃)₄ (tetrakis(triphenylphosphine)palladium(0)) 0.2 g (0.2 mmol) was added and refluxed for 12 hours. After the reaction, it was cooled to room temperature, the organic layer and the water layer were separated, and the organic layer was distilled under reduced pressure. The reduced-pressure distilled filtrate was extracted with chloroform and water. The residual moisture was removed from the organic layer using magnesium sulfate and was distilled under reduced pressure. The target material was separated from the filtrate through silica gel column chromatography to obtain the compound 1-1 3.8 g (7 mmol). (yield 70%)

### 15. Synthesis of the Compound 1-10

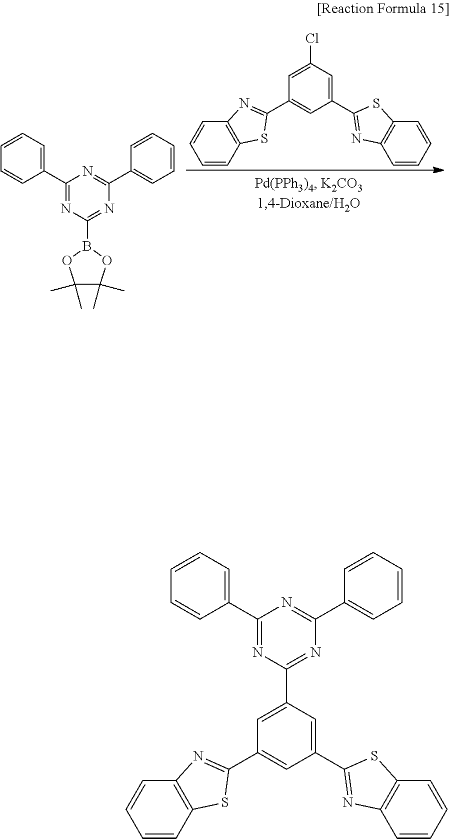

In a nitrogen atmosphere, the intermediate 1 3.6 g (10 mmol) and the intermediate B 4.2 g (11 mmol) were dissolved in 1,4-dioxane 100 mL. Potassium carbonate 2.9 g (21 mmol) was dissolved in distilled water 15 mL, added to the THF solution, and stirred. Pd(PPh₃)₄ (tetrakis(triphenylphosphine)palladium(0)) 0.2 g (0.2 mmol) was added and refluxed for 12 hours. After the reaction, it was cooled to room temperature, the organic layer and the water layer were separated, and the organic layer was distilled under reduced pressure. The reduced-pressure distilled filtrate was extracted with chloroform and water. The residual moisture was removed from the organic layer using magnesium sulfate and was distilled under reduced pressure. The target material was separated from the filtrate through silica gel column chromatography to obtain the compound 1-10 4.0 g (7 mmol). (yield 70%)

### 16. Synthesis of the Compound 1-57

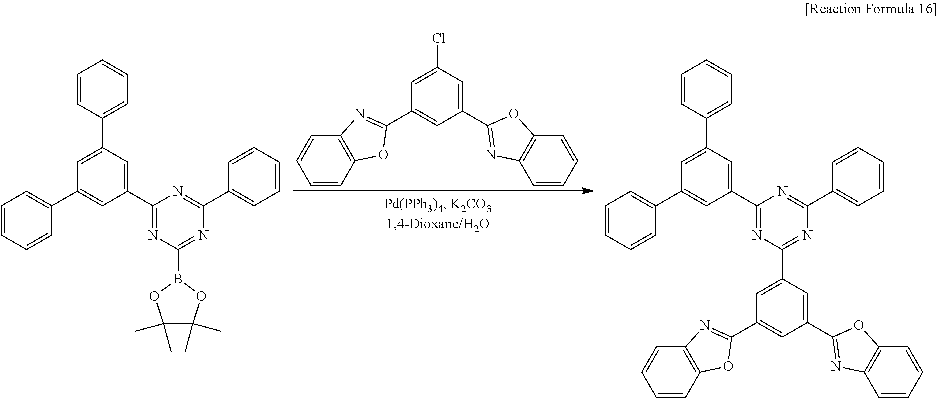

In a nitrogen atmosphere, the intermediate 2 5.0 g (10 mmol) and the intermediate A 3.7 g (11 mmol) were dissolved in 1,4-dioxane 100 mL. Potassium carbonate 2.8 g (21 mmol) was dissolved in distilled water 15 mL, added to the THF solution, and stirred. Pd(PPh₃)₄ (tetrakis(triphenylphosphine)palladium(0)) 0.2 g (0.2 mmol) was added and refluxed for 12 hours. After the reaction, it was cooled to room temperature, the organic layer and the water layer were separated, and the organic layer was distilled under reduced pressure. The reduced-pressure distilled filtrate was extracted with chloroform and water. The residual moisture was removed from the organic layer using magnesium sulfate and was distilled under reduced pressure. The target material was separated from the filtrate through silica gel column chromatography to obtain the compound 1-57 4.8 g (7 mmol). (yield 70%)

### 17. Synthesis of the Compound 1-43

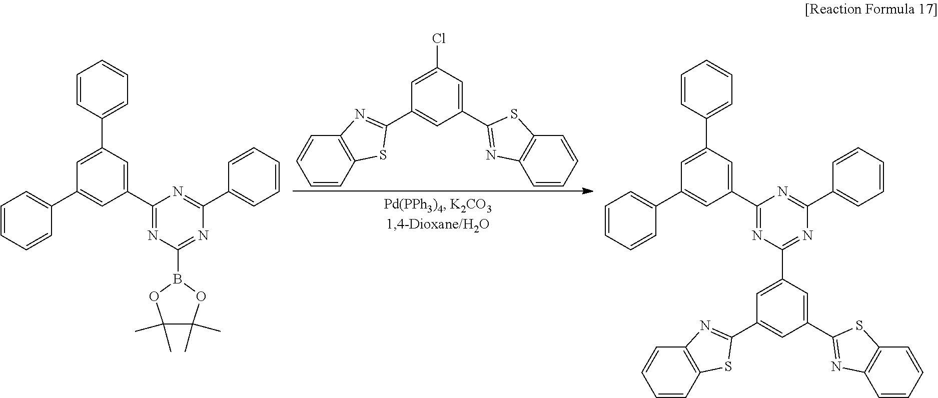

In a nitrogen atmosphere, the intermediate 2 5.0 g (10 mmol) and the intermediate B 4.1 g (11 mmol) were dissolved in 1,4-dioxane 100 mL. Potassium carbonate 2.8 g (21 mmol) was dissolved in distilled water 15 mL, added to the THF solution, and stirred. Pd(PPh₃)₄ (tetrakis(triphenylphosphine)palladium(0)) 0.2 g (0.2 mmol) was added and refluxed for 12 hours. After the reaction, it was cooled to room temperature, the organic layer and the water layer were separated, and the organic layer was distilled under reduced pressure. The reduced-pressure distilled filtrate was extracted with chloroform and water. The residual moisture was removed from the organic layer using magnesium sulfate and was distilled under reduced pressure. The target material was separated from the filtrate through silica gel column chromatography to obtain the compound 1-43 4.0 g (7 mmol). (yield 70%)

### 18. Synthesis of the Compound 3-28

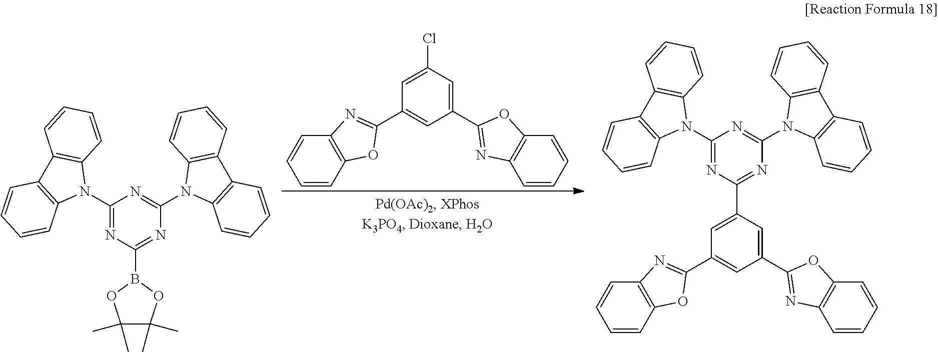

In a nitrogen atmosphere, the intermediate 3 5.5 g (10 mmol) and the intermediate A 3.9 g (11 mmol) were dissolved in 1,4-dioxane 100 mL. Potassium carbonate 3.0 g (21 mmol) was dissolved in distilled water 15 mL, added to the THF solution, and stirred. Pd(PPh₃)₄ (tetrakis(triphenylphosphine)palladium(0)) 0.2 g (0.2 mmol) was added and refluxed for 12 hours. After the reaction, it was cooled to room temperature, the organic layer and the water layer were separated, and the organic layer was distilled under reduced pressure. The reduced-pressure distilled filtrate was extracted with chloroform and water. The residual moisture was removed from the organic layer using magnesium sulfate and was distilled under reduced pressure. The target material was separated from the filtrate through silica gel column chromatography to obtain the compound 3-28 5.2 g (7 mmol). (yield 70%)

### 19. Synthesis of the Compound 3-36

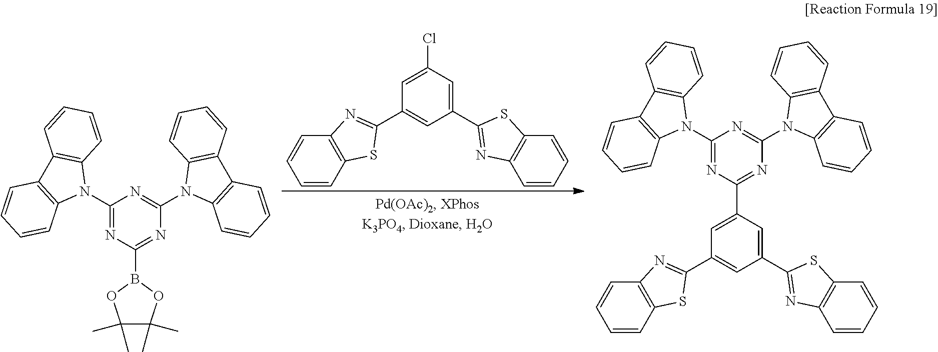

In a nitrogen atmosphere, the intermediate 3 5.4 g (10 mmol) and the intermediate B 4.2 g (11 mmol) were dissolved in 1,4-dioxane 100 mL. Potassium carbonate 2.9 g (21 mmol) was dissolved in distilled water 15 mL, added to the THF solution, and stirred. Pd(PPh₃)₄ (tetrakis(triphenylphosphine)palladium(0)) 0.2 g (0.2 mmol) was added and refluxed for 12 hours. After the reaction, it was cooled to room temperature, the organic layer and the water layer were separated, and the organic layer was distilled under reduced pressure. The reduced-pressure distilled filtrate was extracted with chloroform and water. The residual moisture was removed from the organic layer using magnesium sulfate and was distilled under reduced pressure. The target material was separated from the filtrate through silica gel column chromatography to obtain the compound 3-36 5.3 g (7 mmol). (yield 70%)

### 20. Synthesis of the Compound 3-29

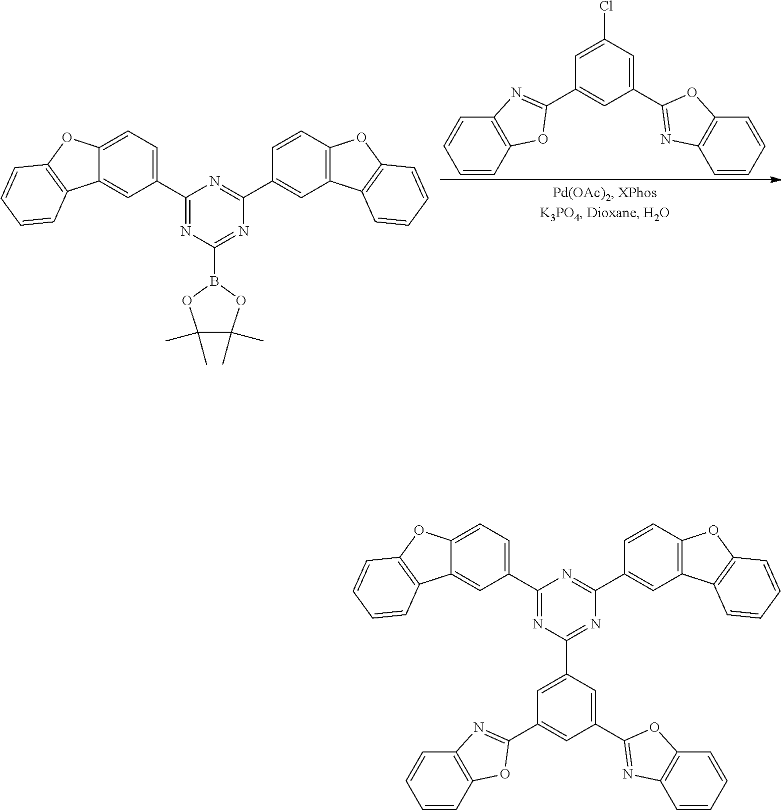

In a nitrogen atmosphere, the intermediate 4 5.4 g (10 mmol) and the intermediate A 3.8 g (11 mmol) were dissolved in 1,4-dioxane 100 mL. Potassium carbonate 2.9 g (21 mmol) was dissolved in distilled water 15 mL, added to the THF solution, and stirred. Pd(PPh₃)₄ (tetrakis(triphenylphosphine)palladium(0)) 0.2 g (0.2 mmol) was added and refluxed for 12 hours. After the reaction, it was cooled to room temperature, the organic layer and the water layer were separated, and the organic layer was distilled under reduced pressure. The reduced-pressure distilled filtrate was extracted with chloroform and water. The residual moisture was removed from the organic layer using magnesium sulfate and was distilled under reduced pressure. The target material was separated from the filtrate through silica gel column chromatography to obtain the compound 3-29 5.1 g (7 mmol). (yield 70%)

### 21. Synthesis of the Compound 3-37

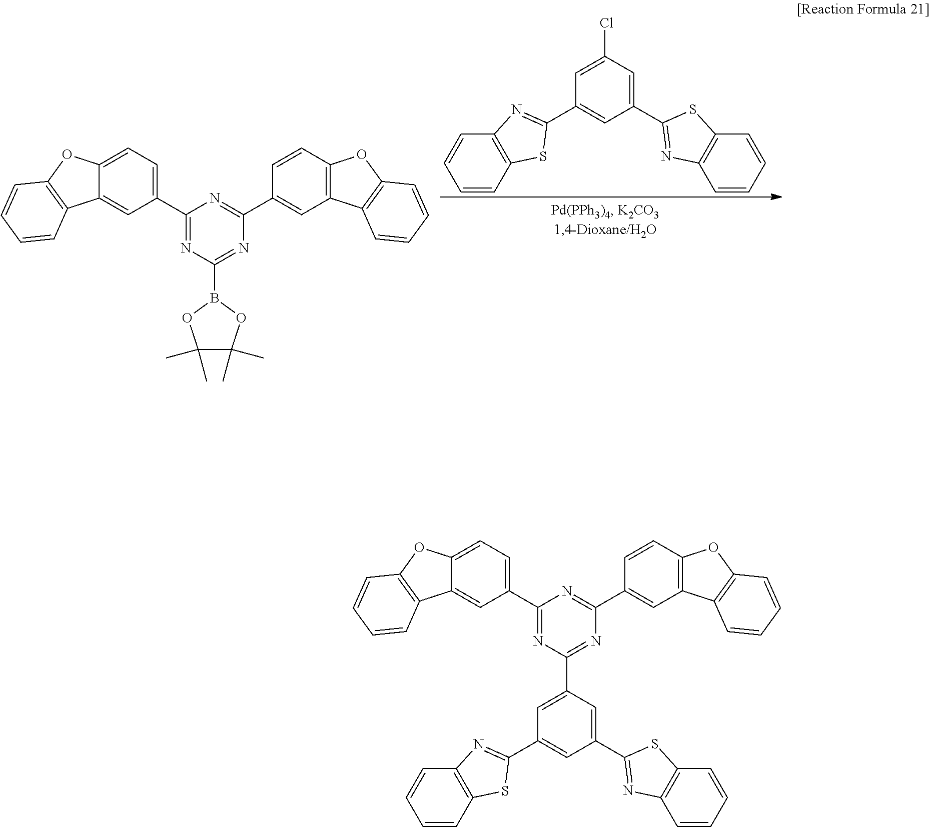

In a nitrogen atmosphere, the intermediate 4 5.4 g (10 mmol) and the intermediate B 4.2 g (11 mmol) were dissolved in 1,4-dioxane 100 mL. Potassium carbonate 2.9 g (21 mmol) was dissolved in distilled water 15 mL, added to the THF solution, and stirred. Pd(PPh₃)₄ (tetrakis(triphenylphosphine)palladium(0)) 0.2 g (0.2 mmol) was added and refluxed for 12 hours. After the reaction, it was cooled to room temperature, the organic layer and the water layer were separated, and the organic layer was distilled under reduced pressure. The reduced-pressure distilled filtrate was extracted with chloroform and water. The residual moisture was removed from the organic layer using magnesium sulfate and was distilled under reduced pressure. The target material was separated from the filtrate through silica gel column chromatography to obtain the compound 3-37 5.3 g (7 mmol). (yield 70%)

### 22. Synthesis of the Compound 2-62

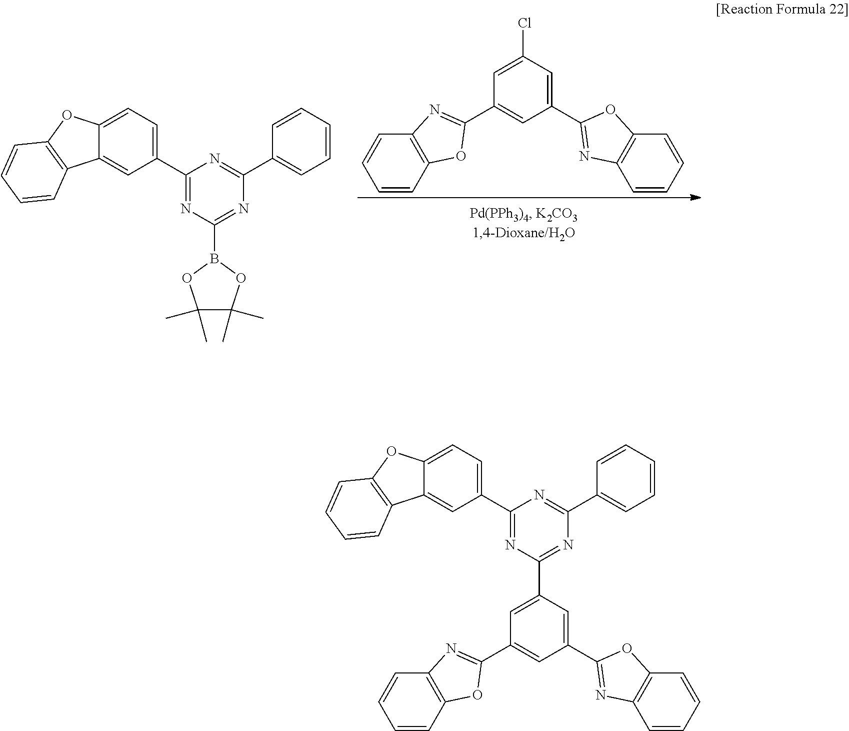

In a nitrogen atmosphere, the intermediate 5 4.5 g (10 mmol) and the intermediate A 3.8 g (11 mmol) were dissolved in 1,4-dioxane 100 mL. Potassium carbonate 2.9 g (21 mmol) was dissolved in distilled water 15 mL, added to the THF solution, and stirred. Pd(PPh₃)₄ (tetrakis(triphenylphosphine)palladium(0)) 0.2 g (0.2 mmol) was added and refluxed for 12 hours. After the reaction, it was cooled to room temperature, the organic layer and the water layer were separated, and the organic layer was distilled under reduced pressure. The reduced-pressure distilled filtrate was extracted with chloroform and water. The residual moisture was removed from the organic layer using magnesium sulfate and was distilled under reduced pressure. The target material was separated from the filtrate through silica gel column chromatography to obtain the compound 2-62 4.4 g (7 mmol). (yield 70%)

### 23. Synthesis of the Compound 3-39

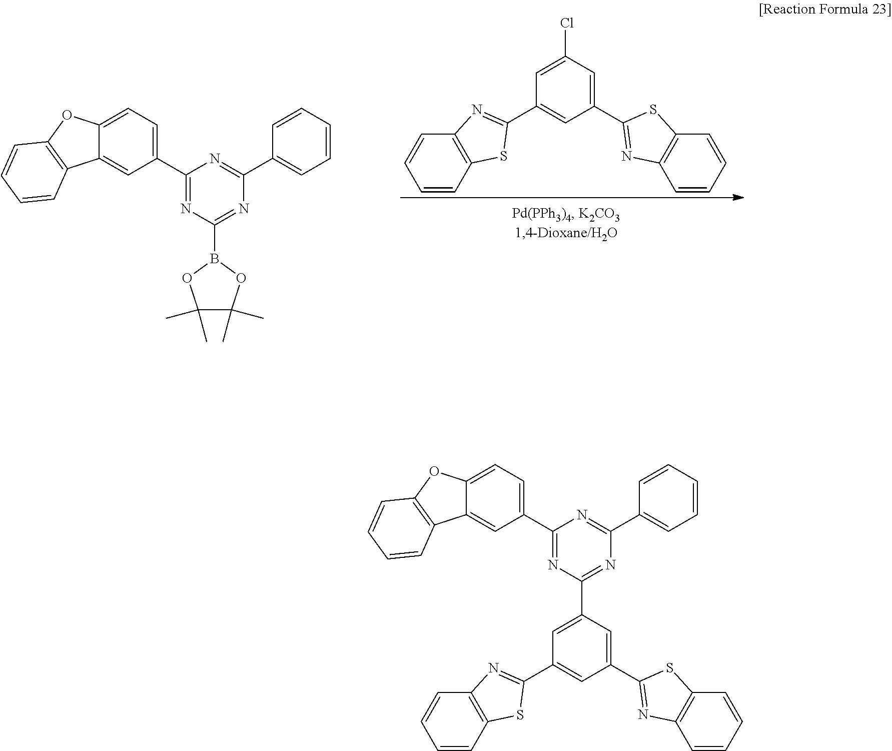 [SMILES: CC1(C)OB(c2nc(-c3ccccc3)nc(-c3ccc4oc5ccccc5c4c3)n2)OC1(C)C ; Clc1cc(-c2nc3ccccc3s2)cc(-c2nc3ccccc3s2)c1 ; c1ccc(-c2nc(-c3cc(-c4nc5ccccc5s4)cc(-c4nc5ccccc5s4)c3)nc(-c3ccc4oc5ccccc5c4c3)n2)cc1]

In a nitrogen atmosphere, the intermediate 5 4.5 g (10 mmol) and the intermediate B 4.2 g (11 mmol) were dissolved in 1,4-dioxane 100 mL. Potassium carbonate 2.9 g (21 mmol) was dissolved in distilled water 15 mL, added to the THF solution, and stirred. Pd(PPh₃)₄ (tetrakis(triphenylphosphine)palladium(0)) 0.2 g (0.2 mmol) was added and refluxed for 12 hours. After the reaction, it was cooled to room temperature, the organic layer and the water layer were separated, and the organic layer was distilled under reduced pressure. The reduced-pressure distilled filtrate was extracted with chloroform and water. The residual moisture was removed from the organic layer using magnesium sulfate and was distilled under reduced pressure. The target material was separated from the filtrate through silica gel column chromatography to obtain the compound 3-39 4.7 g (7 mmol). (yield 70%)

### 24. Synthesis of the Compound 2-1

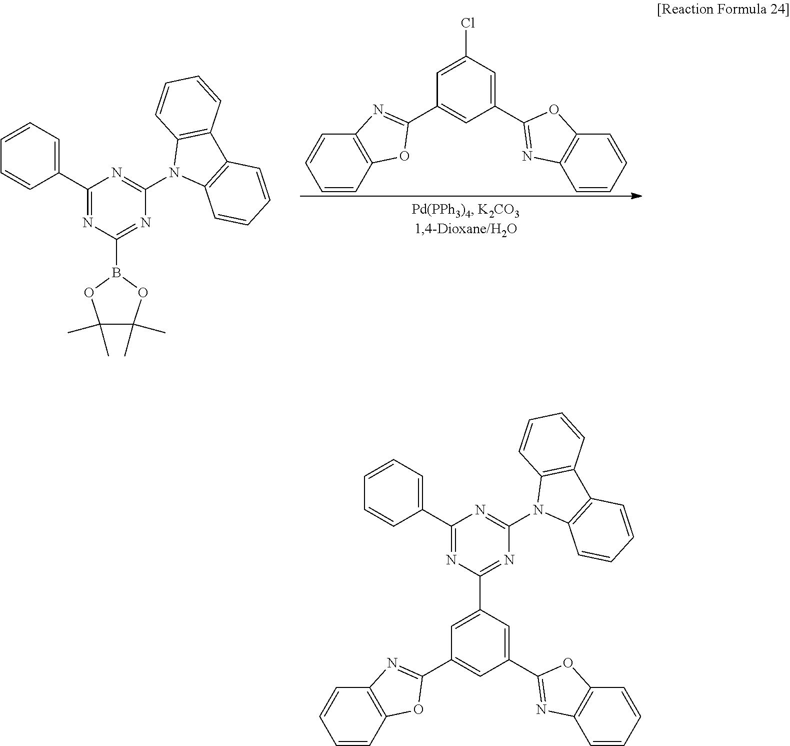

In a nitrogen atmosphere, the intermediate 6 4.5 g (10 mmol) and the intermediate A 3.8 g (11 mmol) were dissolved in 1,4-dioxane 100 mL. Potassium carbonate 2.9 g (21 mmol) was dissolved in distilled water 15 mL, added to the THF solution, and stirred. Pd(PPh₃)₄ (tetrakis(triphenylphosphine)palladium(0)) 0.2 g (0.2 mmol) was added and refluxed for 12 hours. After the reaction, it was cooled to room temperature, the organic layer and the water layer were separated, and the organic layer was distilled under reduced pressure. The reduced-pressure distilled filtrate was extracted with chloroform and water. The residual moisture was removed from the organic layer using magnesium sulfate and was distilled under reduced pressure. The target material was separated from the filtrate through silica gel column chromatography to obtain the compound 2-1 4.4 g (7 mmol). (yield 70%)

### 25. Synthesis of the Compound 3-23

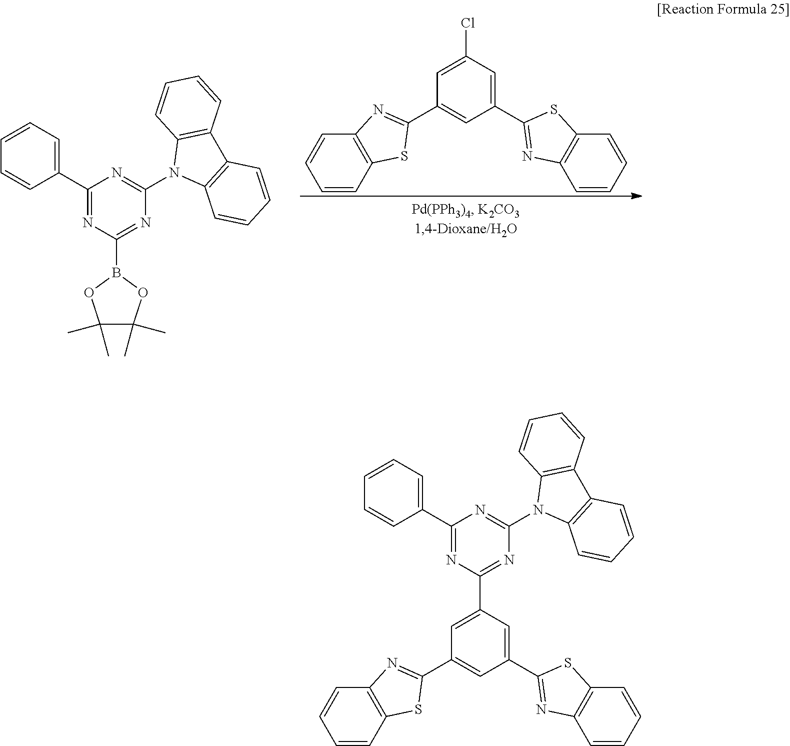

In a nitrogen atmosphere, the intermediate 6 4.5 g (10 mmol) and the intermediate B 4.2 g (11 mmol) were dissolved in 1,4-dioxane 100 mL. Potassium carbonate 2.9 g (21 mmol) was dissolved in distilled water 15 mL, added to the THF solution, and stirred. Pd(PPh₃)₄ (tetrakis(triphenylphosphine)palladium(0)) 0.2 g (0.2 mmol) was added and refluxed for 12 hours. After the reaction, it was cooled to room temperature, the organic layer and the water layer were separated, and the organic layer was distilled under reduced pressure. The reduced-pressure distilled filtrate was extracted with chloroform and water. The residual moisture was removed from the organic layer using magnesium sulfate and was distilled under reduced pressure. The target material was separated from the filtrate through silica gel column chromatography to obtain the compound 3-23 4.7 g (7 mmol). (yield 70%)

### 26. Synthesis of the Compound 1-27

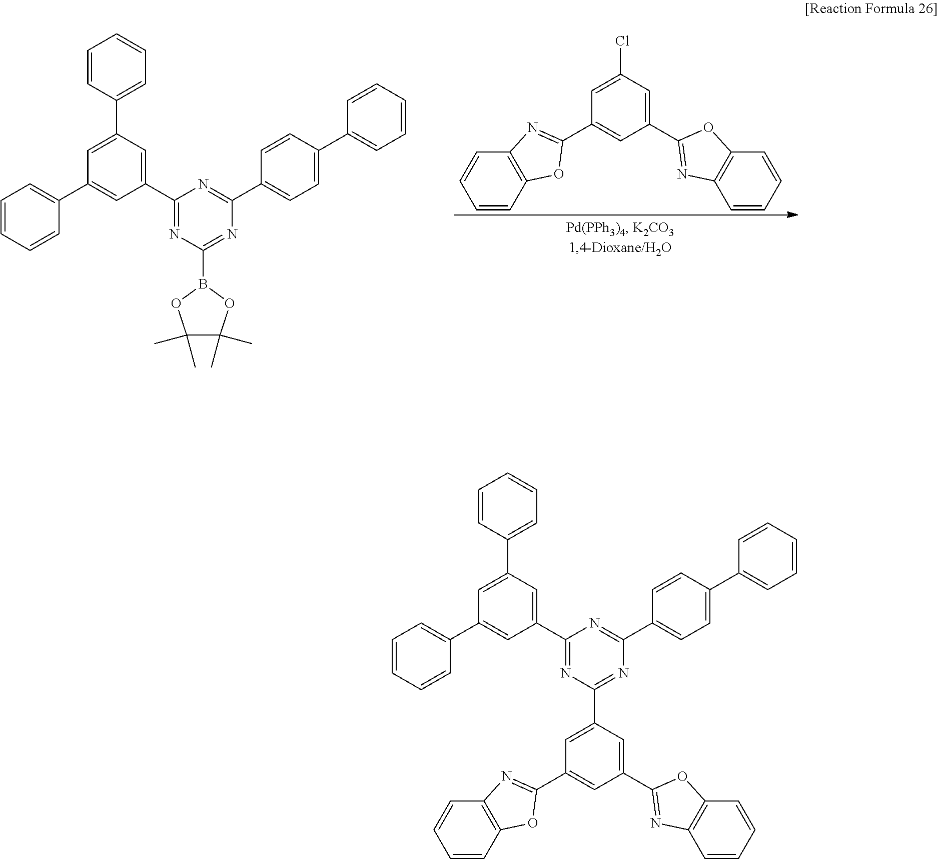 [SMILES: CC1(C)OB(c2nc(-c3ccc(-c4ccccc4)cc3)nc(-c3cc(-c4ccccc4)cc(-c4ccccc4)c3)n2)OC1(C)C ; Clc1cc(-c2nc3ccccc3o2)cc(-c2nc3ccccc3o2)c1 ; c1ccc(-c2ccc(-c3nc(-c4cc(-c5ccccc5)cc(-c5ccccc5)c4)nc(-c4cc(-c5nc6ccccc6o5)cc(-c5nc6ccccc6o5)c4)n3)cc2)cc1]

In a nitrogen atmosphere, the intermediate 7 5.9 g (10 mmol) and the intermediate A 3.8 g (11 mmol) were dissolved in 1,4-dioxane 100 mL. Potassium carbonate 2.9 g (21 mmol) was dissolved in distilled water 15 mL, added to the THF solution, and stirred. Pd(PPh₃)₄ (tetrakis(triphenylphosphine)palladium(0)) 0.2 g (0.2 mmol) was added and refluxed for 12 hours. After the reaction, it was cooled to room temperature, the organic layer and the water layer were separated, and the organic layer was distilled under reduced pressure. The reduced-pressure distilled filtrate was extracted with chloroform and water. The residual moisture was removed from the organic layer using magnesium sulfate and was distilled under reduced pressure. The target material was separated from the filtrate through silica gel column chromatography to obtain the compound 1-27 5.4 g (7 mmol). (yield 70%)

### 27. Synthesis of the Compound 3-40

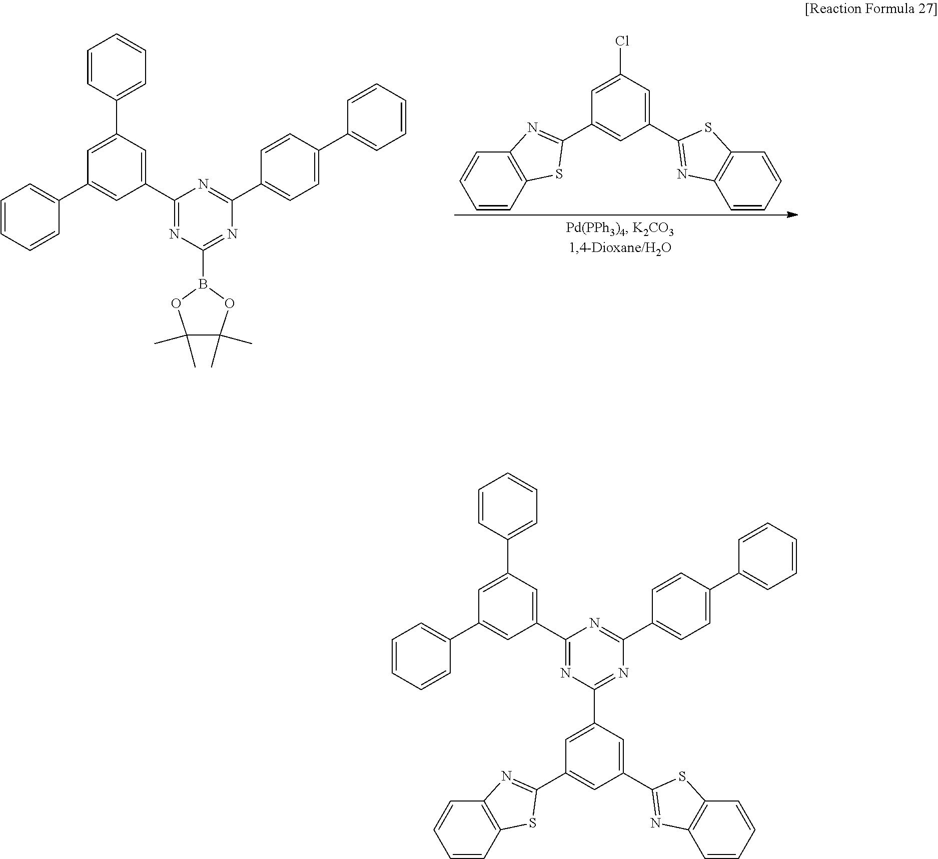 [SMILES: CC1(C)OB(c2nc(-c3ccc(-c4ccccc4)cc3)nc(-c3cc(-c4ccccc4)cc(-c4ccccc4)c3)n2)OC1(C)C ; Clc1cc(-c2nc3ccccc3s2)cc(-c2nc3ccccc3s2)c1 ; c1ccc(-c2ccc(-c3nc(-c4cc(-c5ccccc5)cc(-c5ccccc5)c4)nc(-c4cc(-c5nc6ccccc6s5)cc(-c5nc6ccccc6s5)c4)n3)cc2)cc1]

In a nitrogen atmosphere, the intermediate 7 5.9 g (10 mmol) and the intermediate B 4.2 g (11 mmol) were dissolved in 1,4-dioxane 100 mL. Potassium carbonate 2.9 g (21 mmol) was dissolved in distilled water 15 mL, added to the THF solution, and stirred. Pd(PPh₃)₄ (tetrakis(triphenylphosphine)palladium(0)) 0.2 g (0.2 mmol) was added and refluxed for 12 hours. After the reaction, it was cooled to room temperature, the organic layer and the water layer were separated, and the organic layer was distilled under reduced pressure. The reduced-pressure distilled filtrate was extracted with chloroform and water. The residual moisture was removed from the organic layer using magnesium sulfate and was distilled under reduced pressure. The target material was separated from the filtrate through silica gel column chromatography to obtain the compound 3-40 5.7 g (7 mmol). (yield 70%)

### 28. Synthesis of the Compound 1-46

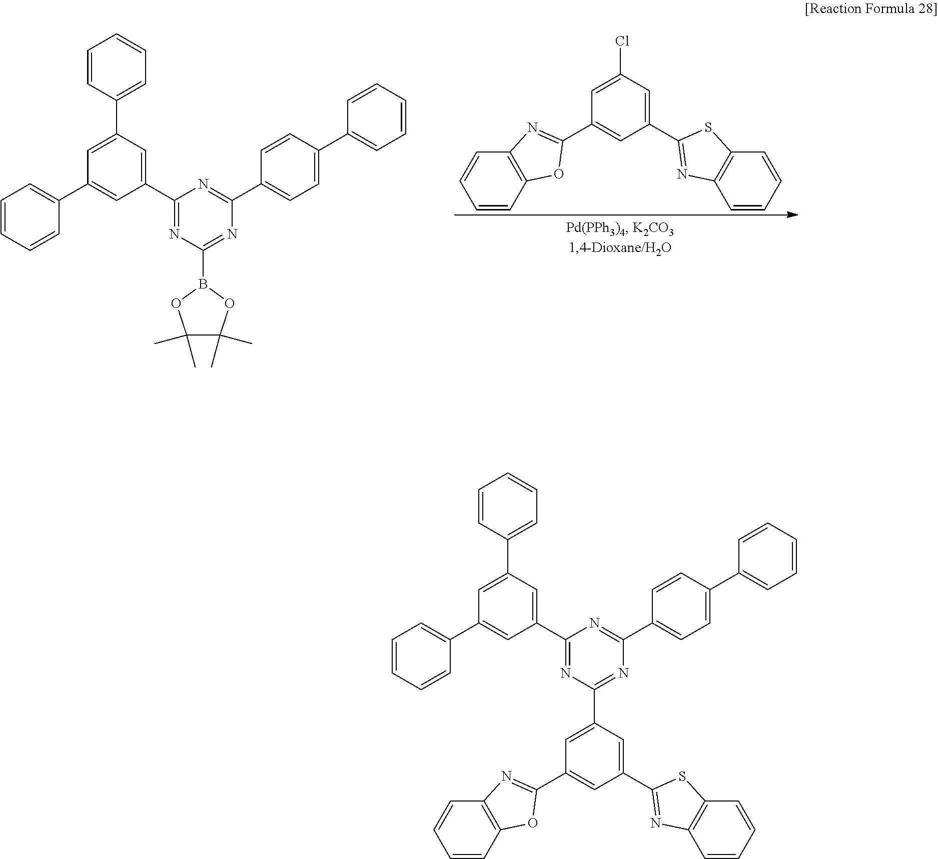 [SMILES: CC1(C)OB(c2nc(-c3ccc(-c4ccccc4)cc3)nc(-c3cc(-c4ccccc4)cc(-c4ccccc4)c3)n2)OC1(C)C ; Clc1cc(-c2nc3ccccc3o2)cc(-c2nc3ccccc3s2)c1 ; c1ccc(-c2ccc(-c3nc(-c4cc(-c5ccccc5)cc(-c5ccccc5)c4)nc(-c4cc(-c5nc6ccccc6o5)cc(-c5nc6ccccc6s5)c4)n3)cc2)cc1]

In a nitrogen atmosphere, the intermediate 7 5.9 g (10 mmol) and the intermediate C 4.0 g (11 mmol) were dissolved in 1,4-dioxane 100 mL. Potassium carbonate 2.9 g (21 mmol) was dissolved in distilled water 15 mL, added to the THF solution, and stirred. Pd(PPh₃)₄ (tetrakis(triphenylphosphine)palladium(0)) 0.2 g (0.2 mmol) was added and refluxed for 12 hours. After the reaction, it was cooled to room temperature, the organic layer and the water layer were separated, and the organic layer was distilled under reduced pressure. The reduced-pressure distilled filtrate was extracted with chloroform and water. The residual moisture was removed from the organic layer using magnesium sulfate and was distilled under reduced pressure. The target material was separated from the filtrate through silica gel column chromatography to obtain the compound 1-46 5.5 g (7 mmol). (yield 70%)

### 29. Synthesis of the Compound 2-12

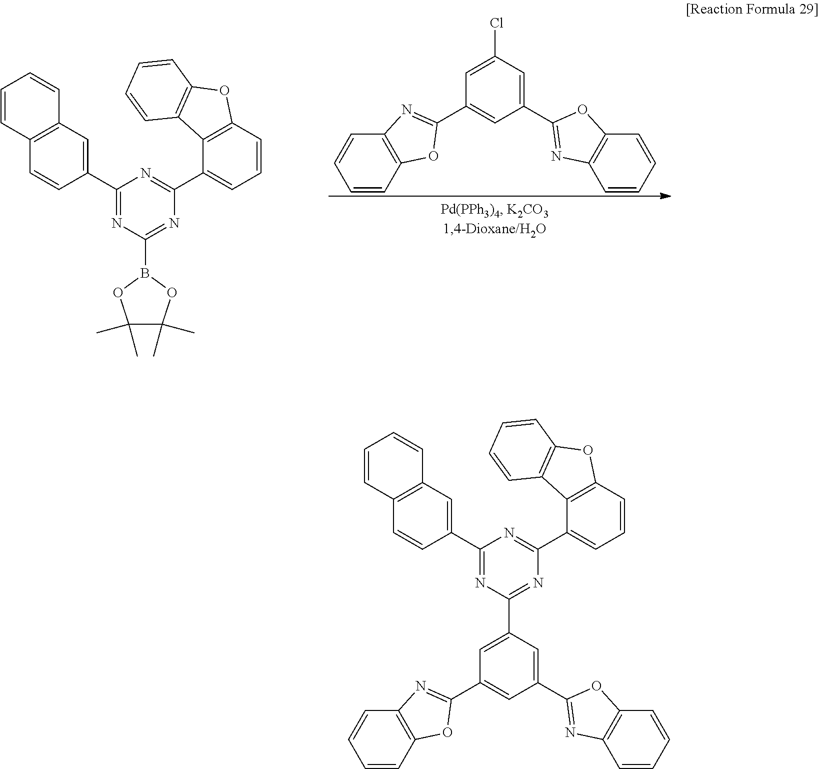 [SMILES: CC1(C)OB(c2nc(-c3ccc4ccccc4c3)nc(-c3cccc4oc5ccccc5c34)n2)OC1(C)C ; Clc1cc(-c2nc3ccccc3o2)cc(-c2nc3ccccc3o2)c1 ; c1ccc2cc(-c3nc(-c4cc(-c5nc6ccccc6o5)cc(-c5nc6ccccc6o5)c4)nc(-c4cccc5oc6ccccc6c45)n3)ccc2c1]

In a nitrogen atmosphere, the intermediate 8 5.0 g (10 mmol) and the intermediate A 3.8 g (11 mmol) were dissolved in 1,4-dioxane 100 mL. Potassium carbonate 2.9 g (21 mmol) was dissolved in distilled water 15 mL, added to the THF solution, and stirred. Pd(PPh₃)₄ (tetrakis(triphenylphosphine)palladium(0)) 0.2 g (0.2 mmol) was added and refluxed for 12 hours. After the reaction, it was cooled to room temperature, the organic layer and the water layer were separated, and the organic layer was distilled under reduced pressure. The reduced-pressure distilled filtrate was extracted with chloroform and water. The residual moisture was removed from the organic layer using magnesium sulfate and was distilled under reduced pressure. The target material was separated from the filtrate through silica gel column chromatography to obtain the compound 2-12 4.8 g (7 mmol). (yield 70%)

### 30. Synthesis of the Compound 3-41

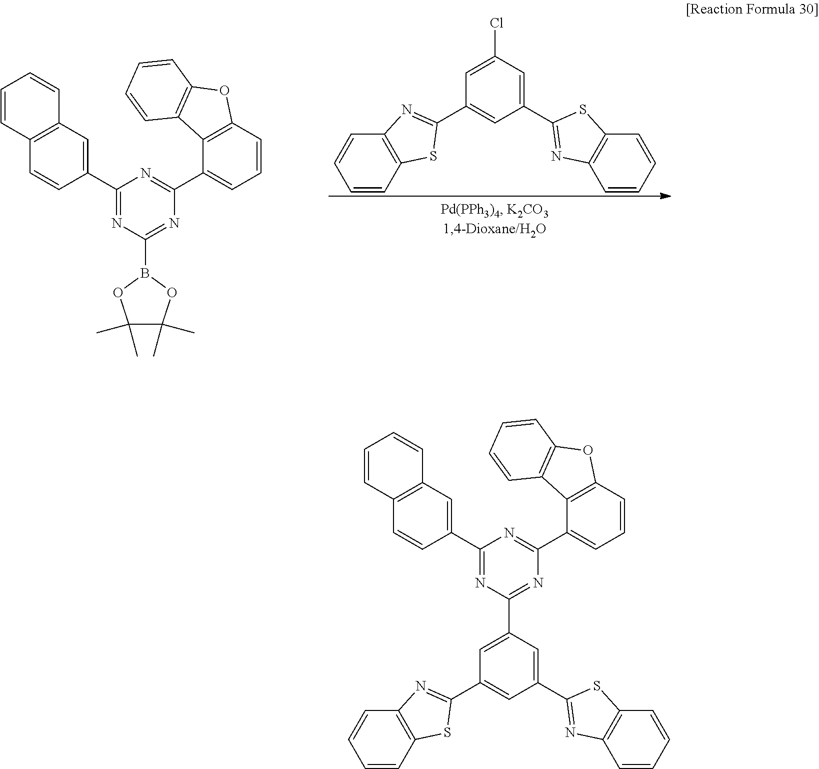 [SMILES: CC1(C)OB(c2nc(-c3ccc4ccccc4c3)nc(-c3cccc4oc5ccccc5c34)n2)OC1(C)C ; Clc1cc(-c2nc3ccccc3s2)cc(-c2nc3ccccc3s2)c1 ; c1ccc2cc(-c3nc(-c4cc(-c5nc6ccccc6s5)cc(-c5nc6ccccc6s5)c4)nc(-c4cccc5oc6ccccc6c45)n3)ccc2c1]

In a nitrogen atmosphere, the intermediate 8 5.0 g (10 mmol) and the intermediate B 4.2 g (11 mmol) were dissolved in 1,4-dioxane 100 mL. Potassium carbonate 2.9 g (21 mmol) was dissolved in distilled water 15 mL, added to the THF solution, and stirred. Pd(PPh₃)₄ (tetrakis(triphenylphosphine)palladium(0)) 0.2 g (0.2 mmol) was added and refluxed for 12 hours. After the reaction, it was cooled to room temperature, the organic layer and the water layer were separated, and the organic layer was distilled under reduced pressure. The reduced-pressure distilled filtrate was extracted with chloroform and water. The residual moisture was removed from the organic layer using magnesium sulfate and was distilled under reduced pressure. The target material was separated from the filtrate through silica gel column chromatography to obtain the compound 3-41 5.0 g (7 mmol). (yield 70%)

### 31. Synthesis of the Compound 2-39

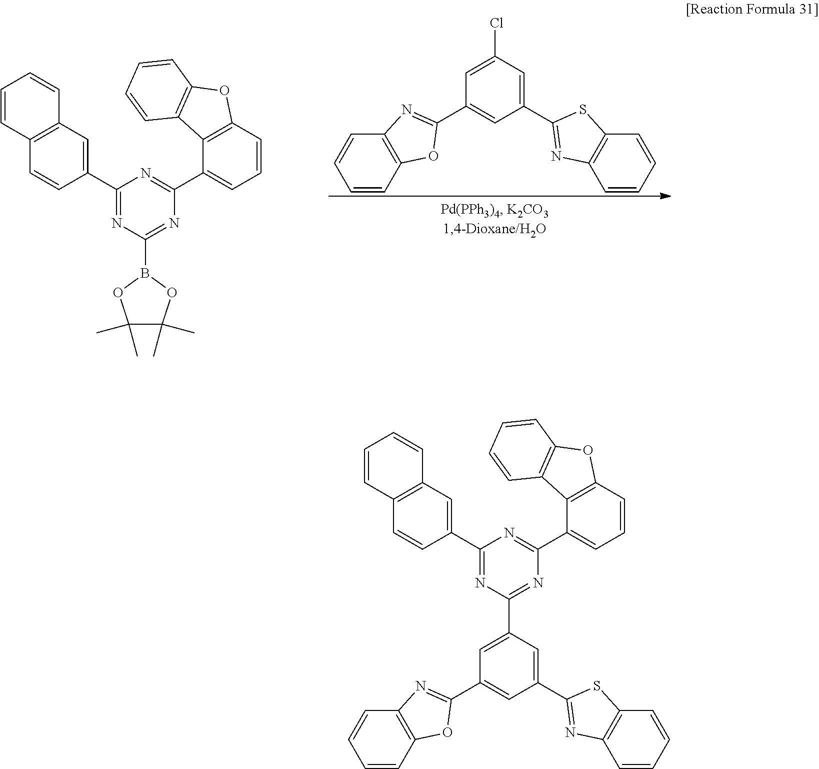 [SMILES: CC1(C)OB(c2nc(-c3ccc4ccccc4c3)nc(-c3cccc4oc5ccccc5c34)n2)OC1(C)C ; Clc1cc(-c2nc3ccccc3o2)cc(-c2nc3ccccc3s2)c1 ; c1ccc2cc(-c3nc(-c4cc(-c5nc6ccccc6o5)cc(-c5nc6ccccc6s5)c4)nc(-c4cccc5oc6ccccc6c45)n3)ccc2c1]

In a nitrogen atmosphere, the intermediate 8 5.0 g (10 mmol) and the intermediate C 4.0 g (11 mmol) were dissolved in 1,4-dioxane 100 mL. Potassium carbonate 2.9 g (21 mmol) was dissolved in distilled water 15 mL, added to the THF solution, and stirred. Pd(PPh₃)₄ (tetrakis(triphenylphosphine)palladium(0)) 0.2 g (0.2 mmol) was added and refluxed for 12 hours. After the reaction, it was cooled to room temperature, the organic layer and the water layer were separated, and the organic layer was distilled under reduced pressure. The reduced-pressure distilled filtrate was extracted with chloroform and water. The residual moisture was removed from the organic layer using magnesium sulfate and was distilled under reduced pressure. The target material was separated from the filtrate through silica gel column chromatography to obtain the compound 2-39 4.9 g (7 mmol). (yield 70%)

### 32. Synthesis of the Compound 3-4

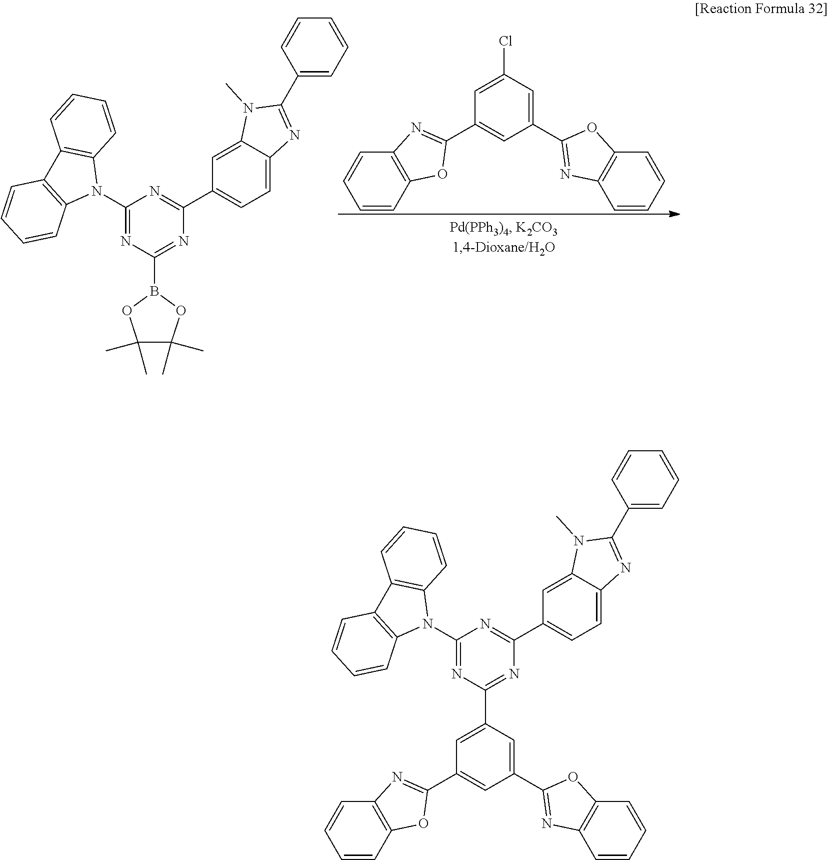 [SMILES: Cn1c(-c2ccccc2)nc2ccc(-c3nc(B4OC(C)(C)C(C)(C)O4)nc(-n4c5ccccc5c5ccccc54)n3)cc21 ; Clc1cc(-c2nc3ccccc3o2)cc(-c2nc3ccccc3o2)c1 ; Cn1c(-c2ccccc2)nc2ccc(-c3nc(-c4cc(-c5nc6ccccc6o5)cc(-c5nc6ccccc6o5)c4)nc(-n4c5ccccc5c5ccccc54)n3)cc21]

In a nitrogen atmosphere, the intermediate 9 5.8 g (10 mmol) and the intermediate A 3.8 g (11 mmol) were dissolved in 1,4-dioxane 100 mL. Potassium carbonate 2.9 g (21 mmol) was dissolved in distilled water 15 mL, added to the THF solution, and stirred. Pd(PPh₃)₄ (tetrakis(triphenylphosphine)palladium(0)) 0.2 g (0.2 mmol) was added and refluxed for 12 hours. After the reaction, it was cooled to room temperature, the organic layer and the water layer were separated, and the organic layer was distilled under reduced pressure. The reduced-pressure distilled filtrate was extracted with chloroform and water. The residual moisture was removed from the organic layer using magnesium sulfate and was distilled under reduced pressure. The target material was separated from the filtrate through silica gel column chromatography to obtain the compound 3-4 5.4 g (7 mmol). (yield 70%)

### 33. Synthesis of the Compound 3-42

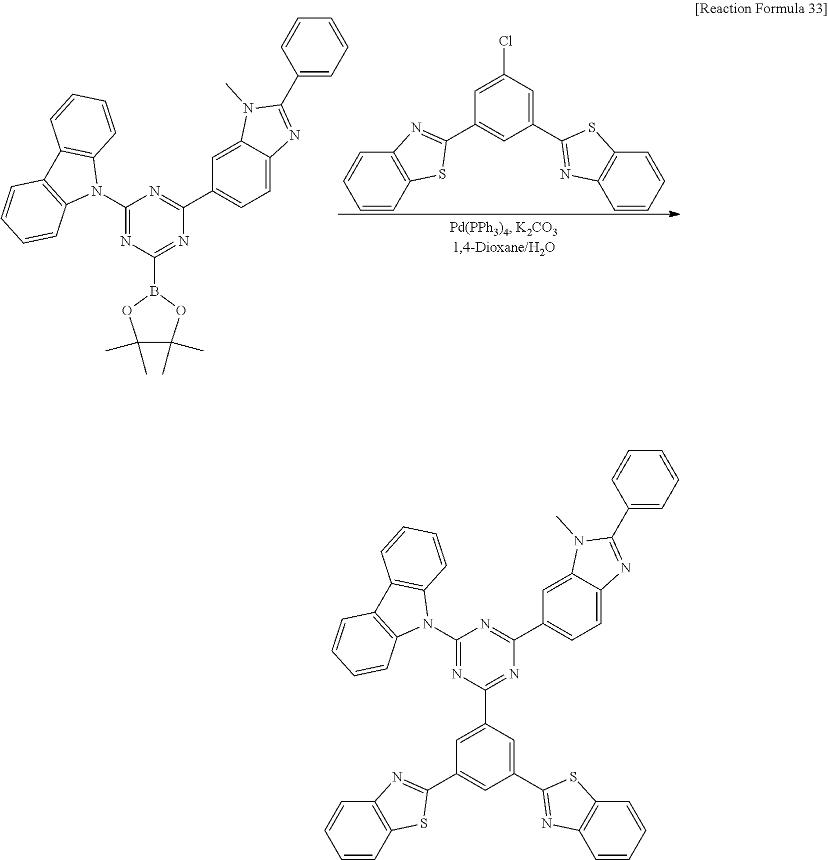 [SMILES: Cn1c(-c2ccccc2)nc2ccc(-c3nc(B4OC(C)(C)C(C)(C)O4)nc(-n4c5ccccc5c5ccccc54)n3)cc21 ; Clc1cc(-c2nc3ccccc3s2)cc(-c2nc3ccccc3s2)c1 ; Cn1c(-c2ccccc2)nc2ccc(-c3nc(-c4cc(-c5nc6ccccc6s5)cc(-c5nc6ccccc6s5)c4)nc(-n4c5ccccc5c5ccccc54)n3)cc21]

In a nitrogen atmosphere, the intermediate 9 5.8 g (10 mmol) and the intermediate B 4.2 g (11 mmol) were dissolved in 1,4-dioxane 100 mL. Potassium carbonate 2.9 g (21 mmol) was dissolved in distilled water 15 mL, added to the THF solution, and stirred. Pd(PPh₃)₄ (tetrakis(triphenylphosphine)palladium(0)) 0.2 g (0.2 mmol) was added and refluxed for 12 hours. After the reaction, it was cooled to room temperature, the organic layer and the water layer were separated, and the organic layer was distilled under reduced pressure. The reduced-pressure distilled filtrate was extracted with chloroform and water. The residual moisture was removed from the organic layer using magnesium sulfate and was distilled under reduced pressure. The target material was separated from the filtrate through silica gel column chromatography to obtain the compound 3-42 5.6 g (7 mmol). (yield 70%)

### 34. Synthesis of the Compound 3-43

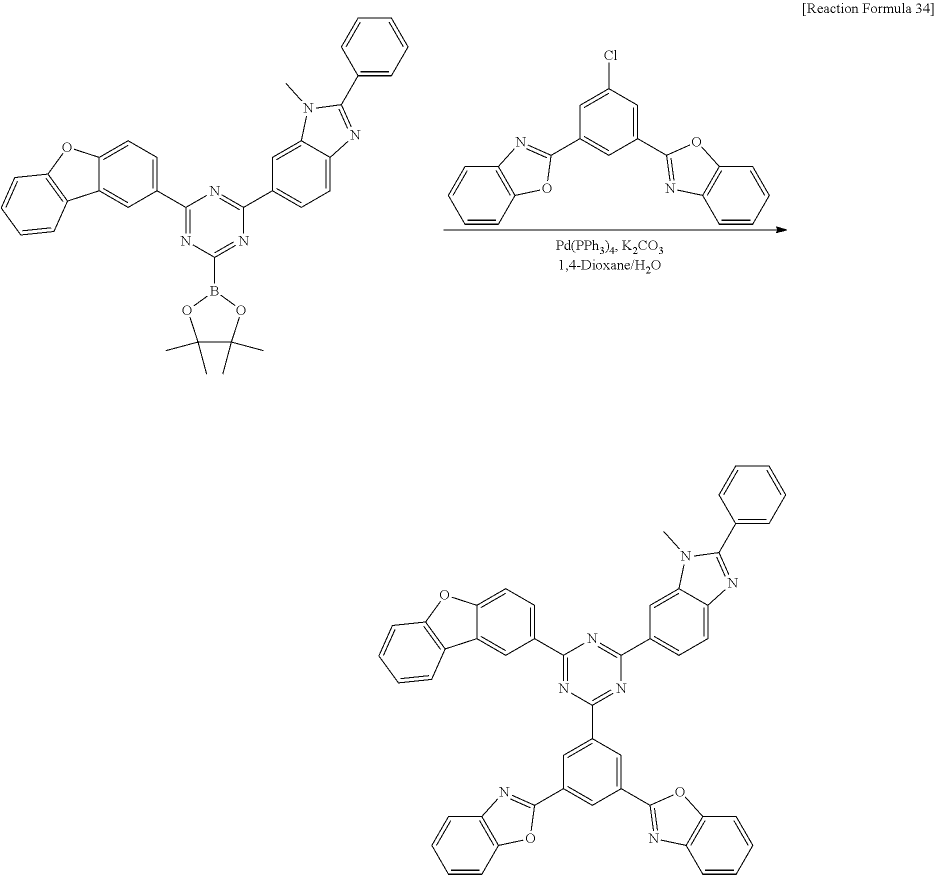 [SMILES: Cn1c(-c2ccccc2)nc2ccc(-c3nc(B4OC(C)(C)C(C)(C)O4)nc(-c4ccc5oc6ccccc6c5c4)n3)cc21 ; Clc1cc(-c2nc3ccccc3o2)cc(-c2nc3ccccc3o2)c1 ; Cn1c(-c2ccccc2)nc2ccc(-c3nc(-c4cc(-c5nc6ccccc6o5)cc(-c5nc6ccccc6o5)c4)nc(-c4ccc5oc6ccccc6c5c4)n3)cc21]

In a nitrogen atmosphere, the intermediate 10 5.8 g (10 mmol) and the intermediate A 3.8 g (11 mmol) were dissolved in 1,4-dioxane 100 mL. Potassium carbonate 2.9 g (21 mmol) was dissolved in distilled water 15 mL, added to the THF solution, and stirred. Pd(PPh₃)₄ (tetrakis(triphenylphosphine)palladium(0)) 0.2 g (0.2 mmol) was added and refluxed for 12 hours. After the reaction, it was cooled to room temperature, the organic layer and the water layer were separated, and the organic layer was distilled under reduced pressure. The reduced-pressure distilled filtrate was extracted with chloroform and water. The residual moisture was removed from the organic layer using magnesium sulfate and was distilled under reduced pressure. The target material was separated from the filtrate through silica gel column chromatography to obtain the compound 3-43 5.4 g (7 mmol). (yield 70%)

### 35. Synthesis of the Compound 3-38

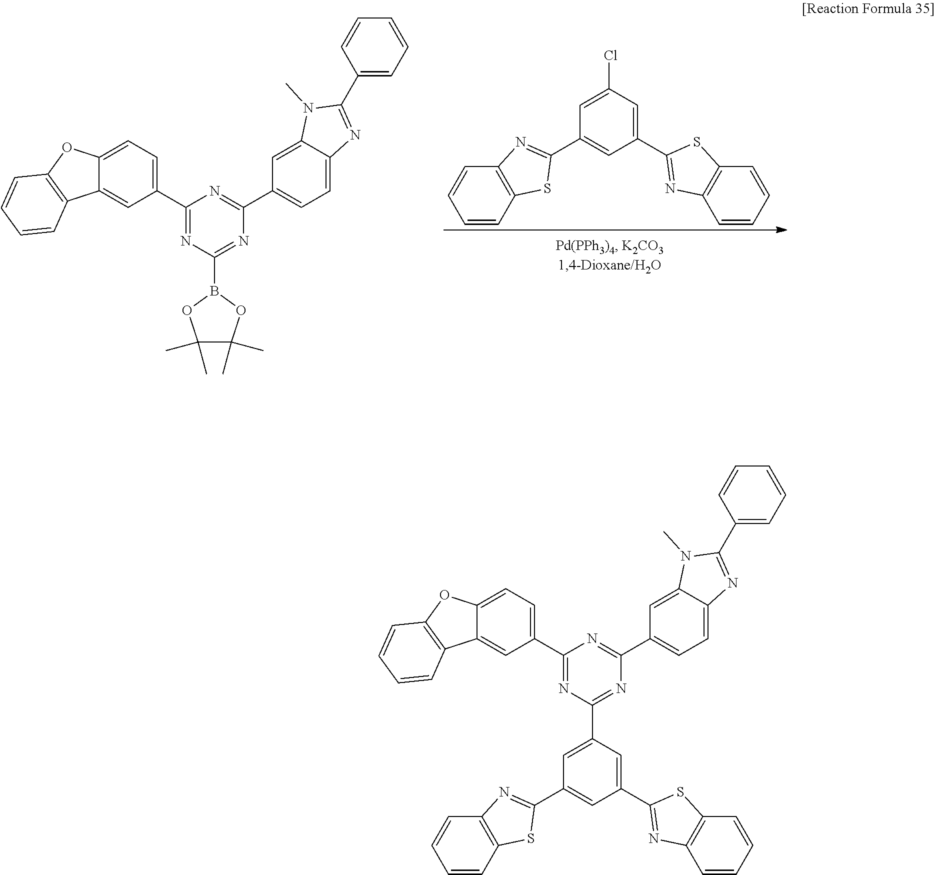 [SMILES: Cn1c(-c2ccccc2)nc2ccc(-c3nc(B4OC(C)(C)C(C)(C)O4)nc(-c4ccc5oc6ccccc6c5c4)n3)cc21 ; Clc1cc(-c2nc3ccccc3s2)cc(-c2nc3ccccc3s2)c1 ; Cn1c(-c2ccccc2)nc2ccc(-c3nc(-c4cc(-c5nc6ccccc6s5)cc(-c5nc6ccccc6s5)c4)nc(-c4ccc5oc6ccccc6c5c4)n3)cc21]

In a nitrogen atmosphere, the intermediate 10 5.8 g (10 mmol) and the intermediate B 4.2 g (11 mmol) were dissolved in 1,4-dioxane 100 mL. Potassium carbonate 2.9 g (21 mmol) was dissolved in distilled water 15 mL, added to the THF solution, and stirred. Pd(PPh₃)₄(tetrakis(triphenylphosphine)palladium(0)) 0.2 g (0.2 mmol) was added and refluxed for 12 hours. After the reaction, it was cooled to room temperature, the organic layer and the water layer were separated, and the organic layer was distilled under reduced pressure. The reduced-pressure distilled filtrate was extracted with chloroform and water. The residual moisture was removed from the organic layer using magnesium sulfate and was distilled under reduced pressure. The target material was separated from the filtrate through silica gel column chromatography to obtain the compound 3-38 5.6 g (7 mmol). (yield 70%)

### <OLED and Organic Light Emitting Device>

The OLED of the present disclosure may include the organic compound of the present disclosure. The OLED may be included in an organic light emitting display device or an organic light emitting lighting device. The explanation below is focused on an example of an organic light emitting display device including the OLED of the present disclosure.

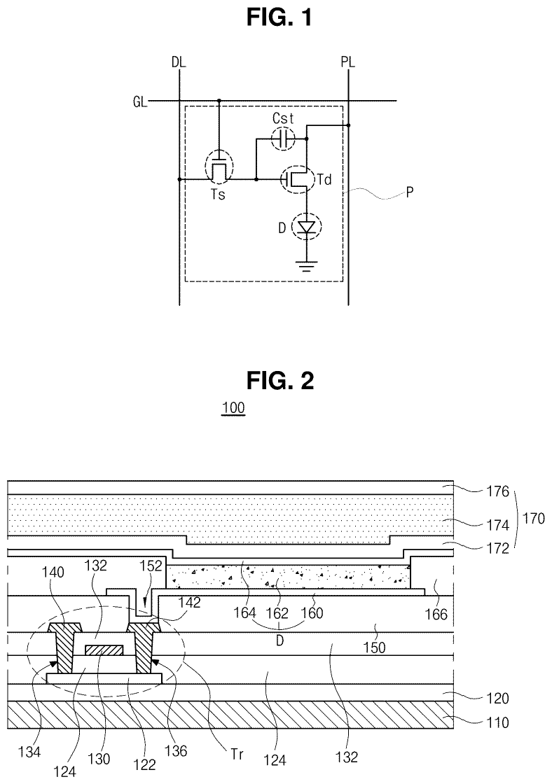

FIG. 1 illustrates a schematic circuit diagram for an organic light emitting display device according to an example embodiment of the present disclosure.

As illustrated in FIG. 1, a gate line GL and a data line DL, which may cross each other to define a pixel region P, and a power line PL may be formed in an organic light display device. A switching thin film transistor (TFT) Ts, a driving thin film transistor (TFT) Td, a storage capacitor Cst, and an OLED D may be formed in the pixel region P. The pixel region P may include a red pixel region, a green pixel region, and a blue pixel region. In addition, the pixel region P may further include a white pixel region.

The switching thin film transistor Ts may be connected to the gate line GL and the data line DL, and the driving thin film transistor Td and the storage capacitor Cst may be connected between the switching thin film transistor Ts and the power line PL. The OLED D may be connected to the driving thin film transistor Td. When the switching thin film transistor Ts is turned on by the gate signal applied through the gate line GL, the data signal applied through the data line DL may be applied to a gate electrode of the driving thin film transistor Td and one electrode of the storage capacitor Cst through the switching thin film transistor Ts.

The driving thin film transistor Td may be turned on by the data signal applied to the gate electrode so that a current proportional to the data signal may be supplied from the power line PL to the OLED D through the driving thin film transistor Td. The OLED D may emit light having a luminance proportional to the current flowing through the driving thin film transistor Td. In this case, the storage capacitor Cst may be charged with a voltage proportional to the data signal so that the voltage of the gate electrode in the driving thin film transistor Td may be kept constant or similar during one frame. Therefore, the organic light emitting display device can display a desired image.

FIG. 2 illustrates a schematic cross-sectional view of an organic light emitting display device according to a first embodiment of the present disclosure.

As illustrated in FIG. 2, the organic light emitting display device 100 may include a substrate 110, a TFT Tr, and an OLED D connected to the TFT Tr. For example, the organic light emitting display device 100 may include a red pixel region, a green pixel region, and a blue pixel region, and the OLED D may be disposed in each of the red, green and blue pixel regions. The organic light emitting display device 100 may further include a yellow-green pixel region, and the OLED D may be disposed in the yellow-green pixel region. For example, the OLEDs D emitting red light, green light, blue light, yellow-green light may be provided in the red, green, blue and yellow-green pixel regions, respectively.

The substrate 110 may be a glass substrate or a flexible substrate. For example, the flexible substrate may be a polyimide (PI) substrate, a polyethersulfone (PES) substrate, a polyethylenenaphthalate (PEN) substrate, a polyethylene terephthalate (PET) substrate or a polycarbonate (PC) substrate.

A buffer layer 120 may be formed on the substrate, and the TFT Tr may be formed on the buffer layer 120. The buffer layer 120 may be formed of an inorganic insulating material, e.g., silicon oxide or silicon nitride. The buffer layer 120 may have a multi-layered structure including a first layer of silicon oxide and a second layer of silicon nitride. The buffer layer 120 may be omitted, and the TFT Tr may be disposed on the substrate 110.

A semiconductor layer 122 may be formed on the buffer layer 120. The semiconductor layer 122 may include an oxide semiconductor material or polycrystalline silicon.

When the semiconductor layer 122 includes the oxide semiconductor material, a light-shielding pattern (not shown) may be formed under the semiconductor layer 122. The light to the semiconductor layer 122 may be shielded or blocked by the light-shielding pattern such that thermal degradation of the semiconductor layer 122 can be prevented or reduced. On the other hand, when the semiconductor layer 122 includes polycrystalline silicon, impurities may be doped into both sides of the semiconductor layer 122.

A gate insulating layer 124 may be formed on the semiconductor layer 122. The gate insulating layer 124 may be formed of an inorganic insulating material such as silicon oxide or silicon nitride.

A gate electrode 130, which may be formed of a conductive material, e.g., metal, may be formed on the gate insulating layer 124 to correspond to a center of the semiconductor layer 122.

In FIG. 2, the gate insulating layer 124 may be formed on an entire surface of the substrate 110. Alternatively, the gate insulating layer 124 may be patterned to have the same shape as the gate electrode 130. However, embodiments of the present disclosure are not limited to such examples.

An interlayer insulating layer 132, which may be formed of an insulating material, may be formed on the gate electrode 130. The interlayer insulating layer 132 may be formed of an inorganic insulating material, e.g., silicon oxide or silicon nitride, or an organic insulating material, e.g., benzocyclobutene or photo-acryl.

The interlayer insulating layer 132 may include first and second contact holes 134 and 136 exposing both sides of the semiconductor layer 122. The first and second contact holes 134 and 136 may not cover a portion of the surface of the semiconductor layer 122 that is nearer to the opposing ends than to a center of the semiconductor layer 122. The first and second contact holes 134 and 136 may be positioned at both sides of the gate electrode 130 to be spaced apart from the gate electrode 130.

The first and second contact holes 134 and 136 may be formed through the interlayer insulating layer 132 and the gate insulating layer 124. Alternatively, when the gate insulating layer 124 is patterned to have the same shape as the gate electrode 130, the first and second contact holes 134 and 136 may be formed only through the interlayer insulating layer 132. However, embodiments of the present disclosure are not limited to such examples.

A source electrode 140 and a drain electrode 142, which may be formed of a conductive material, e.g., metal, may be formed on the interlayer insulating layer 132.

The source electrode 140 and the drain electrode 142 may be spaced apart from each other with respect to the gate electrode 130 and may contact both sides of the semiconductor layer 122 through the first and second contact holes 134 and 136, respectively.

The semiconductor layer 122, the gate electrode 130, the source electrode 140, and the drain electrode 142 may constitute the TFT Tr. The TFT Tr may serve as a driving element. For example, the TFT Tr may correspond to the driving TFT Td (of FIG. 1).

In the TFT Tr, the gate electrode 130, the source electrode 140, and the drain electrode 142 may be positioned on the semiconductor layer 122. For example, the TFT Tr may have a coplanar structure.

Alternatively, in the TFT Tr, the gate electrode may be positioned under the semiconductor layer, and the source and drain electrodes may be positioned on the semiconductor layer such that the TFT Tr may have an inverted staggered structure. In this instance, the semiconductor layer may include amorphous silicon. However, embodiments of the present disclosure are not limited to such examples.

Although not shown, the gate line and the data line may cross each other to define the pixel region, and the switching TFT may be formed to be connected to the gate and data lines. The switching TFT may be connected to the TFT Tr as the driving element.

In addition, the power line, which may be formed to be parallel to and spaced apart from one of the gate and data lines, and the storage capacitor for maintaining the voltage of the gate electrode of the TFT Tr in one frame may be further formed.

A planarization layer (or a passivation layer) 150, which may include a drain contact hole 152 exposing the drain electrode 142 of the TFT Tr, may be formed to cover the TFT Tr. The drain contact hole 152 may not cover the drain electrode 142.

A first electrode 160, which may be connected to the drain electrode 142 of the TFT Tr through the drain contact hole 152, may be separately formed in each pixel region and on the planarization layer 150.

The first electrode 160 may be an anode and may include a transparent conductive oxide material layer formed of a conductive material, e.g., a transparent conductive oxide (TCO), having a relatively high work function. For example, the transparent conductive oxide material layer of the first electrode 160 may include at least one of indium-tin-oxide (ITO) indium-zinc-oxide (IZO), indium-tin-zinc oxide; ITZO), tin oxide (SnO), zinc oxide (ZnO), indium-copper-oxide (ICO) and Al:ZnO (AZO).

When the organic light emitting display device 100 is operated as a bottom-emission type, the first electrode 160 may have a single-layered structure of the transparent conductive oxide material layer.

Alternatively, when the organic light emitting display device 100 is operated as a top-emission type, the first electrode 160 may further include a reflection layer to have a double-layered structure or a triple-layered structure. For example, the reflection layer may be formed of silver (Ag) or aluminium-palladium-copper (APC) alloy. In the top-emission type organic light emitting display device 100, the first electrode 160 may have a double-layered structure of Ag/ITO or APC/ITO or a triple-layered structure of ITO/Ag/ITO or ITO/APC/ITO. However, embodiments of the present disclosure are not limited to such examples.

A bank layer 166 may be formed on the planarization layer 150 to cover an edge of the first electrode 160. For example, the bank layer 166 may be positioned at a boundary of the pixel region and exposes a center of the first electrode 160 in the pixel region.

An organic light emitting layer 162 is disposed on the first electrode 160 and includes the organic compound of the present disclosure.

The organic light emitting layer 162 may include one emitting part including an emitting material layer (EML) and a functional layer. Alternatively, the organic light emitting layer 162 may include a plurality of emitting parts and each emitting part may include the EML and the functional layer. In addition, the organic light emitting layer 162 may further include a charge generation layer between adjacent emitting parts.

The functional layer may include at least one of an electron transporting layer (HTL) and a hole blocking layer (HBL).

The emitting part or each of the emitting parts may further include at least one of a hole injection layer (HIL), a hole transporting layer (HTL), an electron blocking layer (EBL), and an electron injection layer (EIL).

A second electrode 164 may be formed over the substrate 110 where the organic light emitting layer 162 is formed. The second electrode 164 may cover an entire surface of the display area and may be formed of a conductive material having a relatively low work function to serve as a cathode. For example, the second electrode 164 may be formed of aluminium (Al), magnesium (Mg), calcium (Ca), silver (Ag) or their alloy or a combination thereof. In the top-emission type organic light emitting display device 100, the second electrode 164 may have a thin profile (small thickness) to provide a light transmittance property (or a semi-transmittance property).

The first electrode 160, the organic light emitting layer 162, and the second electrode 164 may constitute the OLED D.

An encapsulation layer (e.g., an encapsulation film) 170 may be formed on the second electrode 164 to prevent penetration of moisture into the OLED D. The encapsulation layer 170 may include a first inorganic insulating layer 172, an organic insulating layer 174, and a second inorganic insulating layer 176 sequentially stacked. However, embodiments of the present disclosure are not limited to such examples. The encapsulation layer 170 may be omitted.

In the bottom-emission type organic light emitting display device 100, a metal plate may be further disposed on the encapsulation layer 170.

The organic light emitting display device 100 may further include a color filter layer corresponding to the red, green and blue pixel regions. The color filter layer may include red, green and blue color filter patterns respectively corresponding to the red, green and blue pixel regions. When the organic light emitting display device 100 includes the color filter layer, a color purity of the organic light emitting display device 100 may be improved.

In the bottom-emission type organic light emitting display device 100, the color filter layer may be positioned between the OLED D and the substrate 100, e.g., between the interlayer insulating layer 132 and the planarization layer 150. Alternatively, in the top-emission type organic light emitting display device 100, the color filter layer may be positioned over the OLED D, e.g., over the second electrode 164 or the encapsulation layer 170.

The organic light emitting display device 100 may further include a polarization plate (not shown) for reducing an ambient light reflection. For example, the polarization plate may be a circular polarization plate. In the bottom-emission type organic light emitting display device 100, the polarization plate may be disposed under the substrate 110. In the top-emission type organic light emitting display device 100, the polarization plate may be disposed on or over the encapsulation layer 170.

In addition, in the top-emission type organic light emitting display device 100, a cover window (not shown) may be attached to the encapsulation layer 170 or the polarization plate. In this instance, the substrate 110 and the cover window may have a flexible property such that a flexible organic light emitting display device may be provided.

Moreover, the organic light emitting display device 100 may further include a touch layer or a touch panel. The touch layer or the touch panel may be disposed over the OLED D, e.g., between the OLED D and the cover window, or under the substrate 100.

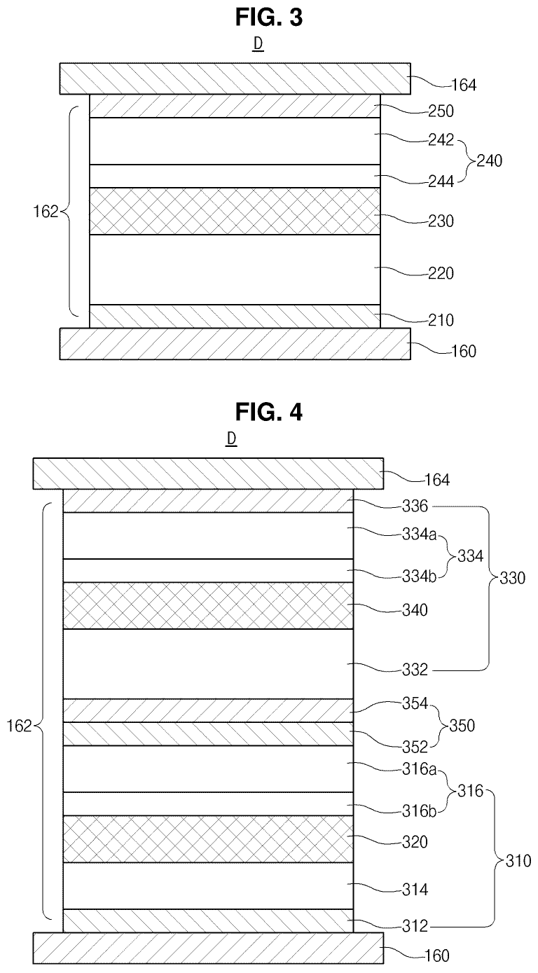

FIG. 3 illustrates a schematic cross-sectional view of an OLED according to a second embodiment of the present disclosure.

As illustrated in FIG. 3, an OLED D may include first and second electrodes 160 and 164, which may face each other, and an organic light emitting layer 162 therebetween. The organic light emitting layer 162 includes an EML 230 and a charge auxiliary layer 240 between the second electrode 164 and the EML 230. The charge auxiliary layer 250 includes at least one of an ETL 242 and an HBL 244. The HBL 244 may be positioned between the EML 230 and the ETL 244.

The organic light emitting display device 100 (of FIG. 2) may include at least one of a red pixel region, a green pixel region and a blue pixel region. The OLED D may be positioned in each of the red, green and blue pixel regions. The EML 230 in the red pixel region is a red EML, the EML 230 in the green pixel region is a green EML, and the EML 230 in the blue pixel region is a blue EML.

The first electrode 160 may be an anode injecting a hole, and the second electrode 164 may be a cathode injecting an electron. In addition, one of the first and second electrodes 160 and 164 may be a reflective electrode, and the other one of the first and second electrodes 160 and 164 may be a transparent (or a semi-transparent) electrode.

For example, the first electrode 160 may include a transparent conductive material layer formed of ITO or IZO. The second electrode 164 may be formed of one of Al, Mg, Ag, AlMg, and MgAg.

The organic light emitting layer 162 may further include an HTL 220 between the first electrode 160 and the EML 230. In addition, the organic light emitting layer 160 may further include an EBL between the EML 230 and the HBL 220.

The organic light emitting layer 162 may further include at least one of an HIL 210 between the first electrode 160 and the HTL 220 and an EIL 250 between the second electrode 164 and the charge auxiliary layer 240.

The charge auxiliary layer 240 includes the organic compound of the present disclosure. Namely, at least one of the HBL 244 and the ETL 242 may include the organic compound of the present disclosure.

For example, the HBL 244 may be formed of only the organic compound of the present disclosure represented by Formula 1 and may have a thickness of 5 to 20 nm, e.g., 5 to 10 nm. In other words, the HBL 244 may consist of the organic compound of the present disclosure represented by Formula 1.

The ETL 242 may include the organic compound of the present disclosure represented by Formula 1 and may optionally further include a compound in Formula 3 (i.e., lithium quinolinato (Liq)). In this case, in the ETL 242, a weight % ratio of the organic compound in Formula 1 to the compound in Formula 3 may be 1:9 to 9:1, e.g., 2:8 to 8:2 or 3:7 to 7:3. For example, in the ETL 242, the organic compound in Formula 1 and the compound in Formula 3 may have the same weight %.

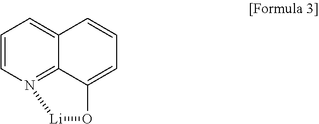

A thickness of the ETL 242 may be greater than that of the HBL 244. The ETL 242 may have a thickness of 10 to 50 nm, e.g., 20 to 40 nm.

When both the HBL 244 and the ETL 242 include the organic compound of the present disclosure, the organic compound in the HBL 244 and the organic compound in ETL 242 may be the same or different.

The EML 230 in the green pixel region, i.e., a green EML, may include the organic compound of the present disclosure represented by Formula 1. For example, the green EML 230 may the organic compound of the present disclosure represented by Formula 1 as a first host, e.g., an n-type host.

Alternatively, the green EML 230 may include a compound in Formula 4 or a compound represented by Formula 5 as a first host, e.g., an n-type host.

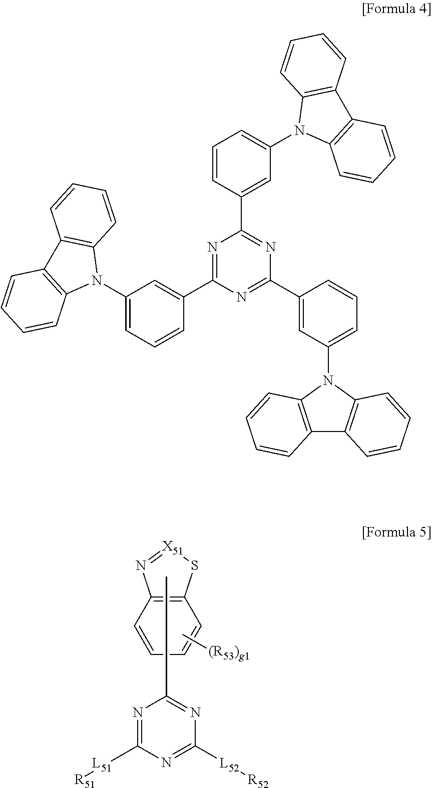

In Formula 5, g1 is an integer of 0 to 4,
each of R₅₁ and R₅₂ is independently selected from the group consisting of a substituted or unsubstituted C6 to C60 aryl group and a substituted or unsubstituted C3 to C60 heteroaryl group,
each R₅₃ is selected from the group consisting of a substituted or unsubstituted C1 to C10 alkyl group, a substituted or unsubstituted C6 to C60 aryl group and a substituted or unsubstituted C3 to C60 heteroaryl group,
X₅₁ is CR₅₄,
R₅₄ is selected from the group consisting of a bonding site (i.e. X₅₁ is a carbon atom directly bonded to the triazine moiety), hydrogen, a substituted or unsubstituted C1 to C10 alkyl group, a substituted or unsubstituted C6 to C60 aryl group and a substituted or unsubstituted C3 to C60 heteroaryl group,
at least one of R₅₁, R₅₂ and R₅₄ is a substituted or unsubstituted carbazolyl group, and
each of L₅₁ and L₅₂ is independently selected from the group consisting of a substituted or unsubstituted C6 to C60 arylene group.

In Formula 5, when g1 is 2 or more, 2 or more R₅₃ groups are the same or different.

In an aspect of the present disclosure, at least one of R₅₁, R₅₂ and R₅₄ may be a carbazolyl group unsubstituted or substituted with a C1 to C20 alkyl group.

In Formula 5, the benzene ring of the benzothiazole moiety may be bonded to the triazine moiety. Namely, the compound represented by Formula 5 may have a structure represented by Formula 5a.

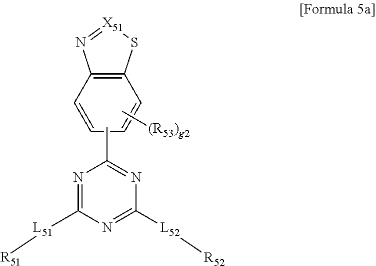

In Formula 5a, the definition of R₅₁, R₅₂, R₅₃, X₅₁, L₅₁ and L₅₂ is the same as for Formula 5, and g2 is an integer of 0 to 3.

In Formula 5, a bonding position of the benzothiazole moiety may be specified. Namely, the compound represented by Formula 5 may have a structure being one of Formulas 5b to 5e.

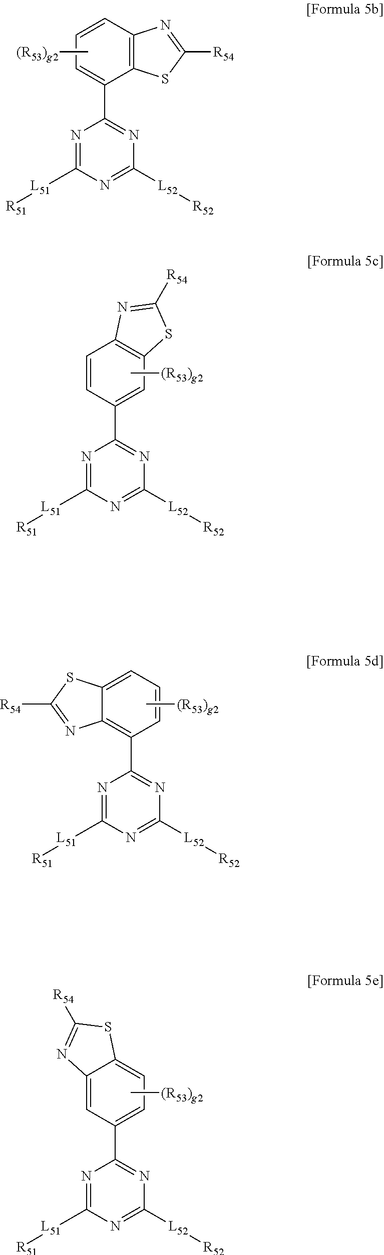

In each of Formulas 5b to 5e, the definition of R₅₁, R₅₂, R₅₃, R₅₄, L₅₁ and L₅₂ is the same as for Formula 5, and g2 is an integer of 0 to 3.

In Formula 5, the thiazole ring of the benzothiazole moiety may be bonded to the triazine moiety. Namely, the compound represented by Formula 5 may have a structure represented by Formula 5f.

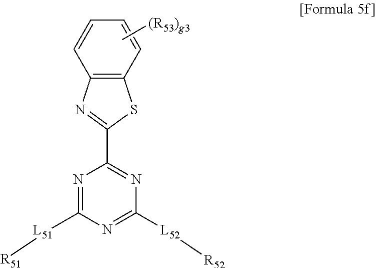

In Formula 5f, the definition of R₅₁, R₅₂, R₅₃, L₅₁ and L₅₂ is the same as for Formula 5, and g3 is an integer of 0 to 4.

In an aspect of the present disclosure, R₅₁ may be a carbazole group unsubstituted or substituted with a C1 to C20 alkyl group, and R₅₂ may be selected from the group consisting of phenyl, pyrenyl, fluorenyl, dibenzofuranyl, dibenzothiophenyl and carbazolyl. R₅₄ may be hydrogen or phenyl unsubstituted or substituted with a C1 to C20 alkyl group, and g1 may be 0.

In an aspect of the present disclosure, R₅₂, which is selected from the group consisting of phenyl, pyrenyl, fluorenyl, dibenzofuranyl, dibenzothiophenyl and carbazolyl, may be unsubstituted or substituted with at least one of a C1 to C20 alkyl group and a C6 to C60 aryl group.

For example, the compound represented by Formula 5 may be one of the compounds in Formula 6.

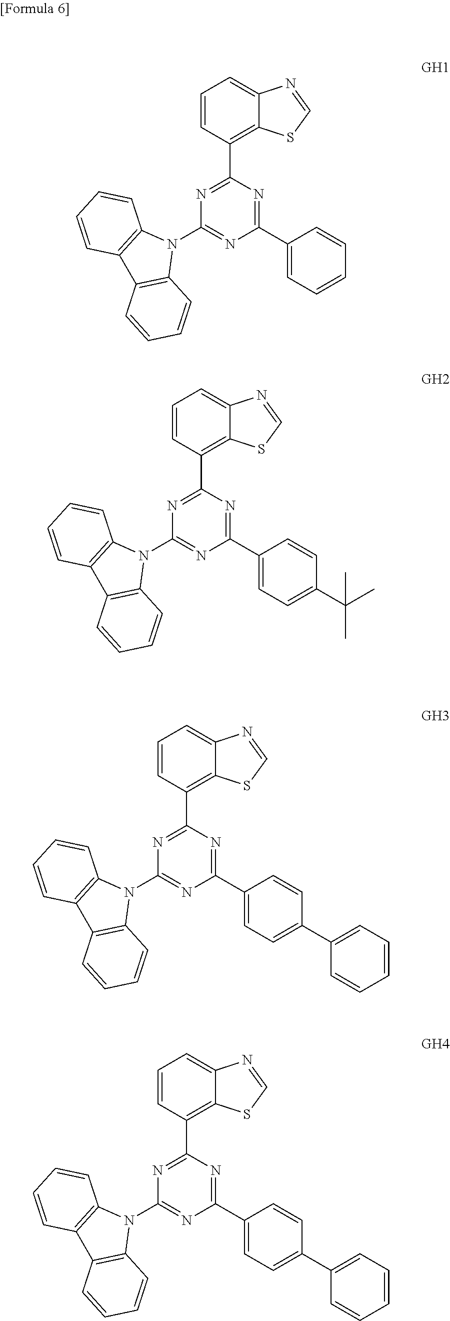 [SMILES: c1ccc(-c2nc(-c3cccc4ncsc34)nc(-n3c4ccccc4c4ccccc43)n2)cc1 ; CC(C)(C)c1ccc(-c2nc(-c3cccc4ncsc34)nc(-n3c4ccccc4c4ccccc43)n2)cc1 ; c1ccc(-c2ccc(-c3nc(-c4cccc5ncsc45)nc(-n4c5ccccc5c5ccccc54)n3)cc2)cc1 ; c1ccc(-c2ccc(-c3nc(-c4cccc5ncsc45)nc(-n4c5ccccc5c5ccccc54)n3)cc2)cc1]

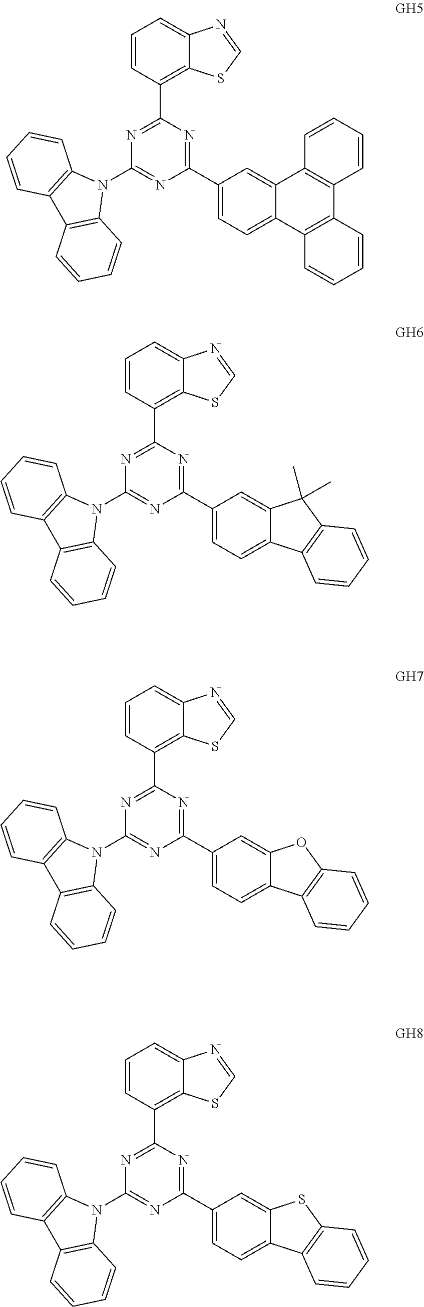 [SMILES: c1cc(-c2nc(-c3ccc4c5ccccc5c5ccccc5c4c3)nc(-n3c4ccccc4c4ccccc43)n2)c2scnc2c1 ; CC1(C)c2ccccc2-c2ccc(-c3nc(-c4cccc5ncsc45)nc(-n4c5ccccc5c5ccccc54)n3)cc21 ; c1cc(-c2nc(-c3ccc4c(c3)oc3ccccc34)nc(-n3c4ccccc4c4ccccc43)n2)c2scnc2c1 ; c1cc(-c2nc(-c3ccc4c(c3)sc3ccccc34)nc(-n3c4ccccc4c4ccccc43)n2)c2scnc2c1]

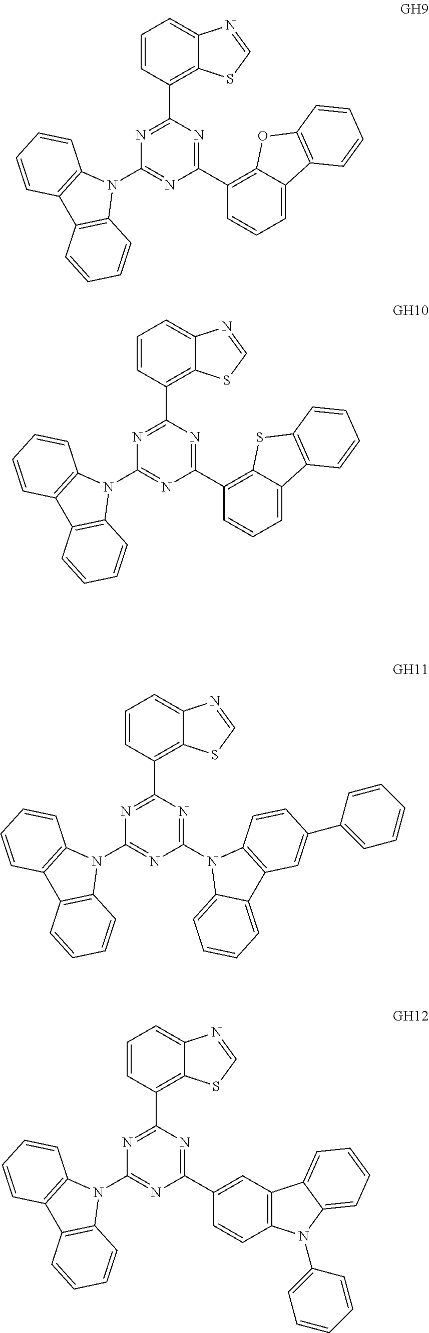 [SMILES: c1cc(-c2nc(-c3cccc4c3oc3ccccc34)nc(-n3c4ccccc4c4ccccc43)n2)c2scnc2c1 ; c1cc(-c2nc(-c3cccc4c3sc3ccccc34)nc(-n3c4ccccc4c4ccccc43)n2)c2scnc2c1 ; c1ccc(-c2ccc3c(c2)c2ccccc2n3-c2nc(-c3cccc4ncsc34)nc(-n3c4ccccc4c4ccccc43)n2)cc1 ; c1ccc(-n2c3ccccc3c3cc(-c4nc(-c5cccc6ncsc56)nc(-n5c6ccccc6c6ccccc65)n4)ccc32)cc1]

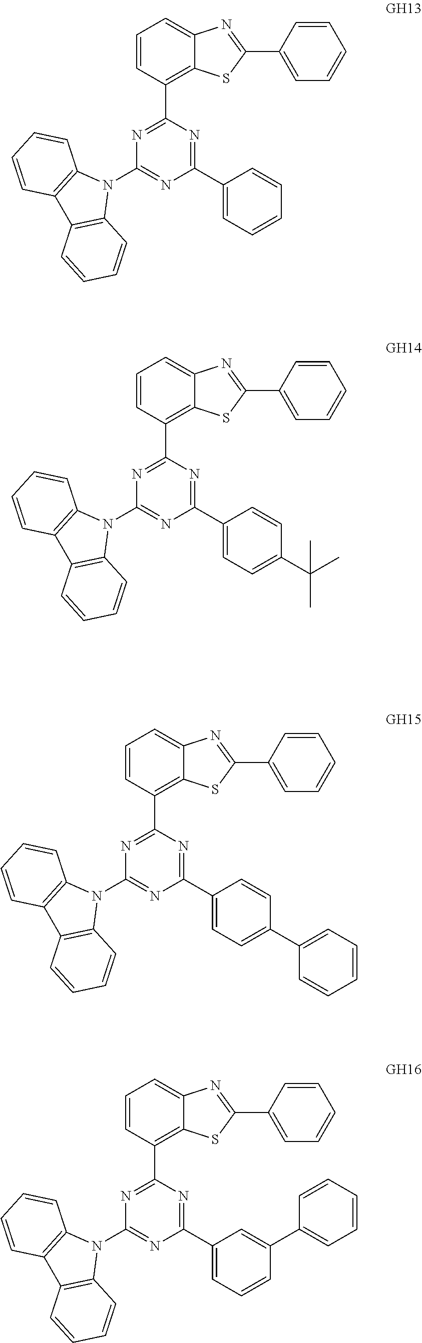 [SMILES: c1ccc(-c2nc(-c3cccc4nc(-c5ccccc5)sc34)nc(-n3c4ccccc4c4ccccc43)n2)cc1 ; CC(C)(C)c1ccc(-c2nc(-c3cccc4nc(-c5ccccc5)sc34)nc(-n3c4ccccc4c4ccccc43)n2)cc1 ; c1ccc(-c2ccc(-c3nc(-c4cccc5nc(-c6ccccc6)sc45)nc(-n4c5ccccc5c5ccccc54)n3)cc2)cc1 ; c1ccc(-c2cccc(-c3nc(-c4cccc5nc(-c6ccccc6)sc45)nc(-n4c5ccccc5c5ccccc54)n3)c2)cc1]

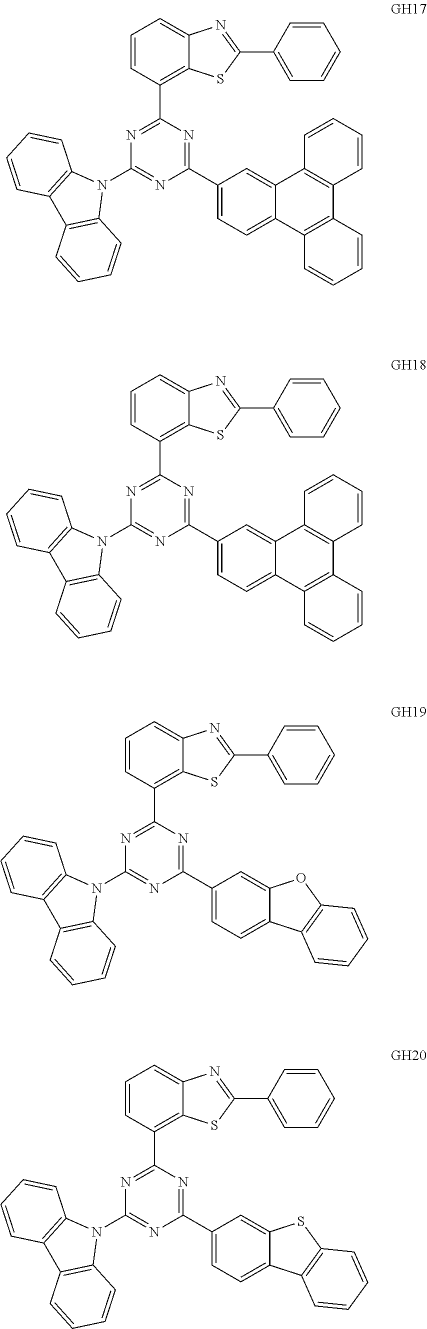 [SMILES: c1ccc(-c2nc3cccc(-c4nc(-c5ccc6c7ccccc7c7ccccc7c6c5)nc(-n5c6ccccc6c6ccccc65)n4)c3s2)cc1 ; c1ccc(-c2nc3cccc(-c4nc(-c5ccc6c7ccccc7c7ccccc7c6c5)nc(-n5c6ccccc6c6ccccc65)n4)c3s2)cc1 ; c1ccc(-c2nc3cccc(-c4nc(-c5ccc6c(c5)oc5ccccc56)nc(-n5c6ccccc6c6ccccc65)n4)c3s2)cc1 ; c1ccc(-c2nc3cccc(-c4nc(-c5ccc6c(c5)sc5ccccc56)nc(-n5c6ccccc6c6ccccc65)n4)c3s2)cc1]

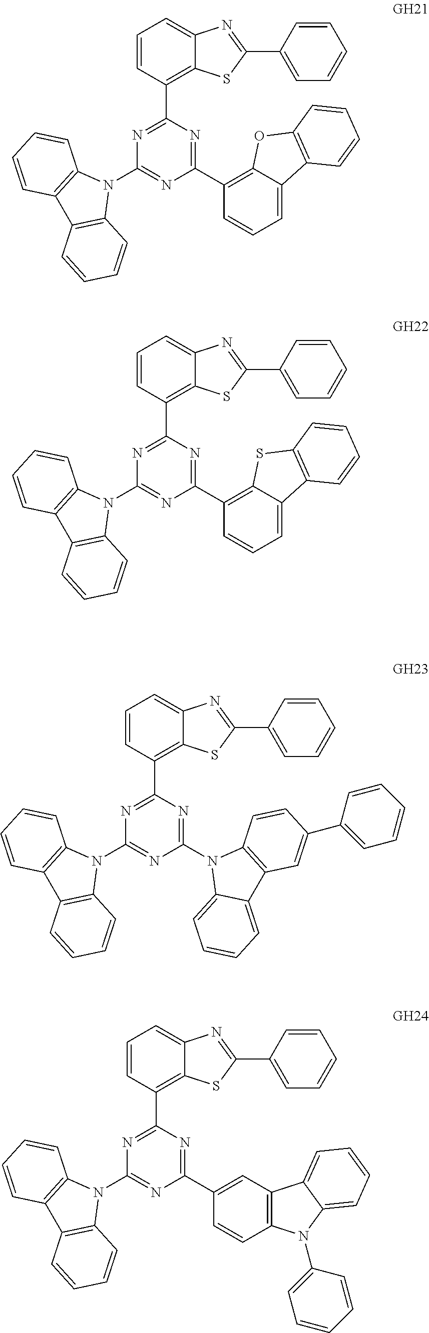 [SMILES: c1ccc(-c2nc3cccc(-c4nc(-c5cccc6c5oc5ccccc56)nc(-n5c6ccccc6c6ccccc65)n4)c3s2)cc1 ; c1ccc(-c2nc3cccc(-c4nc(-c5cccc6c5sc5ccccc56)nc(-n5c6ccccc6c6ccccc65)n4)c3s2)cc1 ; c1ccc(-c2ccc3c(c2)c2ccccc2n3-c2nc(-c3cccc4nc(-c5ccccc5)sc34)nc(-n3c4ccccc4c4ccccc43)n2)cc1 ; c1ccc(-c2nc3cccc(-c4nc(-c5ccc6c(c5)c5ccccc5n6-c5ccccc5)nc(-n5c6ccccc6c6ccccc65)n4)c3s2)cc1]

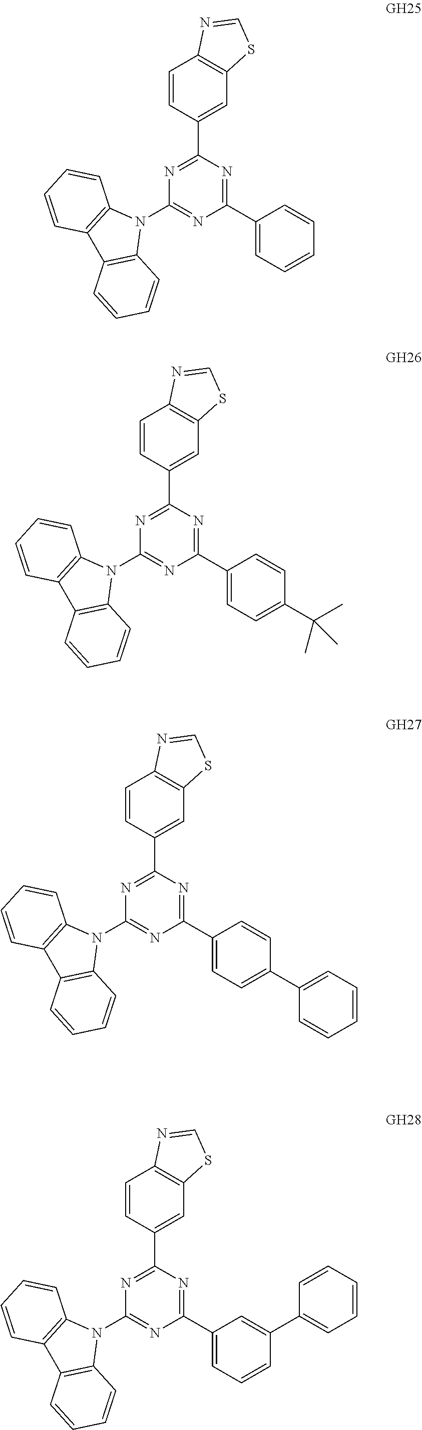 [SMILES: c1ccc(-c2nc(-c3ccc4ncsc4c3)nc(-n3c4ccccc4c4ccccc43)n2)cc1 ; CC(C)(C)c1ccc(-c2nc(-c3ccc4ncsc4c3)nc(-n3c4ccccc4c4ccccc43)n2)cc1 ; c1ccc(-c2ccc(-c3nc(-c4ccc5ncsc5c4)nc(-n4c5ccccc5c5ccccc54)n3)cc2)cc1 ; c1ccc(-c2cccc(-c3nc(-c4ccc5ncsc5c4)nc(-n4c5ccccc5c5ccccc54)n3)c2)cc1]

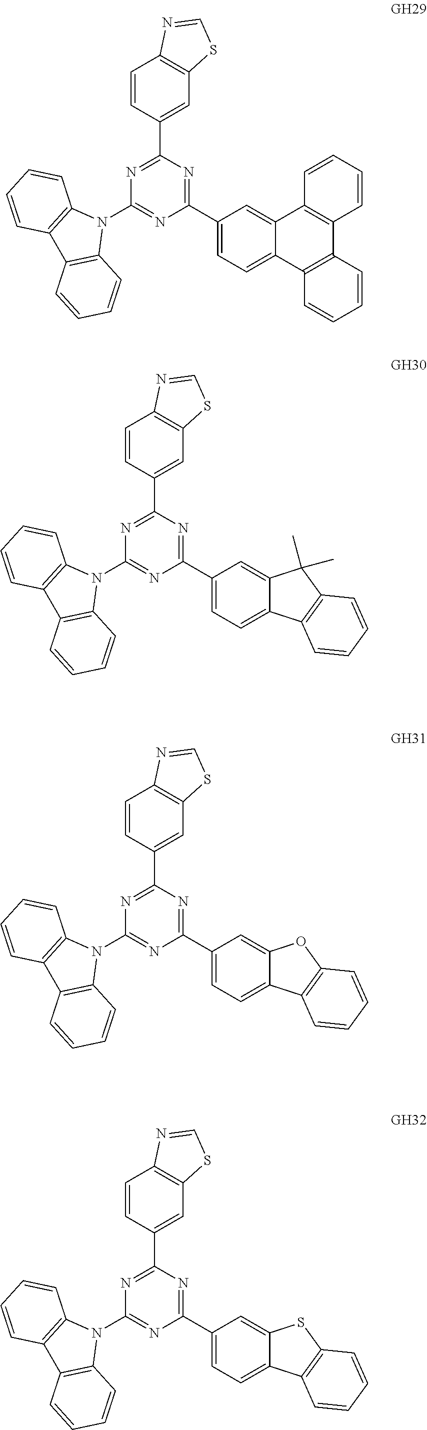 [SMILES: c1ccc2c(c1)c1ccccc1c1cc(-c3nc(-c4ccc5ncsc5c4)nc(-n4c5ccccc5c5ccccc54)n3)ccc21 ; CC1(C)c2ccccc2-c2ccc(-c3nc(-c4ccc5ncsc5c4)nc(-n4c5ccccc5c5ccccc54)n3)cc21 ; c1ccc2c(c1)oc1cc(-c3nc(-c4ccc5ncsc5c4)nc(-n4c5ccccc5c5ccccc54)n3)ccc12 ; c1ccc2c(c1)sc1cc(-c3nc(-c4ccc5ncsc5c4)nc(-n4c5ccccc5c5ccccc54)n3)ccc12]

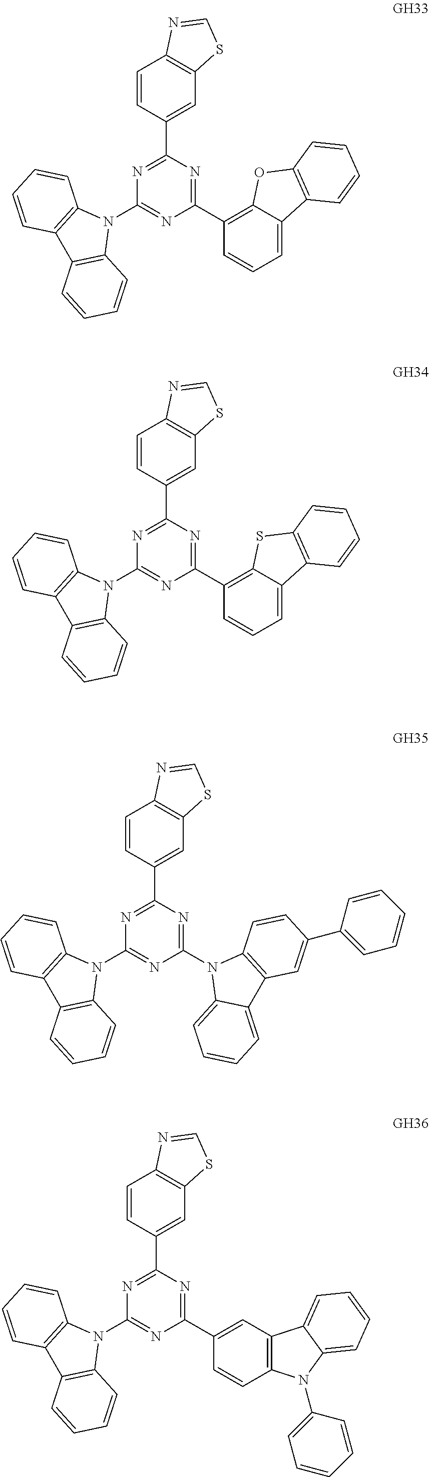 [SMILES: c1ccc2c(c1)oc1c(-c3nc(-c4ccc5ncsc5c4)nc(-n4c5ccccc5c5ccccc54)n3)cccc12 ; c1ccc2c(c1)sc1c(-c3nc(-c4ccc5ncsc5c4)nc(-n4c5ccccc5c5ccccc54)n3)cccc12 ; c1ccc(-c2ccc3c(c2)c2ccccc2n3-c2nc(-c3ccc4ncsc4c3)nc(-n3c4ccccc4c4ccccc43)n2)cc1 ; c1ccc(-n2c3ccccc3c3cc(-c4nc(-c5ccc6ncsc6c5)nc(-n5c6ccccc6c6ccccc65)n4)ccc32)cc1]

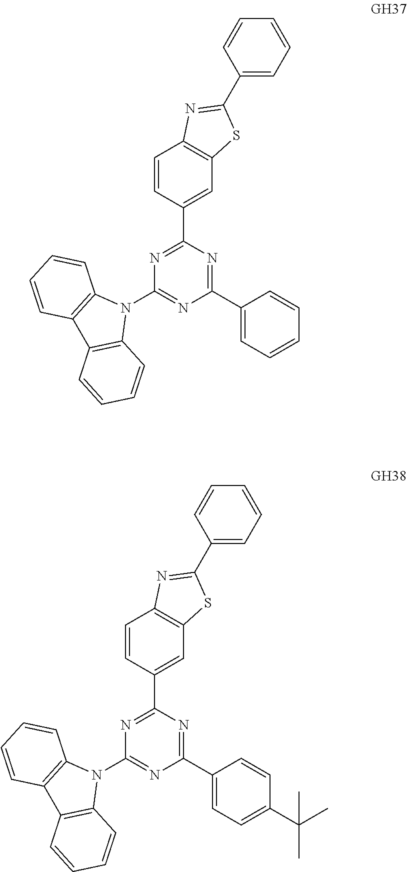 [SMILES: c1ccc(-c2nc(-c3ccc4nc(-c5ccccc5)sc4c3)nc(-n3c4ccccc4c4ccccc43)n2)cc1 ; CC(C)(C)c1ccc(-c2nc(-c3ccc4nc(-c5ccccc5)sc4c3)nc(-n3c4ccccc4c4ccccc43)n2)cc1]

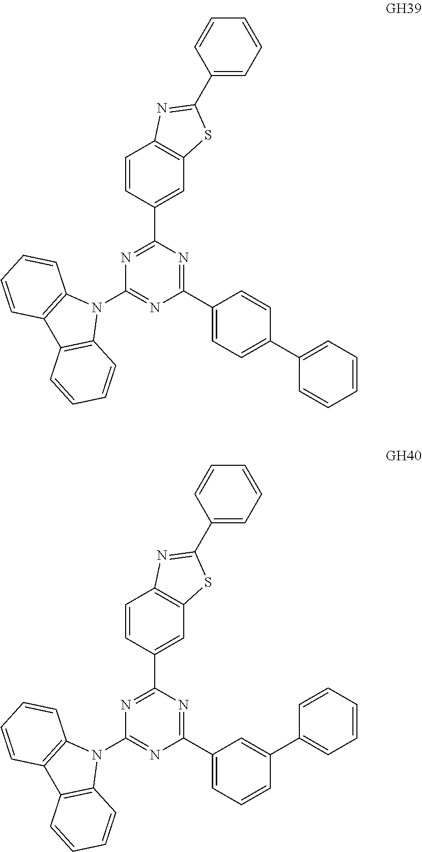 [SMILES: c1ccc(-c2ccc(-c3nc(-c4ccc5nc(-c6ccccc6)sc5c4)nc(-n4c5ccccc5c5ccccc54)n3)cc2)cc1 ; c1ccc(-c2cccc(-c3nc(-c4ccc5nc(-c6ccccc6)sc5c4)nc(-n4c5ccccc5c5ccccc54)n3)c2)cc1]

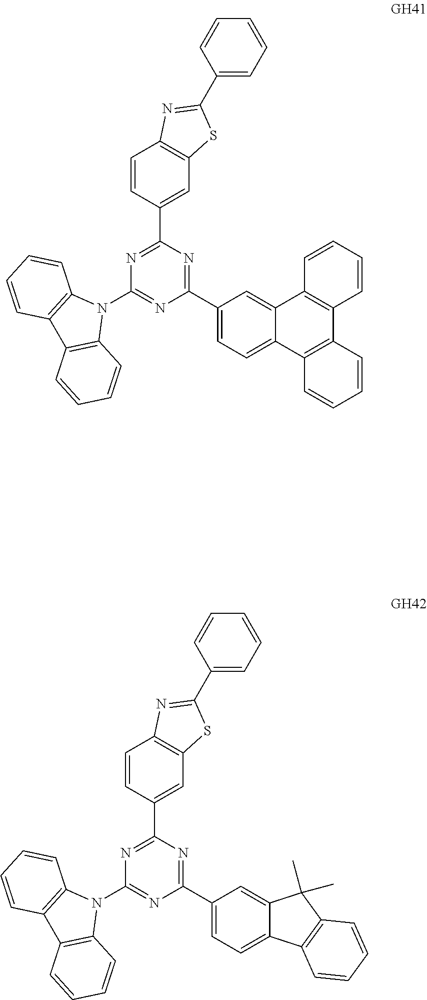 [SMILES: c1ccc(-c2nc3ccc(-c4nc(-c5ccc6c7ccccc7c7ccccc7c6c5)nc(-n5c6ccccc6c6ccccc65)n4)cc3s2)cc1 ; CC1(C)c2ccccc2-c2ccc(-c3nc(-c4ccc5nc(-c6ccccc6)sc5c4)nc(-n4c5ccccc5c5ccccc54)n3)cc21]

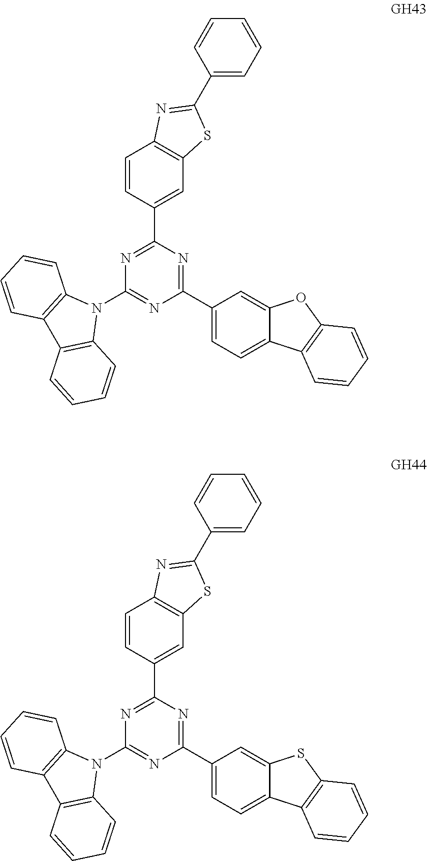 [SMILES: c1ccc(-c2nc3ccc(-c4nc(-c5ccc6c(c5)oc5ccccc56)nc(-n5c6ccccc6c6ccccc65)n4)cc3s2)cc1 ; c1ccc(-c2nc3ccc(-c4nc(-c5ccc6c(c5)sc5ccccc56)nc(-n5c6ccccc6c6ccccc65)n4)cc3s2)cc1]

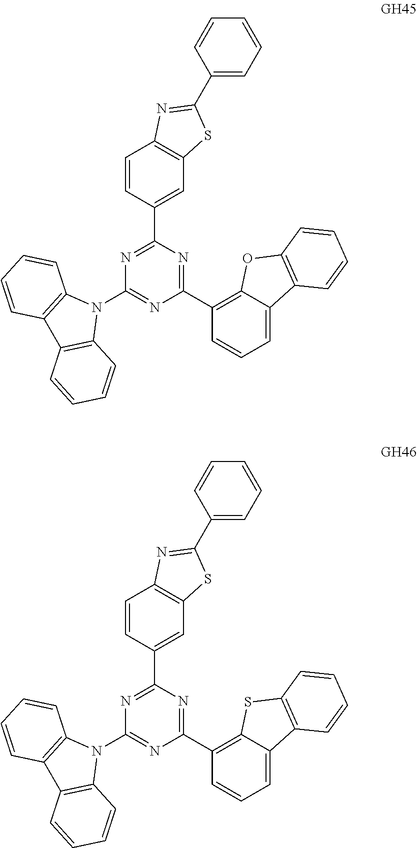 [SMILES: c1ccc(-c2nc3ccc(-c4nc(-c5cccc6c5oc5ccccc56)nc(-n5c6ccccc6c6ccccc65)n4)cc3s2)cc1 ; c1ccc(-c2nc3ccc(-c4nc(-c5cccc6c5sc5ccccc56)nc(-n5c6ccccc6c6ccccc65)n4)cc3s2)cc1]

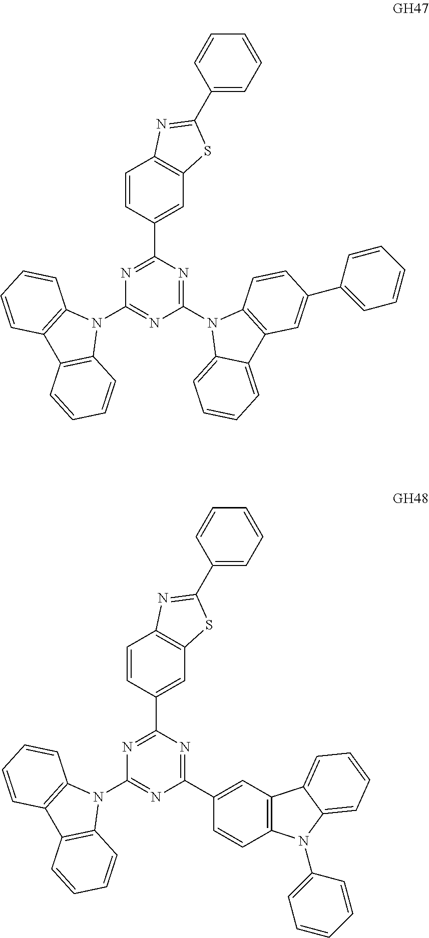 [SMILES: c1ccc(-c2ccc3c(c2)c2ccccc2n3-c2nc(-c3ccc4nc(-c5ccccc5)sc4c3)nc(-n3c4ccccc4c4ccccc43)n2)cc1 ; c1ccc(-c2nc3ccc(-c4nc(-c5ccc6c(c5)c5ccccc5n6-c5ccccc5)nc(-n5c6ccccc6c6ccccc65)n4)cc3s2)cc1]

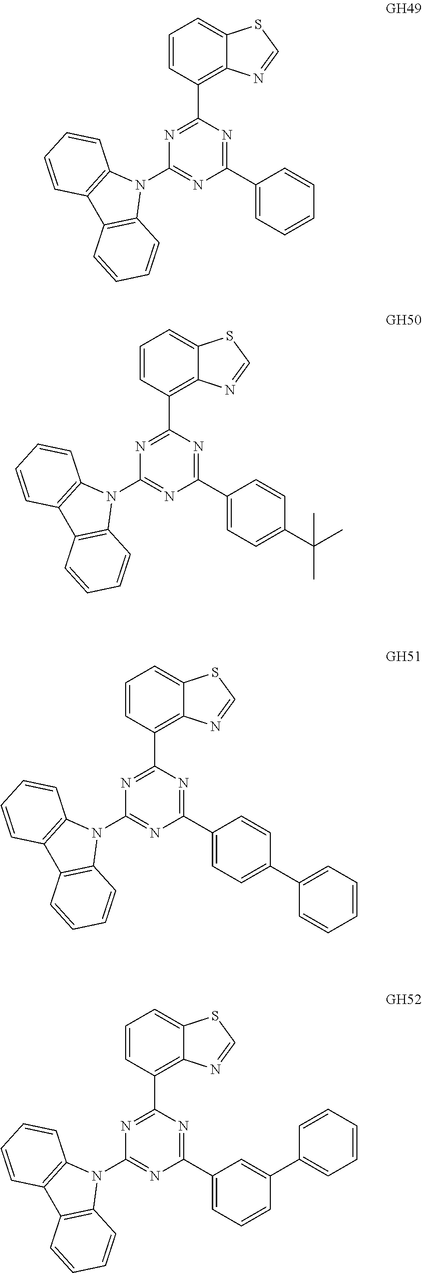 [SMILES: c1ccc(-c2nc(-c3cccc4scnc34)nc(-n3c4ccccc4c4ccccc43)n2)cc1 ; CC(C)(C)c1ccc(-c2nc(-c3cccc4scnc34)nc(-n3c4ccccc4c4ccccc43)n2)cc1 ; c1ccc(-c2ccc(-c3nc(-c4cccc5scnc45)nc(-n4c5ccccc5c5ccccc54)n3)cc2)cc1 ; c1ccc(-c2cccc(-c3nc(-c4cccc5scnc45)nc(-n4c5ccccc5c5ccccc54)n3)c2)cc1]

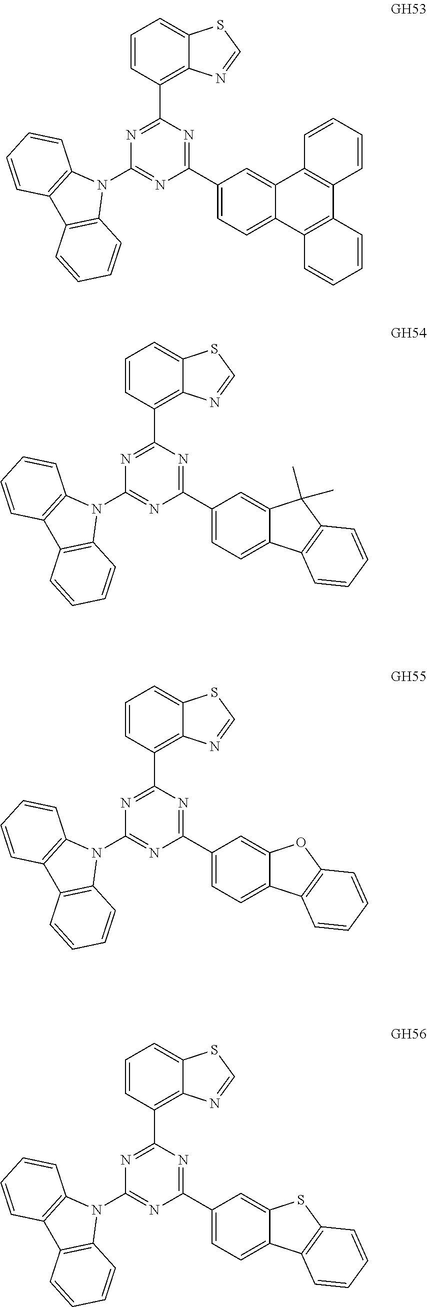 [SMILES: c1cc(-c2nc(-c3ccc4c5ccccc5c5ccccc5c4c3)nc(-n3c4ccccc4c4ccccc43)n2)c2ncsc2c1 ; CC1(C)c2ccccc2-c2ccc(-c3nc(-c4cccc5scnc45)nc(-n4c5ccccc5c5ccccc54)n3)cc21 ; c1ccc2c(c1)oc1cc(-c3nc(-c4cccc5scnc45)nc(-n4c5ccccc5c5ccccc54)n3)ccc12 ; c1cc(-c2nc(-c3ccc4c(c3)sc3ccccc34)nc(-n3c4ccccc4c4ccccc43)n2)c2ncsc2c1]

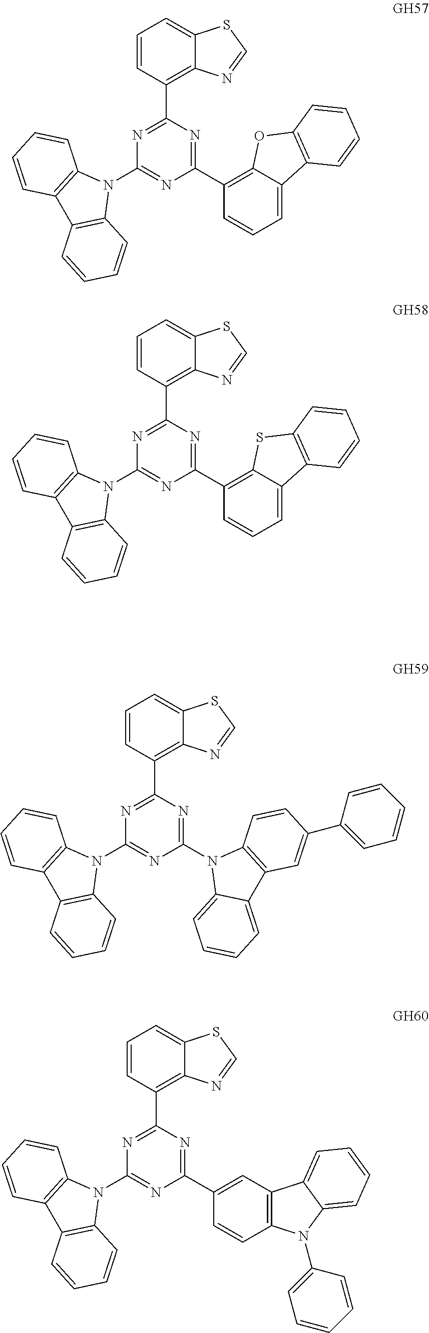 [SMILES: c1ccc2c(c1)oc1c(-c3nc(-c4cccc5scnc45)nc(-n4c5ccccc5c5ccccc54)n3)cccc12 ; c1cc(-c2nc(-c3cccc4c3sc3ccccc34)nc(-n3c4ccccc4c4ccccc43)n2)c2ncsc2c1 ; c1ccc(-c2ccc3c(c2)c2ccccc2n3-c2nc(-c3cccc4scnc34)nc(-n3c4ccccc4c4ccccc43)n2)cc1 ; c1ccc(-n2c3ccccc3c3cc(-c4nc(-c5cccc6scnc56)nc(-n5c6ccccc6c6ccccc65)n4)ccc32)cc1]

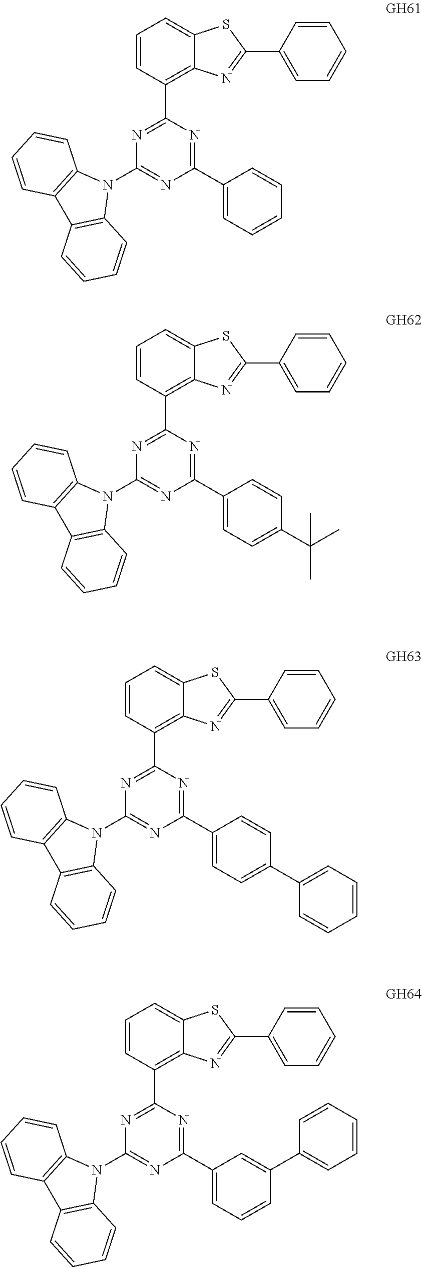 [SMILES: c1ccc(-c2nc(-c3cccc4sc(-c5ccccc5)nc34)nc(-n3c4ccccc4c4ccccc43)n2)cc1 ; CC(C)(C)c1ccc(-c2nc(-c3cccc4sc(-c5ccccc5)nc34)nc(-n3c4ccccc4c4ccccc43)n2)cc1 ; c1ccc(-c2ccc(-c3nc(-c4cccc5sc(-c6ccccc6)nc45)nc(-n4c5ccccc5c5ccccc54)n3)cc2)cc1 ; c1ccc(-c2cccc(-c3nc(-c4cccc5sc(-c6ccccc6)nc45)nc(-n4c5ccccc5c5ccccc54)n3)c2)cc1]

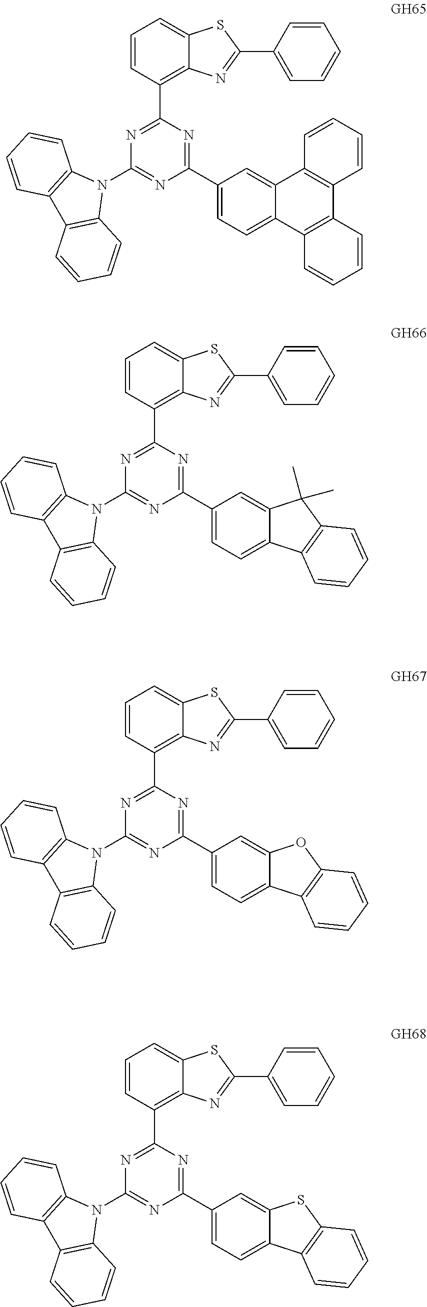 [SMILES: c1ccc(-c2nc3c(-c4nc(-c5ccc6c7ccccc7c7ccccc7c6c5)nc(-n5c6ccccc6c6ccccc65)n4)cccc3s2)cc1 ; CC1(C)c2ccccc2-c2ccc(-c3nc(-c4cccc5sc(-c6ccccc6)nc45)nc(-n4c5ccccc5c5ccccc54)n3)cc21 ; c1ccc(-c2nc3c(-c4nc(-c5ccc6c(c5)oc5ccccc56)nc(-n5c6ccccc6c6ccccc65)n4)cccc3s2)cc1 ; c1ccc(-c2nc3c(-c4nc(-c5ccc6c(c5)sc5ccccc56)nc(-n5c6ccccc6c6ccccc65)n4)cccc3s2)cc1]

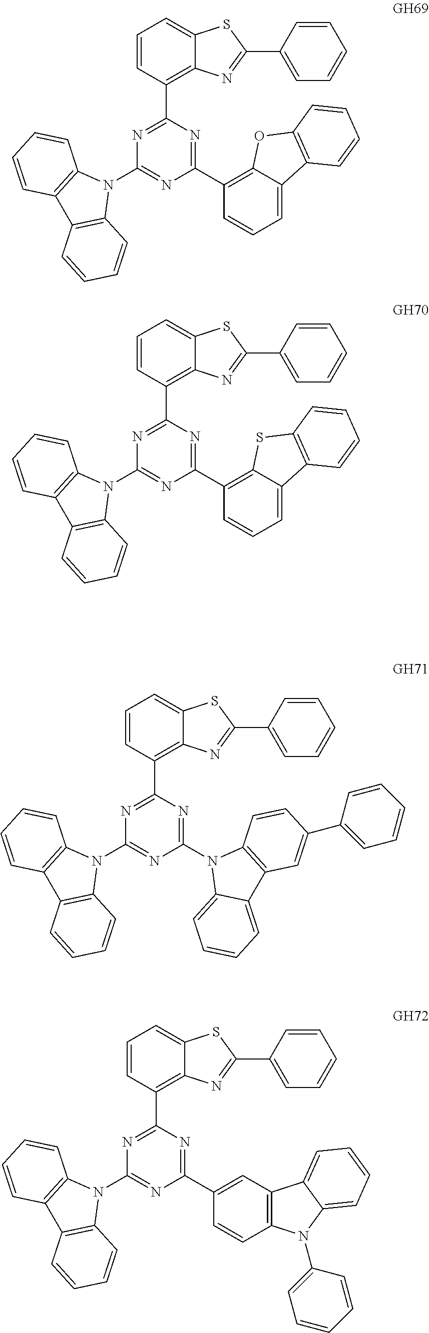 [SMILES: c1ccc(-c2nc3c(-c4nc(-c5cccc6c5oc5ccccc56)nc(-n5c6ccccc6c6ccccc65)n4)cccc3s2)cc1 ; c1ccc(-c2nc3c(-c4nc(-c5cccc6c5sc5ccccc56)nc(-n5c6ccccc6c6ccccc65)n4)cccc3s2)cc1 ; c1ccc(-c2ccc3c(c2)c2ccccc2n3-c2nc(-c3cccc4sc(-c5ccccc5)nc34)nc(-n3c4ccccc4c4ccccc43)n2)cc1 ; c1ccc(-c2nc3c(-c4nc(-c5ccc6c(c5)c5ccccc5n6-c5ccccc5)nc(-n5c6ccccc6c6ccccc65)n4)cccc3s2)cc1]

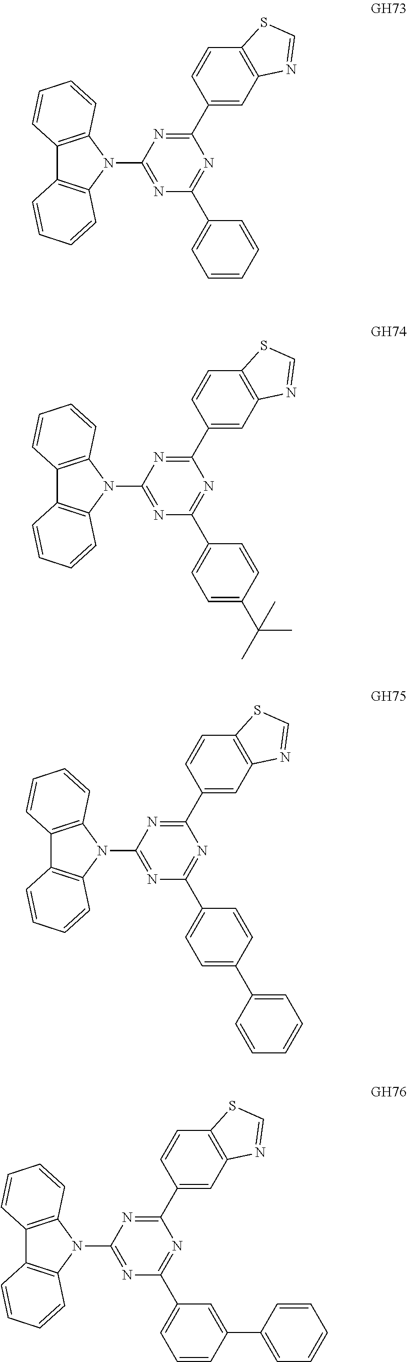 [SMILES: c1ccc(-c2nc(-c3ccc4scnc4c3)nc(-n3c4ccccc4c4ccccc43)n2)cc1 ; CC(C)(C)c1ccc(-c2nc(-c3ccc4scnc4c3)nc(-n3c4ccccc4c4ccccc43)n2)cc1 ; c1ccc(-c2ccc(-c3nc(-c4ccc5scnc5c4)nc(-n4c5ccccc5c5ccccc54)n3)cc2)cc1 ; c1ccc(-c2cccc(-c3nc(-c4ccc5scnc5c4)nc(-n4c5ccccc5c5ccccc54)n3)c2)cc1]

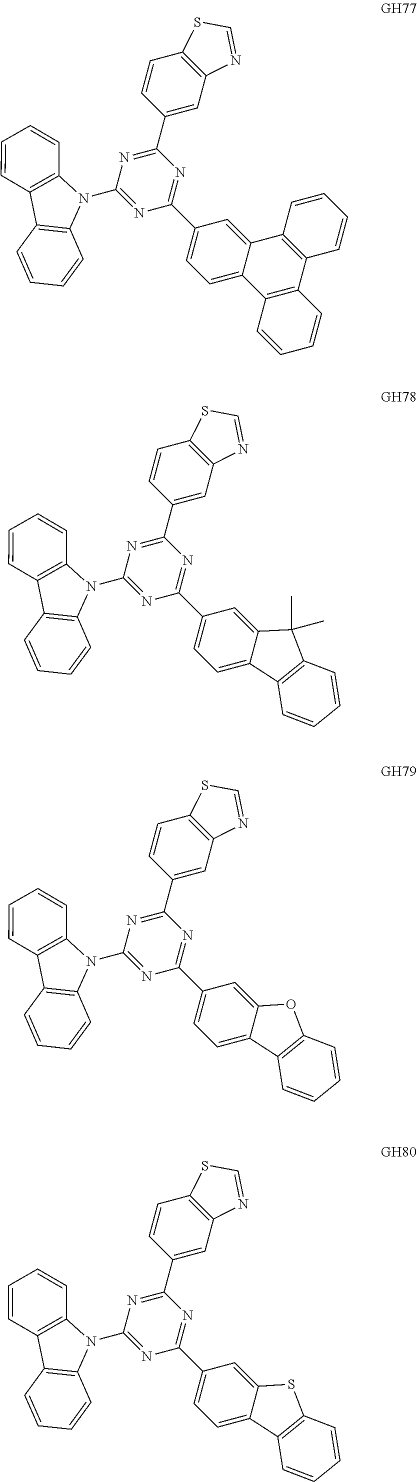 [SMILES: c1ccc2c(c1)c1ccccc1c1cc(-c3nc(-c4ccc5scnc5c4)nc(-n4c5ccccc5c5ccccc54)n3)ccc21 ; CC1(C)c2ccccc2-c2ccc(-c3nc(-c4ccc5scnc5c4)nc(-n4c5ccccc5c5ccccc54)n3)cc21 ; c1ccc2c(c1)oc1cc(-c3nc(-c4ccc5scnc5c4)nc(-n4c5ccccc5c5ccccc54)n3)ccc12 ; c1ccc2c(c1)sc1cc(-c3nc(-c4ccc5scnc5c4)nc(-n4c5ccccc5c5ccccc54)n3)ccc12]

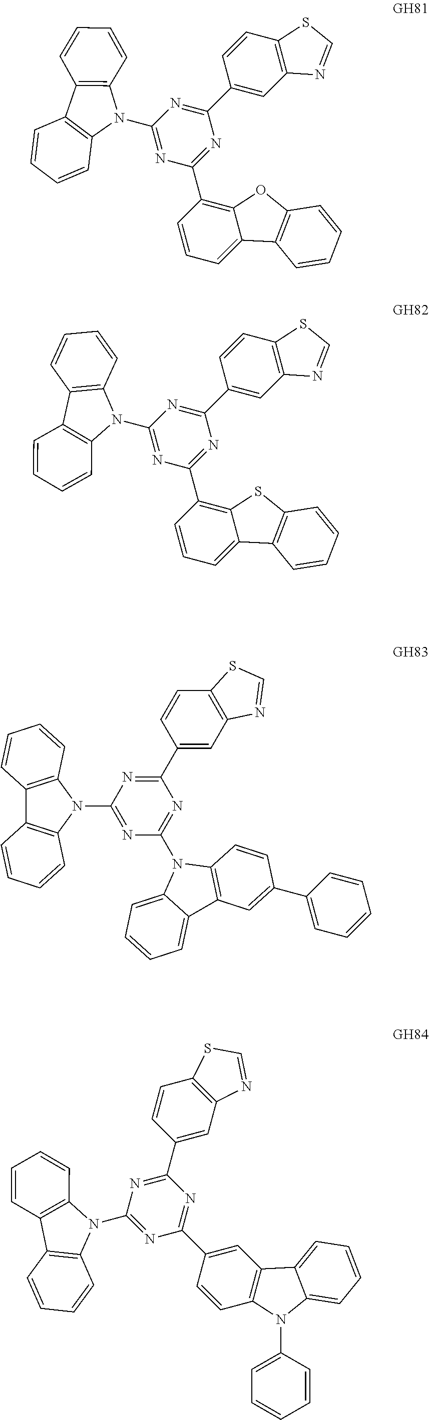 [SMILES: c1ccc2c(c1)oc1c(-c3nc(-c4ccc5scnc5c4)nc(-n4c5ccccc5c5ccccc54)n3)cccc12 ; c1ccc2c(c1)sc1c(-c3nc(-c4ccc5scnc5c4)nc(-n4c5ccccc5c5ccccc54)n3)cccc12 ; c1ccc(-c2ccc3c(c2)c2ccccc2n3-c2nc(-c3ccc4scnc4c3)nc(-n3c4ccccc4c4ccccc43)n2)cc1 ; c1ccc(-n2c3ccccc3c3cc(-c4nc(-c5ccc6scnc6c5)nc(-n5c6ccccc6c6ccccc65)n4)ccc32)cc1]

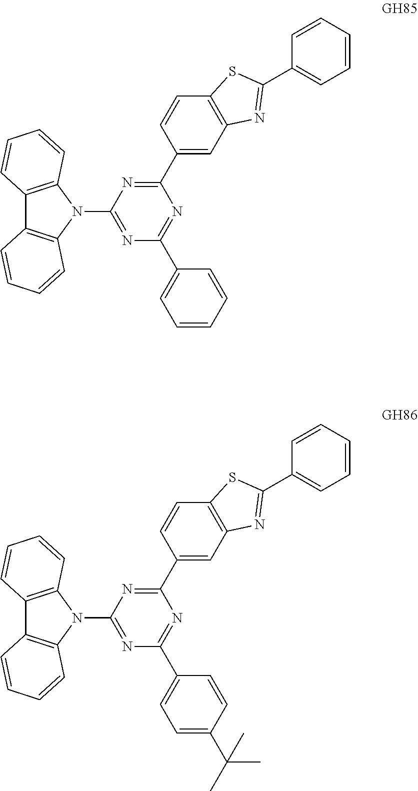 [SMILES: c1ccc(-c2nc(-c3ccc4sc(-c5ccccc5)nc4c3)nc(-n3c4ccccc4c4ccccc43)n2)cc1 ; CC(C)(C)c1ccc(-c2nc(-c3ccc4sc(-c5ccccc5)nc4c3)nc(-n3c4ccccc4c4ccccc43)n2)cc1]

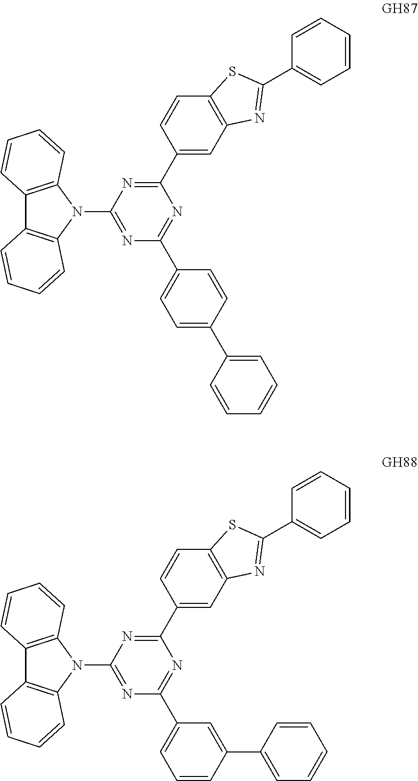 [SMILES: c1ccc(-c2ccc(-c3nc(-c4ccc5sc(-c6ccccc6)nc5c4)nc(-n4c5ccccc5c5ccccc54)n3)cc2)cc1 ; c1ccc(-c2cccc(-c3nc(-c4ccc5sc(-c6ccccc6)nc5c4)nc(-n4c5ccccc5c5ccccc54)n3)c2)cc1]

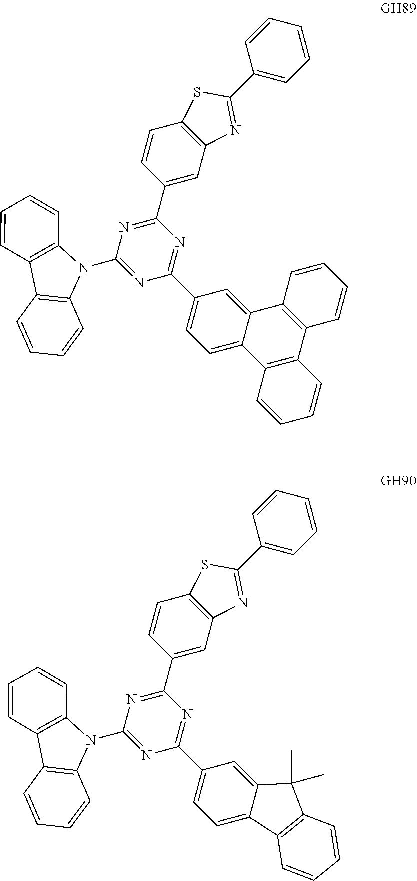 [SMILES: c1ccc(-c2nc3cc(-c4nc(-c5ccc6c7ccccc7c7ccccc7c6c5)nc(-n5c6ccccc6c6ccccc65)n4)ccc3s2)cc1 ; CC1(C)c2ccccc2-c2ccc(-c3nc(-c4ccc5sc(-c6ccccc6)nc5c4)nc(-n4c5ccccc5c5ccccc54)n3)cc21]

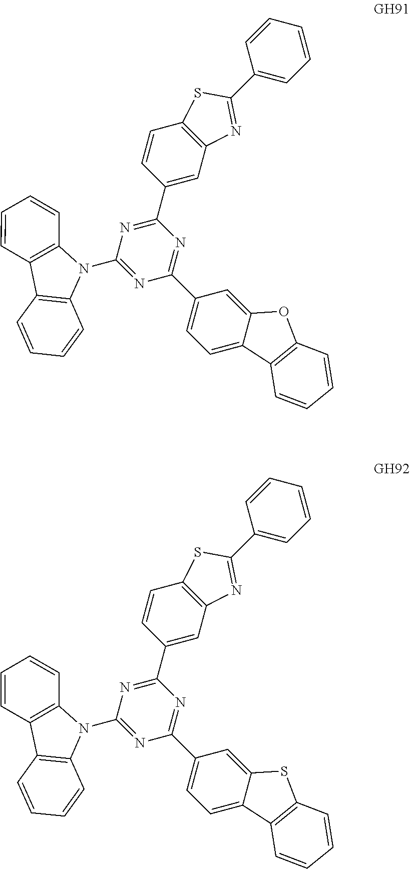 [SMILES: c1ccc(-c2nc3cc(-c4nc(-c5ccc6c(c5)oc5ccccc56)nc(-n5c6ccccc6c6ccccc65)n4)ccc3s2)cc1 ; c1ccc(-c2nc3cc(-c4nc(-c5ccc6c(c5)sc5ccccc56)nc(-n5c6ccccc6c6ccccc65)n4)ccc3s2)cc1]

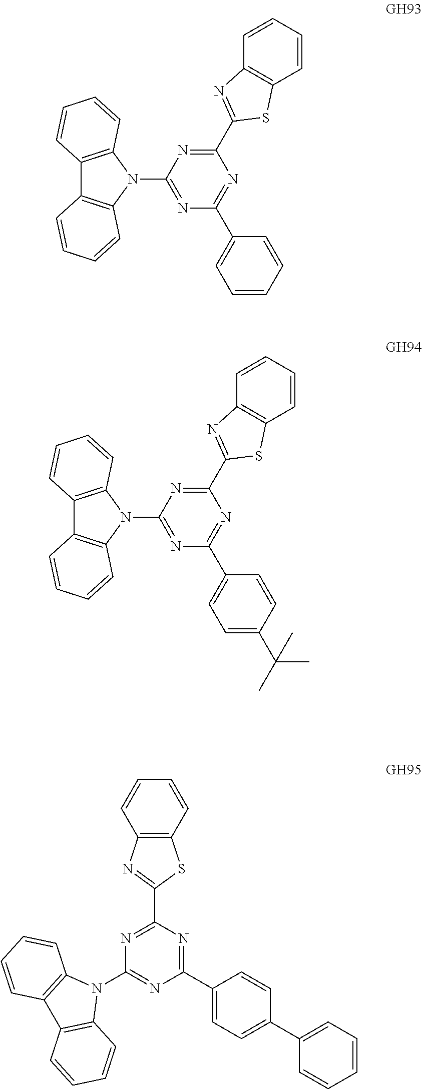 [SMILES: c1ccc(-c2nc(-c3nc4ccccc4s3)nc(-n3c4ccccc4c4ccccc43)n2)cc1 ; CC(C)(C)c1ccc(-c2nc(-c3nc4ccccc4s3)nc(-n3c4ccccc4c4ccccc43)n2)cc1 ; c1ccc(-c2ccc(-c3nc(-c4nc5ccccc5s4)nc(-n4c5ccccc5c5ccccc54)n3)cc2)cc1]

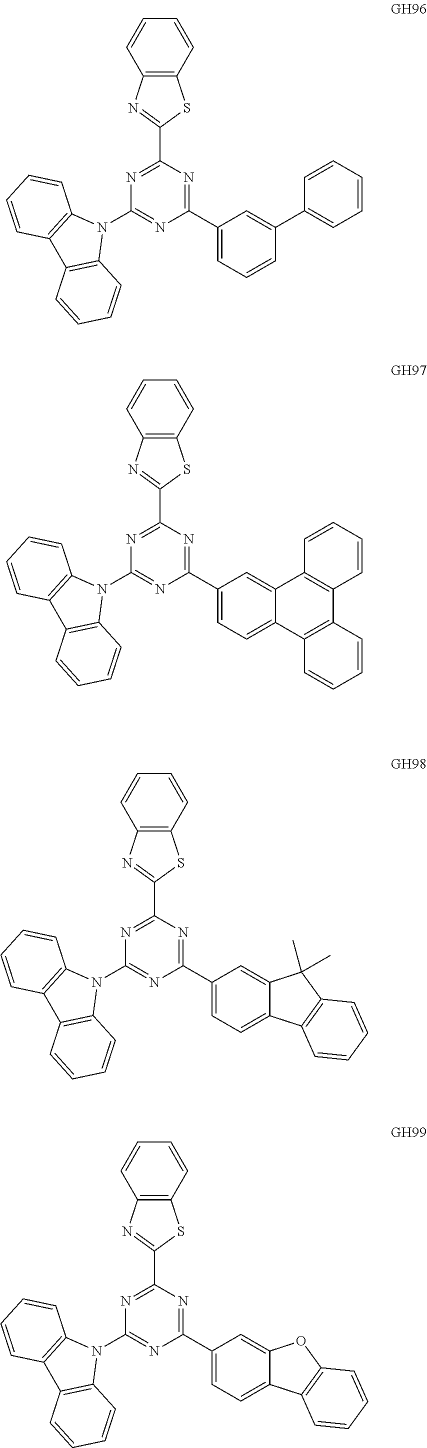 [SMILES: c1ccc(-c2cccc(-c3nc(-c4nc5ccccc5s4)nc(-n4c5ccccc5c5ccccc54)n3)c2)cc1 ; c1ccc2sc(-c3nc(-c4ccc5c6ccccc6c6ccccc6c5c4)nc(-n4c5ccccc5c5ccccc54)n3)nc2c1 ; CC1(C)c2ccccc2-c2ccc(-c3nc(-c4nc5ccccc5s4)nc(-n4c5ccccc5c5ccccc54)n3)cc21 ; c1ccc2sc(-c3nc(-c4ccc5c(c4)oc4ccccc45)nc(-n4c5ccccc5c5ccccc54)n3)nc2c1]

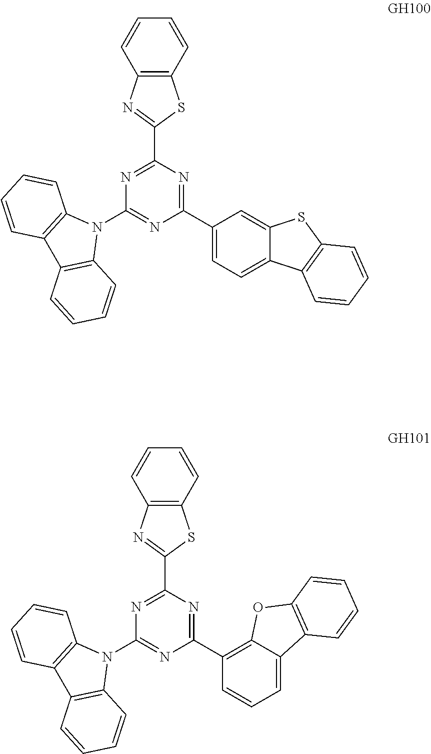 [SMILES: c1ccc2sc(-c3nc(-c4ccc5c(c4)sc4ccccc45)nc(-n4c5ccccc5c5ccccc54)n3)nc2c1 ; c1ccc2sc(-c3nc(-c4cccc5c4oc4ccccc45)nc(-n4c5ccccc5c5ccccc54)n3)nc2c1]

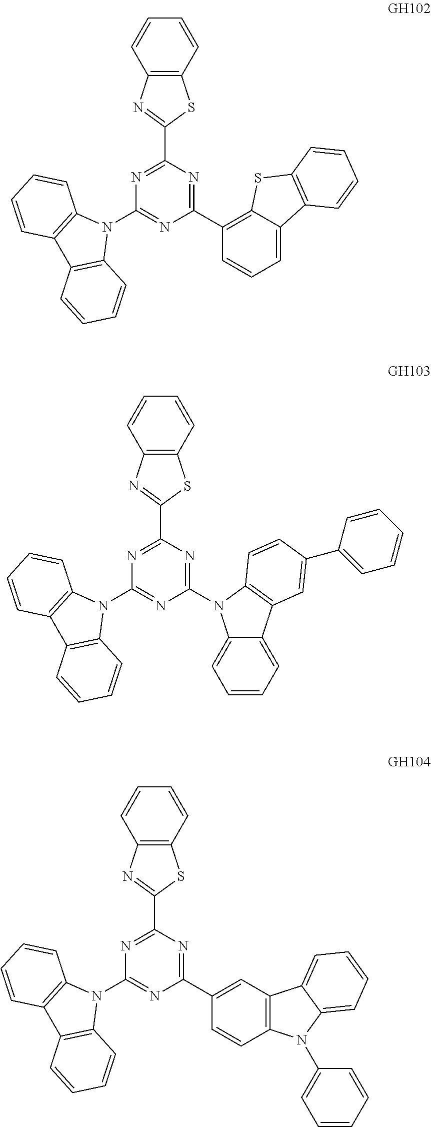 [SMILES: c1ccc2sc(-c3nc(-c4cccc5c4sc4ccccc45)nc(-n4c5ccccc5c5ccccc54)n3)nc2c1 ; c1ccc(-c2ccc3c(c2)c2ccccc2n3-c2nc(-c3nc4ccccc4s3)nc(-n3c4ccccc4c4ccccc43)n2)cc1 ; c1ccc(-n2c3ccccc3c3cc(-c4nc(-c5nc6ccccc6s5)nc(-n5c6ccccc6c6ccccc65)n4)ccc32)cc1]

The green EML 230 may further include a compound represented by Formula 7 as a second host, e.g., a p-type host.

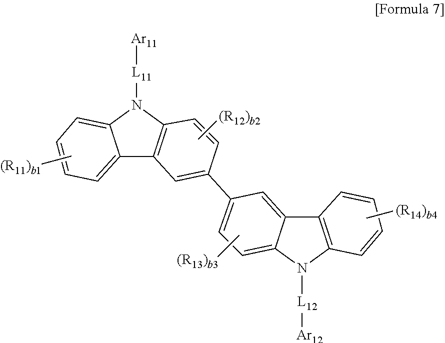

In Formula 7, each of b1 and b4 is independently an integer of 0 to 4, each of b2 and b3 is independently an integer of 0 to 3,
each R₁₁, R₁₂, R₁₃, and R₁₄ is independently selected from the group consisting of deuterium, a substituted or unsubstituted C1 to C10 alkyl group, a substituted or unsubstituted C3 to C30 cycloalkyl group, a substituted or unsubstituted C1 to C10 alkoxy group, a substituted or unsubstituted C6 to C60 aryl group, and a substituted or unsubstituted C3 to C60 heteroaryl group,
each of Ln and L₁₂ is independently selected from the group consisting of a single bond, a substituted or unsubstituted C6 to C60 arylene group, and a substituted or unsubstituted C3 to C60 heteroarylene group, and
each of Ar₁₁ and Ar₁₂ is independently selected from the group consisting of a substituted or unsubstituted C6 to C60 aryl group, and a substituted or unsubstituted C3 to C60 heteroaryl group.

When b1 is 2 or more, 2 or more R₁₁ groups are the same or different. When b2 is 2 or more, 2 or more R₁₂ groups are the same or different. When b3 is 2 or more, 2 or more R₁₃ groups are the same or different. When b4 is 2 or more, 2 or more R₁₄ groups are the same or different.

In Formula 7, each of L₁₁ and L₁₂ may be a single bond, and each of Ar₁₁ and Ar₁₂ may be independently selected from a substituted or unsubstituted phenyl. For example, Formula 7 may be represented by Formula 7a.

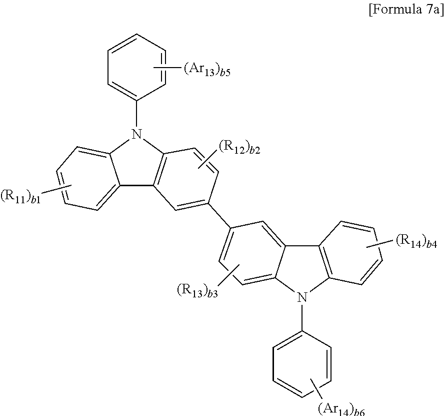

In Formula 7a, the definition of each R₁, R₁₂, R₁₃, R₁₄, b1, b2, b3, and b4 is the same as in Formula 7,
each of Ar₁₃ and Ar₁₄ is independently selected from a substituted or unsubstituted C6 to C30 aryl group, and
each of b5 and b6 is independently an integer of 0 to 5.

For example, in Formula 7a, each of Ar₁₃ and Ar₁₄ may be phenyl, and each of b5 and b6 may be independently 0 or 1.

The second host may be one of the compounds in Formula 8.

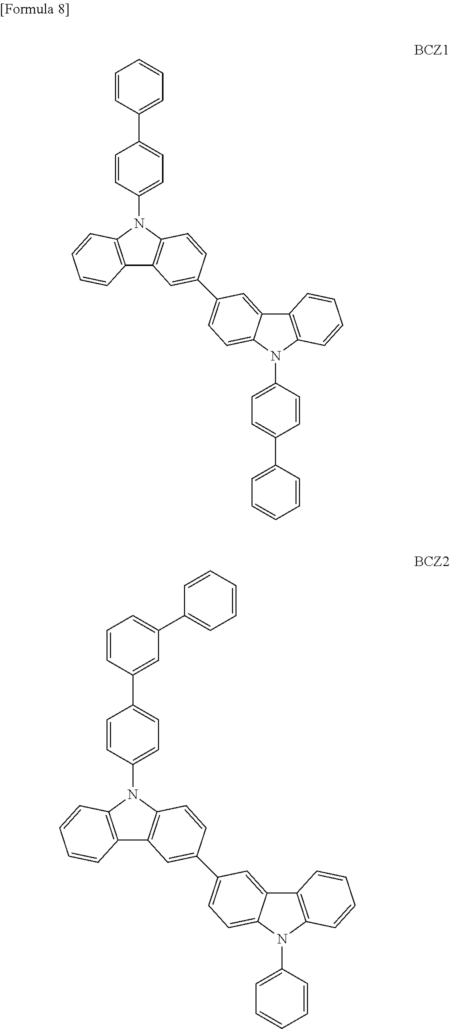 [SMILES: c1ccc(-c2ccc(-n3c4ccccc4c4cc(-c5ccc6c(c5)c5ccccc5n6-c5ccc(-c6ccccc6)cc5)ccc43)cc2)cc1 ; c1ccc(-c2cccc(-c3ccc(-n4c5ccccc5c5cc(-c6ccc7c(c6)c6ccccc6n7-c6ccccc6)ccc54)cc3)c2)cc1]

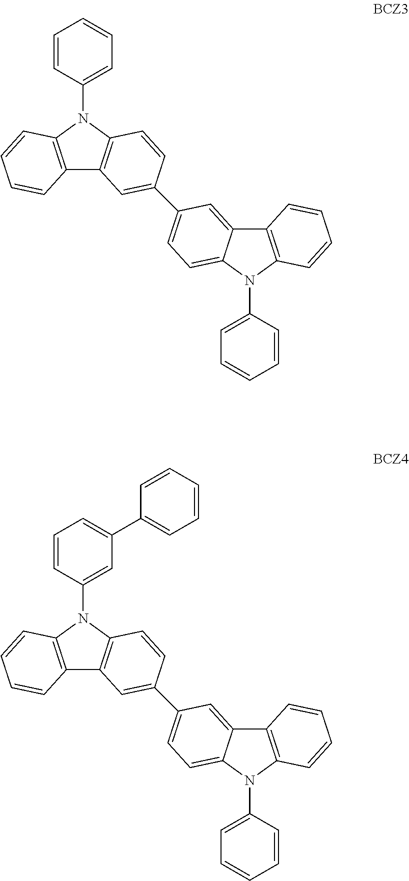 [SMILES: c1ccc(-n2c3ccccc3c3cc(-c4ccc5c(c4)c4ccccc4n5-c4ccccc4)ccc32)cc1 ; c1ccc(-c2cccc(-n3c4ccccc4c4cc(-c5ccc6c(c5)c5ccccc5n6-c5ccccc5)ccc43)c2)cc1]

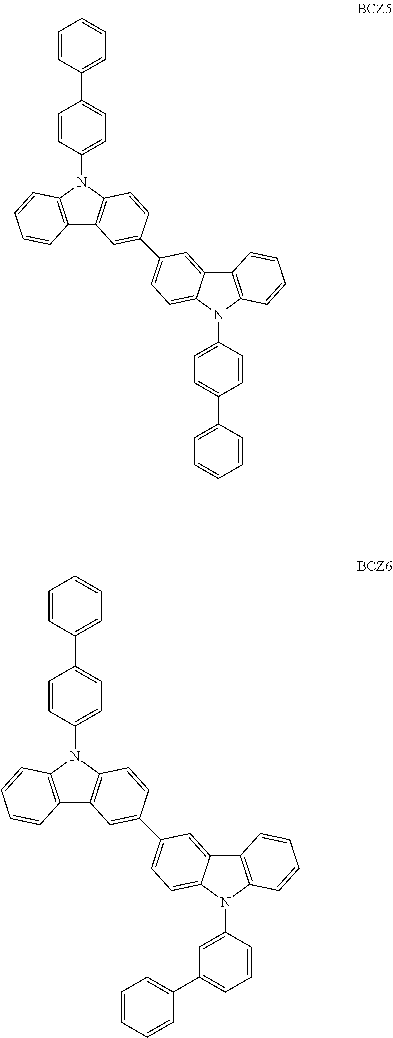 [SMILES: c1ccc(-c2ccc(-n3c4ccccc4c4cc(-c5ccc6c(c5)c5ccccc5n6-c5ccc(-c6ccccc6)cc5)ccc43)cc2)cc1 ; c1ccc(-c2ccc(-n3c4ccccc4c4cc(-c5ccc6c(c5)c5ccccc5n6-c5cccc(-c6ccccc6)c5)ccc43)cc2)cc1]

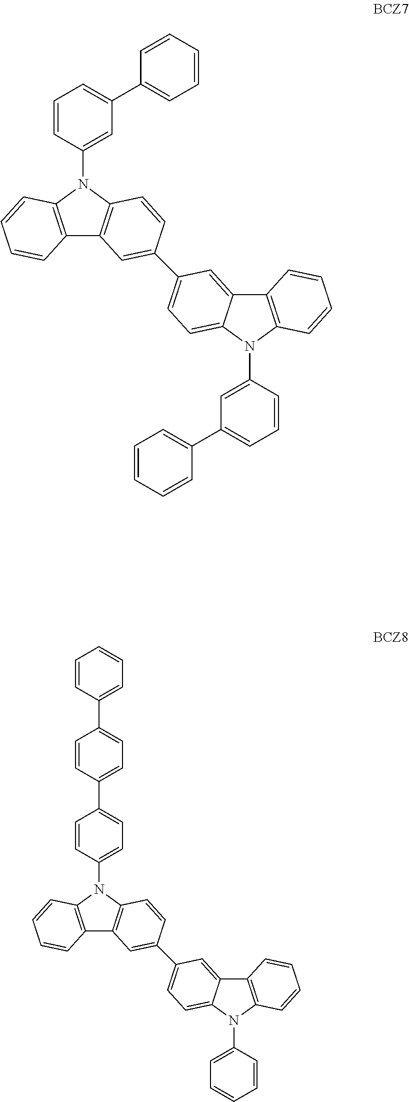 [SMILES: c1ccc(-c2cccc(-n3c4ccccc4c4cc(-c5ccc6c(c5)c5ccccc5n6-c5cccc(-c6ccccc6)c5)ccc43)c2)cc1 ; c1ccc(-c2ccc(-c3ccc(-n4c5ccccc5c5cc(-c6ccc7c(c6)c6ccccc6n7-c6ccccc6)ccc54)cc3)cc2)cc1]

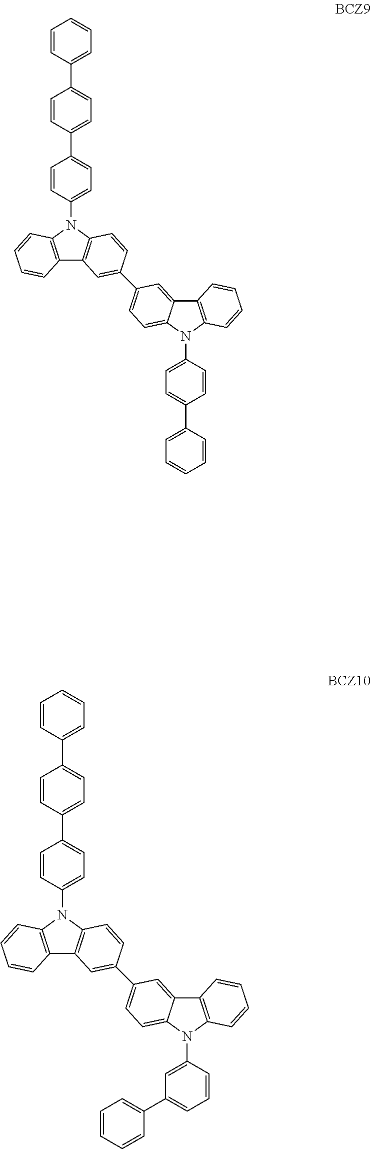 [SMILES: c1ccc(-c2ccc(-c3ccc(-n4c5ccccc5c5cc(-c6ccc7c(c6)c6ccccc6n7-c6ccc(-c7ccccc7)cc6)ccc54)cc3)cc2)cc1 ; c1ccc(-c2ccc(-c3ccc(-n4c5ccccc5c5cc(-c6ccc7c(c6)c6ccccc6n7-c6cccc(-c7ccccc7)c6)ccc54)cc3)cc2)cc1]

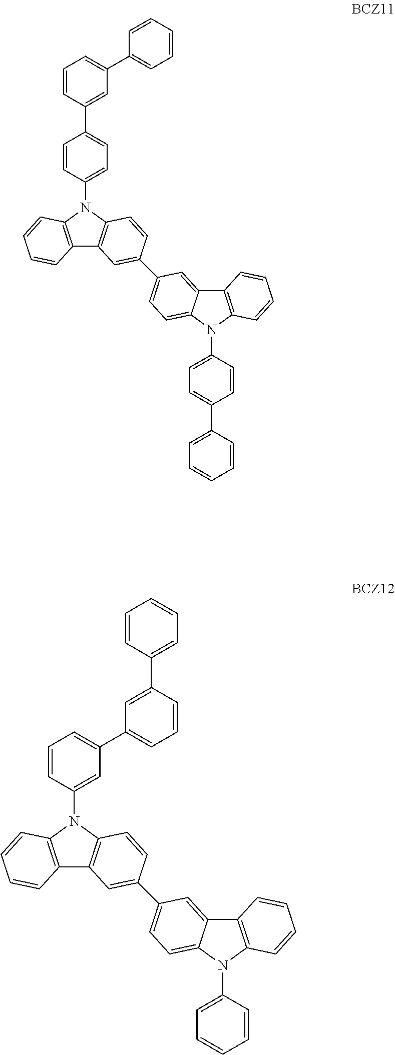 [SMILES: c1ccc(-c2ccc(-n3c4ccccc4c4cc(-c5ccc6c(c5)c5ccccc5n6-c5ccc(-c6cccc(-c7ccccc7)c6)cc5)ccc43)cc2)cc1 ; c1ccc(-c2cccc(-c3cccc(-n4c5ccccc5c5cc(-c6ccc7c(c6)c6ccccc6n7-c6ccccc6)ccc54)c3)c2)cc1]

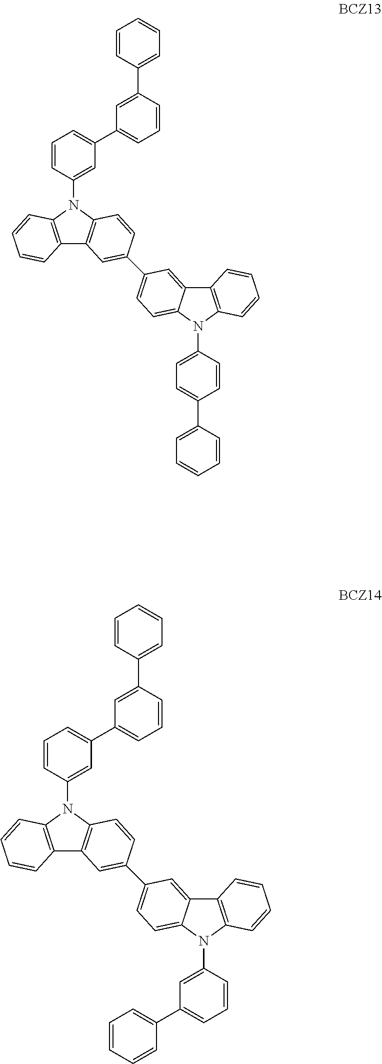 [SMILES: c1ccc(-c2ccc(-n3c4ccccc4c4cc(-c5ccc6c(c5)c5ccccc5n6-c5cccc(-c6cccc(-c7ccccc7)c6)c5)ccc43)cc2)cc1 ; c1ccc(-c2cccc(-c3cccc(-n4c5ccccc5c5cc(-c6ccc7c(c6)c6ccccc6n7-c6cccc(-c7ccccc7)c6)ccc54)c3)c2)cc1]

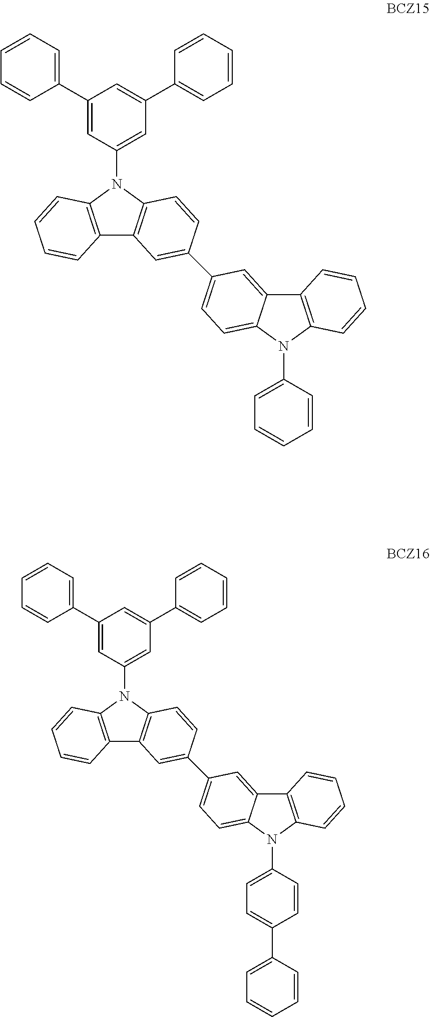 [SMILES: c1ccc(-c2cc(-c3ccccc3)cc(-n3c4ccccc4c4cc(-c5ccc6c(c5)c5ccccc5n6-c5ccccc5)ccc43)c2)cc1 ; c1ccc(-c2ccc(-n3c4ccccc4c4cc(-c5ccc6c(c5)c5ccccc5n6-c5cc(-c6ccccc6)cc(-c6ccccc6)c5)ccc43)cc2)cc1]

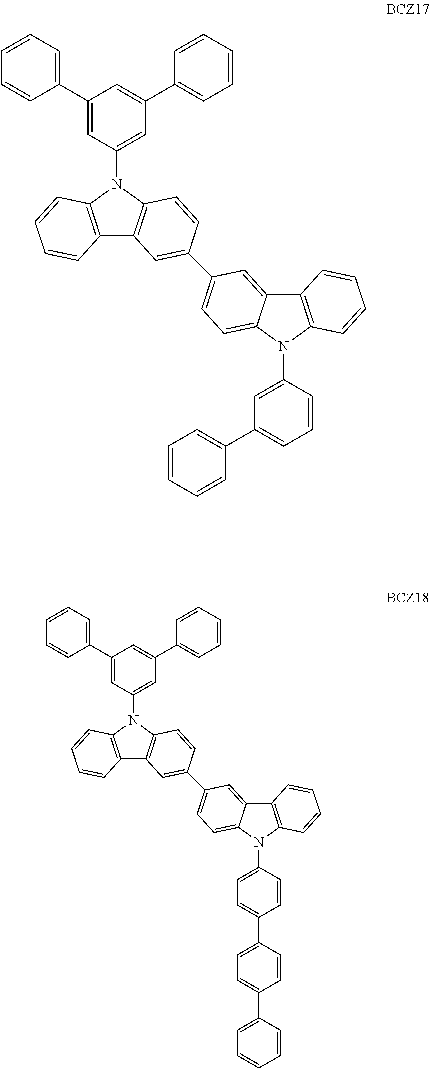 [SMILES: c1ccc(-c2cccc(-n3c4ccccc4c4cc(-c5ccc6c(c5)c5ccccc5n6-c5cc(-c6ccccc6)cc(-c6ccccc6)c5)ccc43)c2)cc1 ; c1ccc(-c2ccc(-c3ccc(-n4c5ccccc5c5cc(-c6ccc7c(c6)c6ccccc6n7-c6cc(-c7ccccc7)cc(-c7ccccc7)c6)ccc54)cc3)cc2)cc1]

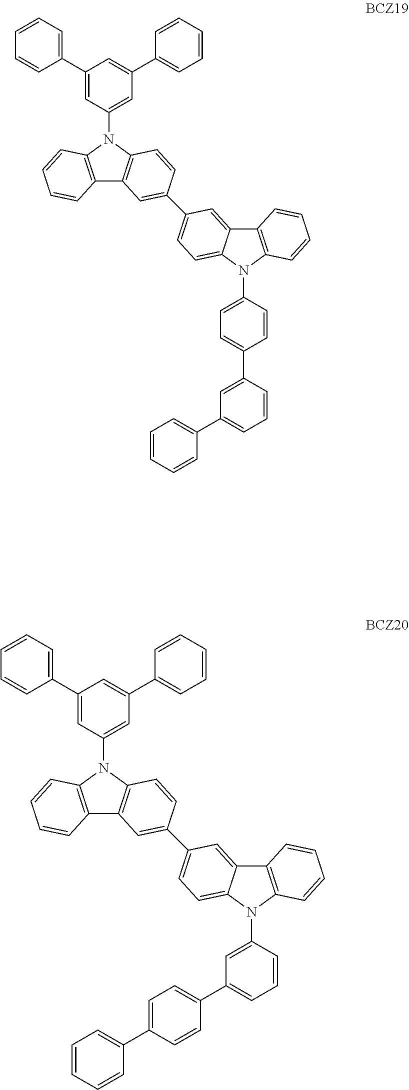 [SMILES: c1ccc(-c2cccc(-c3ccc(-n4c5ccccc5c5cc(-c6ccc7c(c6)c6ccccc6n7-c6cc(-c7ccccc7)cc(-c7ccccc7)c6)ccc54)cc3)c2)cc1 ; c1ccc(-c2ccc(-c3cccc(-n4c5ccccc5c5cc(-c6ccc7c(c6)c6ccccc6n7-c6cc(-c7ccccc7)cc(-c7ccccc7)c6)ccc54)c3)cc2)cc1]

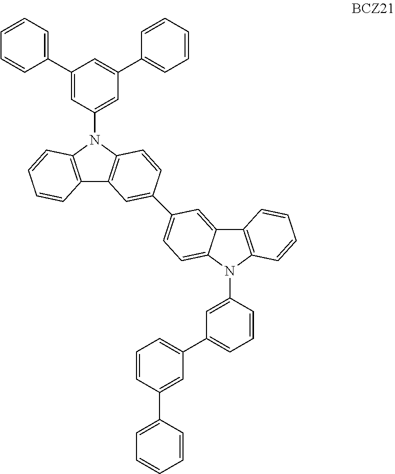 [SMILES: c1ccc(-c2cccc(-c3cccc(-n4c5ccccc5c5cc(-c6ccc7c(c6)c6ccccc6n7-c6cc(-c7ccccc7)cc(-c7ccccc7)c6)ccc54)c3)c2)cc1]

In the green pixel region, the EML 230 may include one of the compounds in Formula 9 as a dopant (e.g., an emitter).

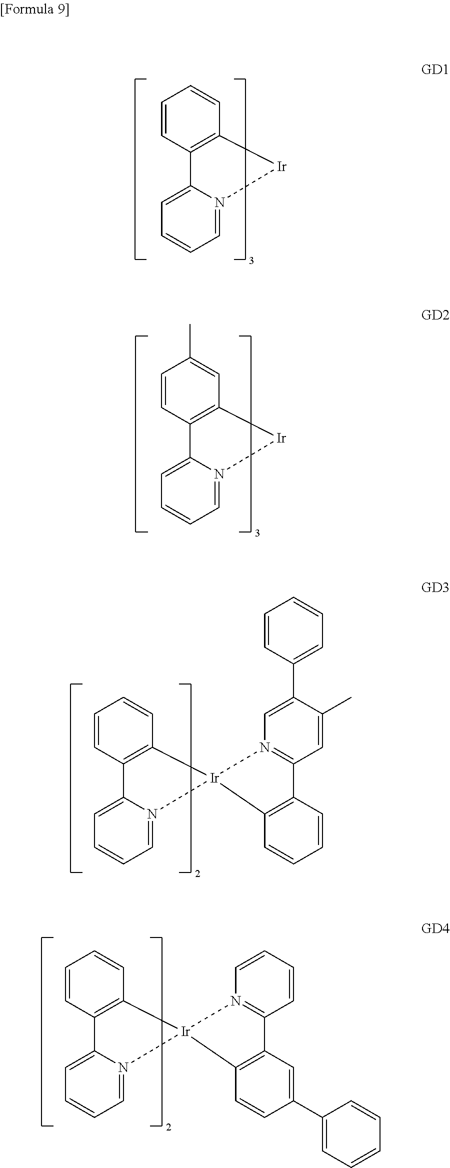

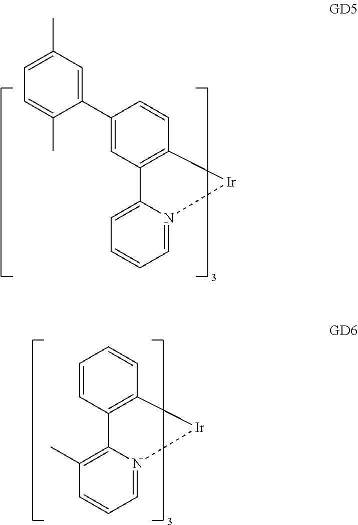

The EML 230 may have a thickness of 10 to 50 nm, e.g., 20 to 40 nm.

In the EML 230, a weight % of each of the first and second hosts may be greater than that of the dopant. The weight % of the first host and the weight % of the second host may be the same or different. In the green EML 230, a weight % ratio of the first host to the second host may be 1:9 to 9:1, 2:8 to 8:2 or 7:3 to 3:7. In some embodiments, the weight % of the first host and the weight % of the second host may be the same. For example, the first host and the second host may be present at the same weight %, and the dopant may be present in an amount of 5 to 25 weight % in the green EML 230, based on a total weight of the components in the green EML 230.

In an aspect of the present disclosure, the HBL 244 may include the organic compound of the present disclosure represented by Formula 1, and the ETL 242 may include at least one of a compound represented by Formula 10, e.g., a first electron transporting material, a compound represented by Formula 11, e.g., a second electron transporting material, and a compound represented by Formula 12, e.g., a third electron transporting material, instead of the organic compound of the present disclosure represented by Formula 1.

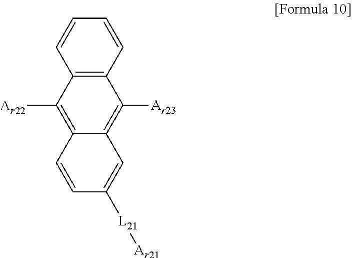

In Formula 10, L₂₁ is selected from the group consisting of a single bond, a substituted or unsubstituted C6 to C60 arylene group, and a substituted or unsubstituted C3 to C60 heteroarylene group,
Ar₂₁ is represented by Formula 10a or Formula 10b,
each of Ar₂₂ and Ar₂₃ is independently selected from the group consisting of hydrogen, deuterium, a substituted or unsubstituted C1 to C10 alkyl group, a substituted or unsubstituted C3 to C30 cycloalkyl group, a substituted or unsubstituted C1 to C10 alkoxy group, a substituted or unsubstituted C6 to C60 aryl group, and a substituted or unsubstituted C3 to C60 heteroaryl group,

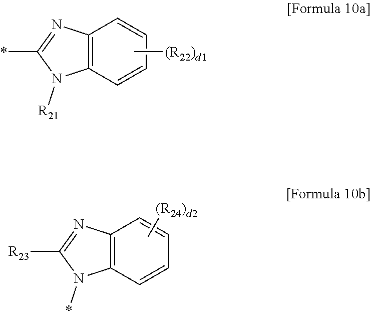

in Formula 10a, d1 is an integer of 0 to 4,
R₂₁ is selected from the group consisting of hydrogen, deuterium, a substituted or unsubstituted C1 to C10 alkyl group, a substituted or unsubstituted C3 to C30 cycloalkyl group, a substituted or unsubstituted C1 to C10 alkoxy group, a substituted or unsubstituted C6 to C60 aryl group, and a substituted or unsubstituted C3 to C60 heteroaryl group,
each R₂₂ is selected from the group consisting of deuterium, a substituted or unsubstituted C1 to C10 alkyl group, a substituted or unsubstituted C3 to C30 cycloalkyl group, a substituted or unsubstituted C1 to C10 alkoxy group, a substituted or unsubstituted C6 to C60 aryl group, and a substituted or unsubstituted C3 to C60 heteroaryl group,
in Formula 10b, d2 is an integer of 0 to 4,
R₂₃ is selected from the group consisting of hydrogen, deuterium, a substituted or unsubstituted C1 to C10 alkyl group, a substituted or unsubstituted C3 to C30 cycloalkyl group, a substituted or unsubstituted C1 to C10 alkoxy group, a substituted or unsubstituted C6 to C60 aryl group, and a substituted or unsubstituted C3 to C60 heteroaryl group,
each R₂₄ is selected from the group consisting of deuterium, a substituted or unsubstituted C1 to C10 alkyl group, a substituted or unsubstituted C3 to C30 cycloalkyl group, a substituted or unsubstituted C1 to C10 alkoxy group, a substituted or unsubstituted C6 to C60 aryl group, and a substituted or unsubstituted C3 to C60 heteroaryl group.

In each of Formulas 10a and 10b, the mark “*” denotes a bonding site.

In an aspect of the present disclosure, each of Ar₂₂ and Ar₂₃ may be independently a C6 to C60 aryl group, e.g., phenyl or naphthyl, unsubstituted or substituted with a C1 to C10 alkyl group, e.g., tert-butyl.

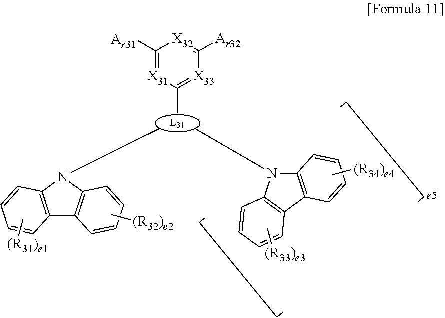

In Formula 11, each of e1, e2, e3 and e4 is independently an integer of 0 to 4, and e5 is 0 or 1,
each R₃₁, R₃₂, R₃₃, and R₃₄ is independently selected from the group consisting of deuterium, a substituted or unsubstituted C1 to C10 alkyl group, a substituted or unsubstituted C3 to C30 cycloalkyl group, a substituted or unsubstituted C1 to C10 alkoxy group, a substituted or unsubstituted C6 to C60 aryl group, and a substituted or unsubstituted C3 to C60 heteroaryl group,
each of X₃₁, X₃₂ and X₃₃ is independently N or CR₃₅, at least two of X₃₁, X₃₂ and X₃₃ is N,
each R₃₅ is selected from the group consisting of hydrogen, deuterium, a substituted or unsubstituted C1 to C10 alkyl group, a substituted or unsubstituted C3 to C30 cycloalkyl group, a substituted or unsubstituted C1 to C10 alkoxy group, a substituted or unsubstituted C6 to C60 aryl group, and a substituted or unsubstituted C3 to C60 heteroaryl group,
each of Ar₃₁ and Ar₃₂ is independently selected from the group consisting of a substituted or unsubstituted C6 to C60 aryl group, and a substituted or unsubstituted C3 to C60 heteroaryl group, and

L₃₁ is selected from the group consisting of a substituted or unsubstituted C6 to C60 arylene group, and a substituted or unsubstituted C3 to C60 heteroarylene group.

In an aspect of the present disclosure, each of e1, e2, e3 and e4 may be 0 or 1.

In an aspect of the present disclosure, each R₃₁, R₃₂, R₃₃, and R₃₄ may be independently a substituted or unsubstituted C6 to C60 aryl group, e.g., phenyl.

In an aspect of the present disclosure, two of X₃₁, X₃₂ and X₃₃ may be N, the other one of X₃₁, X₃₂ and X₃₃ may be CR₃₅, and R₃₅ may be hydrogen.

In an aspect of the present disclosure, each of Ar₃₁ and Ar₃₂ may be independently a substituted or unsubstituted C6 to C60 aryl group, e.g., phenyl or biphenyl.

In an aspect of the present disclosure, L₃₁ may be a substituted or unsubstituted C6 to C60 arylene group, e.g., phenylene.

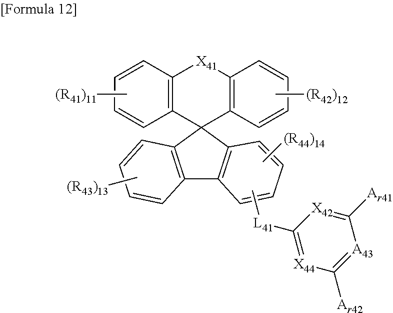

In Formula 12, each of f1, f2 and f3 is independently an integer of 0 to 4, and f4 is an integer of 0 to 3,
each R₄₁, R₄₂, R₄₃, and R₄₄ is independently selected from the group consisting of a substituted or unsubstituted C1 to C10 alkyl group, a substituted or unsubstituted C3 to C30 cycloalkyl group, a substituted or unsubstituted C1 to C10 alkoxy group, a substituted or unsubstituted C6 to C60 aryl group, and a substituted or unsubstituted C3 to C60 heteroaryl group,
X₄₁ is O, S or NR₄₅,
R₄₅ is a substituted or unsubstituted C6 to C60 aryl group and forms a ring with adjacent benzene ring,
each of X₄₂, X₄₃ and X₄₄ is independently N or CR₄₆, at least two of X₄₂, X₄₃ and X₄₄ is N,
each Ar₄₁ and Ar₄₂, R₄₆ is independently selected from the group consisting of hydrogen, a substituted or unsubstituted C6 to C60 aryl group, and a substituted or unsubstituted C3 to C60 heteroaryl group, and
L₄₁ is selected from the group consisting of a single bond, a substituted or unsubstituted C6 to C60 arylene group, and a substituted or unsubstituted C3 to C60 heteroarylene group.

In an aspect of the present disclosure, each of f1, f2, f3 and f4 may be 0.

In an aspect of the present disclosure, R₄₅ may be phenyl and form a carbazole with the nitrogen atom and adjacent benzene ring.

In an aspect of the present disclosure, L₄₁ may be a single bond or a substituted or unsubstituted C6 to C60 arylene group, e.g., phenylene.

In an aspect of the present disclosure, each of Ar₄₁ and Ar₄₂ is independently a substituted or unsubstituted C6 to C60 aryl group, e.g., phenyl, naphthyl or naphthylphenyl.

In an aspect of the present disclosure, R₄₆ may be hydrogen.

The first electron transporting material of Formula 10 may be one of the compounds in Formula 13.

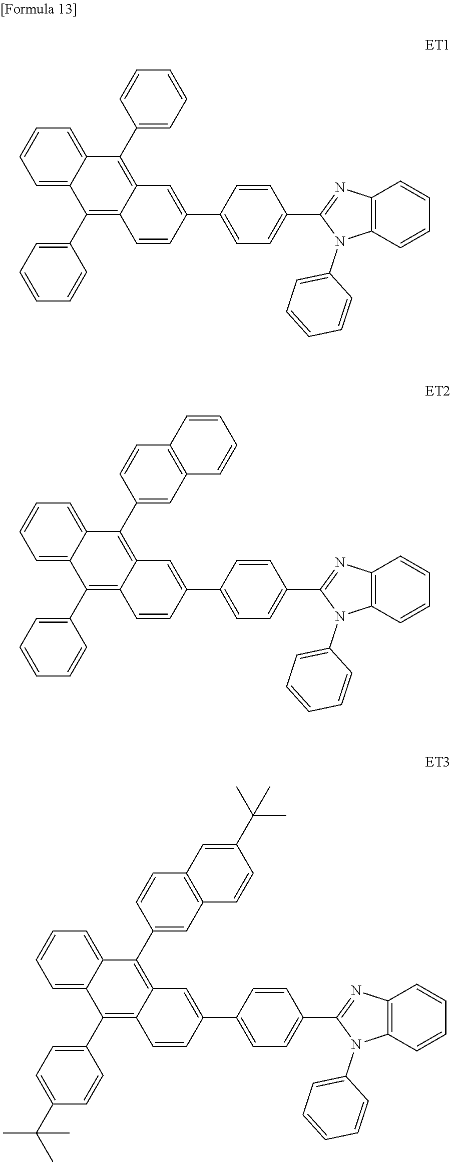 [SMILES: c1ccc(-c2c3ccccc3c(-c3ccccc3)c3cc(-c4ccc(-c5nc6ccccc6n5-c5ccccc5)cc4)ccc23)cc1 ; c1ccc(-c2c3ccccc3c(-c3ccc4ccccc4c3)c3cc(-c4ccc(-c5nc6ccccc6n5-c5ccccc5)cc4)ccc23)cc1 ; CC(C)(C)c1ccc(-c2c3ccccc3c(-c3ccc4cc(C(C)(C)C)ccc4c3)c3cc(-c4ccc(-c5nc6ccccc6n5-c5ccccc5)cc4)ccc23)cc1]

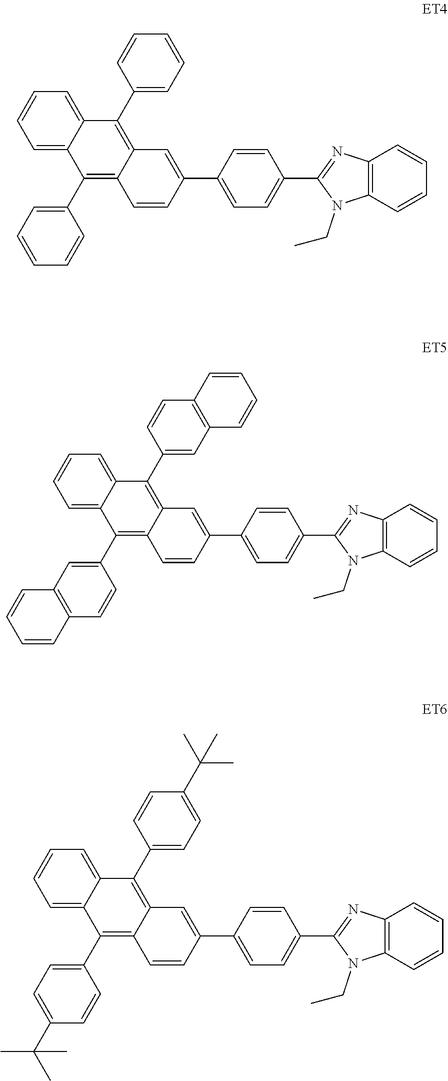 [SMILES: CCn1c(-c2ccc(-c3ccc4c(-c5ccccc5)c5ccccc5c(-c5ccccc5)c4c3)cc2)nc2ccccc21 ; CCn1c(-c2ccc(-c3ccc4c(-c5ccc6ccccc6c5)c5ccccc5c(-c5ccc6ccccc6c5)c4c3)cc2)nc2ccccc21 ; CCn1c(-c2ccc(-c3ccc4c(-c5ccc(C(C)(C)C)cc5)c5ccccc5c(-c5ccc(C(C)(C)C)cc5)c4c3)cc2)nc2ccccc21]

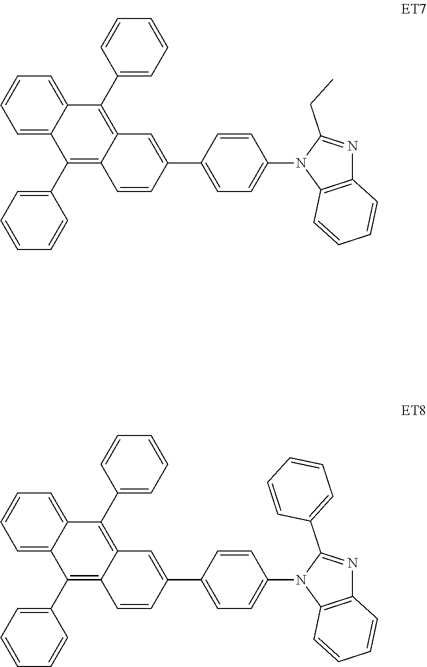 [SMILES: CCc1nc2ccccc2n1-c1ccc(-c2ccc3c(-c4ccccc4)c4ccccc4c(-c4ccccc4)c3c2)cc1 ; c1ccc(-c2c3ccccc3c(-c3ccccc3)c3cc(-c4ccc(-n5c(-c6ccccc6)nc6ccccc65)cc4)ccc23)cc1]

The second electron transporting material of Formula 11 may be one of the compounds in Formula 14.

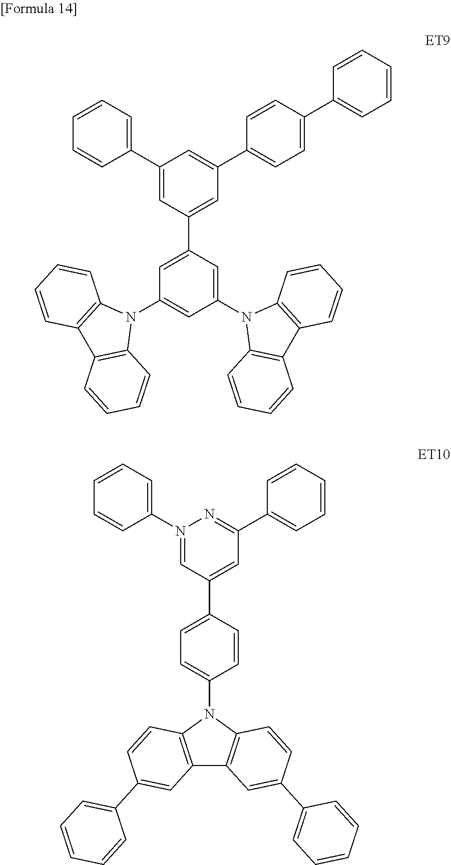

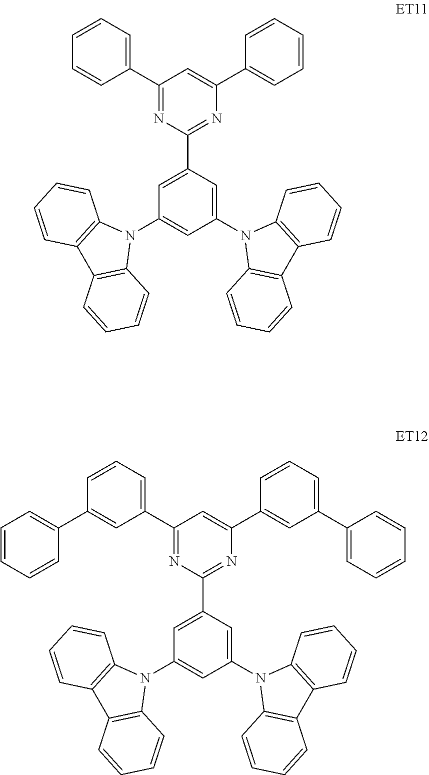 [SMILES: c1ccc(-c2cc(-c3ccccc3)nc(-c3cc(-n4c5ccccc5c5ccccc54)cc(-n4c5ccccc5c5ccccc54)c3)n2)cc1 ; c1ccc(-c2cccc(-c3cc(-c4cccc(-c5ccccc5)c4)nc(-c4cc(-n5c6ccccc6c6ccccc65)cc(-n5c6ccccc6c6ccccc65)c4)n3)c2)cc1]

The third electron transporting material of Formula 12 may be one of the compounds in Formula 15.

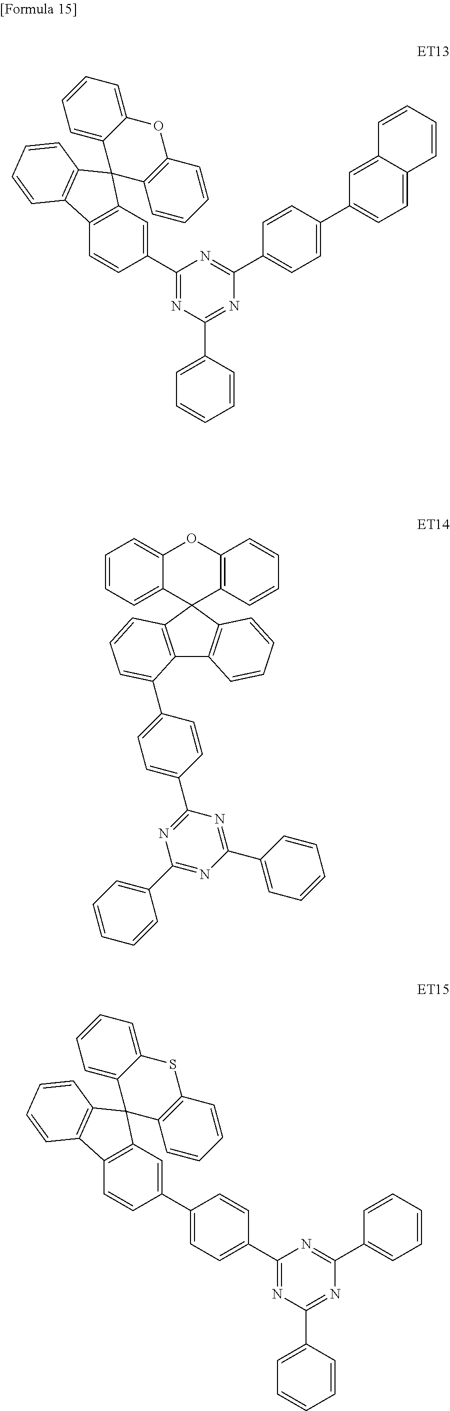 [SMILES: c1ccc(-c2nc(-c3ccc(-c4ccc5ccccc5c4)cc3)nc(-c3ccc4c(c3)C3(c5ccccc5Oc5ccccc53)c3ccccc3-4)n2)cc1 ; c1ccc(-c2nc(-c3ccccc3)nc(-c3ccc(-c4cccc5c4-c4ccccc4C54c5ccccc5Oc5ccccc54)cc3)n2)cc1 ; c1ccc(-c2nc(-c3ccccc3)nc(-c3ccc(-c4ccc5c(c4)C4(c6ccccc6Sc6ccccc64)c4ccccc4-5)cc3)n2)cc1]

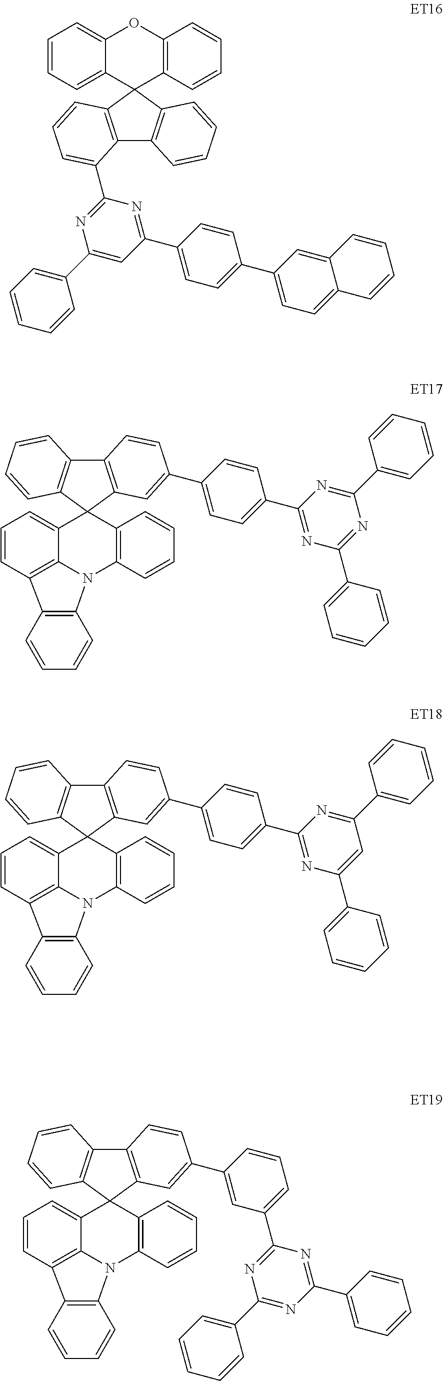 [SMILES: c1ccc(-c2cc(-c3ccc(-c4ccc5ccccc5c4)cc3)nc(-c3cccc4c3-c3ccccc3C43c4ccccc4Oc4ccccc43)n2)cc1 ; c1ccc(-c2nc(-c3ccccc3)nc(-c3ccc(-c4ccc5c(c4)C4(c6ccccc6-5)c5ccccc5-n5c6ccccc6c6cccc4c65)cc3)n2)cc1 ; c1ccc(-c2cc(-c3ccccc3)nc(-c3ccc(-c4ccc5c(c4)C4(c6ccccc6-5)c5ccccc5-n5c6ccccc6c6cccc4c65)cc3)n2)cc1 ; c1ccc(-c2nc(-c3ccccc3)nc(-c3cccc(-c4ccc5c(c4)C4(c6ccccc6-5)c5ccccc5-n5c6ccccc6c6cccc4c65)c3)n2)cc1]

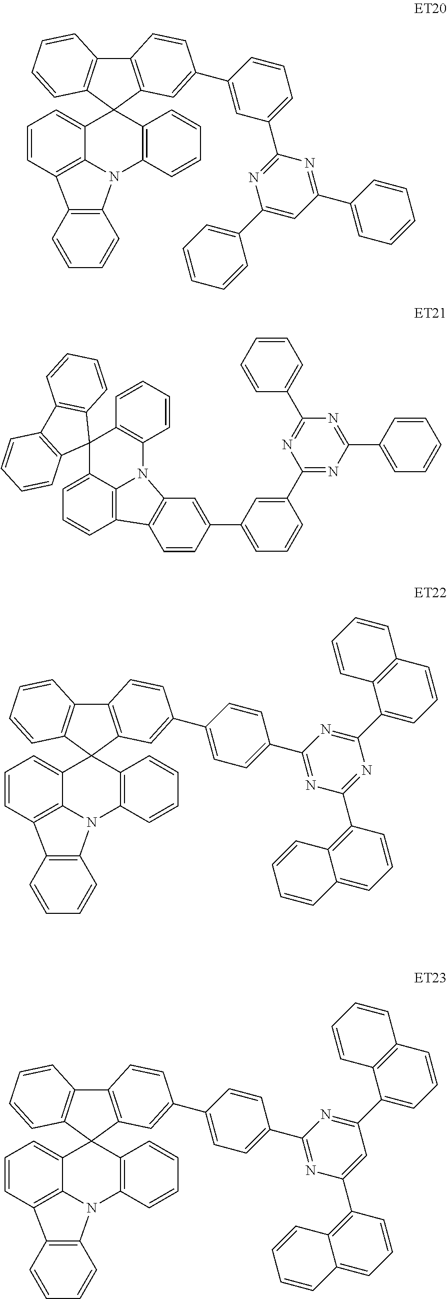 [SMILES: c1ccc(-c2cc(-c3ccccc3)nc(-c3cccc(-c4ccc5c(c4)C4(c6ccccc6-5)c5ccccc5-n5c6ccccc6c6cccc4c65)c3)n2)cc1 ; c1ccc(-c2nc(-c3ccccc3)nc(-c3cccc(-c4ccc5c6cccc7c6n(c5c4)-c4ccccc4C74c5ccccc5-c5ccccc54)c3)n2)cc1 ; c1ccc2c(c1)-c1ccc(-c3ccc(-c4nc(-c5cccc6ccccc56)nc(-c5cccc6ccccc56)n4)cc3)cc1C21c2ccccc2-n2c3ccccc3c3cccc1c32 ; c1ccc2c(c1)-c1ccc(-c3ccc(-c4nc(-c5cccc6ccccc56)cc(-c5cccc6ccccc56)n4)cc3)cc1C21c2ccccc2-n2c3ccccc3c3cccc1c32]

In an aspect of the present disclosure, the ETL 242 may include a compound in Formula 16 instead of the organic compound of the present disclosure represented by Formula 1.

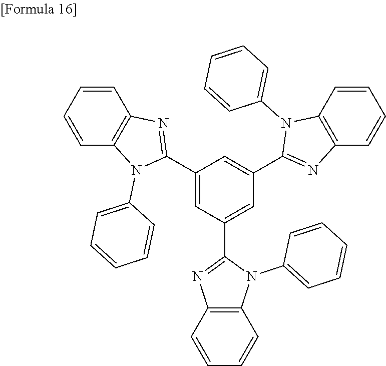 [SMILES: c1ccc(-n2c(-c3cc(-c4nc5ccccc5n4-c4ccccc4)cc(-c4nc5ccccc5n4-c4ccccc4)c3)nc3ccccc32)cc1]

In an aspect of the present disclosure, the ETL 242 may include the organic compound of the present disclosure represented by Formula 1, and the HBL 244 may include a compound in Formula 17 instead of the organic compound of the present disclosure represented by Formula 1.

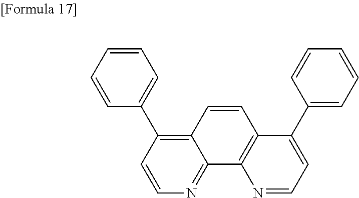 [SMILES: c1ccc(-c2ccnc3c2ccc2c(-c4ccccc4)ccnc23)cc1]

The red EML may include a red host and a red dopant. The red dopant may include at least one of a red phosphorescent compound, a red fluorescent compound, and a red delayed fluorescent compound. In the red EML, the red host may be present at a weight % greater than the red dopant. In the red EML, the red dopant may be present at a weight % of 1 to 10, or 1 to 5, based on a total weight of the components in the red EML.

For example, the red host may be at least one selected from the group consisting of 9,9′-diphenyl-9H,9′H-3,3′-bicarbazole (BCzPh), CBP, 1,3,5-tris(carbazole-9-yl)benzene (TCP), TCTA, 4,4′-bis(carbazole-9-yl)-2,2′-dimethylbipheyl (CDBP), 2,7-bis(carbazole-9-yl)-9,9-dimethylfluorene (DMFL-CBP), 2,2′,7,7′-tetrakis(carbazole-9-yl)-9,9-spiorofluorene (Spiro-CBP), DPEPO, 4′-(9H-carbazol-9-yl)biphenyl-3,5-dicarbonitrile (PCzB-2CN), 3′-(9H-carbazol-9-yl)biphenyl-3,5-dicarbonitrile (mCzB-2CN), 3,6-bis(carbazole-9-yl)-9-(2-ethyl-hexyl)-9H-carbazole (TCzl), bis(2-hydroxylphenyl)-pyridine)beryllium (Bepp₂), bis(10-hydroxylbenzo[h]quinolinato)beryllium (Bebg₂), and 1,3,5-tris(1-pyrenyl)benzene (TPB₃), but it is not limited thereto.

The red dopant may be at least one selected from the group consisting of [bis(2-(4,6-dimethyl)phenylquinoline)](2,2,6,6-tetramethylheptane-3,5-dionate)iridium(III), bis[2-(4-n-hexylphenyl)quinoline](acetylacetonate)iridium(III) (Hex-Ir(phq)₂(acac)), tris[2-(4-n-hexylphenyl)quinoline]iridium(III), (Hex-Ir(phq)₃), tris[2-phenyl-4-methylquinoline]iridium(III) (Ir(Mphq)₃), bis(2-phenylquinoline)(2,2,6,6-tetramethylheptene-3,5-dionate)iridium(III) (Ir(dpm)PQ₂), bis(phenylisoquinoline)(2,2,6,6-tetramethylheptene-3,5-dionate)iridium(III), (Ir(dpm)(piq)₂), bis[(4-n-hexylphenyl)isoquinoline](acetylacetonate)iridium(III) (Hex-Ir(piq)₂(acac)), tris[2-(4-n-hexylphenyl)quinoline]iridium(III) (Hex-Ir(piq)₃), tris(2-(3-methylphenyl)-7-methyl-quinolato)iridium (Ir(dmpq)₃), bis[2-(2-methylphenyl)-7-methyl-quinoline](acetylacetonate)iridium(III) (Jr(dmpq)₂(acac)), and bis[2-(3,5-dimethylphenyl)-4-methyl-quinoline](acetylacetonate)iridium(III) (Ir(mphmq)₂(acac)), but it is not limited thereto.

The blue EML may include a blue host and a blue dopant. The blue dopant may include at least one of a blue phosphorescent compound, a blue fluorescent compound, and a blue delayed fluorescent compound. In the blue EML, the blue host may be present at a weight % greater than the blue dopant. In the blue EML, the blue dopant may be present at a weight % of 1 to 10, or 1 to 5, based on a total weight of the components in the blue EML.

For example, the blue host may be independently at least one selected from the group consisting of mCP, 9-(3-(9H-carbazol-9-yl)phenyl)-9H-carbazole-3-carbonitrile (mCP-CN), mCBP, CBP-CN, 9-(3-(9H-Carbazol-9-yl)phenyl)-3-(diphenylphosphoryl)-9H-carbazole (mCPPO1) 3,5-Di(9H-carbazol-9-yl)biphenyl (Ph-mCP), TSPO1, 9-(3′-(9H-carbazol-9-yl)-[1,1′-biphenyl]-3-yl)-9H-pyrido[2,3-b]indole (CzBPCb), bis(2-methylphenyl)diphenylsilane (UGH-1), 1,4-bis(triphenylsilyl)benzene (UGH-2), 1,3-bis(triphenylsilyl)benzene (UGH-3), 9,9-spiorobifluoren-2-yl-diphenyl-phosphine oxide (SPPO1), and 9,9′-(5-(triphenylsilyl)-1,3-phenylene)bis(9H-carbazole) (SimCP), but it is not limited thereto.

The blue dopant may be independently at least one selected from the group consisting of 4,4′-bis[4-(di-p-tolylamino)styryl]biphenyl (DPAVBi), 4-(di-p-tolylamino)-4-4′-[(di-p-tolylamino)styryl]stilbene (DPAVB), 4,4′-bis[4-(diphenylamino)styryl]biphenyl (BDAVBi), 2,7-bis(4-diphenylamino)styryl)-9,9-spiorfluorene (spiro-DPVBi), [1,4-bis[2-[4-[N,N-di(p-tolyl)amino]phenyl]vinyl]benzene (DSB), 1-4-di-[4-(N,N-diphenyl)amino]styryl-benzene (DSA), 2,5,8,11-tetra-tert-butylperylene (TBPe), bis(2-hydroxylphenyl)-pyridine)beryllium (Bepp₂), 9-(9-Phenylcarbazole-3-yl)-10-(naphthalene-1-yl)anthracene (PCAN), mer-tris(1-phenyl-3-methylimidazolin-2-ylidene-C,C(2)′iridium(III) (mer-Ir(pmi)₃), fac-Tris(1,3-diphenyl-benzimidazolin-2-ylidene-C,C(2)′iridium(III) (fac-Ir(dpbic)₃), bis(3,4,5-trifluoro-2-(2-pyridyl)phenyl-(2-carboxypyridyl)iridium(III) (Ir(tfpd)2pic), tris(2-(4,6-difluorophenyl)pyridine))iridium(III) (Ir(Fppy)₃), and bis[2-(4,6-difluorophenyl)pyridinato-C2,N](picolinato)iridium(III) (FIrpic), but it is not limited thereto.

A thickness of the HTL 220 may be greater than that of each of the EML 230, the ETL 242 and the HBL 244. The HTL 220 may have a thickness of 80 to 129 nm, e.g., 90 to 110 nm.

The HIL 210 may include at least one compound selected from the group consisting of 4,4′,4″-tris(3-methylphenylamino)triphenylamine (MTDATA), 4,4′,4″-tris(N,N-diphenyl-amino)triphenylamine (NATA), 4,4′,4″-tris(N-(naphthalene-1-yl)-N-phenyl-amino)triphenylamine (1T-NATA), 4,4′,4″-tris(N-(naphthalene-2-yl)-N-phenyl-amino)triphenylamine (2T-NATA), copper phthalocyanine(CuPc), tris(4-carbazoyl-9-yl-phenyl)amine (TCTA), N,N′-diphenyl-N,N′-bis(1-naphthyl)-1,1′-biphenyl-4,4″-diamine (NPB or NPD), 1,4,5,8,9,11-hexaazatriphenylenehexacarbonitrile(dipyrazino[2,3-f.2′3′-h]quinoxaline-2,3,6,7,10,11-hexacarbonitrile (HAT-CN), 1,3,5-tris[4-(diphenylamino)phenyl]benzene (TDAPB), poly(3,4-ethylenedioxythiphene)polystyrene sulfonate (PEDOT/PSS), and N-(biphenyl-4-yl)-9,9-dimethyl-N-(4-(9-phenyl-9H-carbazol-3-yl)phenyl)-9H-fluoren-2-amine. The HIL 210 may have a thickness of 1 to 10 nm, e.g., 3 to 7 nm.

The HTL 220 may include at least one compound selected from the group consisting of N,N′-diphenyl-N,N′-bis(3-methylphenyl)-1,1′-biphenyl-4,4′-diamine (TPD), NPB (or NPD), 4,4′-bis(N-carbazolyl)-1,1′-biphenyl (CBP), poly[N,N′-bis(4-butylphenyl)-N,N′-bis(phenyl)-benzidine](poly-TPD), (poly[(9,9-dioctylfluorenyl-2,7-diyl)-co-(4,4′-(N-(4-sec-butylphenyl)diphenylamine))](TFB), di-[4-(N,N-di-p-tolyl-amino)-phenyl]cyclohexane (TAPC), 3,5-di(9H-carbazol-9-yl)-N,N-diphenylaniline (DCDPA), N-(biphenyl-4-yl)-9,9-dimethyl-N-(4-(9-phenyl-9H-carbazol-3-yl)phenyl)-9H-fluoren-2-amine, and N-(biphenyl-4-yl)-N-(4-(9-phenyl-9H-carbazol-3-yl)phenyl)biphenyl-4-amine.

The EIL 250 may include at least one of an alkali metal, such as Li, an alkali halide compound, such as LiF, CsF, NaF, or BaF₂, and an organo-metallic compound, such as Liq, lithium benzoate, or sodium stearate. The EIL 250 may have a thickness of 1 to 10 nm, e.g., 3 to 7 nm.

The EBL may include at least one compound selected from the group consisting of tris(4-carbazoyl-9-yl-phenyl)amine (TCTA), tris[4-(diethylamino)phenyl]amine, N-(biphenyl-4-yl)-9,9-dimethyl-N-(4-(9-phenyl-9H-carbazol-3-yl)phenyl)-9H-fluoren-2-amine, di-[4-(N,N-di-p-tolyl-amino)-phenyl]cyclohexane (TAPC), 4,4′,4″-tris(3-methylphenylamino)triphenylamine (MTDATA), 1,3-bis(carbazol-9-yl)benzene (mCP), 3,3′-bis(N-carbazolyl)-1,1′-biphenyl (mCBP), copper phthalocyanine (CuPc), N,N′-bis[4-[bis(3-methylphenyl)amino]phenyl]-N,N′-diphenyl-[1,1′-biphenyl]-4,4′-diamine (DNTPD), 1,3,5-tris[4-(diphenylamino)phenyl]benzene (TDAPB), 3,5-di(9H-carbazol-9-yl)-N,N-diphenylaniline (DCDPA) and 2,8-bis(9-phenyl-9H-carbazol-3-yl)dibenzo[b,d]thiophene).

As illustrated above, in the OLED D, at least one of the ETL 242 and the HBL 244 includes the organic compound of the present disclosure represented by Formula 1. As a result, the emitting efficiency and the lifespan of the OLED D are improved.

In addition, in the OLED D, the ETL 242 may further include the compound in Formula 3 with the organic compound of the present disclosure represented by Formula 1 so that the emitting efficiency and the lifespan of the OLED D may be further improved.

Moreover, in the OLED D in the green pixel region, the EML 230, the ETL 242 and the HBL 244 may include the organic compound of the present disclosure represented by Formula 1 so that a manufacturing efficiency (production yield) of the OLED may be increased with improving the emitting efficiency and the lifespan. [OLED]

An anode (ITO), an HIL (a compound in Formula 18, 5 nm), an HTL (a compound in Formula 19, 100 nm), an EML (30 nm), an HBL (8 nm), an ETL (the compound in Formula 16 and the compound in Formula 3, 30 nm), an EIL (LiF, 5 nm) and a cathode (Al, 100 nm) was sequentially deposited to form a green OLED.

The EML was formed by using the compound BCZ1 in Formula 8, the compound in Formula 4 and the compound GD1 in Formula 9. The compound BCZ1 in Formula 8 and the compound in Formula 4 was used by the same weight %, and the compound GD1 in Formula 9 was doped by 15 wt %. The compound in Formula 16 and the compound in Formula 3 was used by the same weight %.

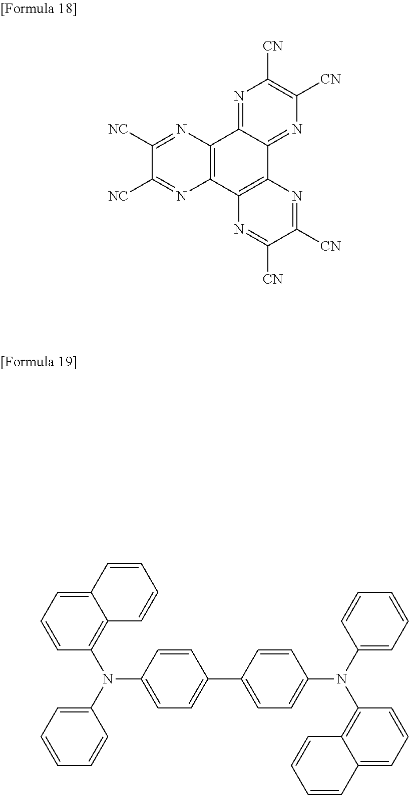 [SMILES: N#Cc1nc2c3nc(C#N)c(C#N)nc3c3nc(C#N)c(C#N)nc3c2nc1C#N ; c1ccc(N(c2ccc(-c3ccc(N(c4ccccc4)c4cccc5ccccc45)cc3)cc2)c2cccc3ccccc23)cc1]

### 1. Comparative Example

### (1) Comparative Example 1 (Ref1)

The compound in Formula 17 was used to form the HBL.

### (2) Comparative Example 2 (Ref2)

The compound in Formula 20 was used to form the HBL.

### (3) Comparative Example 3 (Ref3)

The compound in Formula 21 was used to form the HBL.

### (4) Comparative Example 4 (Ref4)

The compound in Formula 22 was used to form the HBL.

### (5) Comparative Example 5 (Ref5)

The compound in Formula 23 was used to form the HBL.

### (6) Comparative Example 6 (Ref6)

The compound in Formula 24 was used to form the HBL.

### (7) Comparative Example 7 (Ref7)

The compound in Formula 25 was used to form the HBL.

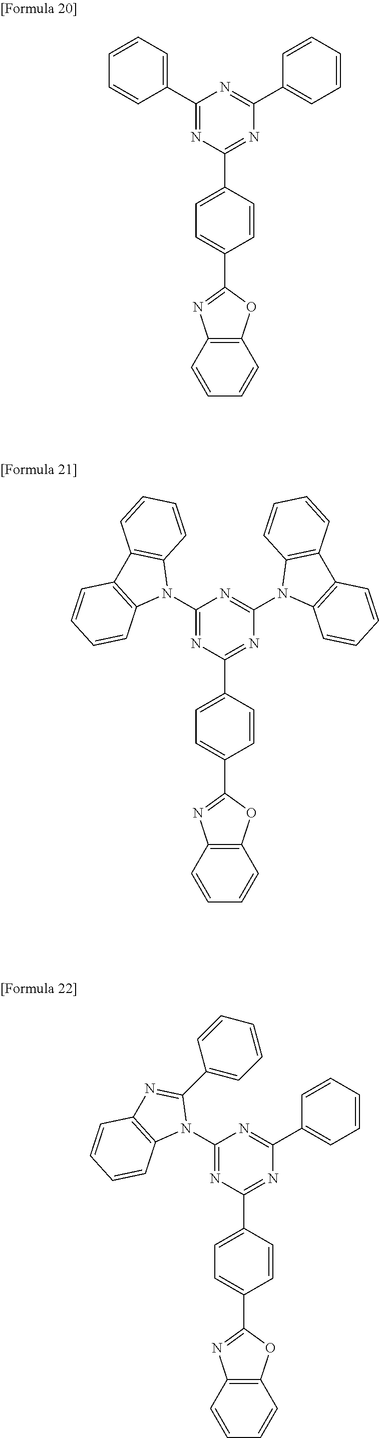 [SMILES: c1ccc(-c2nc(-c3ccccc3)nc(-c3ccc(-c4nc5ccccc5o4)cc3)n2)cc1 ; c1ccc2oc(-c3ccc(-c4nc(-n5c6ccccc6c6ccccc65)nc(-n5c6ccccc6c6ccccc65)n4)cc3)nc2c1 ; c1ccc(-c2nc(-c3ccc(-c4nc5ccccc5o4)cc3)nc(-n3c(-c4ccccc4)nc4ccccc43)n2)cc1]

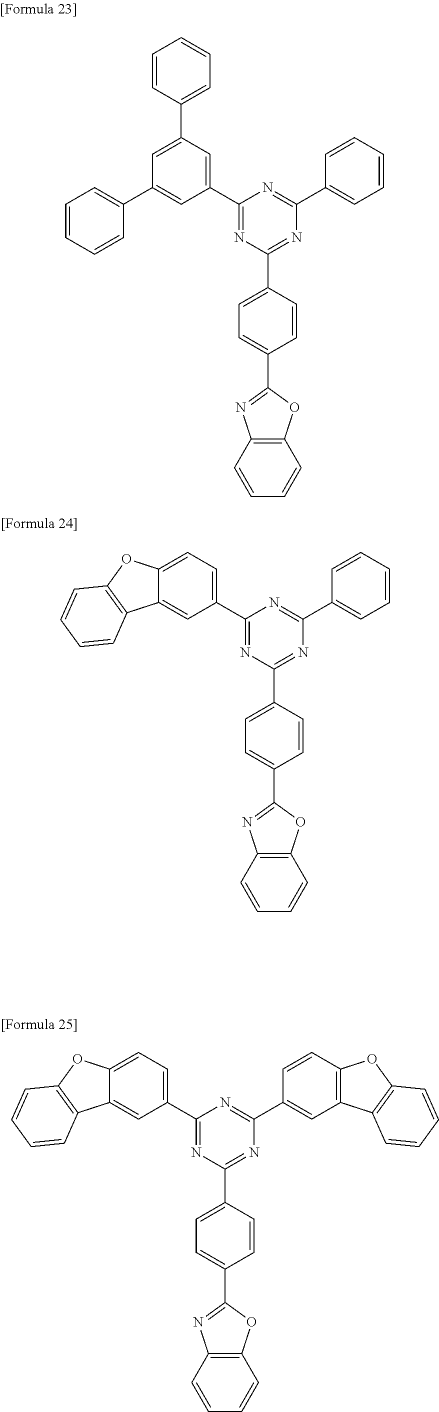 [SMILES: c1ccc(-c2cc(-c3ccccc3)cc(-c3nc(-c4ccccc4)nc(-c4ccc(-c5nc6ccccc6o5)cc4)n3)c2)cc1 ; c1ccc(-c2nc(-c3ccc(-c4nc5ccccc5o4)cc3)nc(-c3ccc4oc5ccccc5c4c3)n2)cc1 ; c1ccc2oc(-c3ccc(-c4nc(-c5ccc6oc7ccccc7c6c5)nc(-c5ccc6oc7ccccc7c6c5)n4)cc3)nc2c1]

### 2. Example

### (1) Example 1 (Ex 1)

The compound 1-1 in Formula 2 was used to form the HBL.

### (2) Example 2 (Ex2)

The compound 1-57 in Formula 2 was used to form the HBL.

### (3) Example 3 (Ex3)

The compound 3-28 in Formula 2 was used to form the HBL.

### (4) Example 4 (Ex4)

The compound 3-29 in Formula 2 was used to form the HBL.

### (5) Example 5 (Ex5)

The compound 3-43 in Formula 2 was used to form the HBL.

### (6) Example 6 (Ex6)

The compound 2-62 in Formula 2 was used to form the HBL.

### (7) Example 7 (Ex7)

The compound 2-1 in Formula 2 was used to form the HBL.

### (8) Example 8 (Ex8)

The compound 1-27 in Formula 2 was used to form the HBL.

### (9) Example 9 (Ex9)

The compound 2-12 in Formula 2 was used to form the HBL.

### (10) Example 10 (Ex10)

The compound 3-4 in Formula 2 was used to form the HBL.

The properties, e.g., the emitting efficiency and the lifespan, of the OLED in Comparative Examples 1 to 7 and Examples 1 to 10 were measured and listed in Table 1.

TABLE 1

| | HBL | Efficiency | Lifespan |
|---|---|---|---|
| Ref1 | Formula 17 | 100% | 100% |
| Ref2 | Formula 20 | 100% | 106% |
| Ref3 | Formula 21 | 101% | 108% |
| Ref4 | Formula 22 | 101% | 105% |
| Ref5 | Formula 23 | 101% | 104% |
| Ref6 | Formula 24 | 100% | 106% |
| Ref7 | Formula 25 | 101% | 107% |
| Ex1 | Compound 1-1 | 102% | 114% |
| Ex2 | Compound1-57 | 104% | 120% |
| Ex3 | Compound 3-28 | 102% | 131% |
| Ex4 | Compound 3-29 | 101% | 130% |
| Ex5 | Compound 3-43 | 103% | 128% |
| Ex6 | Compound 2-62 | 102% | 125% |
| Ex7 | Compound 2-1 | 103% | 124% |
| Ex8 | Compound 1-27 | 104% | 129% |
| Ex9 | Compound 2-12 | 103% | 125% |
| Ex10 | Compound 3-4 | 103% | 130% |

As shown in Table 1, in comparison to the OLED in Ref1 to Ref7, the OLED in Ex1 to Ex10, in which the LML included an example of the organic compound of the present disclosure, has improved emitting efficiency and the lifespan.

For example, the compound in Formula 20 includes a single benzoxazole moiety bonded to a phenylene linker at a para-position with respect to a triazine moiety, while the compound 1-1 in Formula 2 includes two benzoxazole moieties bonded to a phenylene linker at a meta-position with respect to a triazine moiety. In comparison to the OLLD in Ref2 using the compound in Formula 20, the lifespan of the OLLD in Ex1 using the compound 1-1 in Formula 2 is significantly increased.

The compound in Formula 21 includes a single benzoxazole moiety bonded to a phenylene linker at a para-position with respect to a triazine moiety, while the compound 3-28 in Formula 2 includes two benzoxazole moieties bonded to a phenylene linker at a meta-position with respect to a triazine moiety. In comparison to the OLED in Ref3 using the compound in Formula 21, the lifespan of the OLED in Ex3 using the compound 3-28 in Formula 2 is significantly increased.

The compound in Formula 23 includes a single benzoxazole moiety bonded to a phenylene linker at a para-position with respect to a triazine moiety, while the compound 1-57 in Formula 2 includes two benzoxazole moieties bonded to a phenylene linker at a meta-position with respect to a triazine moiety. In comparison to the OLED in Ref5 using the compound in Formula 23, the lifespan of the OLED in Ex2 using the compound 1-57 in Formula 2 is significantly increased.

The compound in Formula 24 includes a single benzoxazole moiety bonded to a phenylene linker at a para-position with respect to a triazine moiety, while the compound 2-62 in Formula 2 includes two benzoxazole moieties bonded to a phenylene linker at a meta-position with respect to a triazine moiety. In comparison to the OLED in Ref6 using the compound in Formula 24, the lifespan of the OLED in Ex6 using the compound 2-62 in Formula 2 is significantly increased.

The compound in Formula 25 includes a single benzoxazole moiety bonded to a phenylene linker at a para-position with respect to a triazine moiety, while the compound 3-29 in Formula 2 includes two benzoxazole moieties bonded to a phenylene linker at a meta-position with respect to a triazine moiety. In comparison to the OLED in Ref7 using the compound in Formula 25, the lifespan of the OLED in Ex2 using the compound 3-29 in Formula 2 is significantly increased.

FIG. 4 illustrates a schematic cross-sectional view of an OLED according to a third embodiment of the present disclosure.

As illustrated in FIG. 4, an OLED D may include first and second electrodes 160 and 164, which may face each other, and an organic light emitting layer 162 therebetween. The organic light emitting layer 162 may include a first emitting part 310 including a first EML 320 and a first charge auxiliary layer 316 and a second emitting part 330 including a second EML 340 and a second charge auxiliary layer 334. The first charge auxiliary layer 316 include at least one of a first ETL 316a and a first HBL 316b, and the second charge auxiliary layer 334 includes at least one of a second ETL 334a and a second HBL 334b. The first HBL 316b may be positioned between the first EML 320 and the first ETL 316a, and the second HBL 334b may be positioned between the second EML 340 and the second ETL 334a.

The organic light emitting layer 162 may further include a CGL 350 between the first and second emitting parts 310 and 330.

The organic light emitting display device 100 (of FIG. 2) may include at least one of a red pixel region, a green pixel region and a blue pixel region. The OLED D may be positioned in each of the red, green and blue pixel regions. Each of the first and second EMLs 320 and 340 in the red pixel region is a red EML. Each of the first and second EMLs 320 and 340 in the green pixel region is a green EML. Each of the first and second EMLs 320 and 340 in the blue pixel region is a blue EML.

The first electrode 160 may act as an anode for injecting a hole and may be formed of a conductive material, e.g., ITO or IZO, having a relatively high work function. The second electrode 164 may act a cathode for injecting an electron and may be formed of a conductive material, e.g., Al, Mg or AlMg, having a relatively low work function.

In the top-emission type OLED D, the first electrode 160 may further include a reflection layer to act as a reflective electrode, and the second electrode 164 may have a thin profile to act as a transparent (e.g. semi-transparent) electrode. Alternatively, in the bottom-emission type OLED D, the first electrode 160 may act as a transparent electrode, and the second electrode 164 may act as a reflective electrode. However, embodiments of the present disclosure are not limited to such examples.

The CGL 350 may be positioned between the first and second emitting parts 310 and 330. The first emitting part 310, the CGL 350, and the second emitting part 330 may be sequentially stacked on the first electrode 160. For example, the first emitting part 310 may be positioned between the first electrode 160 and the CGL 350. The second emitting part 330 may be positioned between the second electrode 164 and the CGL 350.

The first emitting part 310 may further include a first HTL 314 between the first EML 320 and the electrode 160. In addition, the first emitting part 310 may further include an HIL 312 between the first HTL 314 and the first electrode 160. Moreover, the first emitting part 310 may further include at least one of a first EBL between the first EML 320 and the first HTL 314.

The second emitting part 330 may further include a second HTL 332 between the second EML 340 and the CGL or between the second EML 340 and the first emitting part 310. In addition, the second emitting part 330 may further include an EIL 336 between the second charge auxiliary layer 334 and the second electrode 164. Moreover, the second emitting part 330 may further include a second EBL between the second EML 340 and the second HTL 332.

The CGL 350 may be positioned between the first and second emitting parts 310 and 330. For example, the first and second emitting parts 310 and 330 may be connected through the CGL 350. The CGL 350 may be a P-N junction CGL including an N-type CGL 352 and a P-type CGL 354.

The N-type CGL 352 may be positioned between the first charge auxiliary layer 316 and the second HTL 332. The P-type CGL 354 may be positioned between the N-type CGL 352 and the second HTL 332.

The N-type CGL 352 may be an organic layer doped with an alkali metal, e.g., Li, Na, K and Cs, and/or an alkali earth metal, e.g., Mg, Sr, Ba and Ra. For example, the N-type CGL 352 may be formed of an N-type charge generation material including a host being the organic material, e.g., 4,7-dipheny-1,10-phenanthroline (Bphen) and MTDATA, a dopant being an alkali metal and/or an alkali earth metal, and the dopant may be doped with a weight % of 0.01 to 30.

The P-type CGL 354 may be formed of a P-type charge generation material including an inorganic material, e.g., tungsten oxide (WOx), molybdenum oxide (MoOx), beryllium oxide (Be₂O₃) or vanadium oxide (V₂O₅), an organic material, e.g., NPD, HAT-CN, F4TCNQ, TPD, TNB, TCTA, N,N′-dioctyl-3,4,9,10-perylenedicarboximide (PTCDI-C8) or their combination.

At least one of the first charge auxiliary layer 316 and the second charge auxiliary layer 334 includes the organic compound of the present disclosure represented by Formula 1. For example, at least one of the first HBL 316b and the first ETL 316a may include the organic compound of the present disclosure, and at least one of the second HBL 334b and the second ETL 334a may include the organic compound of the present disclosure.

In an aspect of the present disclosure, each of the first HBL 316b and the second HBL 334b may include the organic compound of the present disclosure. Each of the first HBL 316b and the second HBL 334b may consist of the organic compound of the present disclosure. The organic compound in the first HBL 316b and the organic compound in the second HBL 334b may be the same or different.

Each of the first HBL 316b and the second HBL 334b may have a thickness of 5 to 20 nm, e.g., 5 to 10 nm.

In an aspect of the present disclosure, each of the first ETL 316a and the second ETL 334a may include the organic compound of the present disclosure. The organic compound in the first ETL 316a and the organic compound in the second ETL 334a may be the same or different.

In addition, each of the first ETL 316a and the second ETL 334a may further include the compound in Formula 3, e.g., Liq. In this case, in each of the first ETL 316a and the second ETL 334a, a weight % ratio of the organic compound in Formula 1 to the compound in Formula 3 may be 1:9 to 9:1, e.g., 2:8 to 8:2 or 3:7 to 7:3. For example, in each of the first ETL 316a and the second ETL 334a, the organic compound in Formula 1 and the compound in Formula 3 may have the same weight %.

A thickness of the first ETL 316a and a thickness of the second ETL 334a may be greater than that of the first HBL 316b and that of the second HBL 334b, respectively. Each of the first ETL 316a and the second ETL 334a may have a thickness of 10 to 50 nm, e.g., 20 to 40 nm.

Each of the first and second EMLs 320 and 340 in the green pixel region, i.e., first and green EMLs, may include the organic compound of the present disclosure represented by Formula 1. For example, each of the green EMLs 320 and 340 may the organic compound of the present disclosure represented by Formula 1 as a first host, e.g., an n-type host. The organic compound in the first green EML 320 and the organic compound in the second green EML 340 may be the same or different.

Alternatively, at least one of the first and second green EMLs 320 and 340 or each of the first and second green EMLs 320 and 340 may include the compound in Formula 4 or the compound in Formula 5 as a first host, e.g., an n-type host.

Each of the first and second green EMLs 320 and 340 may further include the compound represented by Formula 7 as a second host, e.g., a p-type host. In addition, each of the first and second green EMLs 320 and 340 may further include one of the compounds in Formula 9 as a dopant (e.g., an emitter).

Each of the first and second green EMLs 320 and 340 may have a thickness of 10 to 50 nm, e.g., 20 to 40 nm.

In each of the first and second green EMLs 320 and 340, a weight % of each of the first and second hosts may be greater than that of the dopant. The weight % of the first host and the weight % of the second host may be the same or different. In each of the first and second green EMLs 320 and 340, a weight % ratio of the first host to the second host may be 1:9 to 9:1, 2:8 to 8:2 or 7:3 to 3:7. In some embodiments, the weight % of the first host and the weight % of the second host may be the same. For example, the first host and the second host may be present at the same weight %, and the dopant may be present in an amount of 5 to 25 weight % in each of the first and second green EMLs 320 and 340, based on a total weight of the components in each of the first and second green EMLs 320 and 340.

In an aspect of the present disclosure, each of the first and second HBLs 316b and 334b may include the organic compound of the present disclosure represented by Formula 1, and each of the first and second ETLs 316a and 334a may include at least one of the compound represented by Formula 10, e.g., a first electron transporting material, a compound represented by Formula 11, e.g., a second electron transporting material, and a compound represented by Formula 12, e.g., a third electron transporting material, instead of the organic compound of the present disclosure represented by Formula 1.

In an aspect of the present disclosure, each of the first and second ETLs 316a and 334a may include the organic compound of the present disclosure represented by Formula 1, and each of the first and second HBLs 316b and 334b may include the compound in Formula 17 instead of the organic compound of the present disclosure represented by Formula 1.

In the red pixel region, each of the first and second EMLs 320 and 340 may include a red host and a red dopant. The red dopant may include at least one of a red phosphorescent compound, a red fluorescent compound, and a red delayed fluorescent compound. In each of the first and second EMLs 320 and 340, the red host may be present at a weight % greater than the red dopant. In each of the first and second EMLs 320 and 340, the red dopant may be present at a weight % of 1 to 10, or 1 to 5, based on a total weight of the components in each of the first and second EMLs 320 and 340.

In the blue pixel region, each of the first and second EMLs 320 and 340 may a blue host and a blue dopant. The blue dopant may include at least one of a blue phosphorescent compound, a blue fluorescent compound, and a blue delayed fluorescent compound. In each of the first and second EMLs 320 and 340, the blue host may be present at a weight % greater than the blue dopant. In each of the first and second EMLs 320 and 340, the blue dopant may be present at a weight % of 1 to 10, or 1 to 5, based on a total weight of the components in each of the first and second EMLs 320 and 340.

Each of a thickness of the first HTL 314 and a thickness of the second HTL 332 may be greater than that of each of the first and second EMLs 320 and 340, the first and second ETLs 316a and 334a and the first and second HBLs 316b and 334b. Each of the first and second HTLs 314 and 332 may have a thickness of 80 to 120 nm, e.g., 90 to 110 nm. Each of the first and second HTLs 314 and 332 may include the above-mentioned hole transporting material.

The HIL 312 may include the above-mentioned hole injection material and may have a thickness of 1 to 10 nm, e.g., 3 to 7 nm.

The EIL 336 may include the above-mentioned electron injection material and may have a thickness of 1 to 10 nm, e.g., 3 to 7 nm.

Each of the first and second EBLs may include the above-mentioned electron blocking material.

As described above, in the OLED D, at least one of the first and second ETLs 316 and 334a and the first and second HBLs 316b and 334b includes the organic compound of the present disclosure represented by Formula 1. As a result, the emitting efficiency and the lifespan of the OLED D are improved.

In addition, in the OLED D, each of the first and second ETLs 316 and 334a may further include the compound in Formula 3 with the organic compound of the present disclosure represented by Formula 1 so that the emitting efficiency and the lifespan of the OLED D may be further improved.

Moreover, in the OLED D in the green pixel region, the first and second EMLs 320 and 340, the first and second ETLs 316 and 334a and the first and second HBLs 316b and 334b may include the organic compound of the present disclosure represented by Formula 1 so that a manufacturing efficiency (production yield) of the OLED may be increased with improving the emitting efficiency and the lifespan.

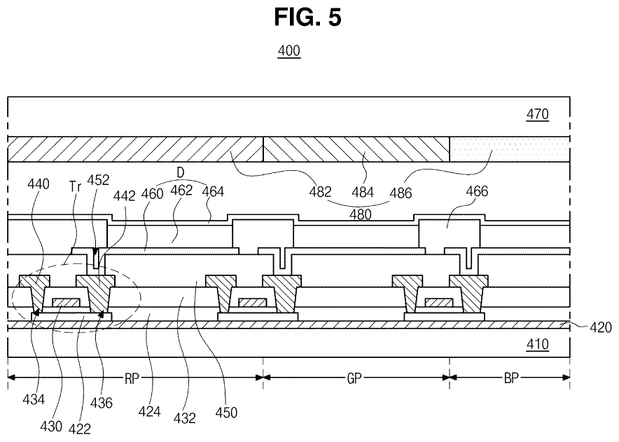

FIG. 5 illustrates a schematic cross-sectional view of an organic light emitting display device according to a fourth embodiment of the present disclosure.

As shown in FIG. 5, an organic light emitting display device 400 may include a first substrate 410, where a red pixel region RP, a green pixel region GP and a blue pixel region BP may be defined, a second substrate 470 facing the first substrate 410, an OLED D, which may be positioned between the first and second substrates 410 and 470 and providing white emission, and a color filter layer 480 between the OLED D and the second substrate 470.

Each of the first and second substrates 410 and 470 may be a glass substrate or a flexible substrate. For example, each of the first and second substrates 410 and 470 may be a polyimide (PI) substrate, a polyethersulfone (PES) substrate, a polyethylenenaphthalate (PEN) substrate, a polyethylene terephthalate (PET) substrate or a polycarbonate (PC) substrate.

A buffer layer 420 may be formed on the substrate. The TFT Tr corresponding to each of the red, green and blue pixel regions RP, GP and BP may be formed on the buffer layer 420. The buffer layer 420 may be formed of an inorganic insulating material, e.g., silicon oxide or silicon nitride. The buffer layer 420 may have a multi-layered structure including a first layer of silicon oxide and a second layer of silicon nitride. The buffer layer 420 may be omitted, and the TFT Tr may be disposed on the substrate 410.

A semiconductor layer 422 may be formed on the buffer layer 420. The semiconductor layer 422 may include an oxide semiconductor material or polycrystalline silicon.

A gate insulating layer 424 may be formed on the semiconductor layer 422. The gate insulating layer 424 may be formed of an inorganic insulating material such as silicon oxide or silicon nitride.

A gate electrode 430, which may be formed of a conductive material, e.g., metal, may be formed on the gate insulating layer 424 to correspond to a center of the semiconductor layer 422.

An interlayer insulating layer 432, which may be formed of an insulating material, may be formed on the gate electrode 430. The interlayer insulating layer 432 may be formed of an inorganic insulating material, e.g., silicon oxide or silicon nitride, or an organic insulating material, e.g., benzocyclobutene or photo-acryl.

The interlayer insulating layer 432 may include first and second contact holes 434 and 436 exposing both sides of the semiconductor layer 422. The first and second contact holes 434 and 436 may not cover a portion of the surface of the semiconductor layer 422 that is nearer to the opposing ends than to a center of the semiconductor layer 422. The first and second contact holes 434 and 436 may be positioned at both sides of the gate electrode 430 to be spaced apart from the gate electrode 430.

A source electrode 440 and a drain electrode 442, which may be formed of a conductive material, e.g., metal, may be formed on the interlayer insulating layer 432.

The source electrode 440 and the drain electrode 442 may be spaced apart from each other with respect to the gate electrode 430 and contact both sides of the semiconductor layer 422 through the first and second contact holes 434 and 436, respectively.

The semiconductor layer 422, the gate electrode 430, the source electrode 440, and the drain electrode 442 may constitute the TFT Tr. The TFT Tr may serve as a driving element. For example, the TFT Tr may correspond to the driving TFT Td (of FIG. 1).

Although not shown, the gate line and the data line may cross each other to define the pixel region. The switching TFT may be formed to be connected to the gate and data lines. The switching TFT may be connected to the TFT Tr as the driving element.

In addition, the power line, which may be formed to be parallel to and spaced apart from one of the gate and data lines, and the storage capacitor for maintaining the voltage of the gate electrode of the TFT Tr in one frame may be further formed.

A planarization layer 450, which may include a drain contact hole 452 exposing the drain electrode 442 of the TFT Tr, may be formed to cover the TFT Tr. The drain contact hole 452 may not cover the drain electrode 442.

A first electrode 460, which may be connected to the drain electrode 442 of the TFT Tr through the drain contact hole 452, may be separately formed in each pixel region and on the planarization layer 450. The first electrode 460 may be an anode and may include a transparent conductive oxide material layer formed of a conductive material, e.g., a transparent conductive oxide (TCO), having a relatively high work function.

For example, the transparent conductive oxide material layer of the first electrode 460 may include at least one of indium-tin-oxide (ITO) indium-zinc-oxide (IZO), indium-tin-zinc oxide; ITZO), tin oxide (SnO), zinc oxide (ZnO), indium-copper-oxide (ICO) and Al:ZnO (AZO).

The first electrode 460 may further include a reflection layer to have a double-layered structure or a triple-layered structure. For example, the reflection layer may be formed of silver (Ag) or aluminium-palladium-copper (APC) alloy. In the top-emission type organic light emitting display device 400, the first electrode 460 may have a double-layered structure of Ag/ITO or APC/ITO or a triple-layered structure of ITO/Ag/ITO or ITO/APC/ITO. However, embodiments of the present disclosure are not limited to such examples.

A bank layer 466 may be formed on the planarization layer 450 to cover an edge of the first electrode 460. For example, the bank layer 466 may be positioned at a boundary of the pixel region and may expose a center of the first electrode 460 in the pixel region. Since the OLED D may emit the white light in the red, green and blue pixel regions RP, GP and BP, the organic light emitting layer 462 may be formed as a common layer in the red, green and blue pixel regions RP, GP and BP without separation. The bank layer 466 may be formed to prevent a current leakage at an edge of the first electrode 460 and may be omitted.

An organic light emitting layer 462 may be formed on the first electrode 460.

In an aspect of the present disclosure, the organic light emitting layer 462 may have a three-stack structure including a first emitting part, which includes a green EML and a first charge auxiliary layer, a second emitting part, which includes a first blue EML and a second charge auxiliary layer, and a third emitting part, includes a second blue EML and a third charge auxiliary layer.

In an aspect of the present disclosure, the organic light emitting layer 462 may have a two-stack structure including a first emitting part, which includes a green EML and a first charge auxiliary layer, and a second emitting part, which includes a first blue EML and a second charge auxiliary layer.

In these configurations, at least one of the first and second charge auxiliary layers includes the organic compound of the present disclosure represented by Formula 1. In addition, the green EML may include the organic compound of the present disclosure represented by Formula 1.

A second electrode 464 may be formed over the substrate 410 where the organic light emitting layer 462 may be formed.

In the organic light emitting display device 400, since the light emitted from the organic light emitting layer 462 may be incident to the color filter layer 480 through the second electrode 464, the second electrode 464 may have a thin profile for transmitting the light.

The first electrode 460, the organic light emitting layer 462, and the second electrode 464 may constitute the OLED D.

The color filter layer 480 may be positioned over the OLED D and may include a red color filter pattern 482, a green color filter pattern 484, and a blue color filter pattern 486 corresponding to the red, green, and blue pixel regions RP, GP, and BP, respectively. The red color filter pattern 482 may include at least one of red dye and red pigment. The green color filter pattern 484 may include at least one of green dye and green pigment. The blue color filter pattern 486 may include at least one of blue dye and blue pigment.

Although not shown, the color filter layer 480 may be attached to the OLED D by an adhesive layer. Alternatively, the color filter layer 480 may be formed directly on the OLED D. However, embodiments of the present disclosure are not limited to such examples.

An encapsulation layer may be formed to prevent penetration of moisture into the OLED D. For example, the encapsulation layer may include a first inorganic insulating layer, an organic insulating layer, and a second inorganic insulating layer sequentially stacked, but it is not limited thereto. The encapsulation layer may be omitted.

For example, the color filter layer 480 may be positioned on the encapsulation layer. In addition, a touch electrode layer including a touch electrode may be positioned between the color filter layer 480 and the encapsulation layer.

A polarization plate for reducing an ambient light reflection may be disposed over the top-emission type OLED D. For example, the polarization plate may be a circular polarization plate.

In the OLED of FIG. 5, the first and second electrodes 460 and 464 may be a reflective electrode and a transparent (or semi-transparent) electrode, respectively. The color filter layer 480 may be disposed over the OLED D. Alternatively, when the first and second electrodes 460 and 464 are a transparent (or semi-transparent) electrode and a reflective electrode, respectively, the color filter layer 480 may be disposed between the OLED D and the first substrate 410. However, embodiments of the present disclosure are not limited to such examples.

A color conversion layer (not shown) may be formed between the OLED D and the color filter layer 480. The color conversion layer may include a red color conversion layer, a green color conversion layer, and a blue color conversion layer corresponding to the red, green, and blue pixel regions RP, GP, and BP, respectively. The white light from the OLED D may be converted into the red light, the green light, and the blue light by the red, green, and blue color conversion layers, respectively. For example, the color conversion layer may include a quantum dot. Accordingly, the color purity of the organic light emitting display device 400 may be further improved.

The color conversion layer may be included instead of the color filter layer 480.

As described above, in the organic light emitting display device 400, the OLED D in the red, green, and blue pixel regions RP, GP, and BP may emit the white light. The white light from the organic light emitting diode D may pass through the red color filter pattern 482, the green color filter pattern 484, and the blue color filter pattern 486. As a result, the red light, the green light and the blue light may be provided from the red pixel region RP, the green pixel region GP, and the blue pixel region BP, respectively.

In FIG. 5, the OLED D emitting the white light may be used for a display device. Alternatively, the OLED D may be formed on an entire surface of a substrate without at least one of the driving element and the color filter layer to be used for a lighting device. The display device and the lighting device each including an example of the OLED D of the present disclosure may be referred to as an organic light emitting device. However, embodiments of the present disclosure are not limited to such examples.

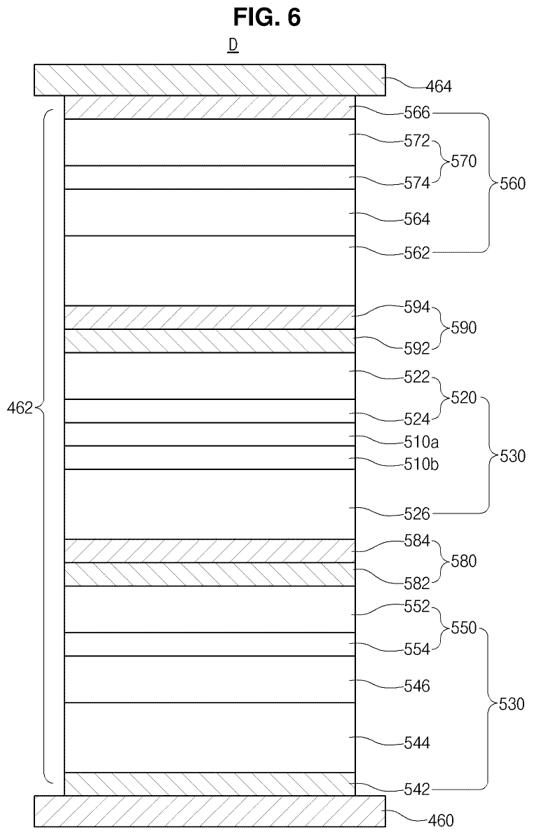

FIG. 6 illustrates a schematic cross-sectional view of an OLED according to a fifth embodiment of the present disclosure.

As illustrated in FIG. 6, the organic light emitting layer 462 may include a first emitting part 530 including a green EML 510a and a first charge auxiliary layer 520, a second emitting part 540 including a first blue EML 546 and a second charge auxiliary layer 550, and a third emitting part 560 including a second blue EML 564 and a third charge auxiliary layer 570. The first charge auxiliary layer 520 include at least one of a first ETL 522 and a first HBL 524, the second charge auxiliary layer 550 includes at least one of a second ETL 552 and a second HBL 554, and the third charge auxiliary layer 570 includes at least one of a third ETL 572 and a third HBL 574. The first HBL 524 may be positioned between the green EML 510a and the first ETL 522, the second HBL 554 may be positioned between the first blue EML 546 and the second ETL 552, and the third HBL 574 may be positioned between the second blue EML 564 and the third ETL 572.

In addition, the organic light emitting layer 462 may further include a first CGL 580 between the first and second emitting parts 530 and 540 and a second CGL 590 between the first and third emitting parts 530 and 560.

The organic light emitting display device 400 (of FIG. 5) may include red, green and blue pixel regions, and the OLED D may be positioned in each of the red, green and blue pixel region and emit a white light.

The first electrode 460 may act as an anode for injecting a hole and may be formed of a conductive material, e.g., ITO or IZO, having a relatively high work function. The second electrode 464 may act a cathode for injecting an electron and may be formed of a conductive material, e.g., Al, Mg or AlMg, having a relatively low work function.

In the top-emission type OLED D, the first electrode 460 may further include a reflection layer to act as a reflective electrode, and the second electrode 464 may have a thin profile to act as a transparent (e.g. semi-transparent) electrode. Alternatively, in the bottom-emission type OLED D, the first electrode 460 may act as a transparent electrode, and the second electrode 464 may act as a reflective electrode. However, embodiments of the present disclosure are not limited to such examples.

The second emitting part 540 may be positioned between the first electrode 460 and the first emitting part 530. The third emitting part 560 may be positioned between the first emitting part 530 and the second electrode 464. The second emitting part 540 may be positioned between the first electrode 460 and the first CGL 580. The third emitting part 560 may be positioned between the second CGL 590 and the second electrode 464. For example, the second emitting part 540, the first CGL 580, the first emitting part 530, the second CGL 590, and the third emitting part 560 may be sequentially stacked on the first electrode 460.

The first and second emitting parts 530 and 540 may be connected through the first CGL 580. The first and third emitting parts 530 and 560 may be connected through the second CGL 590. The first CGL 580 may be a P-N junction CGL including a first N-type CGL 582 and a first P-type CGL 584. The second CGL 590 may be a P-N junction CGL including a second N-type CGL 592 and a second P-type CGL 594.

In the first CGL 580, the first N-type CGL 582 may be positioned between the first HTL 526 and the second charge auxiliary layer 550. The first P-type CGL 584 may be positioned between the first N-type CGL 582 and the first HTL 526.

In the second CGL 590, the second N-type CGL 592 may be positioned between the third HTL 562 and the first charge auxiliary layer 520. The second P-type CGL 594 may be positioned between the second N-type CGL 592 and the third HTL 562.

Each of the first and second N-type CGLs 582 and 592 may include the above-mentioned N-type charge generation material, and each of the first and second P-type CGLs 584 and 594 may include the above-mentioned P-type charge generation material.

The first emitting part 530 may further include a red EML 510b. In the first emitting part 530, the red EML 510b may be disposed under the green EML 510a.

The first emitting part 530 may further include a first HTL 526 disposed under the red EML 510b. In addition, the first emitting part 530 may further include a first EBL between the red EML 510b and the first HTL 526.

For example, in the first emitting part 530, the red EML 510b may be positioned between the first HTL 526 and the green EML 510a. The green EML 510a may be positioned between the red EML 510b and the first charge auxiliary layer 520.

The second emitting part 540 may further include a second HTL 544 disposed under the first blue EML 546. In addition, the second emitting part 540 may further include an HIL 542 between the first electrode 460 and the second HTL 544. Moreover, the second emitting part 540 may further include a first EBL between the second HTL 544 and the first blue EML 546.

The third emitting part 560 may further include at least one of a third HTL 562 disposed under the second blue EML 564. In addition, the third emitting part 560 may further include an EIL 566 between the second electrode 464 and the third charge auxiliary layer 570. Moreover, the third emitting part 560 may further include a second EBL between the third HTL 562 and the second blue EML 564.

At least one of the first charge auxiliary layer 520, the second charge auxiliary layer 550 and the third charge auxiliary layer 570 includes the organic compound of the present disclosure represented by Formula 1. For example, at least one of the first HBL 524 and the first ETL 522 may include the organic compound of the present disclosure. At least one of the second HBL 554 and the second ETL 552 may include the organic compound of the present disclosure. At least one of the third HBL 574 and the third ETL 572 may include the organic compound of the present disclosure.

In an aspect of the present disclosure, each of the first HBL 524, the second HBL 554 and the third HBL 574 may include the organic compound of the present disclosure. Each of the first HBL 524, the second HBL 554 and the third HBL 574 may consist of the organic compound of the present disclosure. The organic compound in the first HBL 524, the organic compound in the second HBL 554 and the organic compound in the third HBL 574 may be the same or different.

Each of the first HBL 524, the second HBL 554 and the third HBL 574 may have a thickness of 5 to 20 nm, e.g., 5 to 10 nm.

In an aspect of the present disclosure, each of the first ETL 522, the second ETL 552 and the third ETL 572 may include the organic compound of the present disclosure. The organic compound in the first ETL 522, the organic compound in the second ETL 552 and the organic compound in the third ETL 572 may be the same or different.

In addition, each of the first ETL 522, the second ETL 552 and the third ETL 572 may further include the compound in Formula 3, e.g., Liq. In this case, in each of the first ETL 522, the second ETL 552 and the third ETL 572, a weight % ratio of the organic compound in Formula 1 to the compound in Formula 3 may be 1:9 to 9:1, e.g., 2:8 to 8:2 or 3:7 to 7:3. For example, in each of the first ETL 522, the second ETL 552 and the third ETL 572, the organic compound in Formula 1 and the compound in Formula 3 may have the same weight %.

A thickness of the first ETL 522, a thickness of the second ETL 552 and a thickness of the third ETL 572 may be greater than that of the first HBL 524, that of the second HBL 554 and that of the third HBL 574, respectively. Each of the first ETL 522, the second ETL 552 and the third ETL 572 may have a thickness of 10 to 50 nm, e.g., 20 to 40 nm.

The green EML 510a may include the organic compound of the present disclosure represented by Formula 1. For example, the green EML 510a may the organic compound of the present disclosure represented by Formula 1 as a first host, e.g., an n-type host.

Alternatively, the green EML 510a may include the compound in Formula 4 or the compound in Formula 5 as a first host, e.g., an n-type host.

The green EML 510a may further include the compound represented by Formula 7 as a second host, e.g., a p-type host. In addition, the green EML 510a may further include one of the compounds in Formula 9 as a dopant (e.g., an emitter).

The green EML 510a may have a thickness of 10 to 50 nm, e.g., 20 to 40 nm.

In the green EML 510a, a weight % of each of the first and second hosts may be greater than that of the dopant. The weight % of the first host and the weight % of the second host may be the same or different. In the green EML 510a, a weight % ratio of the first host to the second host may be 1:9 to 9:1, 2:8 to 8:2 or 7:3 to 3:7. In some embodiments, the weight % of the first host and the weight % of the second host may be the same. For example, the first host and the second host may be present at the same weight %, and the dopant may be present in an amount of 5 to 25 weight % in the green EML 510a, based on a total weight of the components in the green EML 510a.

In an aspect of the present disclosure, each of the first to third HBLs 522, 552 and 572 may include the organic compound of the present disclosure represented by Formula 1, and each of the first to third ETLs 524, 554 and 574 may include at least one of the compound represented by Formula 10, e.g., a first electron transporting material, a compound represented by Formula 11, e.g., a second electron transporting material, and a compound represented by Formula 12, e.g., a third electron transporting material, instead of the organic compound of the present disclosure represented by Formula 1.

In an aspect of the present disclosure, each of the first to third ETLs 524, 554 and 574 may include the organic compound of the present disclosure represented by Formula 1, and each of the first to third HBLs 522, 552 and 572 may include the compound in Formula 17 instead of the organic compound of the present disclosure represented by Formula 1.

The red EML 510b may include a red host and a red dopant. The red dopant may include at least one of a red phosphorescent compound, a red fluorescent compound, and a red delayed fluorescent compound. In the red EML 510b, the red host may be present at a weight % greater than the red dopant. In the red EML 510b, the red dopant may be present at a weight % of 1 to 10, or 1 to 5, based on a total weight of the components in the red EML 510b.

The first emitting part 530 may further include a yellow-green EML between the red EML 510b and the green EML 510a. The yellow-green EML may include a yellow-green host and a yellow-green dopant. Each of the red dopant, the green dopant and the yellow-green dopant may be a fluorescent compound, a phosphorescent compound and a delayed fluorescent compound.

For example, the yellow-green host may be selected from the group consisting of mCP-CN, CBP, mCBP, mCP, DPEPO, 2,8-bis(diphenylphosphoryl)dibenzothiophene (PPT), TmPyPB, PYD-2Cz, 2,8-di(9H-carbazol-9-yl)dibenzothiophene (DCzDBT), 3′,5′-di(carbazol-9-yl)-[1,1′-bipheyl]-3,5-dicarbonitrile (DCzTPA), 4′-(9H-carbazol-9-yl)biphenyl-3,5-dicarbonitrile (pCzB-2CN), 3′-(9H-carbazol-9-yl)biphenyl-3,5-dicarbonitrile (mCzB-2CN), TSPO1, and 9-(9-phenyl-9H-carbazol-6-yl)-9H-carbazole (CCP), but it is not limited thereto.

For example, the yellow-green dopant may be selected from the group consisting of 5,6,11,12-tetraphenylnaphthalene (Rubrene), 2,8-di-tert-butyl-5,11-bis(4-tert-butylphenyl)-6,12-diphenyltetracene (TBRb), bis(2-phenylbenzothiazolato)(acetylacetonate)irdium(III) (Ir(BT)₂(acac)), bis(2-(9,9-diethytl-fluoren-2-yl)-1-phenyl-1H-benzo[d]imdiazolato)(acetylacetonate)iridium(III) (Ir(fbi)₂(acac)), bis(2-phenylpyridine)(3-(pyridine-2-yl)-2H-chromen-2-onate)iridium(III) (fac-Ir(ppy)2Pc), bis(2-(2,4-difluorophenyl)quinoline)(picolinate)iridium(III) (FPQIrpic), and bis(4-phenylthieno[3,2-c]pyridinato-N,C2′) (acetylacetonate) iridium(III); PO-01), but it is not limited thereto.

Each of the first blue EML 546 in the second emitting part 540 and the second blue EML 564 in the third emitting part 560 may include the above-mentioned blue host and the above-mentioned blue dopant. The blue dopant may include at least one of a blue phosphorescent compound, a blue fluorescent compound, and a blue delayed fluorescent compound. In each of the first and second blue EMLs 546 and 546, the blue host may be present at a weight % greater than the blue dopant. In each of the first and second blue EMLs 546 and 546, the blue dopant may be present at a weight % of 1 to 10, or 1 to 5, based on a total weight of the components in each of the first and second blue EMLs 546 and 546.

Each of a thickness of the first HTL 526, a thickness of the second HTL 544 and a thickness of the third HTL 562 may be greater than that of each of the green EML 510a, the red EML 510b, the first and second blue EMLs 546 and 564, the first to third ETLs 522, 552 and 572 and the first to third HTLs 526, 544 and 562. Each of the first to third HTLs 526, 544 and 562 may have a thickness of 80 to 120 nm, e.g., 90 to 110 nm. Each of the first to third HTLs 526, 544 and 562 may include the above-mentioned hole transporting material.

The HIL 542 may include the above-mentioned hole injection material and may have a thickness of 1 to 10 nm, e.g., 3 to 7 nm.

The EIL 566 may include the above-mentioned electron injection material and may have a thickness of 1 to 10 nm, e.g., 3 to 7 nm.

Each of the first to third EBLs may include the above-mentioned electron blocking material.

As described above, in the OLED D, at least one of the first to third ETLs 522, 552 and 572 and the first to third HBLs 524, 554 and 574 includes the organic compound of the present disclosure represented by Formula 1. As a result, the emitting efficiency and the lifespan of the OLED D are improved.

In addition, in the OLED D, each of the first to third ETLs 522, 552 and 572 may further include the compound in Formula 3 with the organic compound of the present disclosure represented by Formula 1 so that the emitting efficiency and the lifespan of the OLED D may be further improved.

Moreover, in the OLED D, the first to third ETLs 522, 552 and 572 and the first to third HBLs 524, 554 and 574 may include the organic compound of the present disclosure represented by Formula 1 so that a manufacturing efficiency (production yield) of the OLED may be increased with improving the emitting efficiency and the lifespan.

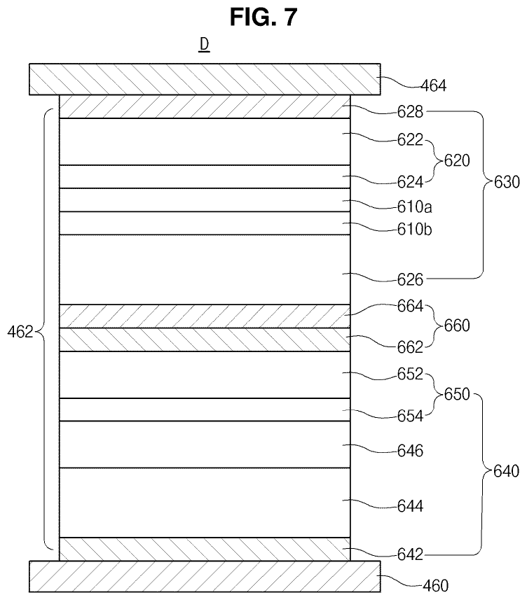

FIG. 7 illustrates a schematic cross-sectional view of an OLED according to a sixth embodiment of the present disclosure.

As illustrated in FIG. 7, the organic light emitting layer 462 may include a first emitting part 630 including a green EML 610a and a first charge auxiliary layer 620 and a second emitting part 640 including a blue EML 646 and a second charge auxiliary layer 650. The first charge auxiliary layer 620 includes at least one of a first ETL 622 and a first HBL 624, and the second charge auxiliary layer 650 includes at least one of a second ETL 652 and a second HBL 654. The first HBL 624 may be positioned between the green EML 610a and the first ETL 622, and the second HBL 654 may be positioned between the first blue EML 646 and the second ETL 652.

In addition, the organic light emitting layer 462 may further include a first CGL 660 between the first and second emitting parts 630 and 640.

The organic light emitting display device 400 (of FIG. 5) may include red, green and blue pixel regions, and the OLED D may be positioned in each of the red, green and blue pixel region and emit a white light.

The first electrode 460 may act as an anode for injecting a hole and may be formed of a conductive material, e.g., ITO or IZO, having a relatively high work function. The second electrode 464 may act a cathode for injecting an electron and may be formed of a conductive material, e.g., Al, Mg or AlMg, having a relatively low work function.

In the top-emission type OLED D, the first electrode 460 may further include a reflection layer to act as a reflective electrode, and the second electrode 464 may have a thin profile to act as a transparent (e.g. semi-transparent) electrode. Alternatively, in the bottom-emission type OLED D, the first electrode 460 may act as a transparent electrode, and the second electrode 464 may act as a reflective electrode. However, embodiments of the present disclosure are not limited to such examples.

The first emitting part 630 may be positioned between the second electrode 464 and the CGL 660, and the second emitting part 640 may be positioned between the first electrode 460 and the CGL 660. For example, the second emitting part 640, the CGL 660 and the first emitting part 630 may be sequentially stacked on the first electrode 460.

The first and second emitting parts 630 and 640 may be connected through the CGL 660. The CGL 660 may be a P-N junction CGL including an N-type CGL 662 and a P-type CGL 664.

In the CGL 660, the N-type CGL 662 may be positioned between the first HTL 626 and the second charge auxiliary layer 650. The P-type CGL 664 may be positioned between the N-type CGL 662 and the first HTL 626.

The N-type CGL 662 may include the above-mentioned N-type charge generation material, and the P-type CGL 664 may include the above-mentioned P-type charge generation material.

The first emitting part 630 may further include a red EML 610b. In the first emitting part 630, the red EML 610b may be disposed under the green EML 610a.

The first emitting part 630 may further include a first HTL 626 disposed under the red EML 610b. In addition, the first emitting part 630 may further include a first EBL between the red EML 610b and the first HTL 626.

For example, in the first emitting part 630, the red EML 610b may be positioned between the first HTL 626 and the green EML 610a. The green EML 610a may be positioned between the red EML 610b and the first charge auxiliary layer 620.

The second emitting part 640 may further include a second HTL 644 disposed under the blue EML 646. In addition, the second emitting part 640 may further include an HIL 642 between the first electrode 460 and the second HTL 644. Moreover, the second emitting part 640 may further include a first EBL between the second HTL 644 and the blue EML 646.

At least one of the first charge auxiliary layer 620 and the second charge auxiliary layer 650 includes the organic compound of the present disclosure represented by Formula 1. For example, at least one of the first HBL 624 and the first ETL 622 may include the organic compound of the present disclosure. At least one of the second HBL 654 and the second ETL 652 may include the organic compound of the present disclosure.

In an aspect of the present disclosure, each of the first HBL 624 and the second HBL 654 may include the organic compound of the present disclosure. Each of the first HBL 624 and the second HBL 654 may consist of the organic compound of the present disclosure. The organic compound in the first HBL 624 and the organic compound in the second HBL 654 may be the same or different.

Each of the first HBL 624 and the second HBL 654 may have a thickness of 5 to 20 nm, e.g., 5 to 10 nm.

In an aspect of the present disclosure, each of the first ETL 622 and the second ETL 652 may include the organic compound of the present disclosure. The organic compound in the first ETL 622 and the organic compound in the second ETL 652 may be the same or different.

In addition, each of the first ETL 622 and the second ETL 652 may further include the compound in Formula 3, e.g., Liq. In this case, in each of the first ETL 622 and the second ETL 652, a weight % ratio of the organic compound in Formula 1 to the compound in Formula 3 may be 1:9 to 9:1, e.g., 2:8 to 8:2 or 3:7 to 7:3. For example, in each of the first ETL 622 and the second ETL 652, the organic compound in Formula 1 and the compound in Formula 3 may have the same weight %.

A thickness of the first ETL 622 and a thickness of the second ETL 552 may be greater than that of the first HBL 624 and that of the second HBL 654, respectively. Each of the first ETL 622 and the second ETL 652 may have a thickness of 10 to 50 nm, e.g., 20 to 40 nm.

The green EML 610a may include the organic compound of the present disclosure represented by Formula 1. For example, the green EML 610a may the organic compound of the present disclosure represented by Formula 1 as a first host, e.g., an n-type host.

Alternatively, the green EML 610a may include the compound in Formula 4 or the compound in Formula 5 as a first host, e.g., an n-type host.

The green EML 610a may further include the compound represented by Formula 7 as a second host, e.g., a p-type host. In addition, the green EML 610a may further include one of the compounds in Formula 9 as a dopant (e.g., an emitter).

The green EML 610a may have a thickness of 10 to 50 nm, e.g., 20 to 40 nm.

In the green EML 610a, a weight % of each of the first and second hosts may be greater than that of the dopant. The weight % of the first host and the weight % of the second host may be the same or different. In the green EML 610a, a weight % ratio of the first host to the second host may be 1:9 to 9:1, 2:8 to 8:2 or 7:3 to 3:7. In some embodiments, the weight % of the first host and the weight % of the second host may be the same. For example, the first host and the second host may be present at the same weight %, and the dopant may be present in an amount of 5 to 25 weight % in the green EML 610a, based on a total weight of the components in the green EML 610a.

In an aspect of the present disclosure, each of the first and third HBLs 622 and 652 may include the organic compound of the present disclosure represented by Formula 1, and each of the first and third ETLs 624 and 654 may include at least one of the compound represented by Formula 10, e.g., a first electron transporting material, a compound represented by Formula 11, e.g., a second electron transporting material, and a compound represented by Formula 12, e.g., a third electron transporting material, instead of the organic compound of the present disclosure represented by Formula 1.

In an aspect of the present disclosure, each of the first and third ETLs 624 and 654 may include the organic compound of the present disclosure represented by Formula 1, and each of the first and third HBLs 622 and 652 may include the compound in Formula 17 instead of the organic compound of the present disclosure represented by Formula 1.

The red EML 610b may include a red host and a red dopant. The red dopant may include at least one of a red phosphorescent compound, a red fluorescent compound, and a red delayed fluorescent compound. In the red EML 610b, the red host may be present at a weight % greater than the red dopant. In the red EML 610b, the red dopant may be present at a weight % of 1 to 10, or 1 to 5, based on a total weight of the components in the red EML 610b.

The first emitting part 630 may further include a yellow-green EML between the red EML 610b and the green EML 610a. The yellow-green EML may include a yellow-green host and a yellow-green dopant. Each of the red dopant, the green dopant and the yellow-green dopant may be a fluorescent compound, a phosphorescent compound and a delayed fluorescent compound.

The blue EML 646 in the second emitting part 640 may include the above-mentioned blue host and the above-mentioned blue dopant. The blue dopant may include at least one of a blue phosphorescent compound, a blue fluorescent compound, and a blue delayed fluorescent compound. In the blue EML 646, the blue host may be present at a weight % greater than the blue dopant. In the blue EML 646, the blue dopant may be present at a weight % of 1 to 10, or 1 to 5, based on a total weight of the components in the blue EML 646.

Each of a thickness of the first HTL 626 and a thickness of the second HTL 644 may be greater than that of each of the green EML 610a, the red EML 610b, the blue EML 646, the first and second ETLs 622 and 652 and the first and second HBLs 624 and 654. Each of the first and second HTLs 626 and 644 may have a thickness of 80 to 120 nm, e.g., 90 to 110 nm. Each of the first to third HTLs 526 and 644 may include the above-mentioned hole transporting material.

The HIL 642 may include the above-mentioned hole injection material and may have a thickness of 1 to 10 nm, e.g., 3 to 7 nm.

The EIL 628 may include the above-mentioned electron injection material and may have a thickness of 1 to 10 nm, e.g., 3 to 7 nm.

Each of the first and second EBLs may include the above-mentioned electron blocking material.

As described above, in the OLED D, at least one of the first and second ETLs 622 and 622 and the first and second HBLs 624 and 654 includes the organic compound of the present disclosure represented by Formula 1. As a result, the emitting efficiency and the lifespan of the OLED D are improved.

In addition, in the OLED D, each of the first and second ETLs 622 and 622 may further include the compound in Formula 3 with the organic compound of the present disclosure represented by Formula 1 so that the emitting efficiency and the lifespan of the OLED D may be further improved.

Moreover, in the OLED D, the first and second ETLs 622 and 622 and the first and second HBLs 624 and 654 may include the organic compound of the present disclosure represented by Formula 1 so that a manufacturing efficiency (production yield) of the OLED may be increased with improving the emitting efficiency and the lifespan.

It will be apparent to those skilled in the art that various modifications and variations can be made in the embodiments of the present disclosure without departing from the spirit or scope of the present disclosure. Thus, it is intended that the modifications and variations cover this disclosure provided they come within the scope of the appended claims and their equivalents.

1. An organic compound represented by Formula 1: 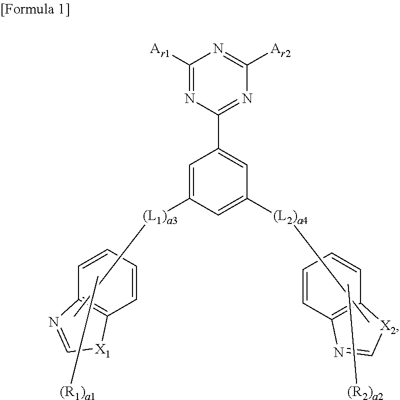
wherein each of a1 and a2 is independently an integer of 0 to 4, and each of a3 and a4 is independently 0 or 1,
each of X1 and X2 is independently O or S,
each of Ar₁ and Ar₂ is independently selected from the group consisting of a substituted or unsubstituted C6 to C60 aryl group and a substituted or unsubstituted C3 to C60 heteroaryl group containing one of N, O and S,
each R₁ and R₂ is independently selected from the group consisting of a substituted or unsubstituted C1 to C10 alkyl group, a substituted or unsubstituted C3 to C30 cycloalkyl group, a substituted or unsubstituted C1 to C10 alkoxy group, a substituted or unsubstituted aryl group and a substituted or unsubstituted C3 to C60 heteroaryl group containing one of N, O and S, and
each of L₁ and L₂ is independently selected from the group consisting of a substituted or unsubstituted C6 to C60 arylene group and a substituted or unsubstituted C3 to C60 heteroarylene group containing one of N, O and S.

2. The organic compound according to claim 1, wherein Ar₁ and Ar₂ are different.

3. The organic compound according to claim 1, wherein each of Ar₁ and Ar₂ is independently selected from Formula 1a:

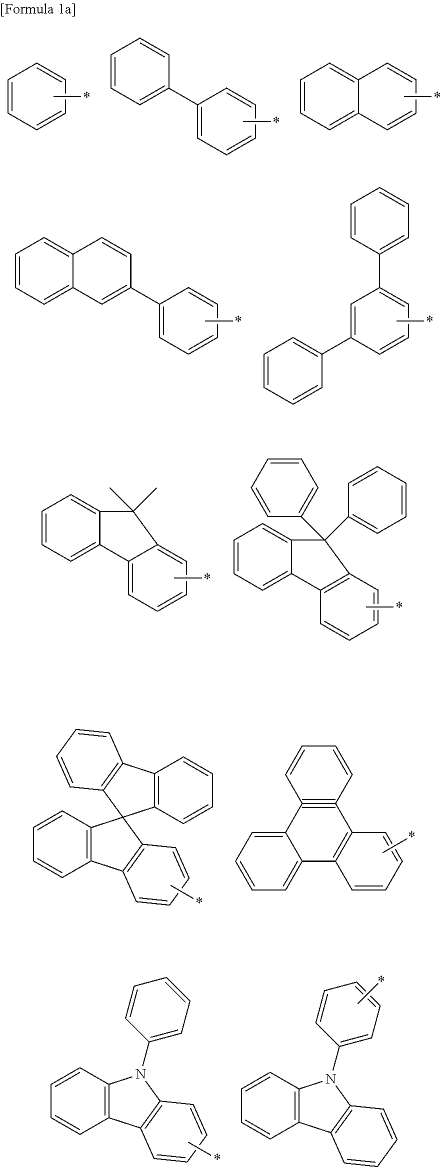

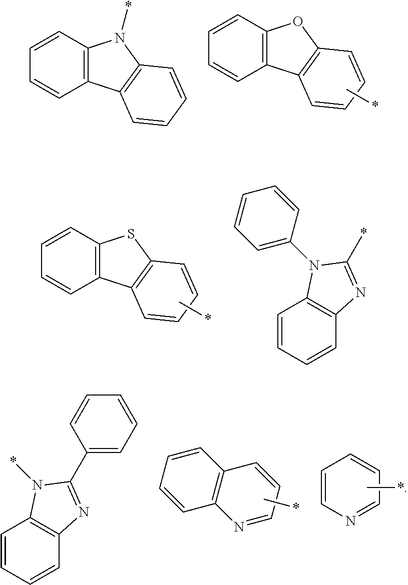

4. The organic compound according to claim 1, wherein Formula 1 is represented by Formula 1b-1: 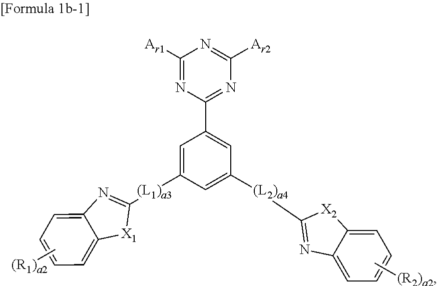
wherein the definition of a1, a2, a3, a4, X1, X₂, Ar₂, Ar₂, R₁, R₂, L₁ and L₂ is the same as for Formula 1.

5. The organic compound according to claim 1, wherein Formula 1 is represented by Formula 1b-2: 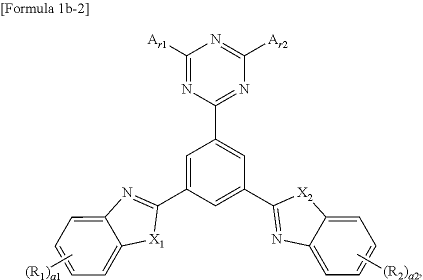
wherein the definition of a1, a2, X1, X₂, Ar₁, Ar₂, R₁ and R₂ is the same as for Formula 1.

6. The organic compound according to claim 1, wherein the organic compound is one of the compounds in Formula 2:

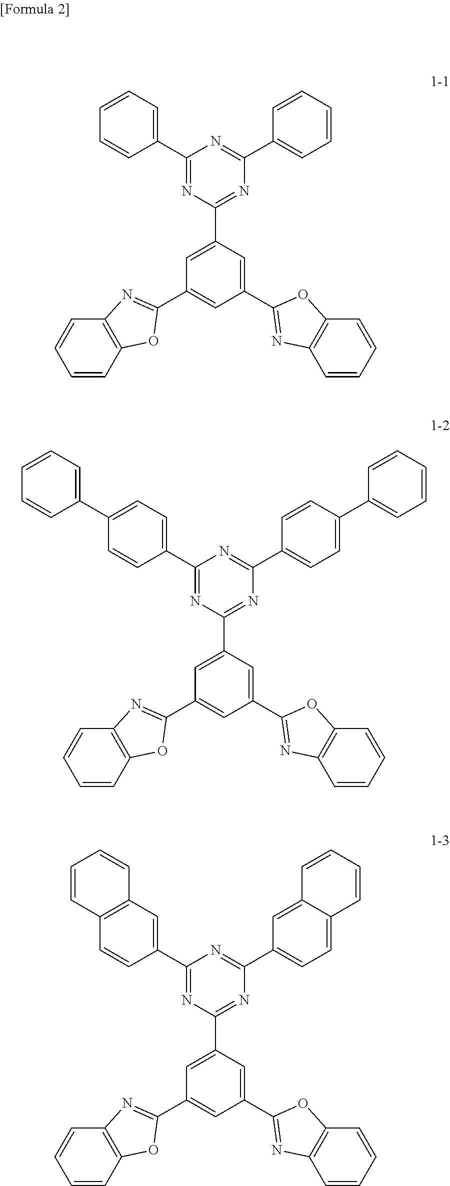 [SMILES: c1ccc(-c2nc(-c3ccccc3)nc(-c3cc(-c4nc5ccccc5o4)cc(-c4nc5ccccc5o4)c3)n2)cc1 ; c1ccc(-c2ccc(-c3nc(-c4ccc(-c5ccccc5)cc4)nc(-c4cc(-c5nc6ccccc6o5)cc(-c5nc6ccccc6o5)c4)n3)cc2)cc1 ; c1ccc2cc(-c3nc(-c4cc(-c5nc6ccccc6o5)cc(-c5nc6ccccc6o5)c4)nc(-c4ccc5ccccc5c4)n3)ccc2c1]

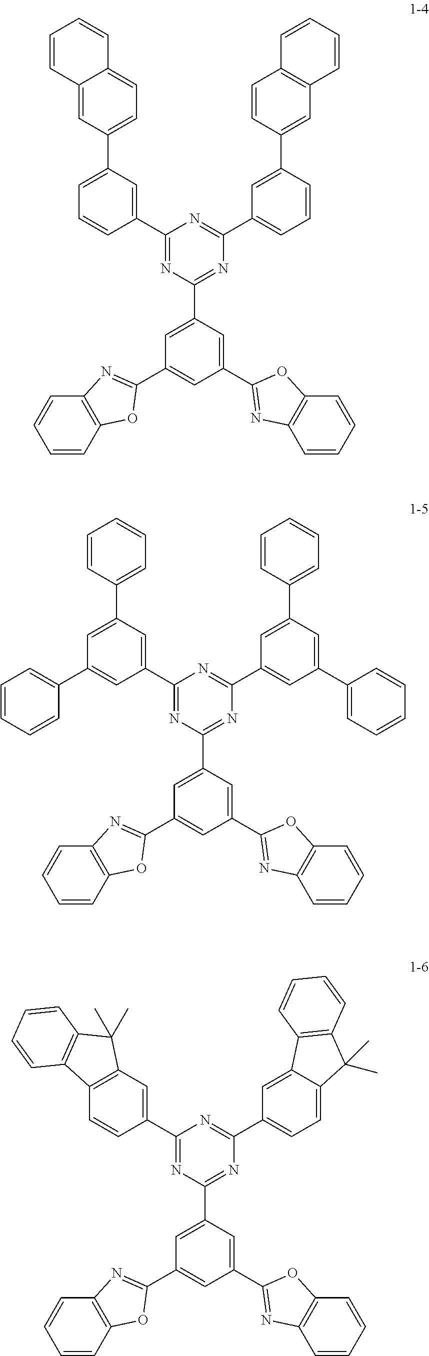 [SMILES: c1cc(-c2ccc3ccccc3c2)cc(-c2nc(-c3cccc(-c4ccc5ccccc5c4)c3)nc(-c3cc(-c4nc5ccccc5o4)cc(-c4nc5ccccc5o4)c3)n2)c1 ; c1ccc(-c2cc(-c3ccccc3)cc(-c3nc(-c4cc(-c5ccccc5)cc(-c5ccccc5)c4)nc(-c4cc(-c5nc6ccccc6o5)cc(-c5nc6ccccc6o5)c4)n3)c2)cc1 ; CC1(C)c2ccccc2-c2cc(-c3nc(-c4cc(-c5nc6ccccc6o5)cc(-c5nc6ccccc6o5)c4)nc(-c4ccc5c(c4)C(C)(C)c4ccccc4-5)n3)ccc21]

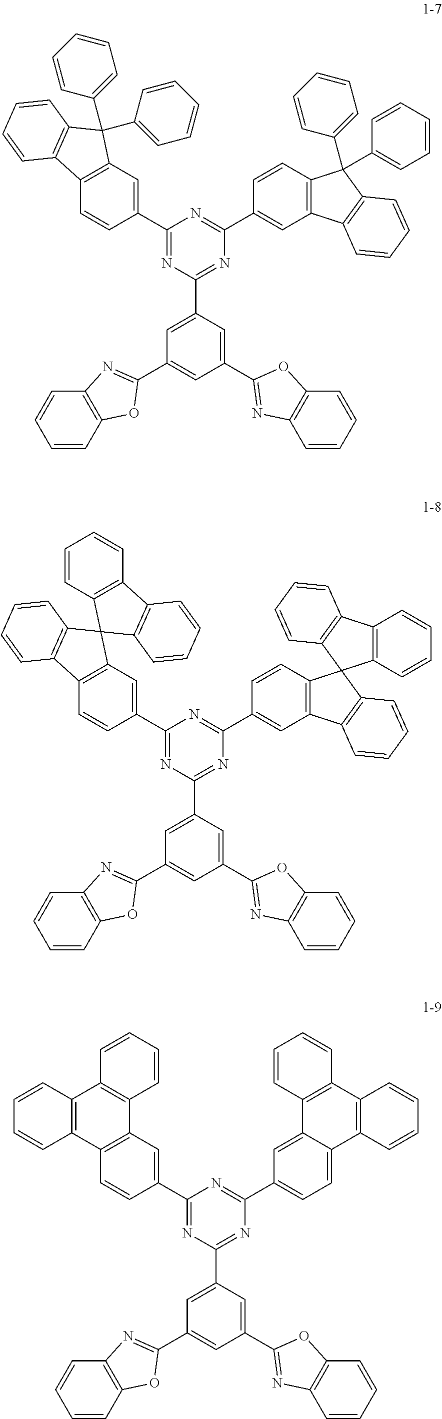 [SMILES: c1ccc(C2(c3ccccc3)c3ccccc3-c3cc(-c4nc(-c5cc(-c6nc7ccccc7o6)cc(-c6nc7ccccc7o6)c5)nc(-c5ccc6c(c5)C(c5ccccc5)(c5ccccc5)c5ccccc5-6)n4)ccc32)cc1 ; c1ccc2c(c1)-c1ccccc1C21c2ccccc2-c2cc(-c3nc(-c4cc(-c5nc6ccccc6o5)cc(-c5nc6ccccc6o5)c4)nc(-c4ccc5c(c4)C4(c6ccccc6-c6ccccc64)c4ccccc4-5)n3)ccc21 ; c1ccc2oc(-c3cc(-c4nc(-c5ccc6c7ccccc7c7ccccc7c6c5)nc(-c5ccc6c7ccccc7c7ccccc7c6c5)n4)cc(-c4nc5ccccc5o4)c3)nc2c1]

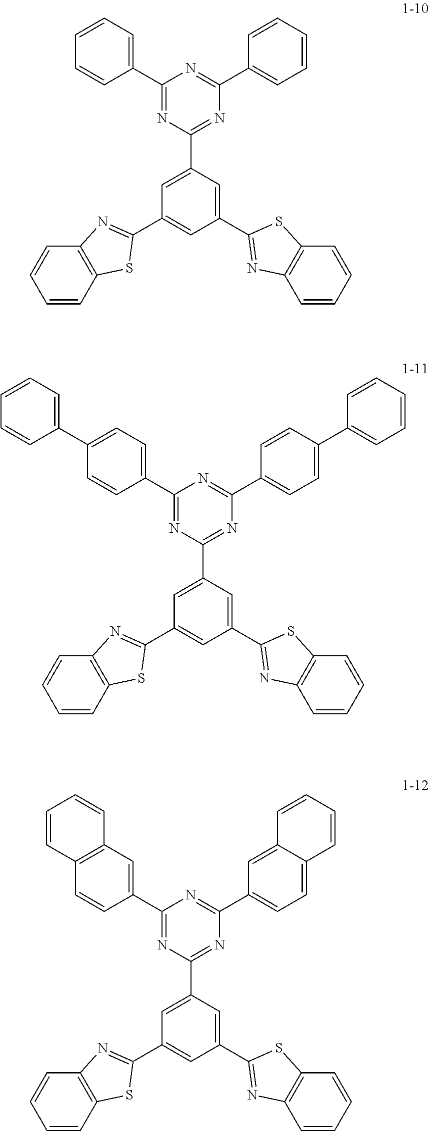 [SMILES: c1ccc(-c2nc(-c3ccccc3)nc(-c3cc(-c4nc5ccccc5s4)cc(-c4nc5ccccc5s4)c3)n2)cc1 ; c1ccc(-c2ccc(-c3nc(-c4ccc(-c5ccccc5)cc4)nc(-c4cc(-c5nc6ccccc6s5)cc(-c5nc6ccccc6s5)c4)n3)cc2)cc1 ; c1ccc2cc(-c3nc(-c4cc(-c5nc6ccccc6s5)cc(-c5nc6ccccc6s5)c4)nc(-c4ccc5ccccc5c4)n3)ccc2c1]

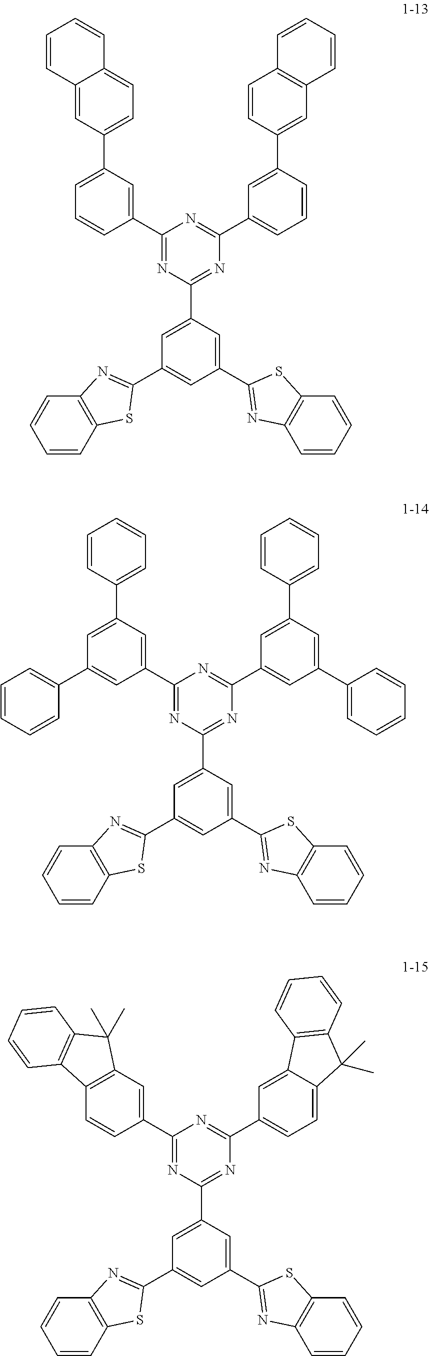 [SMILES: c1cc(-c2ccc3ccccc3c2)cc(-c2nc(-c3cccc(-c4ccc5ccccc5c4)c3)nc(-c3cc(-c4nc5ccccc5s4)cc(-c4nc5ccccc5s4)c3)n2)c1 ; c1ccc(-c2cc(-c3ccccc3)cc(-c3nc(-c4cc(-c5ccccc5)cc(-c5ccccc5)c4)nc(-c4cc(-c5nc6ccccc6s5)cc(-c5nc6ccccc6s5)c4)n3)c2)cc1 ; CC1(C)c2ccccc2-c2cc(-c3nc(-c4cc(-c5nc6ccccc6s5)cc(-c5nc6ccccc6s5)c4)nc(-c4ccc5c(c4)C(C)(C)c4ccccc4-5)n3)ccc21]

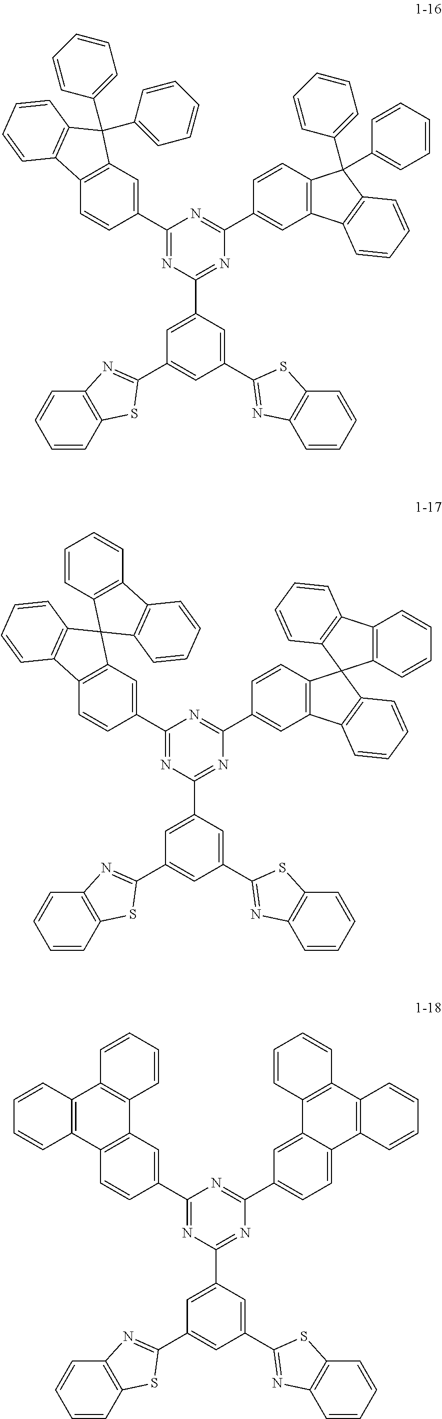 [SMILES: c1ccc(C2(c3ccccc3)c3ccccc3-c3cc(-c4nc(-c5cc(-c6nc7ccccc7s6)cc(-c6nc7ccccc7s6)c5)nc(-c5ccc6c(c5)C(c5ccccc5)(c5ccccc5)c5ccccc5-6)n4)ccc32)cc1 ; c1ccc2c(c1)-c1ccccc1C21c2ccccc2-c2cc(-c3nc(-c4cc(-c5nc6ccccc6s5)cc(-c5nc6ccccc6s5)c4)nc(-c4ccc5c(c4)C4(c6ccccc6-c6ccccc64)c4ccccc4-5)n3)ccc21 ; c1ccc2sc(-c3cc(-c4nc(-c5ccc6c7ccccc7c7ccccc7c6c5)nc(-c5ccc6c7ccccc7c7ccccc7c6c5)n4)cc(-c4nc5ccccc5s4)c3)nc2c1]

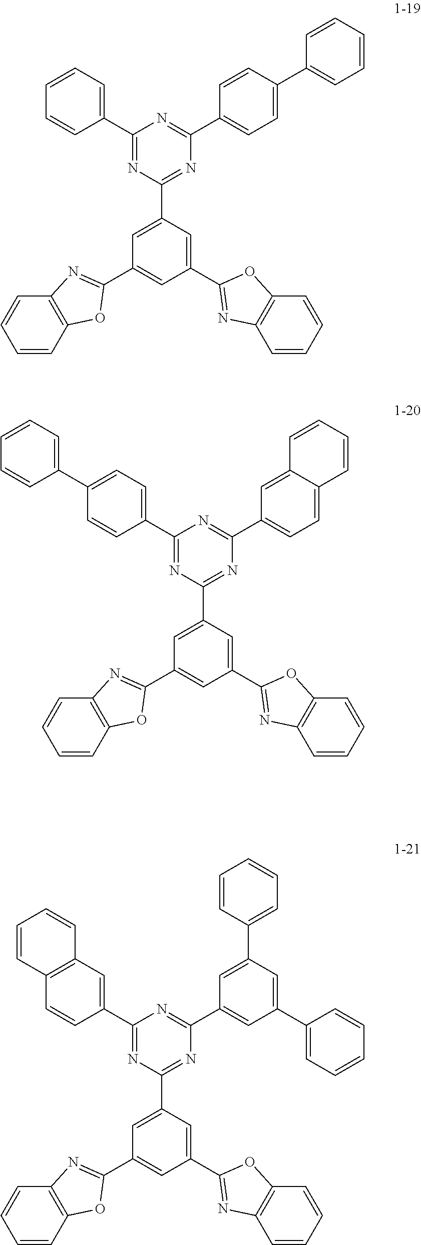 [SMILES: c1ccc(-c2ccc(-c3nc(-c4ccccc4)nc(-c4cc(-c5nc6ccccc6o5)cc(-c5nc6ccccc6o5)c4)n3)cc2)cc1 ; c1ccc(-c2ccc(-c3nc(-c4cc(-c5nc6ccccc6o5)cc(-c5nc6ccccc6o5)c4)nc(-c4ccc5ccccc5c4)n3)cc2)cc1 ; c1ccc(-c2cc(-c3ccccc3)cc(-c3nc(-c4cc(-c5nc6ccccc6o5)cc(-c5nc6ccccc6o5)c4)nc(-c4ccc5ccccc5c4)n3)c2)cc1]

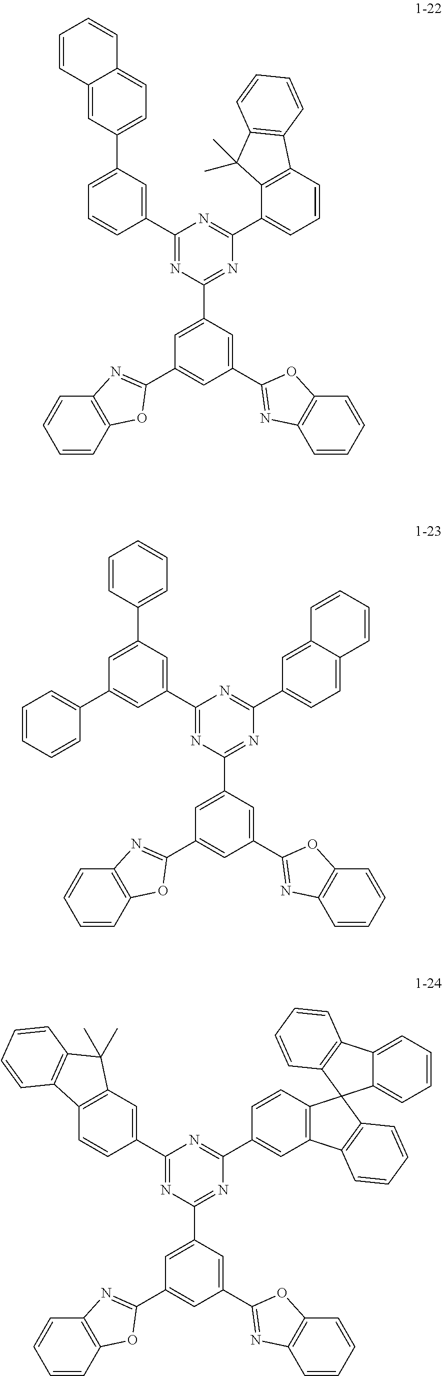 [SMILES: CC1(C)c2ccccc2-c2cccc(-c3nc(-c4cccc(-c5ccc6ccccc6c5)c4)nc(-c4cc(-c5nc6ccccc6o5)cc(-c5nc6ccccc6o5)c4)n3)c21 ; c1ccc(-c2cc(-c3ccccc3)cc(-c3nc(-c4cc(-c5nc6ccccc6o5)cc(-c5nc6ccccc6o5)c4)nc(-c4ccc5ccccc5c4)n3)c2)cc1 ; CC1(C)c2ccccc2-c2ccc(-c3nc(-c4cc(-c5nc6ccccc6o5)cc(-c5nc6ccccc6o5)c4)nc(-c4ccc5c(c4)-c4ccccc4C54c5ccccc5-c5ccccc54)n3)cc21]

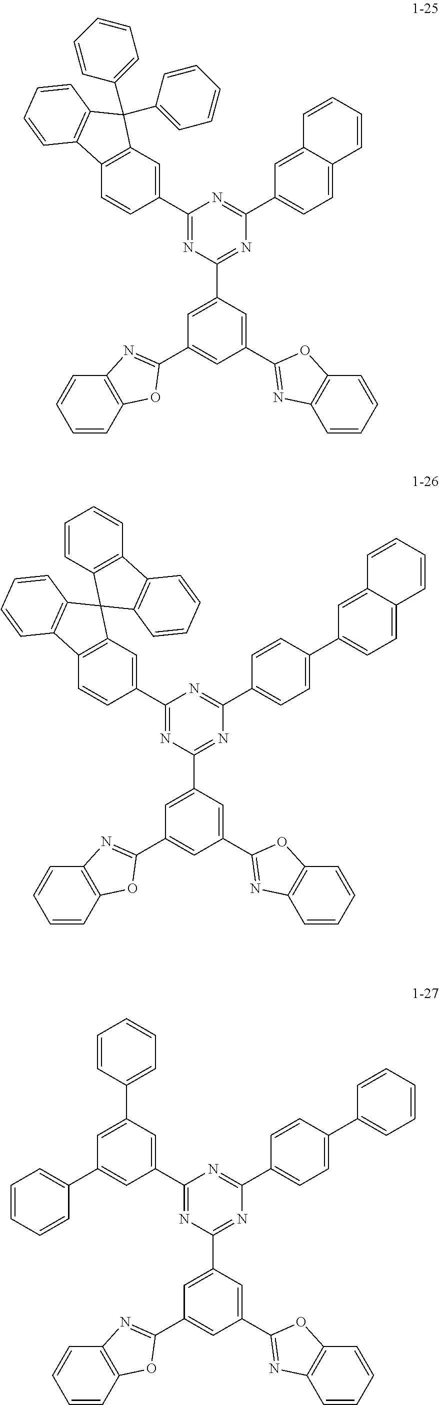 [SMILES: c1ccc(C2(c3ccccc3)c3ccccc3-c3ccc(-c4nc(-c5cc(-c6nc7ccccc7o6)cc(-c6nc7ccccc7o6)c5)nc(-c5ccc6ccccc6c5)n4)cc32)cc1 ; c1ccc2c(c1)-c1ccccc1C21c2ccccc2-c2ccc(-c3nc(-c4ccc(-c5ccc6ccccc6c5)cc4)nc(-c4cc(-c5nc6ccccc6o5)cc(-c5nc6ccccc6o5)c4)n3)cc21 ; c1ccc(-c2ccc(-c3nc(-c4cc(-c5ccccc5)cc(-c5ccccc5)c4)nc(-c4cc(-c5nc6ccccc6o5)cc(-c5nc6ccccc6o5)c4)n3)cc2)cc1]

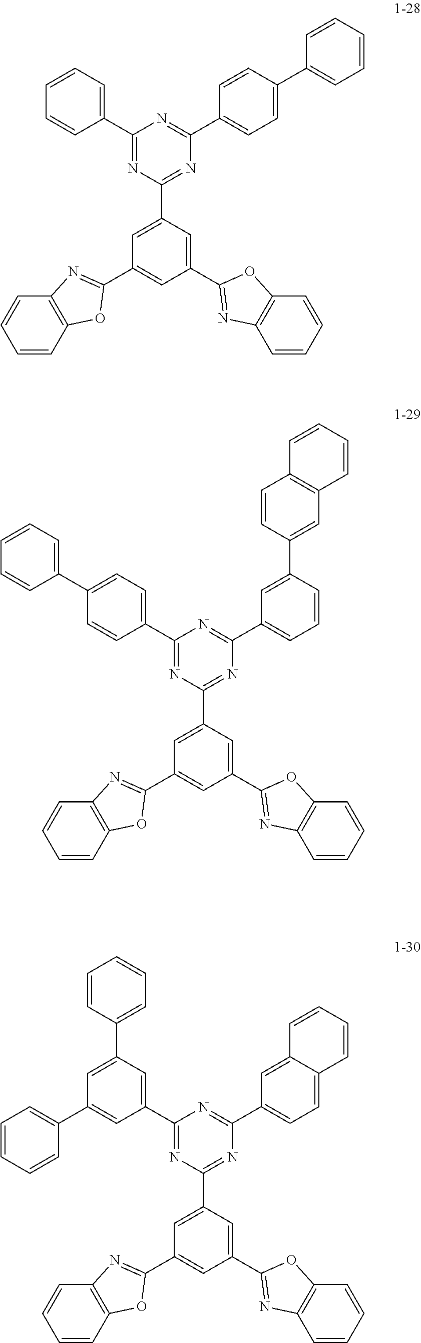 [SMILES: c1ccc(-c2ccc(-c3nc(-c4ccccc4)nc(-c4cc(-c5nc6ccccc6o5)cc(-c5nc6ccccc6o5)c4)n3)cc2)cc1 ; c1ccc(-c2ccc(-c3nc(-c4cccc(-c5ccc6ccccc6c5)c4)nc(-c4cc(-c5nc6ccccc6o5)cc(-c5nc6ccccc6o5)c4)n3)cc2)cc1 ; c1ccc(-c2cc(-c3ccccc3)cc(-c3nc(-c4cc(-c5nc6ccccc6o5)cc(-c5nc6ccccc6o5)c4)nc(-c4ccc5ccccc5c4)n3)c2)cc1]

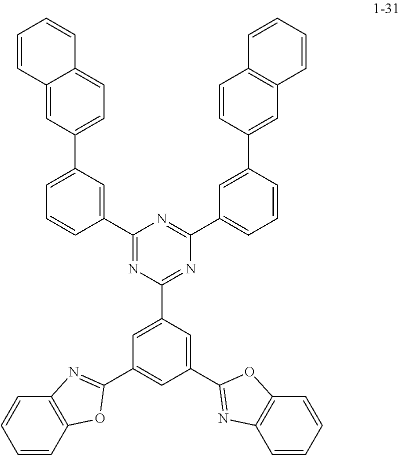 [SMILES: c1cc(-c2ccc3ccccc3c2)cc(-c2nc(-c3cccc(-c4ccc5ccccc5c4)c3)nc(-c3cc(-c4nc5ccccc5o4)cc(-c4nc5ccccc5o4)c3)n2)c1]

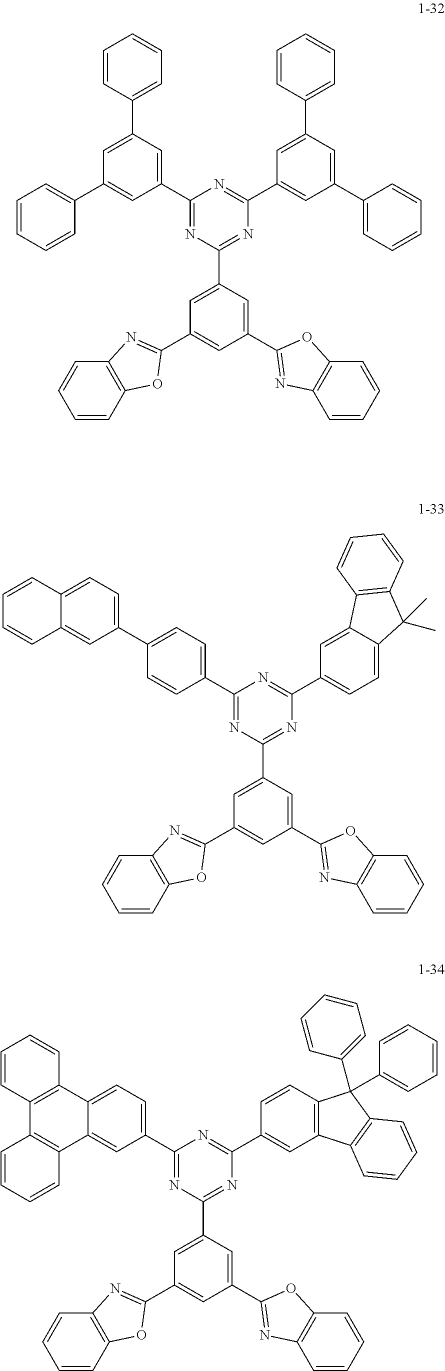 [SMILES: c1ccc(-c2cc(-c3ccccc3)cc(-c3nc(-c4cc(-c5ccccc5)cc(-c5ccccc5)c4)nc(-c4cc(-c5nc6ccccc6o5)cc(-c5nc6ccccc6o5)c4)n3)c2)cc1 ; CC1(C)c2ccccc2-c2cc(-c3nc(-c4ccc(-c5ccc6ccccc6c5)cc4)nc(-c4cc(-c5nc6ccccc6o5)cc(-c5nc6ccccc6o5)c4)n3)ccc21 ; c1ccc(C2(c3ccccc3)c3ccccc3-c3cc(-c4nc(-c5cc(-c6nc7ccccc7o6)cc(-c6nc7ccccc7o6)c5)nc(-c5ccc6c7ccccc7c7ccccc7c6c5)n4)ccc32)cc1]

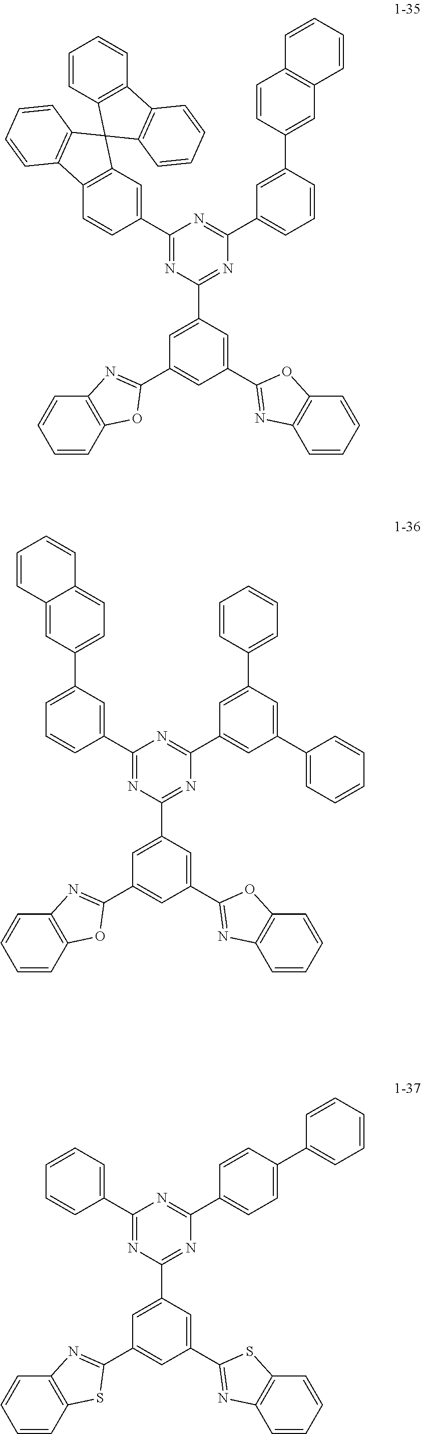 [SMILES: c1cc(-c2ccc3ccccc3c2)cc(-c2nc(-c3cc(-c4nc5ccccc5o4)cc(-c4nc5ccccc5o4)c3)nc(-c3ccc4c(c3)C3(c5ccccc5-c5ccccc53)c3ccccc3-4)n2)c1 ; c1ccc(-c2cc(-c3ccccc3)cc(-c3nc(-c4cccc(-c5ccc6ccccc6c5)c4)nc(-c4cc(-c5nc6ccccc6o5)cc(-c5nc6ccccc6o5)c4)n3)c2)cc1 ; c1ccc(-c2ccc(-c3nc(-c4ccccc4)nc(-c4cc(-c5nc6ccccc6s5)cc(-c5nc6ccccc6s5)c4)n3)cc2)cc1]

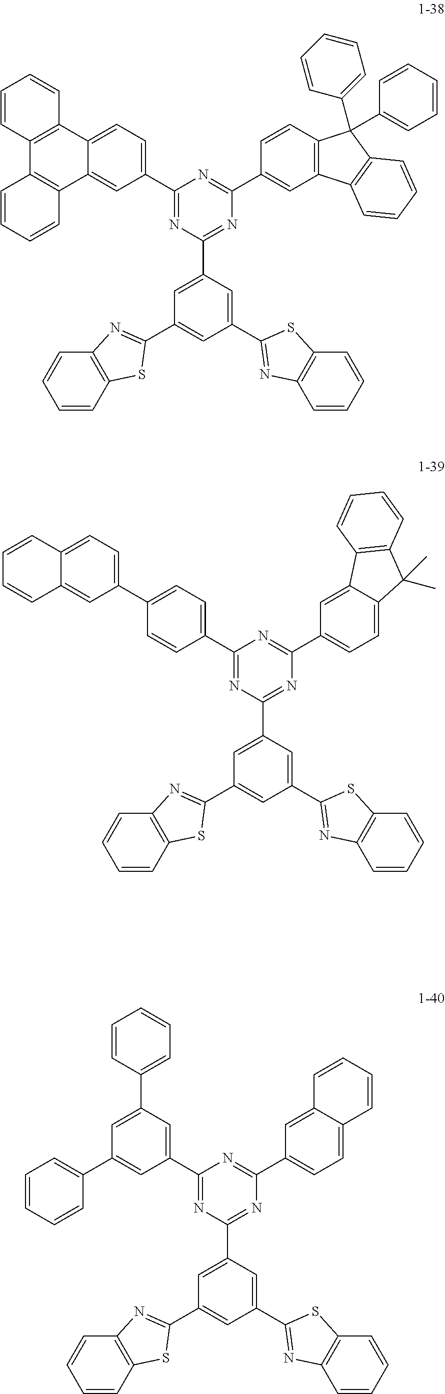 [SMILES: c1ccc(C2(c3ccccc3)c3ccccc3-c3cc(-c4nc(-c5cc(-c6nc7ccccc7s6)cc(-c6nc7ccccc7s6)c5)nc(-c5ccc6c7ccccc7c7ccccc7c6c5)n4)ccc32)cc1 ; CC1(C)c2ccccc2-c2cc(-c3nc(-c4ccc(-c5ccc6ccccc6c5)cc4)nc(-c4cc(-c5nc6ccccc6s5)cc(-c5nc6ccccc6s5)c4)n3)ccc21 ; c1ccc(-c2cc(-c3ccccc3)cc(-c3nc(-c4cc(-c5nc6ccccc6s5)cc(-c5nc6ccccc6s5)c4)nc(-c4ccc5ccccc5c4)n3)c2)cc1]

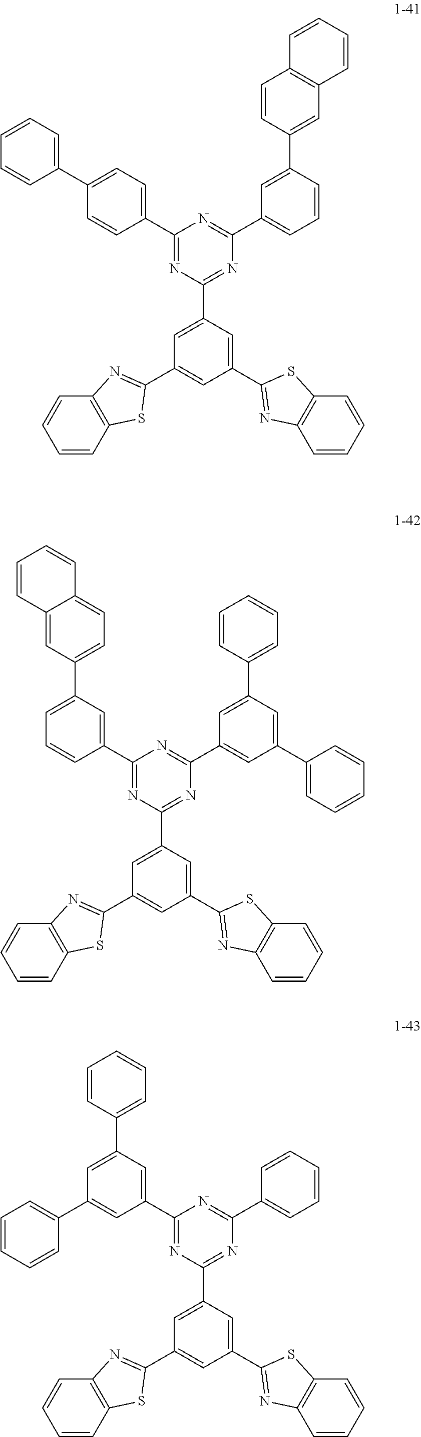 [SMILES: c1ccc(-c2ccc(-c3nc(-c4cccc(-c5ccc6ccccc6c5)c4)nc(-c4cc(-c5nc6ccccc6s5)cc(-c5nc6ccccc6s5)c4)n3)cc2)cc1 ; c1ccc(-c2cc(-c3ccccc3)cc(-c3nc(-c4cccc(-c5ccc6ccccc6c5)c4)nc(-c4cc(-c5nc6ccccc6s5)cc(-c5nc6ccccc6s5)c4)n3)c2)cc1 ; c1ccc(-c2cc(-c3ccccc3)cc(-c3nc(-c4ccccc4)nc(-c4cc(-c5nc6ccccc6s5)cc(-c5nc6ccccc6s5)c4)n3)c2)cc1]

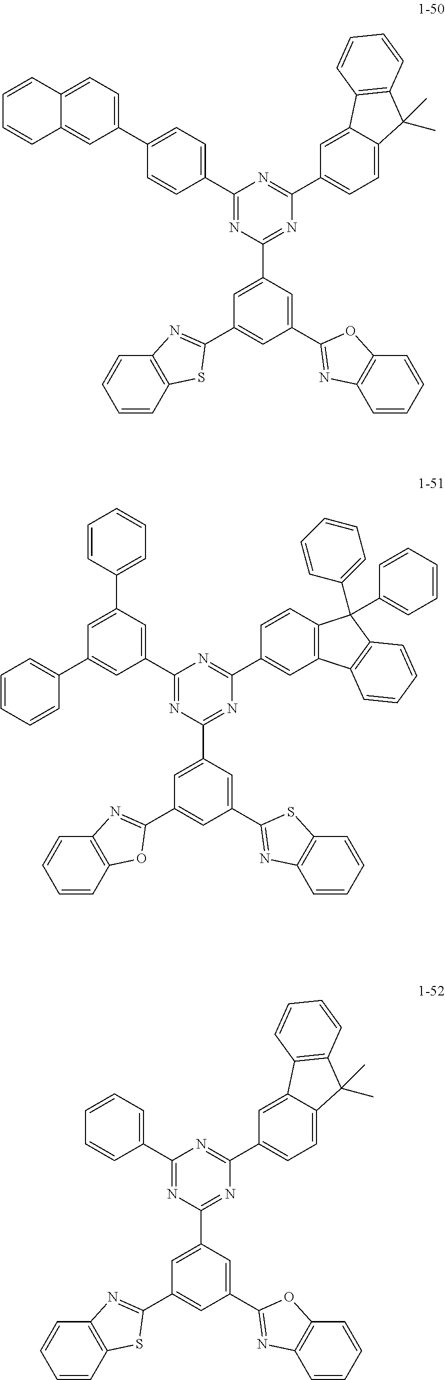 [SMILES: CC1(C)c2ccccc2-c2cc(-c3nc(-c4ccc(-c5ccc6ccccc6c5)cc4)nc(-c4cc(-c5nc6ccccc6o5)cc(-c5nc6ccccc6s5)c4)n3)ccc21 ; c1ccc(-c2cc(-c3ccccc3)cc(-c3nc(-c4cc(-c5nc6ccccc6o5)cc(-c5nc6ccccc6s5)c4)nc(-c4ccc5c(c4)-c4ccccc4C5(c4ccccc4)c4ccccc4)n3)c2)cc1 ; CC1(C)c2ccccc2-c2cc(-c3nc(-c4ccccc4)nc(-c4cc(-c5nc6ccccc6o5)cc(-c5nc6ccccc6s5)c4)n3)ccc21]

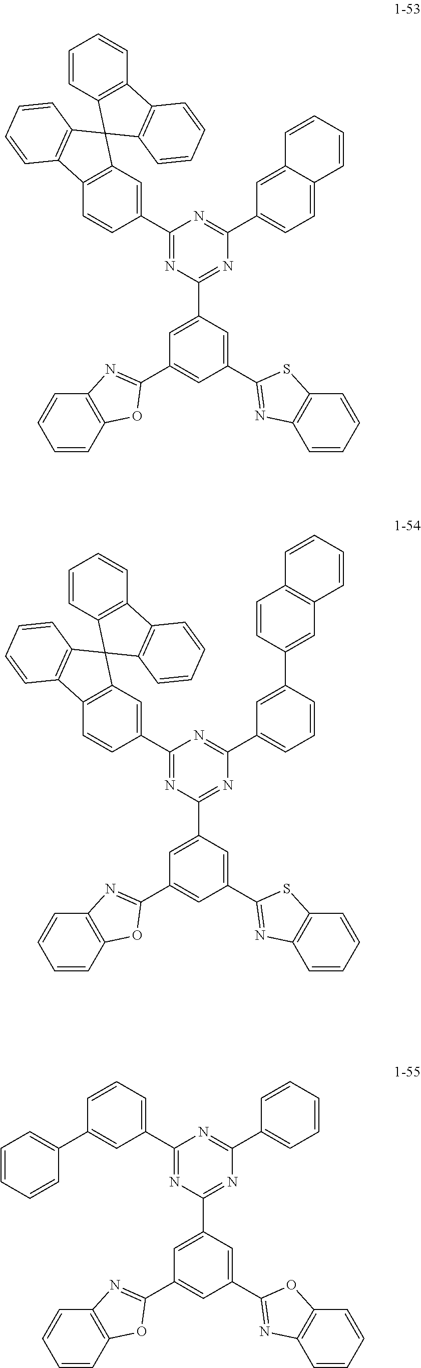 [SMILES: c1ccc2c(c1)-c1ccccc1C21c2ccccc2-c2ccc(-c3nc(-c4cc(-c5nc6ccccc6o5)cc(-c5nc6ccccc6s5)c4)nc(-c4ccc5ccccc5c4)n3)cc21 ; c1cc(-c2ccc3ccccc3c2)cc(-c2nc(-c3cc(-c4nc5ccccc5o4)cc(-c4nc5ccccc5s4)c3)nc(-c3ccc4c(c3)C3(c5ccccc5-c5ccccc53)c3ccccc3-4)n2)c1 ; c1ccc(-c2cccc(-c3nc(-c4ccccc4)nc(-c4cc(-c5nc6ccccc6o5)cc(-c5nc6ccccc6o5)c4)n3)c2)cc1]

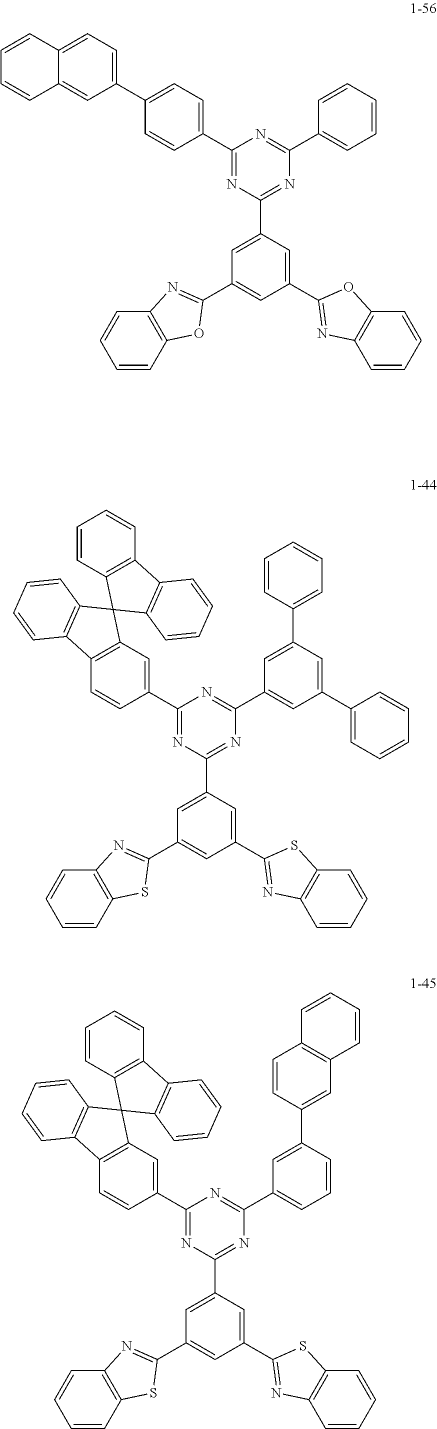 [SMILES: c1ccc(-c2nc(-c3ccc(-c4ccc5ccccc5c4)cc3)nc(-c3cc(-c4nc5ccccc5o4)cc(-c4nc5ccccc5o4)c3)n2)cc1 ; c1ccc(-c2cc(-c3ccccc3)cc(-c3nc(-c4cc(-c5nc6ccccc6s5)cc(-c5nc6ccccc6s5)c4)nc(-c4ccc5c(c4)C4(c6ccccc6-c6ccccc64)c4ccccc4-5)n3)c2)cc1 ; c1cc(-c2ccc3ccccc3c2)cc(-c2nc(-c3cc(-c4nc5ccccc5s4)cc(-c4nc5ccccc5s4)c3)nc(-c3ccc4c(c3)C3(c5ccccc5-c5ccccc53)c3ccccc3-4)n2)c1]

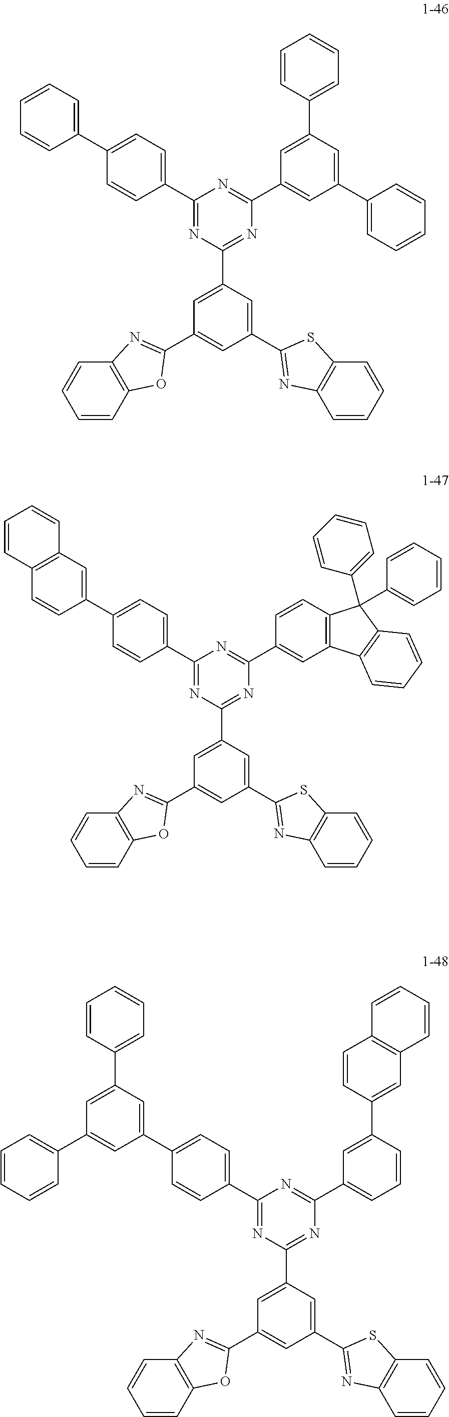 [SMILES: c1ccc(-c2ccc(-c3nc(-c4cc(-c5ccccc5)cc(-c5ccccc5)c4)nc(-c4cc(-c5nc6ccccc6o5)cc(-c5nc6ccccc6s5)c4)n3)cc2)cc1 ; c1ccc(C2(c3ccccc3)c3ccccc3-c3cc(-c4nc(-c5ccc(-c6ccc7ccccc7c6)cc5)nc(-c5cc(-c6nc7ccccc7o6)cc(-c6nc7ccccc7s6)c5)n4)ccc32)cc1 ; c1ccc(-c2cc(-c3ccccc3)cc(-c3ccc(-c4nc(-c5cccc(-c6ccc7ccccc7c6)c5)nc(-c5cc(-c6nc7ccccc7o6)cc(-c6nc7ccccc7s6)c5)n4)cc3)c2)cc1]

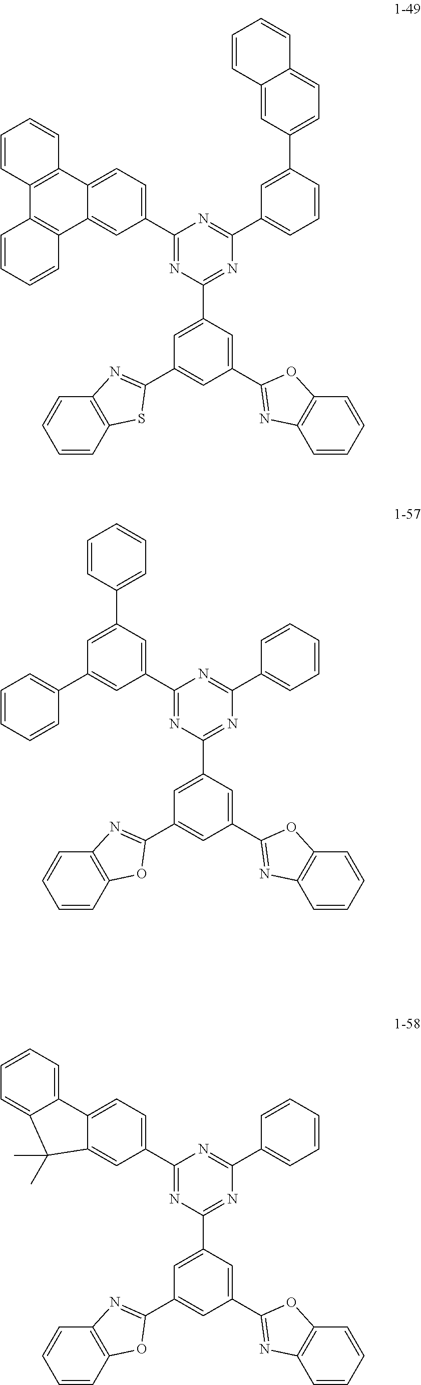 [SMILES: c1cc(-c2ccc3ccccc3c2)cc(-c2nc(-c3cc(-c4nc5ccccc5o4)cc(-c4nc5ccccc5s4)c3)nc(-c3ccc4c5ccccc5c5ccccc5c4c3)n2)c1 ; c1ccc(-c2cc(-c3ccccc3)cc(-c3nc(-c4ccccc4)nc(-c4cc(-c5nc6ccccc6o5)cc(-c5nc6ccccc6o5)c4)n3)c2)cc1 ; CC1(C)c2ccccc2-c2ccc(-c3nc(-c4ccccc4)nc(-c4cc(-c5nc6ccccc6o5)cc(-c5nc6ccccc6o5)c4)n3)cc21]

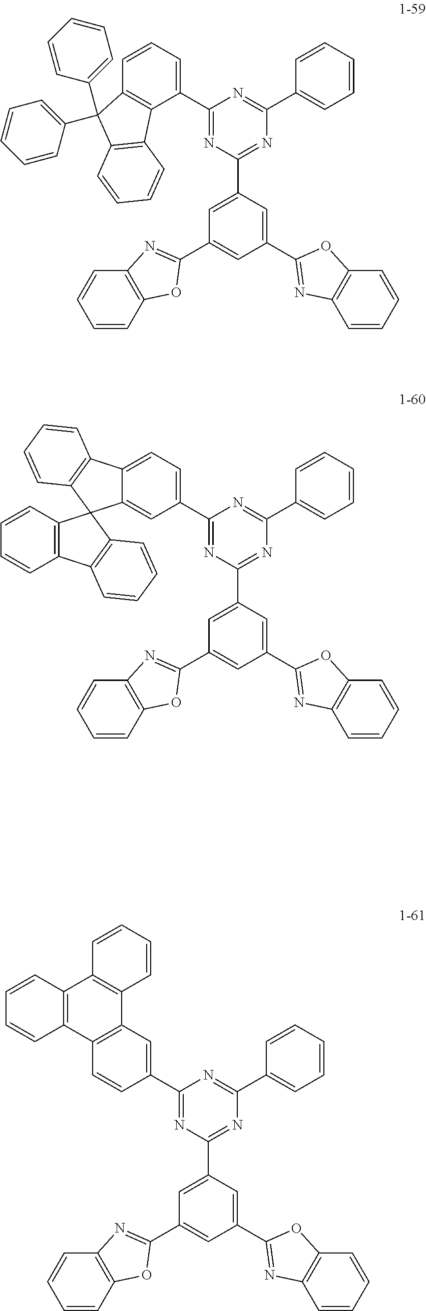 [SMILES: c1ccc(-c2nc(-c3cc(-c4nc5ccccc5o4)cc(-c4nc5ccccc5o4)c3)nc(-c3cccc4c3-c3ccccc3C4(c3ccccc3)c3ccccc3)n2)cc1 ; c1ccc(-c2nc(-c3cc(-c4nc5ccccc5o4)cc(-c4nc5ccccc5o4)c3)nc(-c3ccc4c(c3)C3(c5ccccc5-c5ccccc53)c3ccccc3-4)n2)cc1 ; c1ccc(-c2nc(-c3cc(-c4nc5ccccc5o4)cc(-c4nc5ccccc5o4)c3)nc(-c3ccc4c5ccccc5c5ccccc5c4c3)n2)cc1]

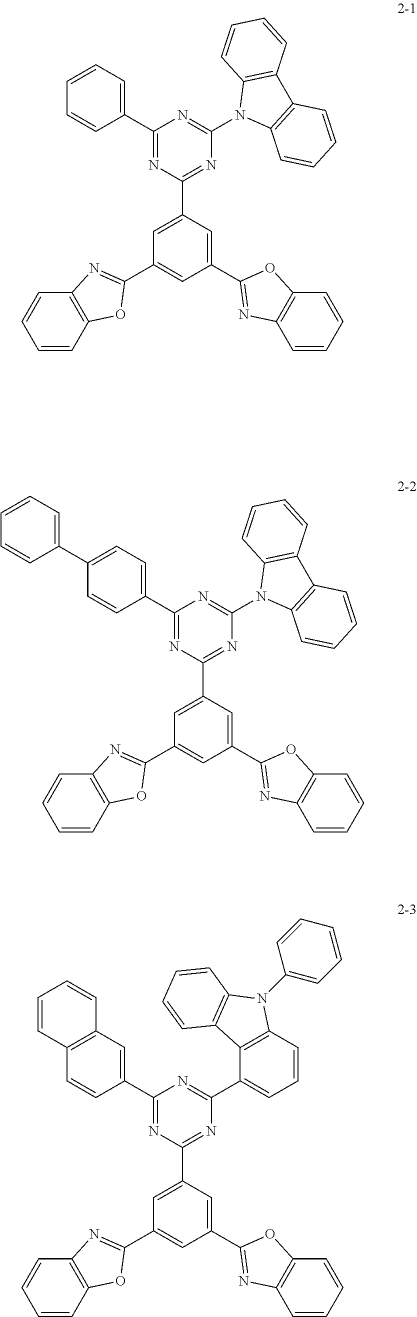 [SMILES: c1ccc(-c2nc(-c3cc(-c4nc5ccccc5o4)cc(-c4nc5ccccc5o4)c3)nc(-n3c4ccccc4c4ccccc43)n2)cc1 ; c1ccc(-c2ccc(-c3nc(-c4cc(-c5nc6ccccc6o5)cc(-c5nc6ccccc6o5)c4)nc(-n4c5ccccc5c5ccccc54)n3)cc2)cc1 ; c1ccc(-n2c3ccccc3c3c(-c4nc(-c5cc(-c6nc7ccccc7o6)cc(-c6nc7ccccc7o6)c5)nc(-c5ccc6ccccc6c5)n4)cccc32)cc1]

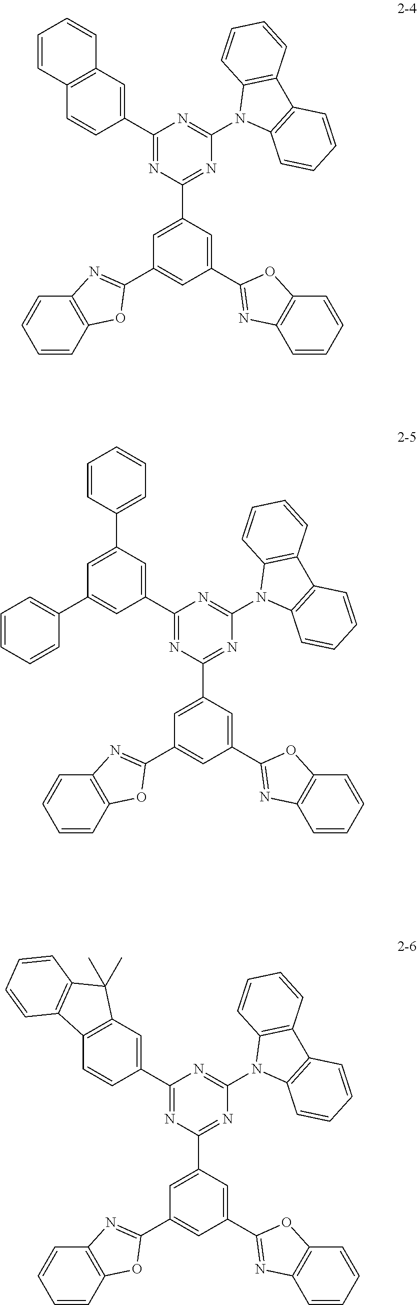 [SMILES: c1ccc2cc(-c3nc(-c4cc(-c5nc6ccccc6o5)cc(-c5nc6ccccc6o5)c4)nc(-n4c5ccccc5c5ccccc54)n3)ccc2c1 ; c1ccc(-c2cc(-c3ccccc3)cc(-c3nc(-c4cc(-c5nc6ccccc6o5)cc(-c5nc6ccccc6o5)c4)nc(-n4c5ccccc5c5ccccc54)n3)c2)cc1 ; CC1(C)c2ccccc2-c2ccc(-c3nc(-c4cc(-c5nc6ccccc6o5)cc(-c5nc6ccccc6o5)c4)nc(-n4c5ccccc5c5ccccc54)n3)cc21]

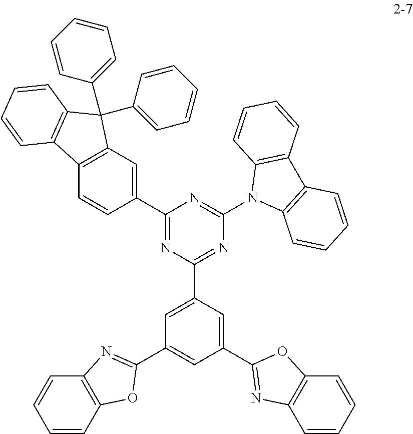 [SMILES: c1ccc(C2(c3ccccc3)c3ccccc3-c3ccc(-c4nc(-c5cc(-c6nc7ccccc7o6)cc(-c6nc7ccccc7o6)c5)nc(-n5c6ccccc6c6ccccc65)n4)cc32)cc1]

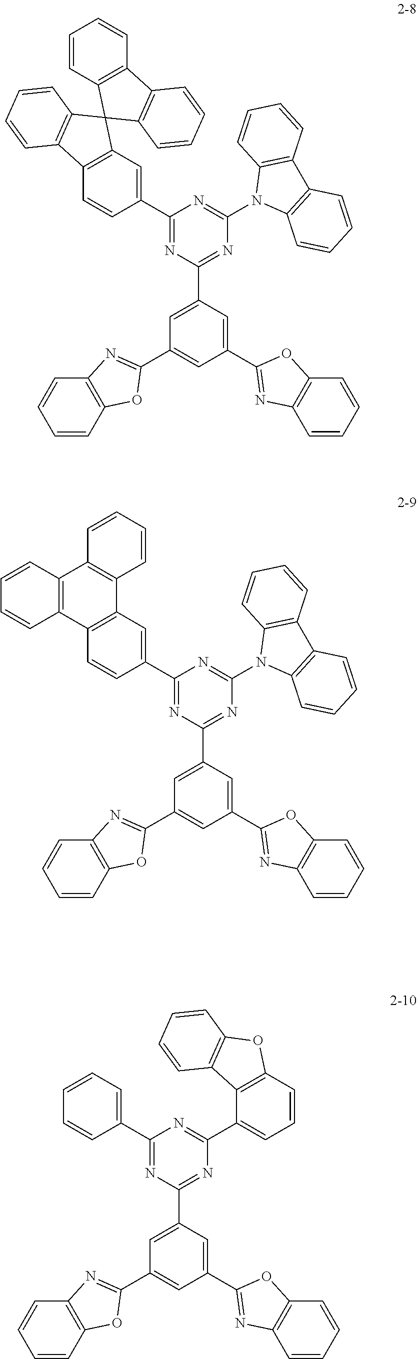 [SMILES: c1ccc2c(c1)-c1ccccc1C21c2ccccc2-c2ccc(-c3nc(-c4cc(-c5nc6ccccc6o5)cc(-c5nc6ccccc6o5)c4)nc(-n4c5ccccc5c5ccccc54)n3)cc21 ; c1ccc2oc(-c3cc(-c4nc(-c5ccc6c7ccccc7c7ccccc7c6c5)nc(-n5c6ccccc6c6ccccc65)n4)cc(-c4nc5ccccc5o4)c3)nc2c1 ; c1ccc(-c2nc(-c3cc(-c4nc5ccccc5o4)cc(-c4nc5ccccc5o4)c3)nc(-c3cccc4oc5ccccc5c34)n2)cc1]

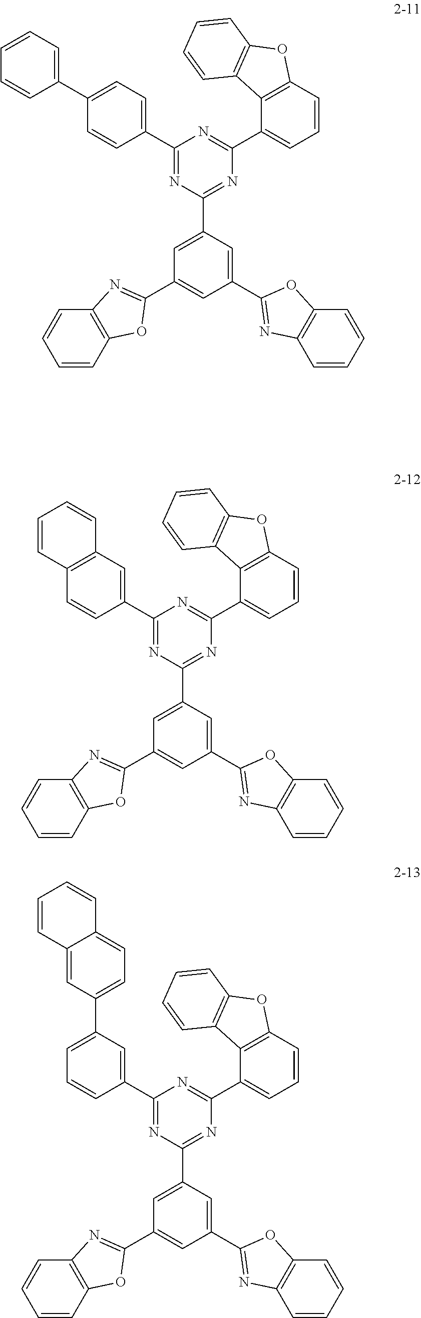

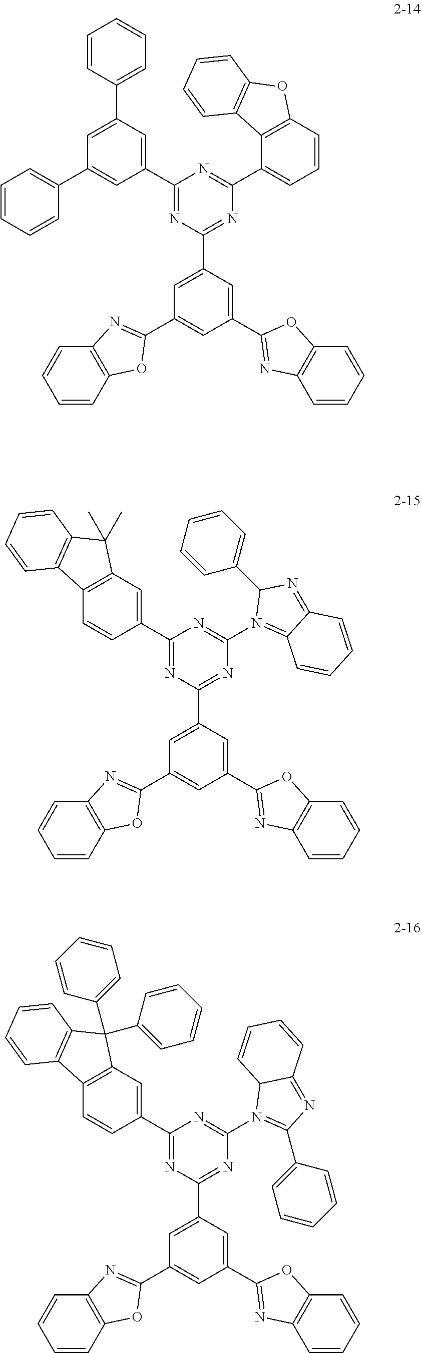

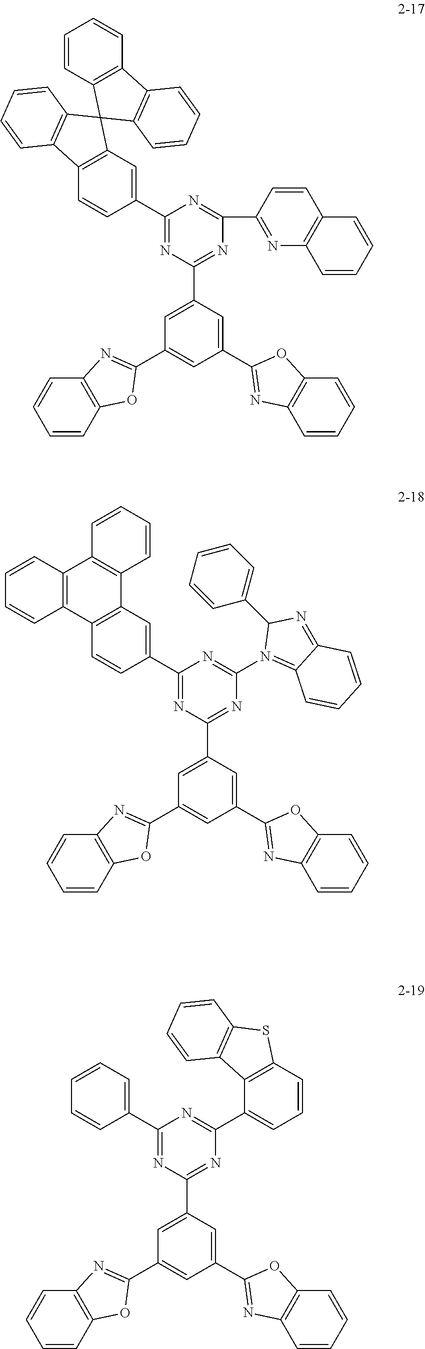

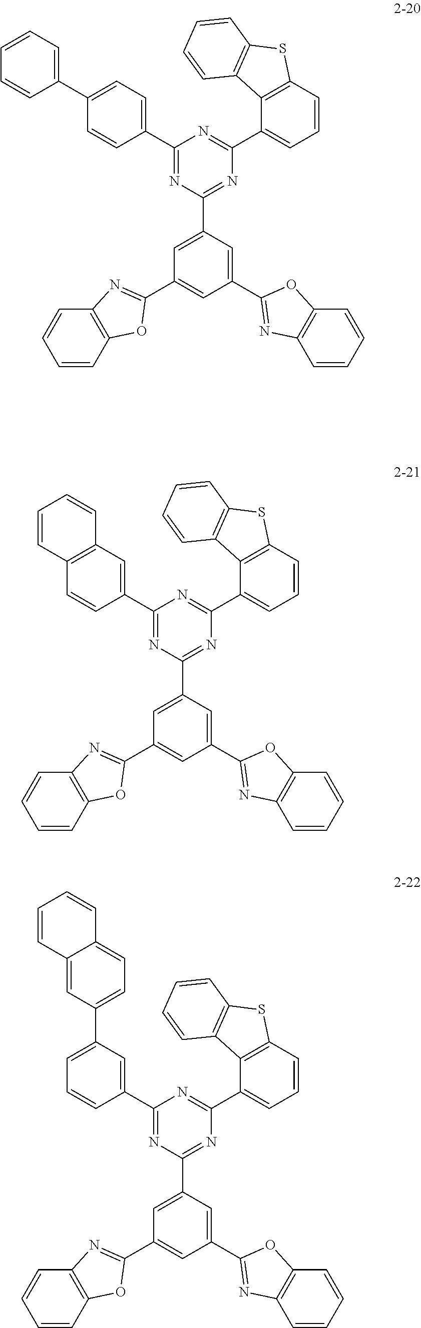 [SMILES: c1ccc(-c2ccc(-c3nc(-c4cc(-c5nc6ccccc6o5)cc(-c5nc6ccccc6o5)c4)nc(-c4cccc5sc6ccccc6c45)n3)cc2)cc1 ; c1ccc2cc(-c3nc(-c4cc(-c5nc6ccccc6o5)cc(-c5nc6ccccc6o5)c4)nc(-c4cccc5sc6ccccc6c45)n3)ccc2c1 ; c1cc(-c2ccc3ccccc3c2)cc(-c2nc(-c3cc(-c4nc5ccccc5o4)cc(-c4nc5ccccc5o4)c3)nc(-c3cccc4sc5ccccc5c34)n2)c1]

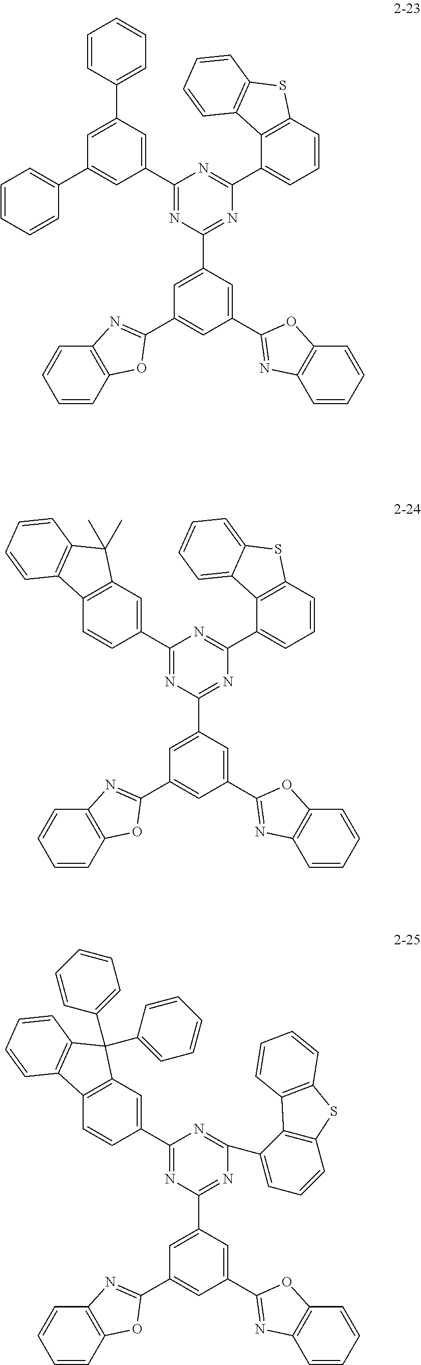 [SMILES: c1ccc(-c2cc(-c3ccccc3)cc(-c3nc(-c4cc(-c5nc6ccccc6o5)cc(-c5nc6ccccc6o5)c4)nc(-c4cccc5sc6ccccc6c45)n3)c2)cc1 ; CC1(C)c2ccccc2-c2ccc(-c3nc(-c4cc(-c5nc6ccccc6o5)cc(-c5nc6ccccc6o5)c4)nc(-c4cccc5sc6ccccc6c45)n3)cc21 ; c1ccc(C2(c3ccccc3)c3ccccc3-c3ccc(-c4nc(-c5cc(-c6nc7ccccc7o6)cc(-c6nc7ccccc7o6)c5)nc(-c5cccc6sc7ccccc7c56)n4)cc32)cc1]

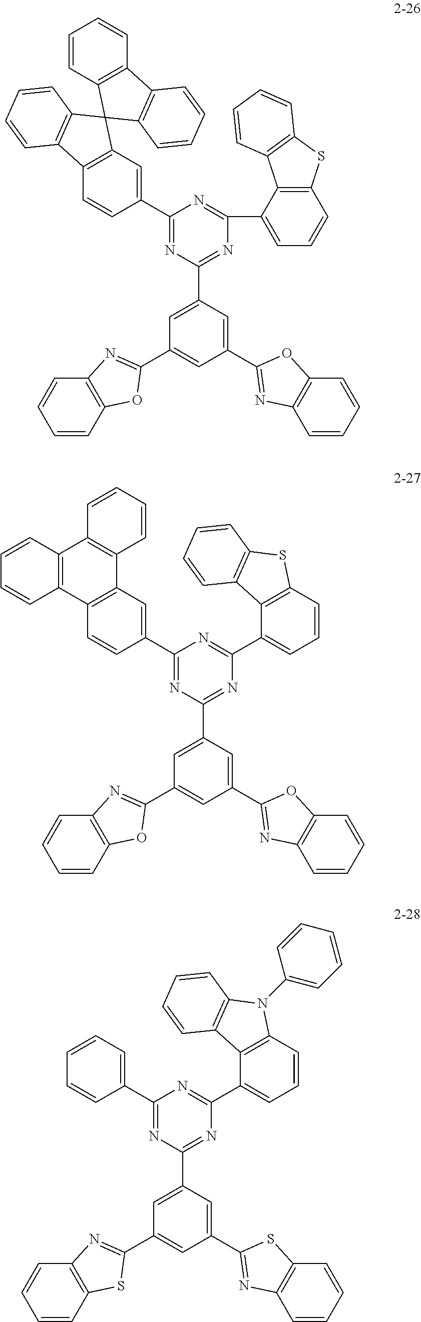 [SMILES: c1ccc2c(c1)-c1ccccc1C21c2ccccc2-c2ccc(-c3nc(-c4cc(-c5nc6ccccc6o5)cc(-c5nc6ccccc6o5)c4)nc(-c4cccc5sc6ccccc6c45)n3)cc21 ; c1ccc2oc(-c3cc(-c4nc(-c5ccc6c7ccccc7c7ccccc7c6c5)nc(-c5cccc6sc7ccccc7c56)n4)cc(-c4nc5ccccc5o4)c3)nc2c1 ; c1ccc(-c2nc(-c3cc(-c4nc5ccccc5s4)cc(-c4nc5ccccc5s4)c3)nc(-c3cccc4c3c3ccccc3n4-c3ccccc3)n2)cc1]

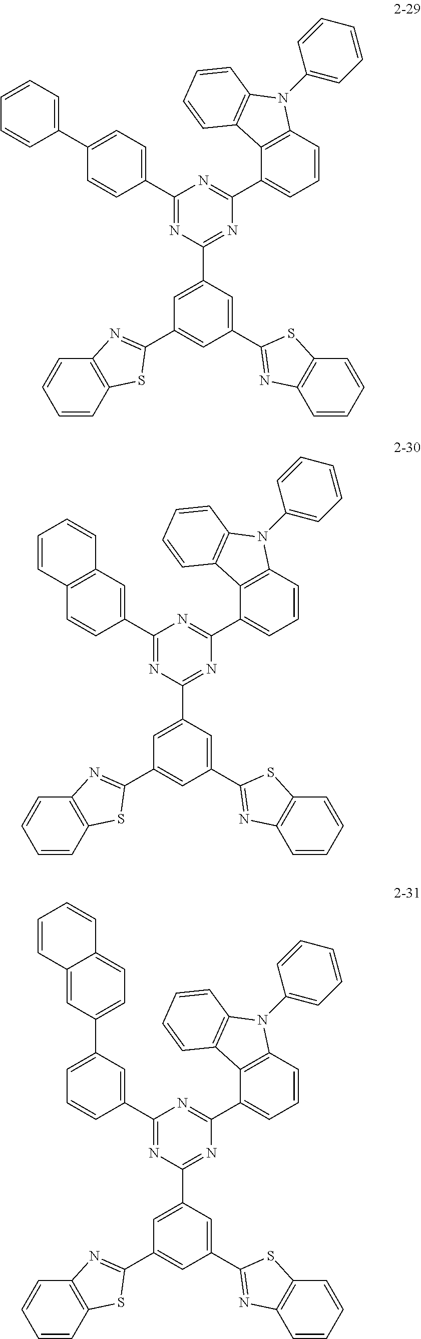

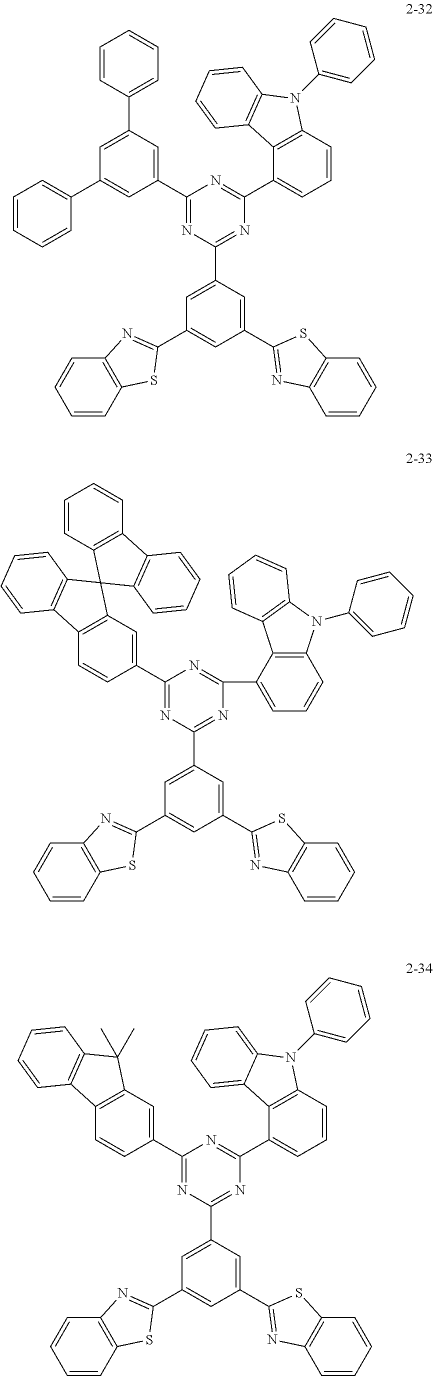 [SMILES: c1ccc(-c2cc(-c3ccccc3)cc(-c3nc(-c4cc(-c5nc6ccccc6s5)cc(-c5nc6ccccc6s5)c4)nc(-c4cccc5c4c4ccccc4n5-c4ccccc4)n3)c2)cc1 ; c1ccc(-n2c3ccccc3c3c(-c4nc(-c5cc(-c6nc7ccccc7s6)cc(-c6nc7ccccc7s6)c5)nc(-c5ccc6c(c5)C5(c7ccccc7-c7ccccc75)c5ccccc5-6)n4)cccc32)cc1 ; CC1(C)c2ccccc2-c2ccc(-c3nc(-c4cc(-c5nc6ccccc6s5)cc(-c5nc6ccccc6s5)c4)nc(-c4cccc5c4c4ccccc4n5-c4ccccc4)n3)cc21]

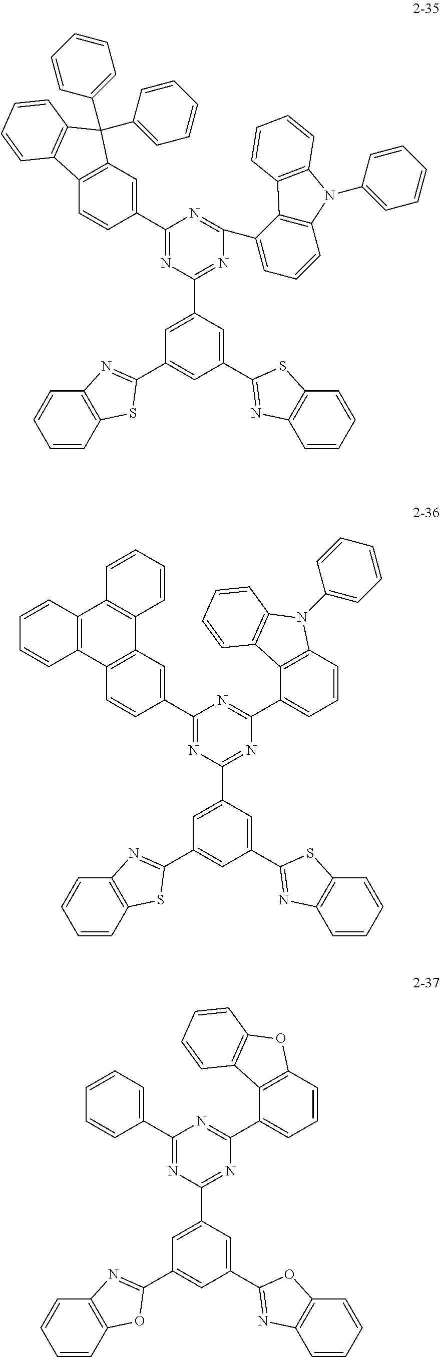 [SMILES: c1ccc(-n2c3ccccc3c3c(-c4nc(-c5cc(-c6nc7ccccc7s6)cc(-c6nc7ccccc7s6)c5)nc(-c5ccc6c(c5)C(c5ccccc5)(c5ccccc5)c5ccccc5-6)n4)cccc32)cc1 ; c1ccc(-n2c3ccccc3c3c(-c4nc(-c5cc(-c6nc7ccccc7s6)cc(-c6nc7ccccc7s6)c5)nc(-c5ccc6c7ccccc7c7ccccc7c6c5)n4)cccc32)cc1 ; c1ccc(-c2nc(-c3cc(-c4nc5ccccc5o4)cc(-c4nc5ccccc5o4)c3)nc(-c3cccc4oc5ccccc5c34)n2)cc1]

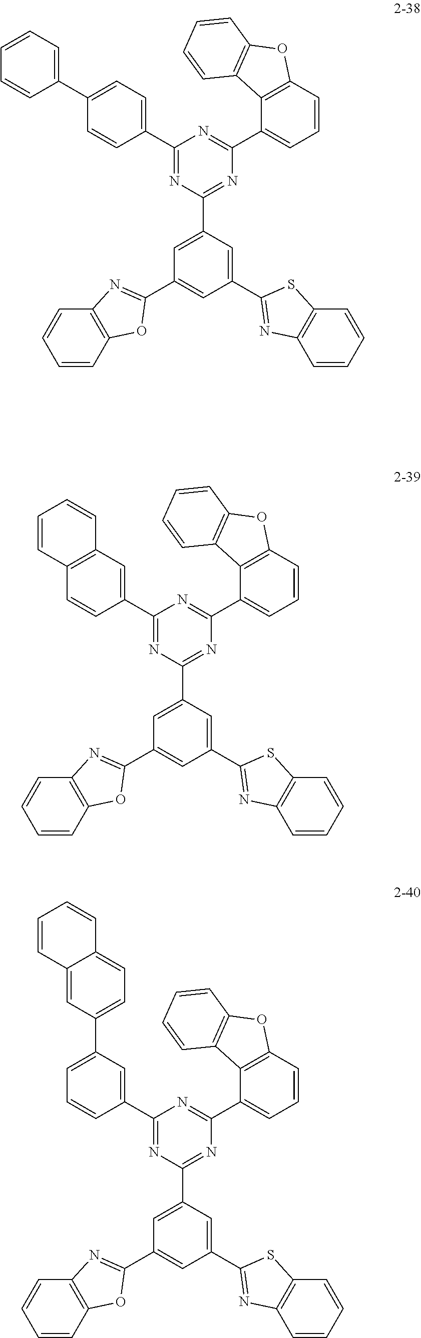 [SMILES: c1ccc(-c2ccc(-c3nc(-c4cc(-c5nc6ccccc6o5)cc(-c5nc6ccccc6s5)c4)nc(-c4cccc5oc6ccccc6c45)n3)cc2)cc1 ; c1ccc2cc(-c3nc(-c4cc(-c5nc6ccccc6o5)cc(-c5nc6ccccc6s5)c4)nc(-c4cccc5oc6ccccc6c45)n3)ccc2c1 ; c1cc(-c2ccc3ccccc3c2)cc(-c2nc(-c3cc(-c4nc5ccccc5o4)cc(-c4nc5ccccc5s4)c3)nc(-c3cccc4oc5ccccc5c34)n2)c1]

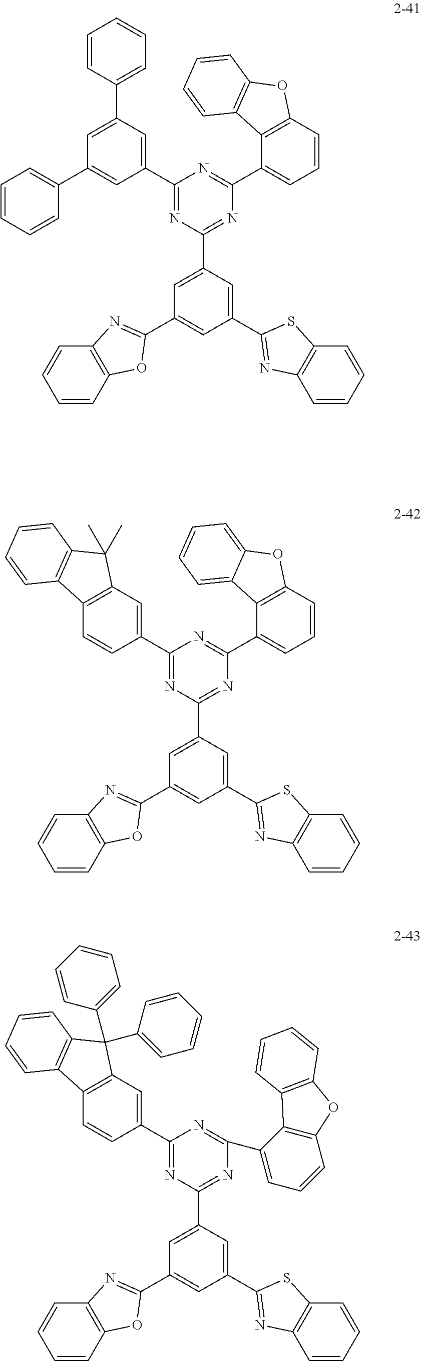 [SMILES: c1ccc(-c2cc(-c3ccccc3)cc(-c3nc(-c4cc(-c5nc6ccccc6o5)cc(-c5nc6ccccc6s5)c4)nc(-c4cccc5oc6ccccc6c45)n3)c2)cc1 ; CC1(C)c2ccccc2-c2ccc(-c3nc(-c4cc(-c5nc6ccccc6o5)cc(-c5nc6ccccc6s5)c4)nc(-c4cccc5oc6ccccc6c45)n3)cc21 ; c1ccc(C2(c3ccccc3)c3ccccc3-c3ccc(-c4nc(-c5cc(-c6nc7ccccc7o6)cc(-c6nc7ccccc7s6)c5)nc(-c5cccc6oc7ccccc7c56)n4)cc32)cc1]

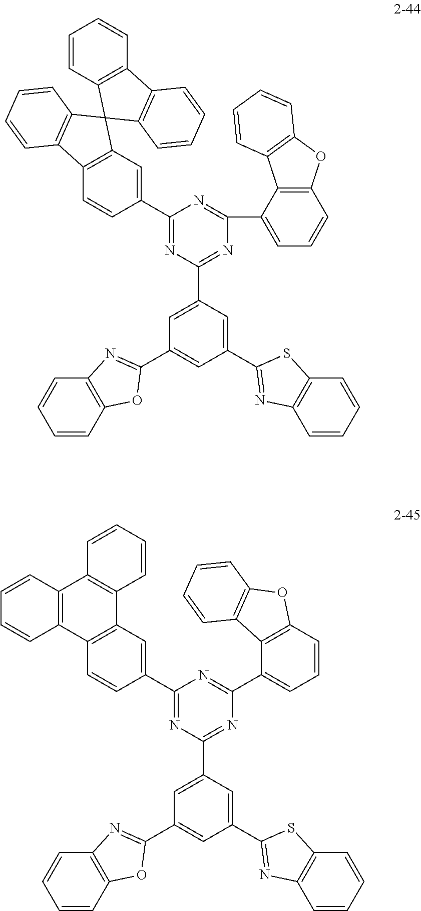 [SMILES: c1ccc2c(c1)-c1ccccc1C21c2ccccc2-c2ccc(-c3nc(-c4cc(-c5nc6ccccc6o5)cc(-c5nc6ccccc6s5)c4)nc(-c4cccc5oc6ccccc6c45)n3)cc21 ; c1ccc2oc(-c3cc(-c4nc(-c5ccc6c7ccccc7c7ccccc7c6c5)nc(-c5cccc6oc7ccccc7c56)n4)cc(-c4nc5ccccc5s4)c3)nc2c1]

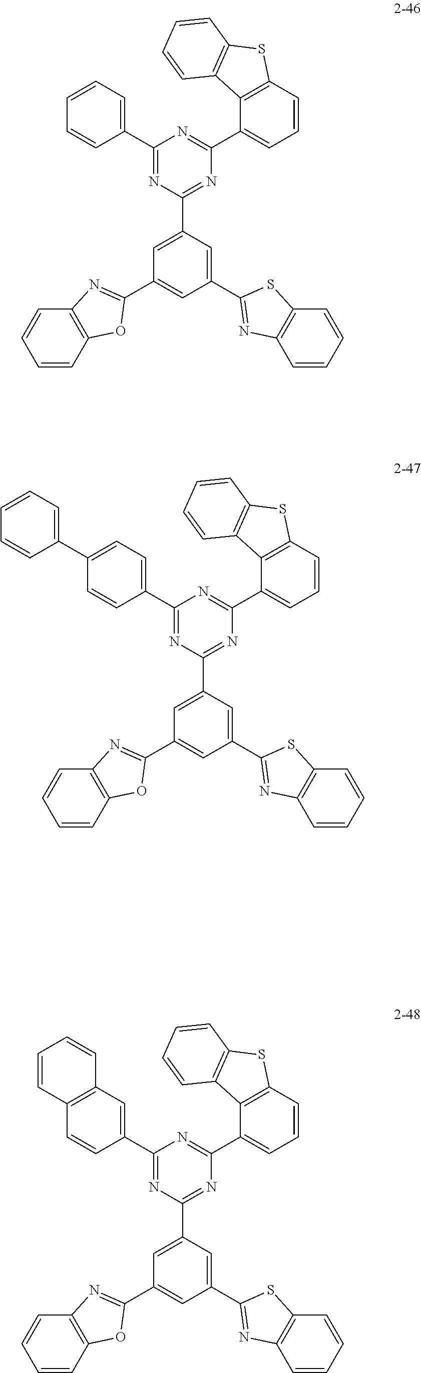 [SMILES: c1ccc(-c2nc(-c3cc(-c4nc5ccccc5o4)cc(-c4nc5ccccc5s4)c3)nc(-c3cccc4sc5ccccc5c34)n2)cc1 ; c1ccc(-c2ccc(-c3nc(-c4cc(-c5nc6ccccc6o5)cc(-c5nc6ccccc6s5)c4)nc(-c4cccc5sc6ccccc6c45)n3)cc2)cc1 ; c1ccc2cc(-c3nc(-c4cc(-c5nc6ccccc6o5)cc(-c5nc6ccccc6s5)c4)nc(-c4cccc5sc6ccccc6c45)n3)ccc2c1]

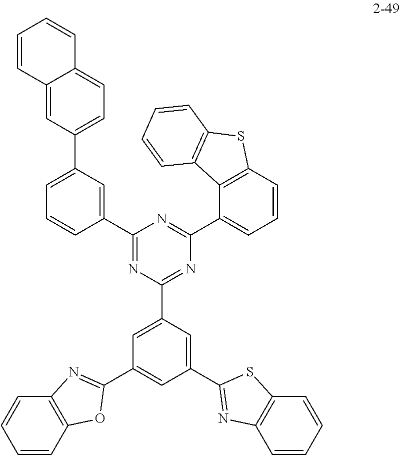 [SMILES: c1cc(-c2ccc3ccccc3c2)cc(-c2nc(-c3cc(-c4nc5ccccc5o4)cc(-c4nc5ccccc5s4)c3)nc(-c3cccc4sc5ccccc5c34)n2)c1]

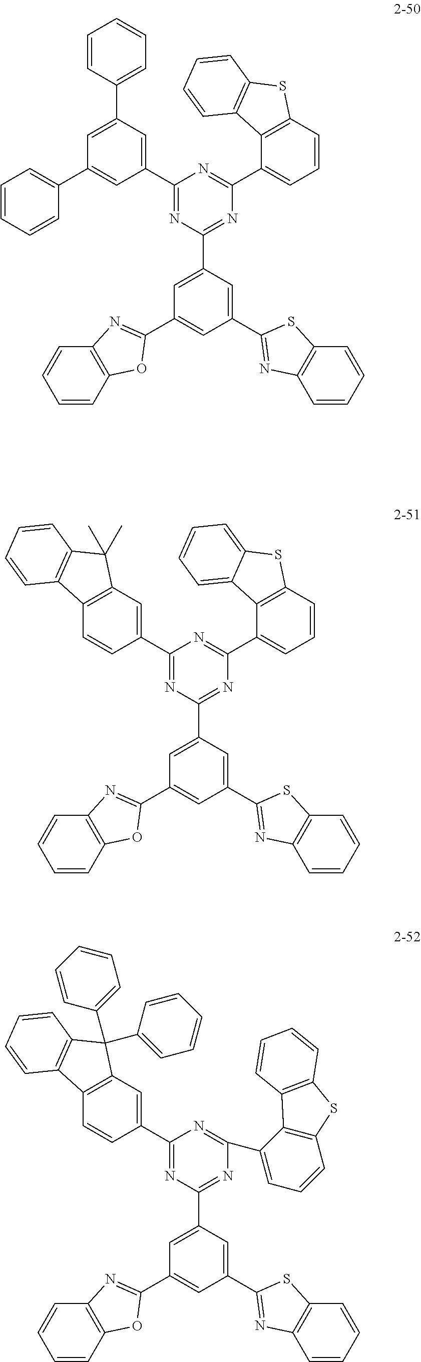 [SMILES: c1ccc(-c2cc(-c3ccccc3)cc(-c3nc(-c4cc(-c5nc6ccccc6o5)cc(-c5nc6ccccc6s5)c4)nc(-c4cccc5sc6ccccc6c45)n3)c2)cc1 ; CC1(C)c2ccccc2-c2ccc(-c3nc(-c4cc(-c5nc6ccccc6o5)cc(-c5nc6ccccc6s5)c4)nc(-c4cccc5sc6ccccc6c45)n3)cc21 ; c1ccc(C2(c3ccccc3)c3ccccc3-c3ccc(-c4nc(-c5cc(-c6nc7ccccc7o6)cc(-c6nc7ccccc7s6)c5)nc(-c5cccc6sc7ccccc7c56)n4)cc32)cc1]

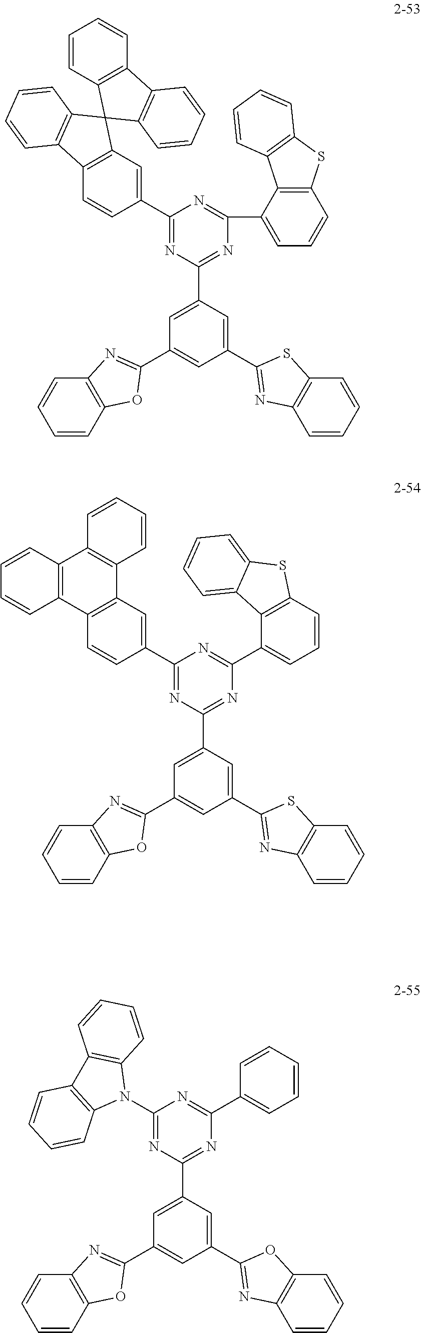 [SMILES: c1ccc2c(c1)-c1ccccc1C21c2ccccc2-c2ccc(-c3nc(-c4cc(-c5nc6ccccc6o5)cc(-c5nc6ccccc6s5)c4)nc(-c4cccc5sc6ccccc6c45)n3)cc21 ; c1ccc2oc(-c3cc(-c4nc(-c5ccc6c7ccccc7c7ccccc7c6c5)nc(-c5cccc6sc7ccccc7c56)n4)cc(-c4nc5ccccc5s4)c3)nc2c1 ; c1ccc(-c2nc(-c3cc(-c4nc5ccccc5o4)cc(-c4nc5ccccc5o4)c3)nc(-n3c4ccccc4c4ccccc43)n2)cc1]

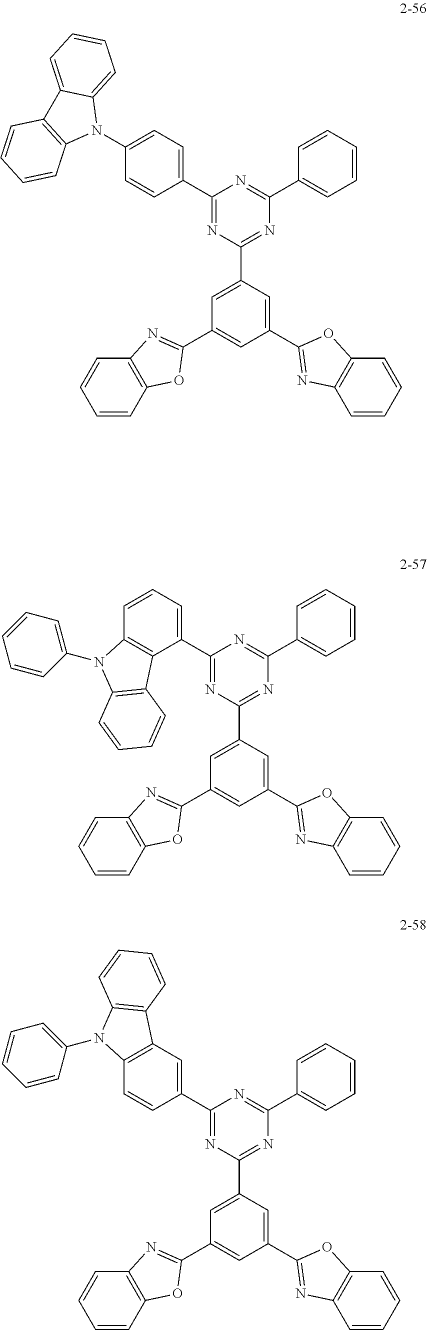 [SMILES: c1ccc(-c2nc(-c3ccc(-n4c5ccccc5c5ccccc54)cc3)nc(-c3cc(-c4nc5ccccc5o4)cc(-c4nc5ccccc5o4)c3)n2)cc1 ; c1ccc(-c2nc(-c3cc(-c4nc5ccccc5o4)cc(-c4nc5ccccc5o4)c3)nc(-c3cccc4c3c3ccccc3n4-c3ccccc3)n2)cc1 ; c1ccc(-c2nc(-c3cc(-c4nc5ccccc5o4)cc(-c4nc5ccccc5o4)c3)nc(-c3ccc4c(c3)c3ccccc3n4-c3ccccc3)n2)cc1]

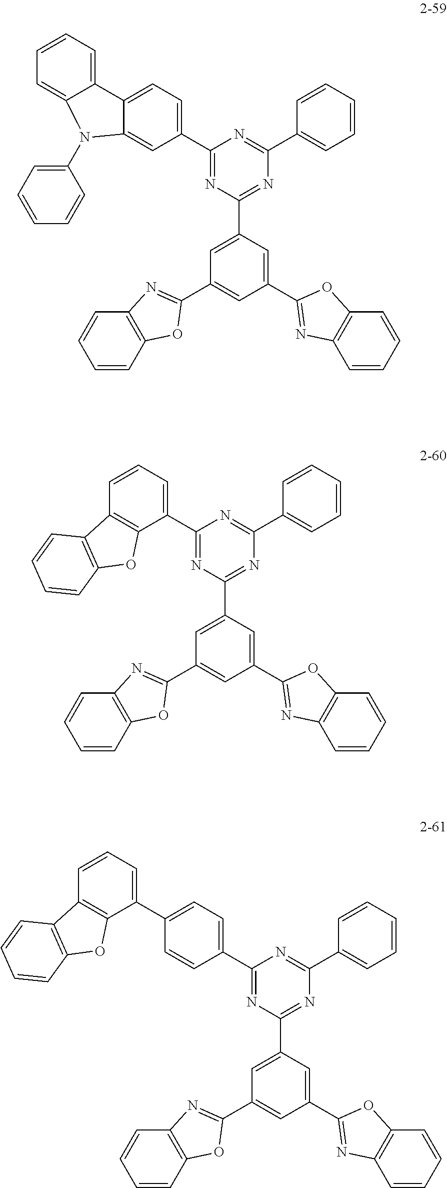 [SMILES: c1ccc(-c2nc(-c3cc(-c4nc5ccccc5o4)cc(-c4nc5ccccc5o4)c3)nc(-c3ccc4c5ccccc5n(-c5ccccc5)c4c3)n2)cc1 ; c1ccc(-c2nc(-c3cc(-c4nc5ccccc5o4)cc(-c4nc5ccccc5o4)c3)nc(-c3cccc4c3oc3ccccc34)n2)cc1 ; c1ccc(-c2nc(-c3ccc(-c4cccc5c4oc4ccccc45)cc3)nc(-c3cc(-c4nc5ccccc5o4)cc(-c4nc5ccccc5o4)c3)n2)cc1]

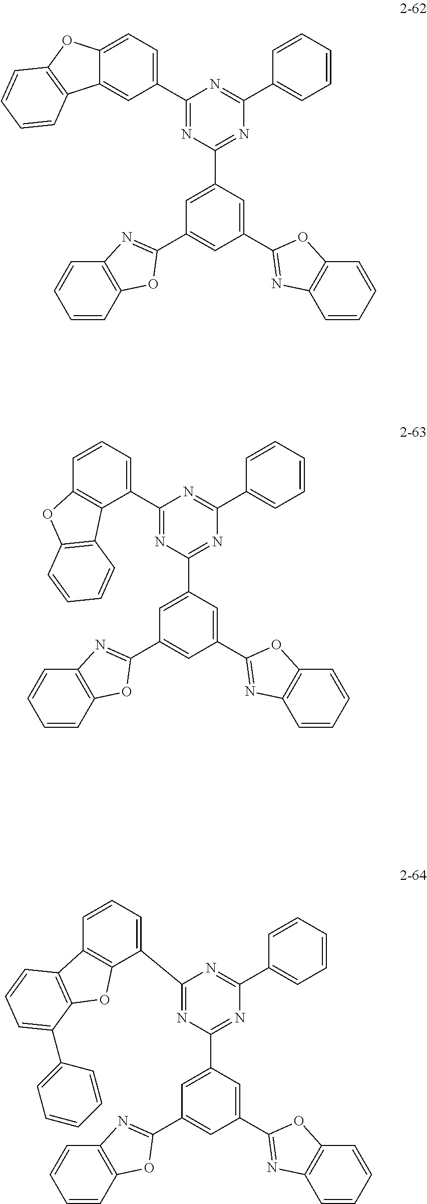 [SMILES: c1ccc(-c2nc(-c3cc(-c4nc5ccccc5o4)cc(-c4nc5ccccc5o4)c3)nc(-c3ccc4oc5ccccc5c4c3)n2)cc1 ; c1ccc(-c2nc(-c3cc(-c4nc5ccccc5o4)cc(-c4nc5ccccc5o4)c3)nc(-c3cccc4oc5ccccc5c34)n2)cc1 ; c1ccc(-c2nc(-c3cc(-c4nc5ccccc5o4)cc(-c4nc5ccccc5o4)c3)nc(-c3cccc4c3oc3c(-c5ccccc5)cccc34)n2)cc1]

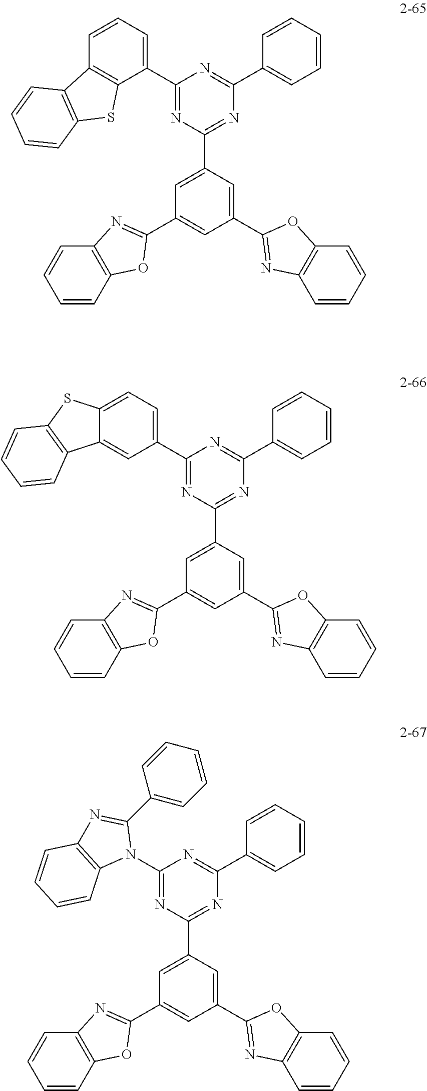 [SMILES: c1ccc(-c2nc(-c3cc(-c4nc5ccccc5o4)cc(-c4nc5ccccc5o4)c3)nc(-c3cccc4c3sc3ccccc34)n2)cc1 ; c1ccc(-c2nc(-c3cc(-c4nc5ccccc5o4)cc(-c4nc5ccccc5o4)c3)nc(-c3ccc4sc5ccccc5c4c3)n2)cc1 ; c1ccc(-c2nc(-c3cc(-c4nc5ccccc5o4)cc(-c4nc5ccccc5o4)c3)nc(-n3c(-c4ccccc4)nc4ccccc43)n2)cc1]

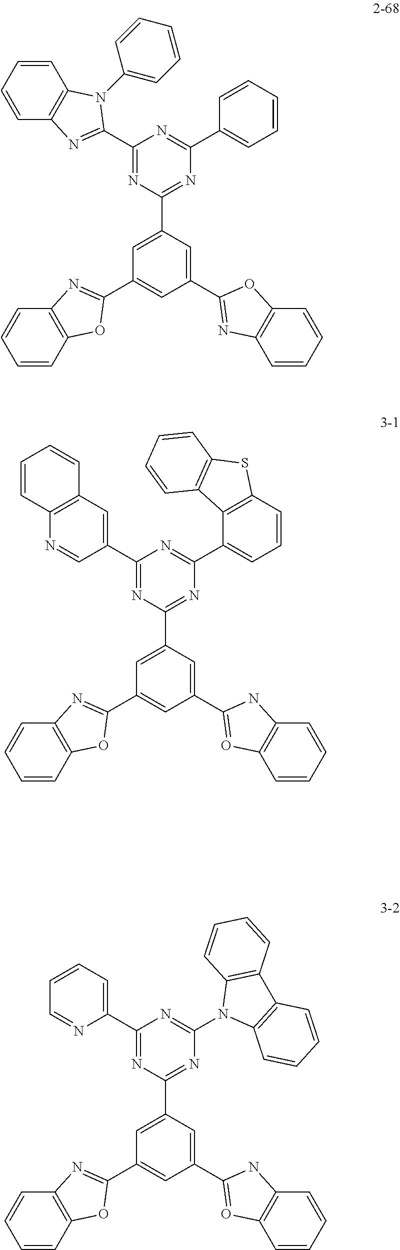

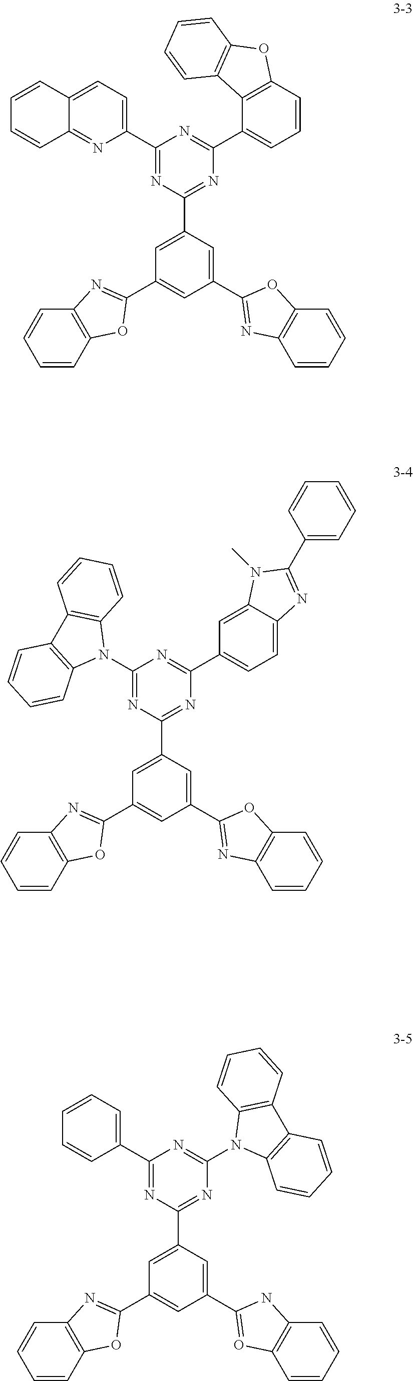

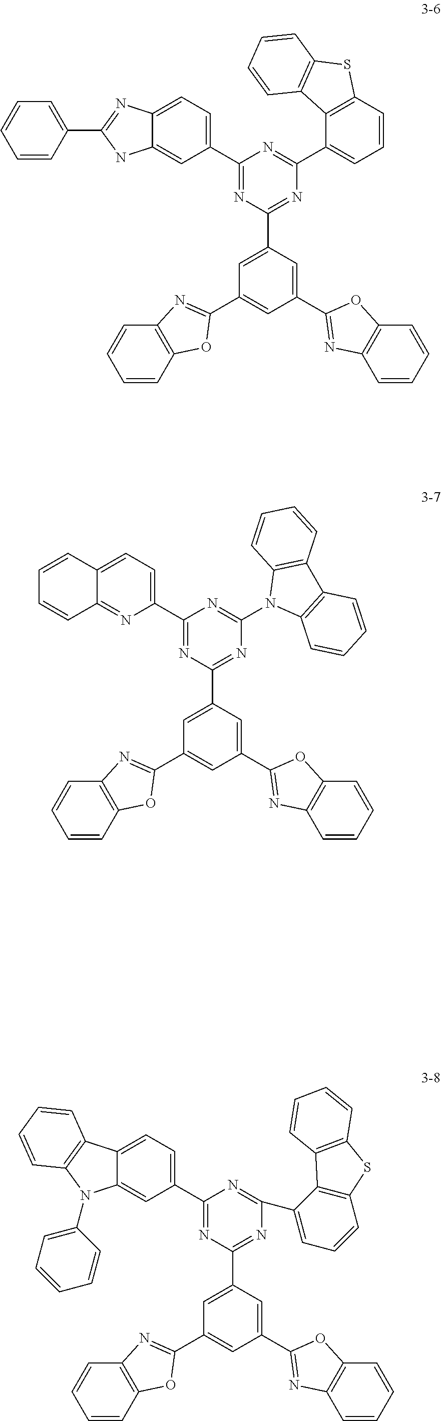 [SMILES: c1ccc(-c2nc3ccc(-c4nc(-c5cc(-c6nc7ccccc7o6)cc(-c6nc7ccccc7o6)c5)nc(-c5cccc6sc7ccccc7c56)n4)cc3[nH]2)cc1 ; c1ccc2nc(-c3nc(-c4cc(-c5nc6ccccc6o5)cc(-c5nc6ccccc6o5)c4)nc(-n4c5ccccc5c5ccccc54)n3)ccc2c1 ; c1ccc(-n2c3ccccc3c3ccc(-c4nc(-c5cc(-c6nc7ccccc7o6)cc(-c6nc7ccccc7o6)c5)nc(-c5cccc6sc7ccccc7c56)n4)cc32)cc1]

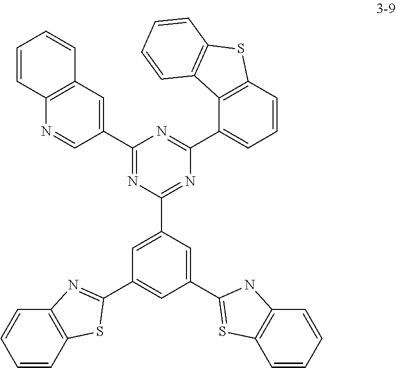

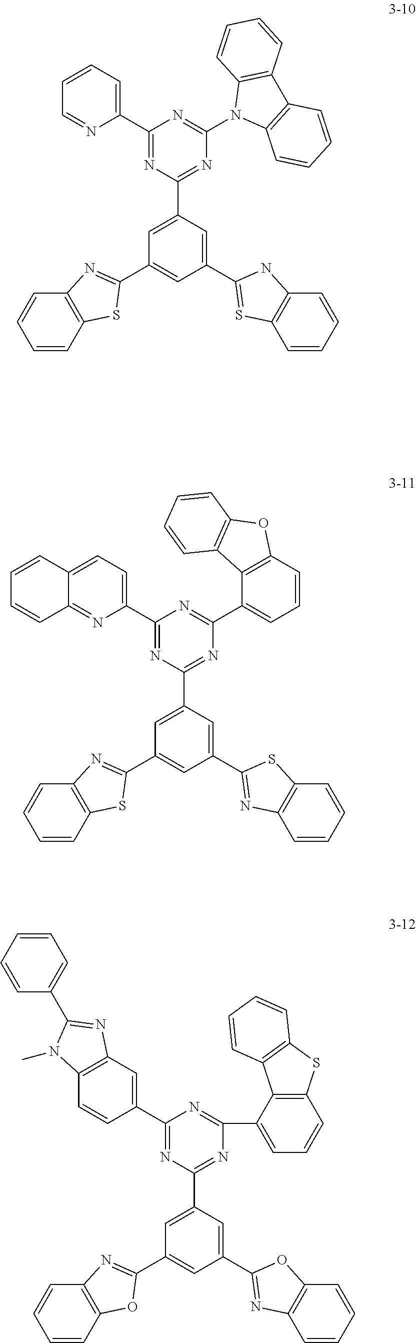

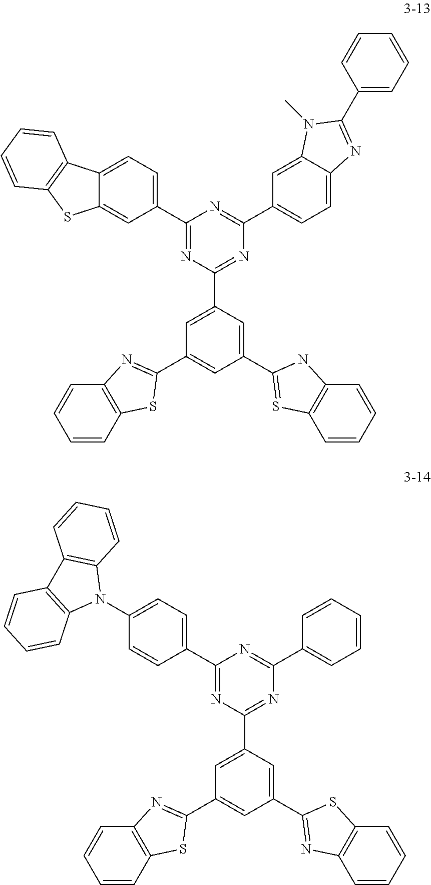

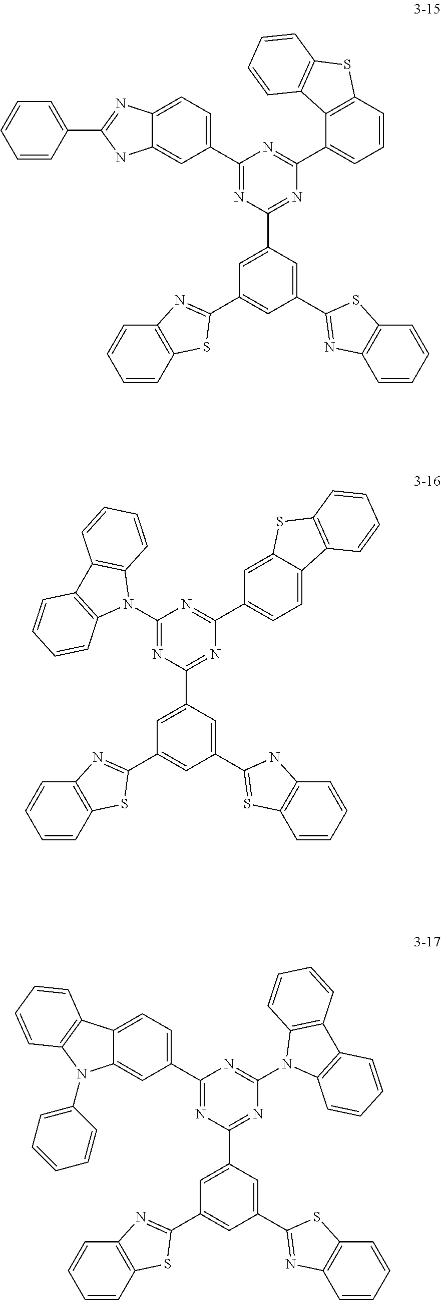

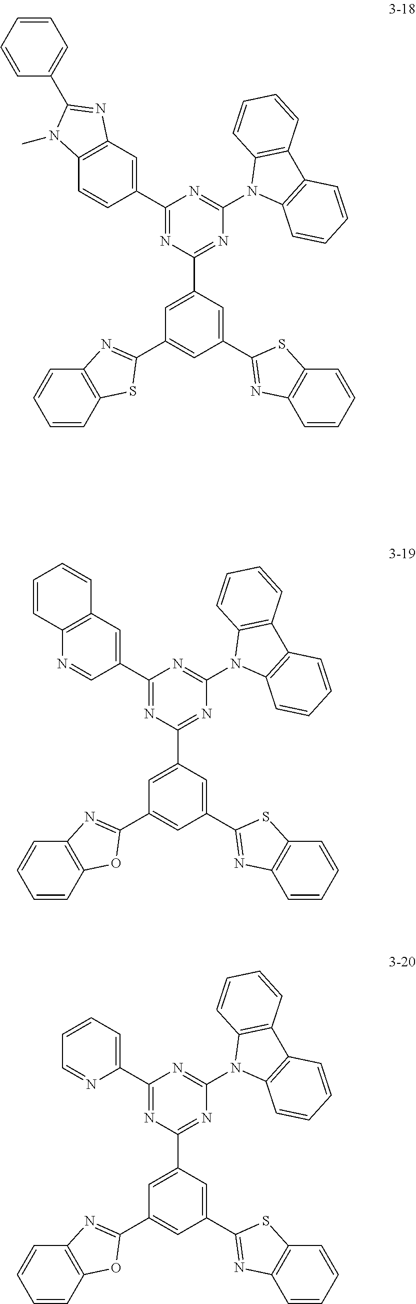 [SMILES: Cn1c(-c2ccccc2)nc2cc(-c3nc(-c4cc(-c5nc6ccccc6s5)cc(-c5nc6ccccc6s5)c4)nc(-n4c5ccccc5c5ccccc54)n3)ccc21 ; c1ccc2ncc(-c3nc(-c4cc(-c5nc6ccccc6o5)cc(-c5nc6ccccc6s5)c4)nc(-n4c5ccccc5c5ccccc54)n3)cc2c1 ; c1ccc(-c2nc(-c3cc(-c4nc5ccccc5o4)cc(-c4nc5ccccc5s4)c3)nc(-n3c4ccccc4c4ccccc43)n2)nc1]

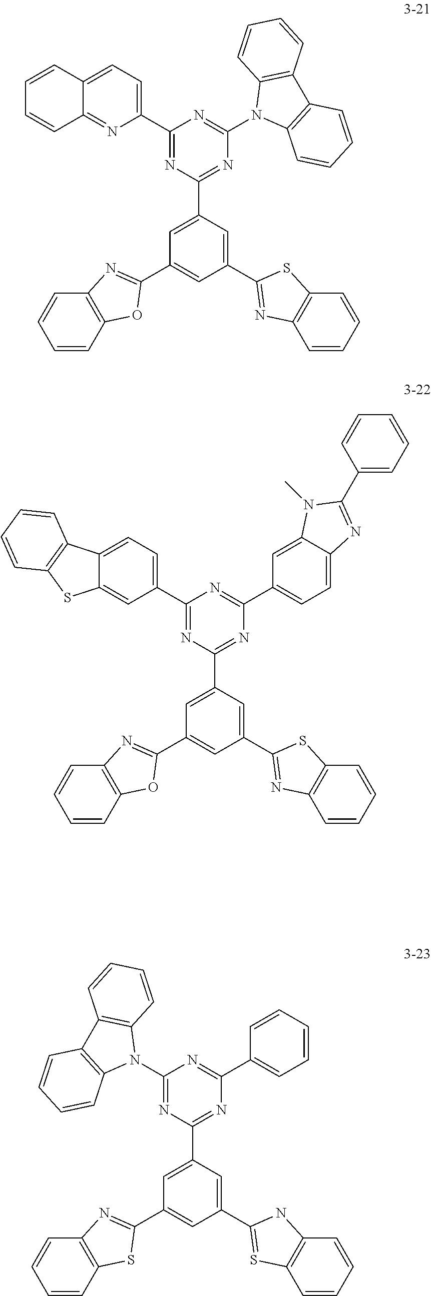

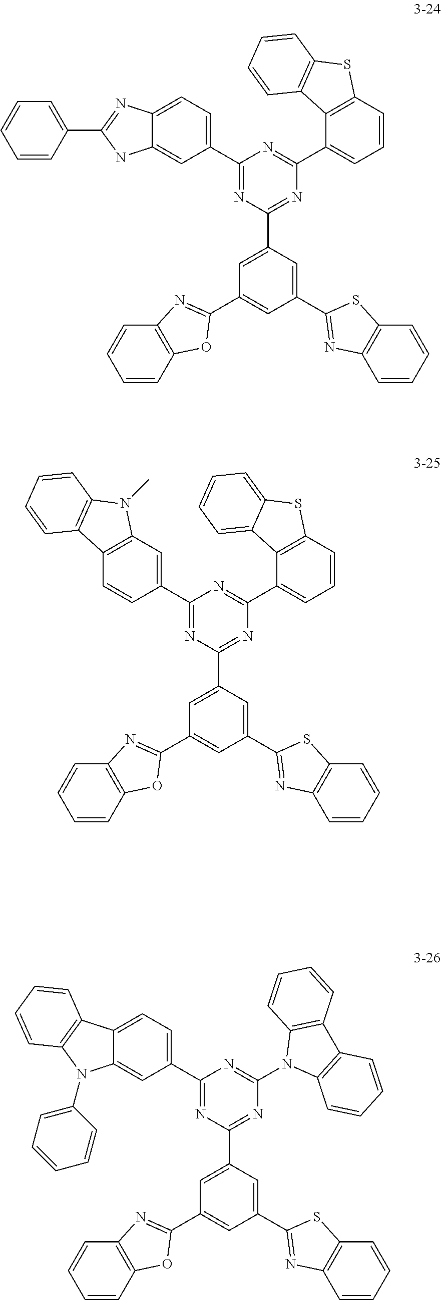 [SMILES: c1ccc(-c2nc3ccc(-c4nc(-c5cc(-c6nc7ccccc7o6)cc(-c6nc7ccccc7s6)c5)nc(-c5cccc6sc7ccccc7c56)n4)cc3[nH]2)cc1 ; Cn1c2ccccc2c2ccc(-c3nc(-c4cc(-c5nc6ccccc6o5)cc(-c5nc6ccccc6s5)c4)nc(-c4cccc5sc6ccccc6c45)n3)cc21 ; c1ccc(-n2c3ccccc3c3ccc(-c4nc(-c5cc(-c6nc7ccccc7o6)cc(-c6nc7ccccc7s6)c5)nc(-n5c6ccccc6c6ccccc65)n4)cc32)cc1]

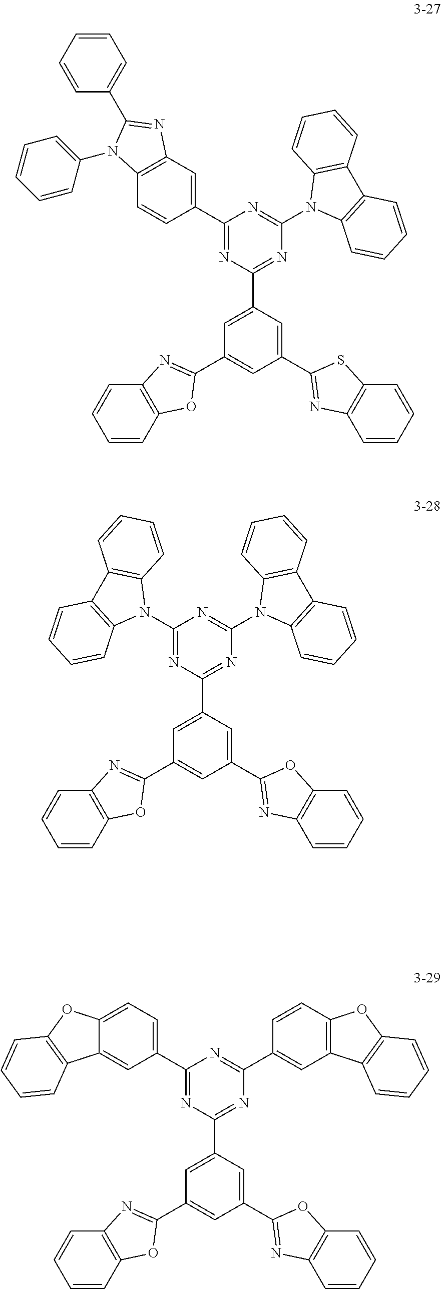 [SMILES: c1ccc(-c2nc3cc(-c4nc(-c5cc(-c6nc7ccccc7o6)cc(-c6nc7ccccc7s6)c5)nc(-n5c6ccccc6c6ccccc65)n4)ccc3n2-c2ccccc2)cc1 ; c1ccc2oc(-c3cc(-c4nc(-n5c6ccccc6c6ccccc65)nc(-n5c6ccccc6c6ccccc65)n4)cc(-c4nc5ccccc5o4)c3)nc2c1 ; c1ccc2oc(-c3cc(-c4nc(-c5ccc6oc7ccccc7c6c5)nc(-c5ccc6oc7ccccc7c6c5)n4)cc(-c4nc5ccccc5o4)c3)nc2c1]

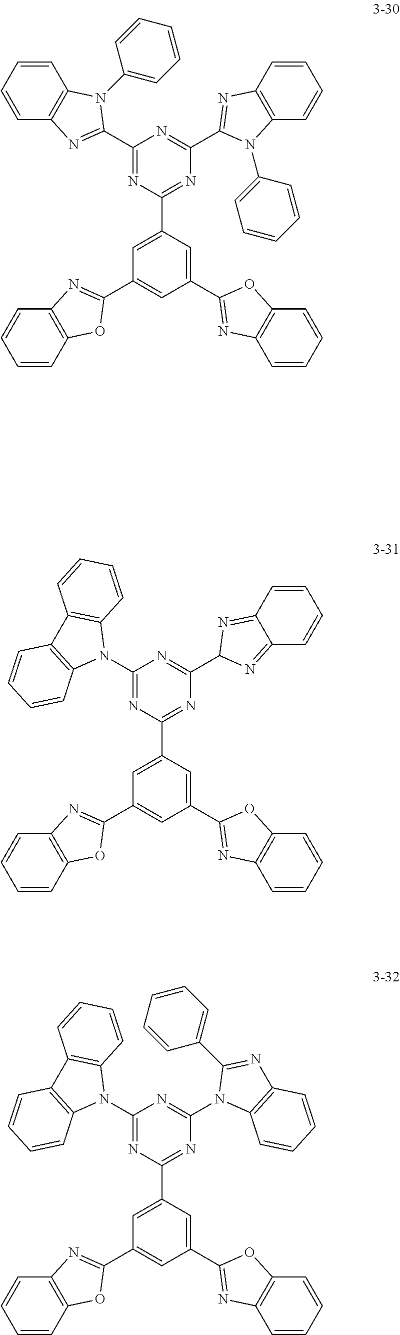 [SMILES: c1ccc(-n2c(-c3nc(-c4cc(-c5nc6ccccc6o5)cc(-c5nc6ccccc6o5)c4)nc(-c4nc5ccccc5n4-c4ccccc4)n3)nc3ccccc32)cc1 ; c1ccc2c(c1)=NC(c1nc(-c3cc(-c4nc5ccccc5o4)cc(-c4nc5ccccc5o4)c3)nc(-n3c4ccccc4c4ccccc43)n1)N=2 ; c1ccc(-c2nc3ccccc3n2-c2nc(-c3cc(-c4nc5ccccc5o4)cc(-c4nc5ccccc5o4)c3)nc(-n3c4ccccc4c4ccccc43)n2)cc1]

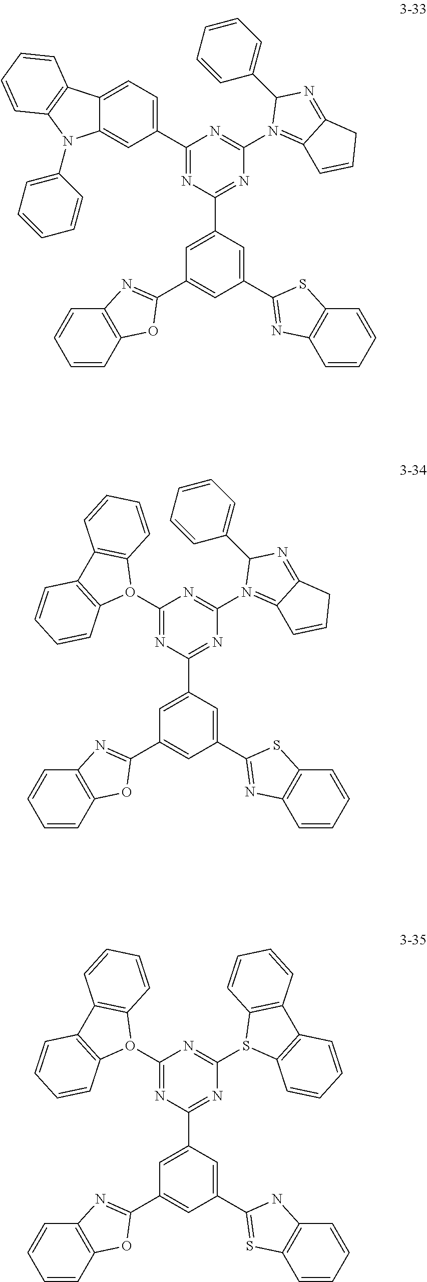

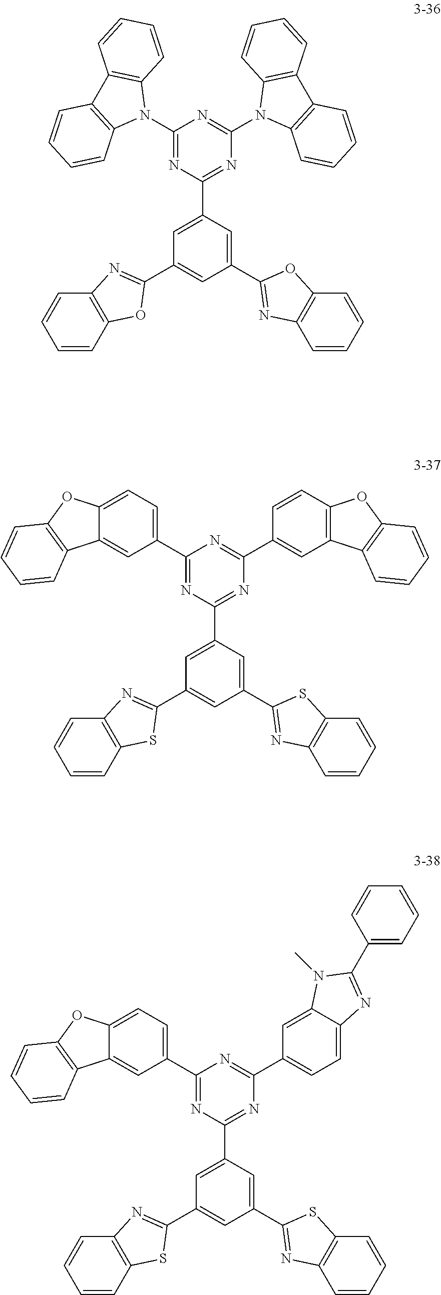 [SMILES: c1ccc2oc(-c3cc(-c4nc(-n5c6ccccc6c6ccccc65)nc(-n5c6ccccc6c6ccccc65)n4)cc(-c4nc5ccccc5o4)c3)nc2c1 ; c1ccc2sc(-c3cc(-c4nc(-c5ccc6oc7ccccc7c6c5)nc(-c5ccc6oc7ccccc7c6c5)n4)cc(-c4nc5ccccc5s4)c3)nc2c1 ; Cn1c(-c2ccccc2)nc2ccc(-c3nc(-c4cc(-c5nc6ccccc6s5)cc(-c5nc6ccccc6s5)c4)nc(-c4ccc5oc6ccccc6c5c4)n3)cc21]

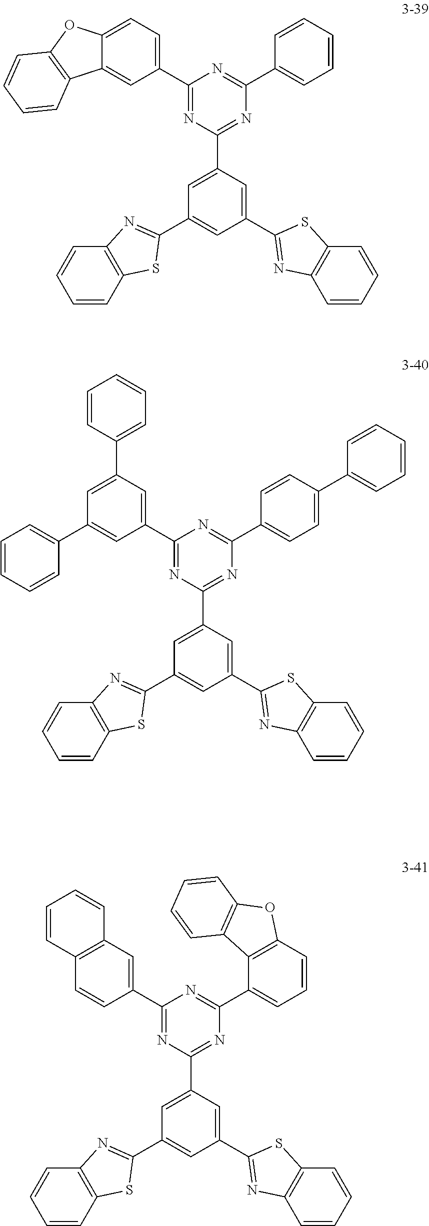 [SMILES: c1ccc(-c2nc(-c3cc(-c4nc5ccccc5s4)cc(-c4nc5ccccc5s4)c3)nc(-c3ccc4oc5ccccc5c4c3)n2)cc1 ; c1ccc(-c2ccc(-c3nc(-c4cc(-c5ccccc5)cc(-c5ccccc5)c4)nc(-c4cc(-c5nc6ccccc6s5)cc(-c5nc6ccccc6s5)c4)n3)cc2)cc1 ; c1ccc2cc(-c3nc(-c4cc(-c5nc6ccccc6s5)cc(-c5nc6ccccc6s5)c4)nc(-c4cccc5oc6ccccc6c45)n3)ccc2c1]

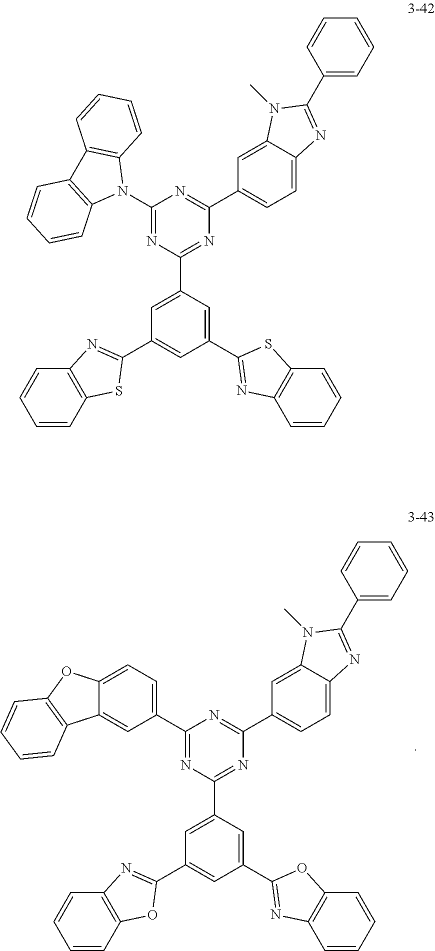 [SMILES: Cn1c(-c2ccccc2)nc2ccc(-c3nc(-c4cc(-c5nc6ccccc6s5)cc(-c5nc6ccccc6s5)c4)nc(-n4c5ccccc5c5ccccc54)n3)cc21 ; Cn1c(-c2ccccc2)nc2ccc(-c3nc(-c4cc(-c5nc6ccccc6o5)cc(-c5nc6ccccc6o5)c4)nc(-c4ccc5oc6ccccc6c5c4)n3)cc21]

7. An organic light emitting device, comprising:
a substrate; and
an organic light emitting diode positioned on the substrate and including:
a first electrode;
a second electrode facing the first electrode; and
a first emitting part between the first electrode and the second electrode, the first emitting part including a first emitting material layer, a first electron transporting layer, and a first hole blocking layer,
wherein the first electron transporting layer is positioned between the first emitting material layer and the second electrode, and the first hole blocking layer is positioned between the first emitting material layer and the first electron transporting layer, and
wherein at least one of the first electron transporting layer and the first hole blocking layer includes a first compound being the organic compound according to claim 1.

8. The organic light emitting device according to claim 7, wherein the first electron transporting layer includes the first compound and a second compound represented by Formula 3:

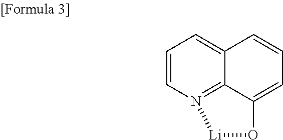

9. The organic light emitting device according to claim 8, wherein a weight % ratio of the first compound to the second compound is 1:9 to 9:1, 2:8 to 8:2 or 3:7 to 7:3.

10. The organic light emitting device according to claim 8, wherein the first compound and the second compound have the same weight %.

11. The organic light emitting device according to claim 7, wherein each of the first emitting material layer, the first electron transporting layer, and the first hole blocking layer includes the first compound.

12. The organic light emitting device according to claim 7, wherein the first emitting material layer includes a first host being a compound represented by Formula 7: 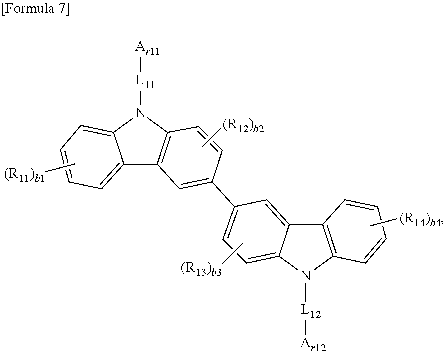
wherein each of b1 and b4 is independently an integer of 0 to 4, each of b2 and b3 is independently an integer of 0 to 3,
each R₁₁, R₁₂, R₁₃, and R₁₄ is independently selected from the group consisting of deuterium, a substituted or unsubstituted C1 to C10 alkyl group, a substituted or unsubstituted C3 to C30 cycloalkyl group, a substituted or unsubstituted C1 to C10 alkoxy group, a substituted or unsubstituted C6 to C60 aryl group, and a substituted or unsubstituted C3 to C60 heteroaryl group,
each of L₁₁ and L₁₂ is independently selected from the group consisting of a single bond, a substituted or unsubstituted C6 to C60 arylene group, and a substituted or unsubstituted C3 to C60 heteroarylene group, and
each of Ar₁₁ and Ar₁₂ is independently selected from the group consisting of a substituted or unsubstituted C6 to C60 aryl group, and a substituted or unsubstituted C3 to C60 heteroaryl group.

13. The organic light emitting device according to claim 12, wherein the first host is one of the compounds in Formula 8:

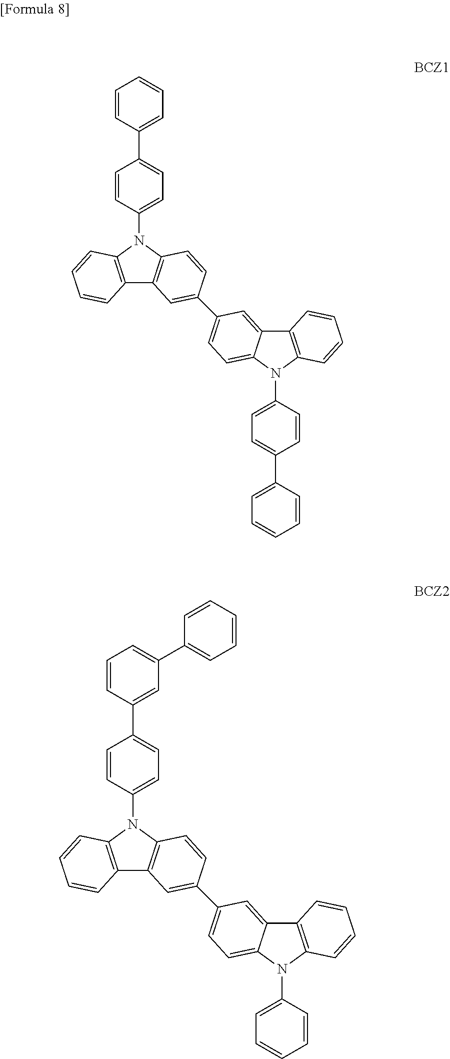 [SMILES: c1ccc(-c2ccc(-n3c4ccccc4c4cc(-c5ccc6c(c5)c5ccccc5n6-c5ccc(-c6ccccc6)cc5)ccc43)cc2)cc1 ; c1ccc(-c2cccc(-c3ccc(-n4c5ccccc5c5cc(-c6ccc7c(c6)c6ccccc6n7-c6ccccc6)ccc54)cc3)c2)cc1]

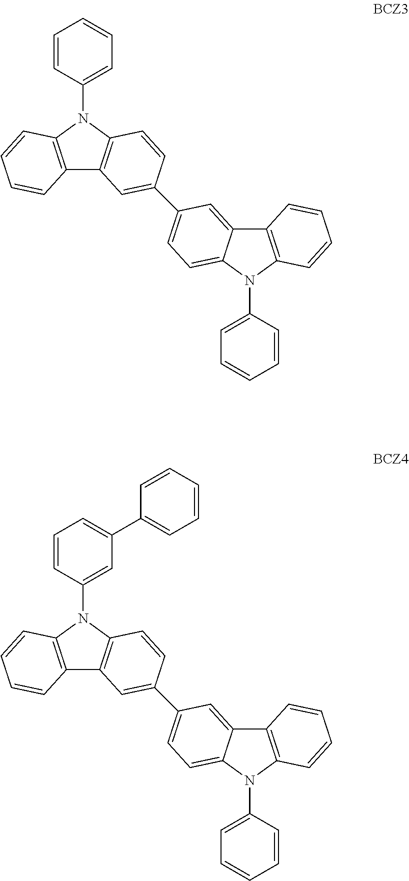 [SMILES: c1ccc(-n2c3ccccc3c3cc(-c4ccc5c(c4)c4ccccc4n5-c4ccccc4)ccc32)cc1 ; c1ccc(-c2cccc(-n3c4ccccc4c4cc(-c5ccc6c(c5)c5ccccc5n6-c5ccccc5)ccc43)c2)cc1]

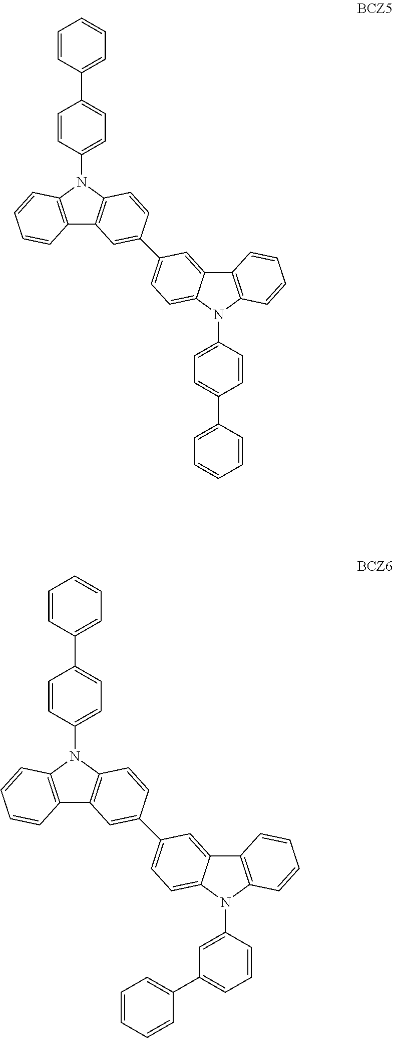 [SMILES: c1ccc(-c2ccc(-n3c4ccccc4c4cc(-c5ccc6c(c5)c5ccccc5n6-c5ccc(-c6ccccc6)cc5)ccc43)cc2)cc1 ; c1ccc(-c2ccc(-n3c4ccccc4c4cc(-c5ccc6c(c5)c5ccccc5n6-c5cccc(-c6ccccc6)c5)ccc43)cc2)cc1]

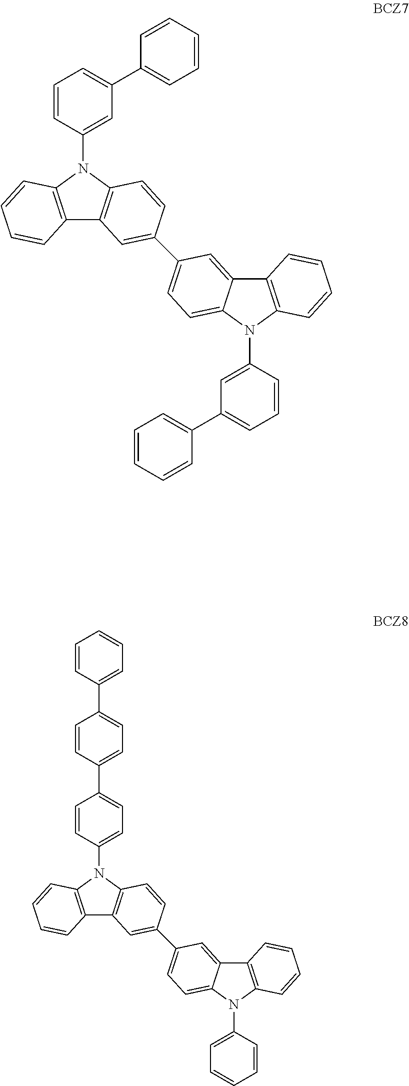 [SMILES: c1ccc(-c2cccc(-n3c4ccccc4c4cc(-c5ccc6c(c5)c5ccccc5n6-c5cccc(-c6ccccc6)c5)ccc43)c2)cc1 ; c1ccc(-c2ccc(-c3ccc(-n4c5ccccc5c5cc(-c6ccc7c(c6)c6ccccc6n7-c6ccccc6)ccc54)cc3)cc2)cc1]

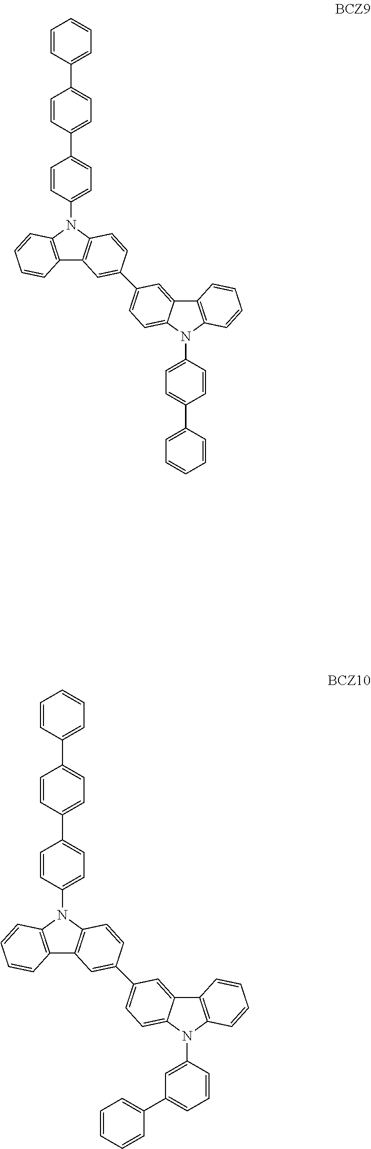 [SMILES: c1ccc(-c2ccc(-c3ccc(-n4c5ccccc5c5cc(-c6ccc7c(c6)c6ccccc6n7-c6ccc(-c7ccccc7)cc6)ccc54)cc3)cc2)cc1 ; c1ccc(-c2ccc(-c3ccc(-n4c5ccccc5c5cc(-c6ccc7c(c6)c6ccccc6n7-c6cccc(-c7ccccc7)c6)ccc54)cc3)cc2)cc1]

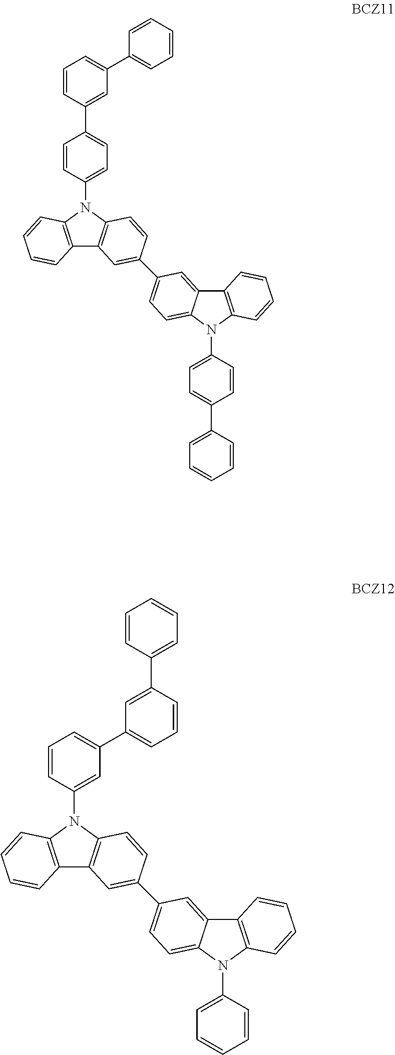 [SMILES: c1ccc(-c2ccc(-n3c4ccccc4c4cc(-c5ccc6c(c5)c5ccccc5n6-c5ccc(-c6cccc(-c7ccccc7)c6)cc5)ccc43)cc2)cc1 ; c1ccc(-c2cccc(-c3cccc(-n4c5ccccc5c5cc(-c6ccc7c(c6)c6ccccc6n7-c6ccccc6)ccc54)c3)c2)cc1]

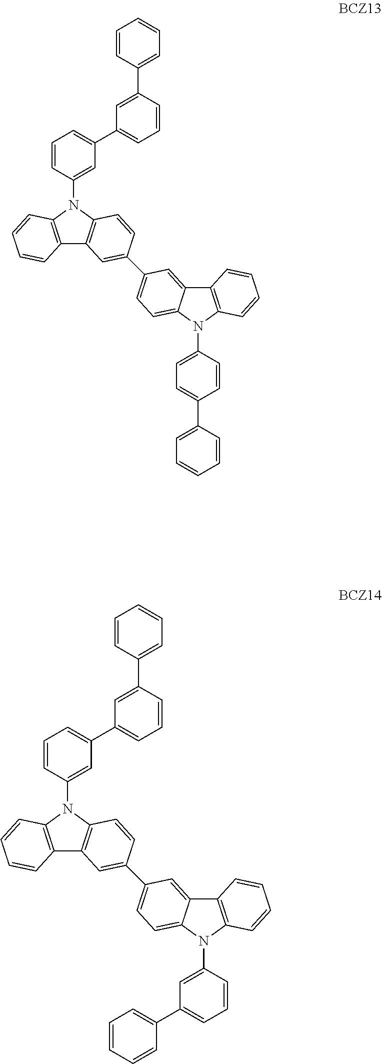 [SMILES: c1ccc(-c2ccc(-n3c4ccccc4c4cc(-c5ccc6c(c5)c5ccccc5n6-c5cccc(-c6cccc(-c7ccccc7)c6)c5)ccc43)cc2)cc1 ; c1ccc(-c2cccc(-c3cccc(-n4c5ccccc5c5cc(-c6ccc7c(c6)c6ccccc6n7-c6cccc(-c7ccccc7)c6)ccc54)c3)c2)cc1]

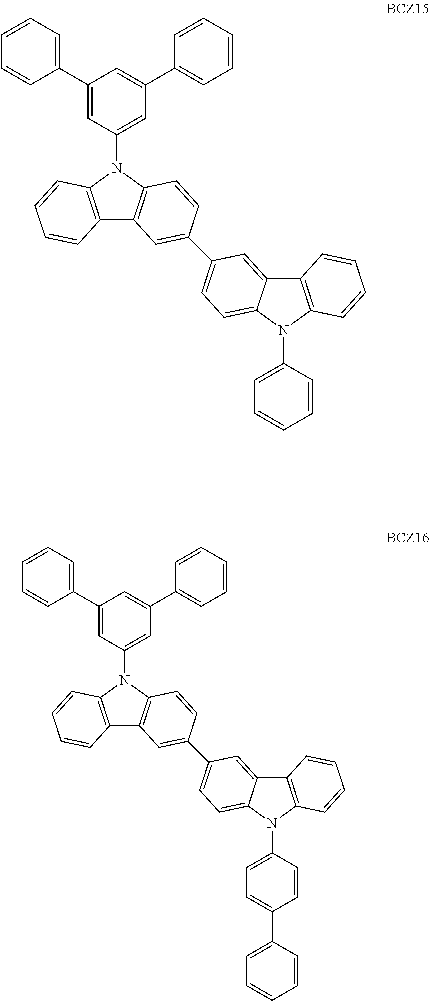 [SMILES: c1ccc(-c2cc(-c3ccccc3)cc(-n3c4ccccc4c4cc(-c5ccc6c(c5)c5ccccc5n6-c5ccccc5)ccc43)c2)cc1 ; c1ccc(-c2ccc(-n3c4ccccc4c4cc(-c5ccc6c(c5)c5ccccc5n6-c5cc(-c6ccccc6)cc(-c6ccccc6)c5)ccc43)cc2)cc1]

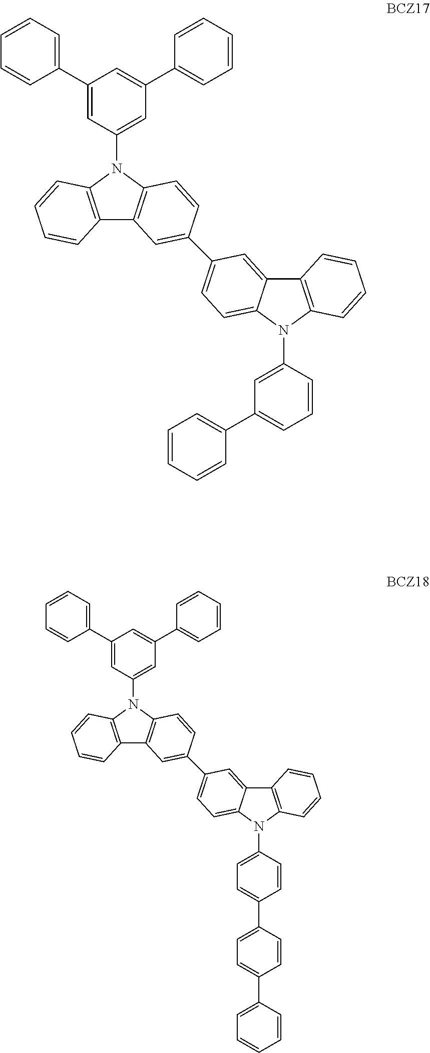 [SMILES: c1ccc(-c2cccc(-n3c4ccccc4c4cc(-c5ccc6c(c5)c5ccccc5n6-c5cc(-c6ccccc6)cc(-c6ccccc6)c5)ccc43)c2)cc1 ; c1ccc(-c2ccc(-c3ccc(-n4c5ccccc5c5cc(-c6ccc7c(c6)c6ccccc6n7-c6cc(-c7ccccc7)cc(-c7ccccc7)c6)ccc54)cc3)cc2)cc1]

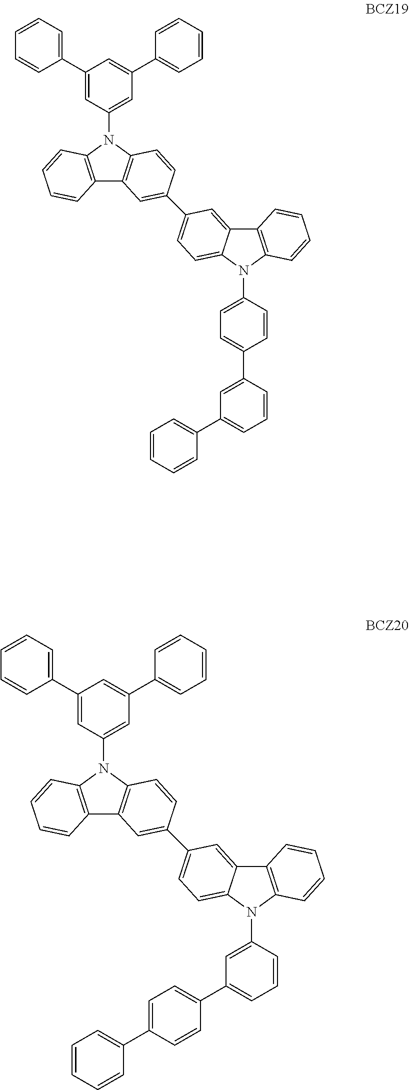 [SMILES: c1ccc(-c2cccc(-c3ccc(-n4c5ccccc5c5cc(-c6ccc7c(c6)c6ccccc6n7-c6cc(-c7ccccc7)cc(-c7ccccc7)c6)ccc54)cc3)c2)cc1 ; c1ccc(-c2ccc(-c3cccc(-n4c5ccccc5c5cc(-c6ccc7c(c6)c6ccccc6n7-c6cc(-c7ccccc7)cc(-c7ccccc7)c6)ccc54)c3)cc2)cc1]

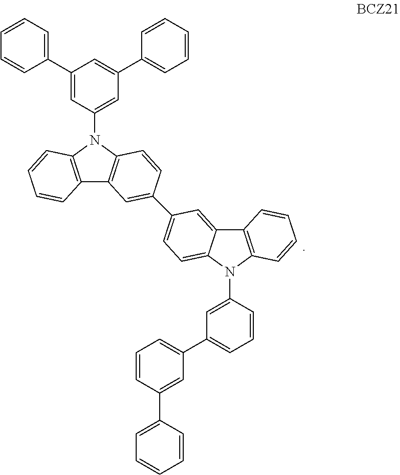 [SMILES: c1ccc(-c2cccc(-c3cccc(-n4c5ccccc5c5cc(-c6ccc7c(c6)c6ccccc6n7-c6cc(-c7ccccc7)cc(-c7ccccc7)c6)ccc54)c3)c2)cc1]

14. The organic light emitting device according to claim 12, wherein the first emitting material layer further includes a second host being an organic compound represented by Formula 1, wherein the second host is the same as or different to the first compound.

15. The organic light emitting device according to claim 12, wherein the first emitting material layer further includes a second host being an organic compound according to Formula 4:

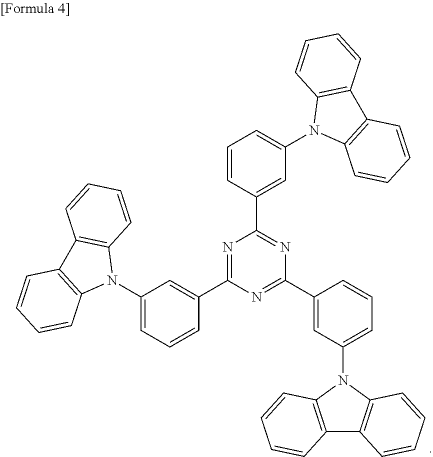 [SMILES: c1cc(-c2nc(-c3cccc(-n4c5ccccc5c5ccccc54)c3)nc(-c3cccc(-n4c5ccccc5c5ccccc54)c3)n2)cc(-n2c3ccccc3c3ccccc32)c1]

16. The organic light emitting device according to claim 12, wherein the first emitting material layer further includes a second host being an organic compound represented by Formula 5: 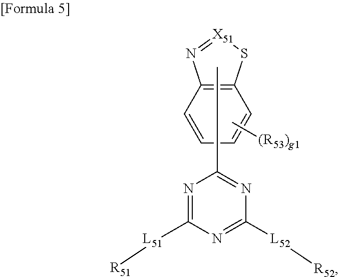
wherein g1 integer of 0 to 4,
each of R₅₁ and R₅₂ is independently selected from the group consisting of a substituted or unsubstituted C6 to C60 aryl group and a substituted or unsubstituted C3 to C60 heteroaryl group,
each R₅₃ is selected from the group consisting of a substituted or unsubstituted C1 to C10 alkyl group, a substituted or unsubstituted C6 to C60 aryl group and a substituted or unsubstituted C3 to C60 heteroaryl group,
X₅₁ is CR₅₄,
R₅₄ is selected from the group consisting of a bonding site, hydrogen, a substituted or unsubstituted C1 to C10 alkyl group, a substituted or unsubstituted C6 to C60 aryl group and a substituted or unsubstituted C3 to C60 heteroaryl group,
at least one of R₅₁, R₅₂ and R₅₄ is a substituted or unsubstituted carbazolyl group, and
each of L₅₁ and L₅₂ is independently selected from the group consisting of a single bond and a substituted or unsubstituted C6 to C60 arylene group.

17. The organic light emitting device according to claim 16, wherein the second host is an organic compound represented by Formula 5a: 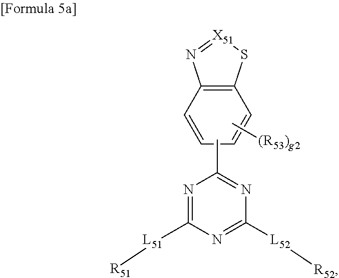
wherein the definition of R₅₁, R₅₂, R₅₃, X₅₁, L₅₁ and L₅₂ is the same as for Formula 5, and g2 is an integer of 0 to 3.

18. The organic light emitting device according to claim 16, wherein the second host is an organic compound represented by one of Formulas 5b to 5f: 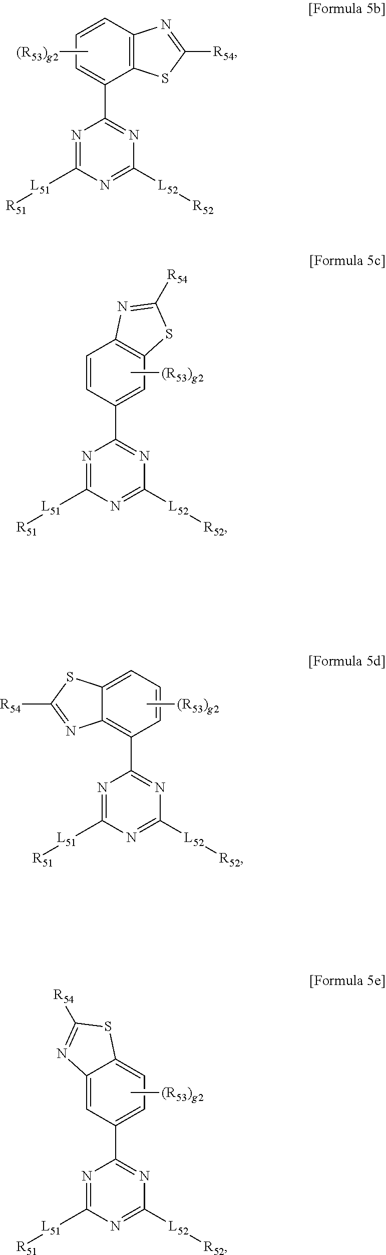 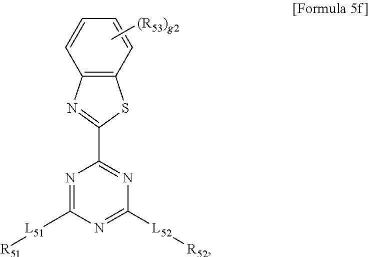
wherein in each of Formulas 5b to 5e, the definition of R₅₁, R₅₂, R₅₃, R₅₄, L₅₁ and L₅₂ is the same as for Formula 5, and g2 is an integer of 0 to 3, and in Formula 5f, the definition of R₅₁, R₅₂, R₅₃, L₅₁ and L₅₂ is the same as for Formula 5, and g3 is an integer of 0 to 4, optionally wherein R₅₁ is a carbazole group unsubstituted or substituted with a C1 to C20 alkyl group, and R₅₂ is selected from the group consisting of phenyl, pyrenyl, fluorenyl, dibenzofuranyl, dibenzothiophenyl and carbazolyl, and may be unsubstituted or substituted with at least one of a C1 to C20 alkyl group and a C6 to C60 aryl group.

19. The organic light emitting device according to claim 16, wherein the second host is one of the compounds in Formula 6:

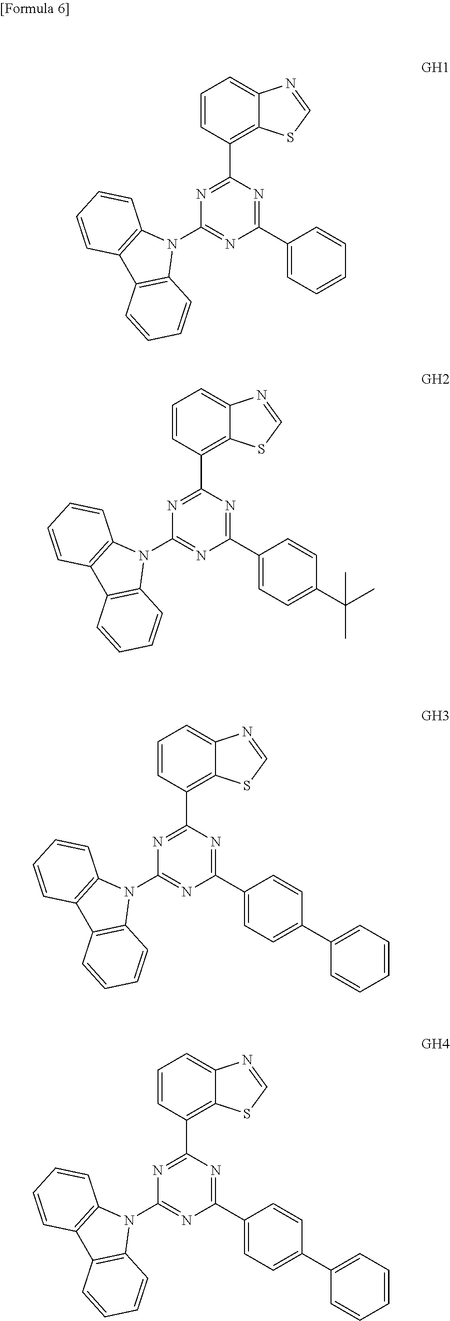 [SMILES: c1ccc(-c2nc(-c3cccc4ncsc34)nc(-n3c4ccccc4c4ccccc43)n2)cc1 ; CC(C)(C)c1ccc(-c2nc(-c3cccc4ncsc34)nc(-n3c4ccccc4c4ccccc43)n2)cc1 ; c1ccc(-c2ccc(-c3nc(-c4cccc5ncsc45)nc(-n4c5ccccc5c5ccccc54)n3)cc2)cc1 ; c1ccc(-c2ccc(-c3nc(-c4cccc5ncsc45)nc(-n4c5ccccc5c5ccccc54)n3)cc2)cc1]

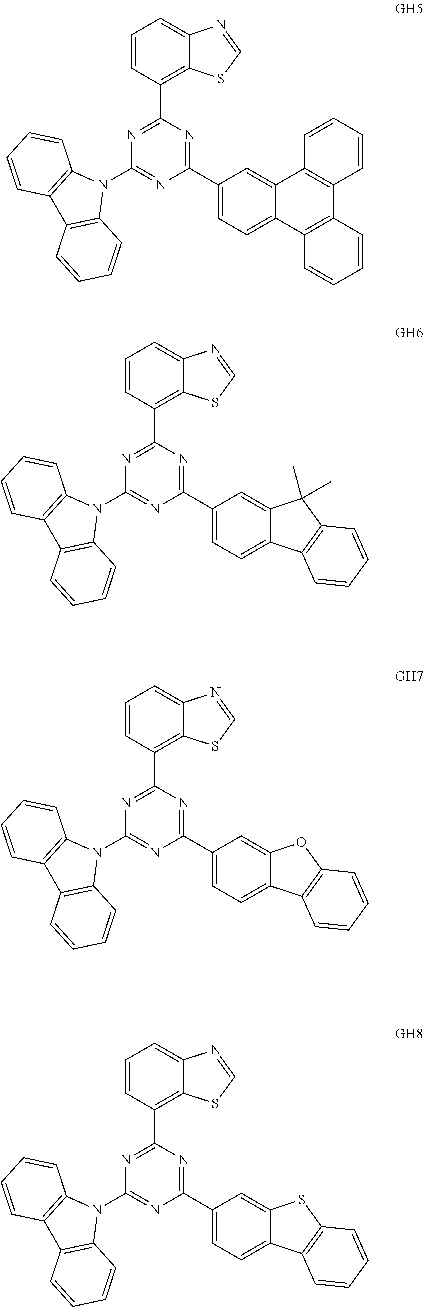 [SMILES: c1cc(-c2nc(-c3ccc4c5ccccc5c5ccccc5c4c3)nc(-n3c4ccccc4c4ccccc43)n2)c2scnc2c1 ; CC1(C)c2ccccc2-c2ccc(-c3nc(-c4cccc5ncsc45)nc(-n4c5ccccc5c5ccccc54)n3)cc21 ; c1cc(-c2nc(-c3ccc4c(c3)oc3ccccc34)nc(-n3c4ccccc4c4ccccc43)n2)c2scnc2c1 ; c1cc(-c2nc(-c3ccc4c(c3)sc3ccccc34)nc(-n3c4ccccc4c4ccccc43)n2)c2scnc2c1]

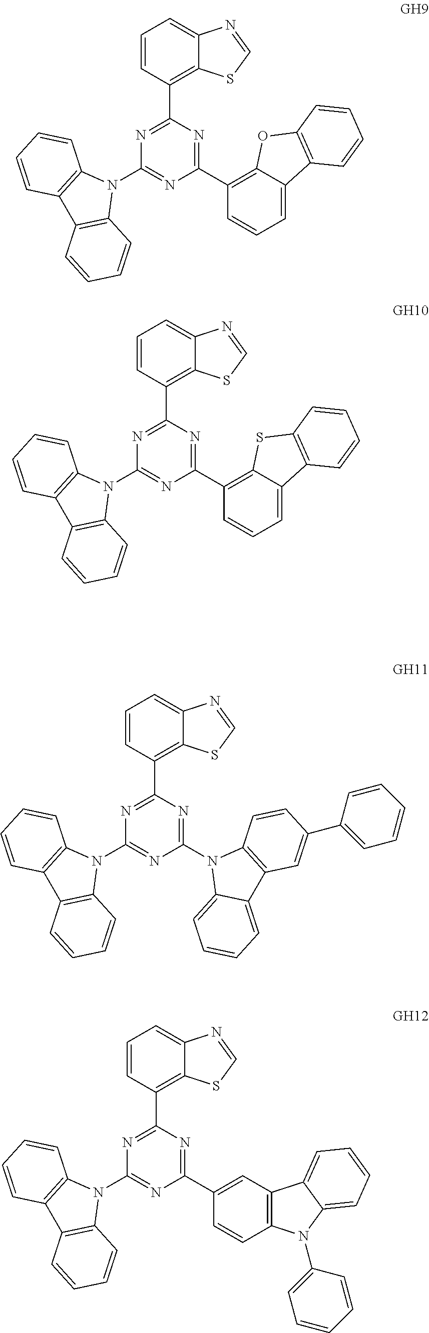 [SMILES: c1cc(-c2nc(-c3cccc4c3oc3ccccc34)nc(-n3c4ccccc4c4ccccc43)n2)c2scnc2c1 ; c1cc(-c2nc(-c3cccc4c3sc3ccccc34)nc(-n3c4ccccc4c4ccccc43)n2)c2scnc2c1 ; c1ccc(-c2ccc3c(c2)c2ccccc2n3-c2nc(-c3cccc4ncsc34)nc(-n3c4ccccc4c4ccccc43)n2)cc1 ; c1ccc(-n2c3ccccc3c3cc(-c4nc(-c5cccc6ncsc56)nc(-n5c6ccccc6c6ccccc65)n4)ccc32)cc1]

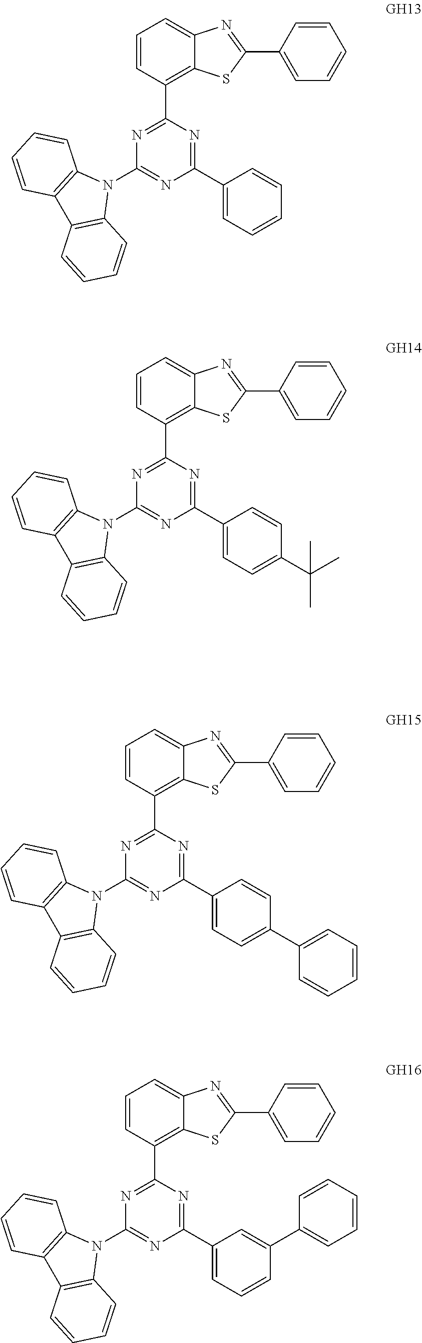 [SMILES: c1ccc(-c2nc(-c3cccc4nc(-c5ccccc5)sc34)nc(-n3c4ccccc4c4ccccc43)n2)cc1 ; CC(C)(C)c1ccc(-c2nc(-c3cccc4nc(-c5ccccc5)sc34)nc(-n3c4ccccc4c4ccccc43)n2)cc1 ; c1ccc(-c2ccc(-c3nc(-c4cccc5nc(-c6ccccc6)sc45)nc(-n4c5ccccc5c5ccccc54)n3)cc2)cc1 ; c1ccc(-c2cccc(-c3nc(-c4cccc5nc(-c6ccccc6)sc45)nc(-n4c5ccccc5c5ccccc54)n3)c2)cc1]

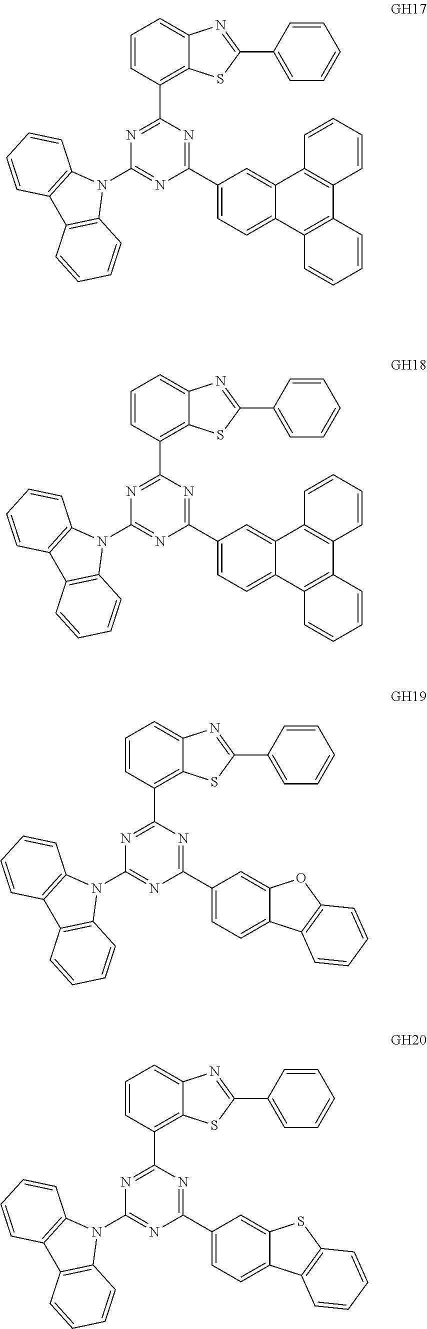 [SMILES: c1ccc(-c2nc3cccc(-c4nc(-c5ccc6c7ccccc7c7ccccc7c6c5)nc(-n5c6ccccc6c6ccccc65)n4)c3s2)cc1 ; c1ccc(-c2nc3cccc(-c4nc(-c5ccc6c7ccccc7c7ccccc7c6c5)nc(-n5c6ccccc6c6ccccc65)n4)c3s2)cc1 ; c1ccc(-c2nc3cccc(-c4nc(-c5ccc6c(c5)oc5ccccc56)nc(-n5c6ccccc6c6ccccc65)n4)c3s2)cc1 ; c1ccc(-c2nc3cccc(-c4nc(-c5ccc6c(c5)sc5ccccc56)nc(-n5c6ccccc6c6ccccc65)n4)c3s2)cc1]

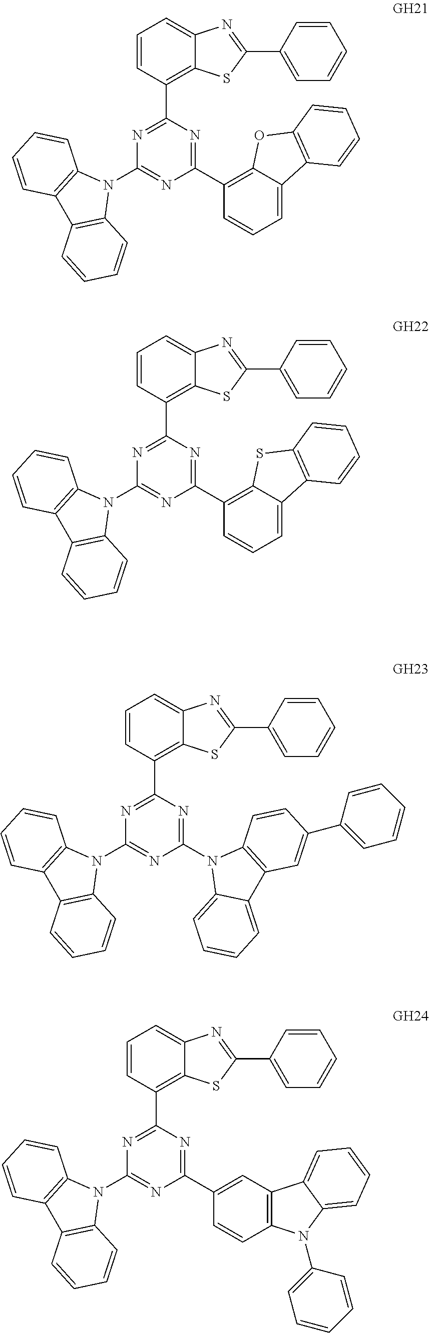 [SMILES: c1ccc(-c2nc3cccc(-c4nc(-c5cccc6c5oc5ccccc56)nc(-n5c6ccccc6c6ccccc65)n4)c3s2)cc1 ; c1ccc(-c2nc3cccc(-c4nc(-c5cccc6c5sc5ccccc56)nc(-n5c6ccccc6c6ccccc65)n4)c3s2)cc1 ; c1ccc(-c2ccc3c(c2)c2ccccc2n3-c2nc(-c3cccc4nc(-c5ccccc5)sc34)nc(-n3c4ccccc4c4ccccc43)n2)cc1 ; c1ccc(-c2nc3cccc(-c4nc(-c5ccc6c(c5)c5ccccc5n6-c5ccccc5)nc(-n5c6ccccc6c6ccccc65)n4)c3s2)cc1]

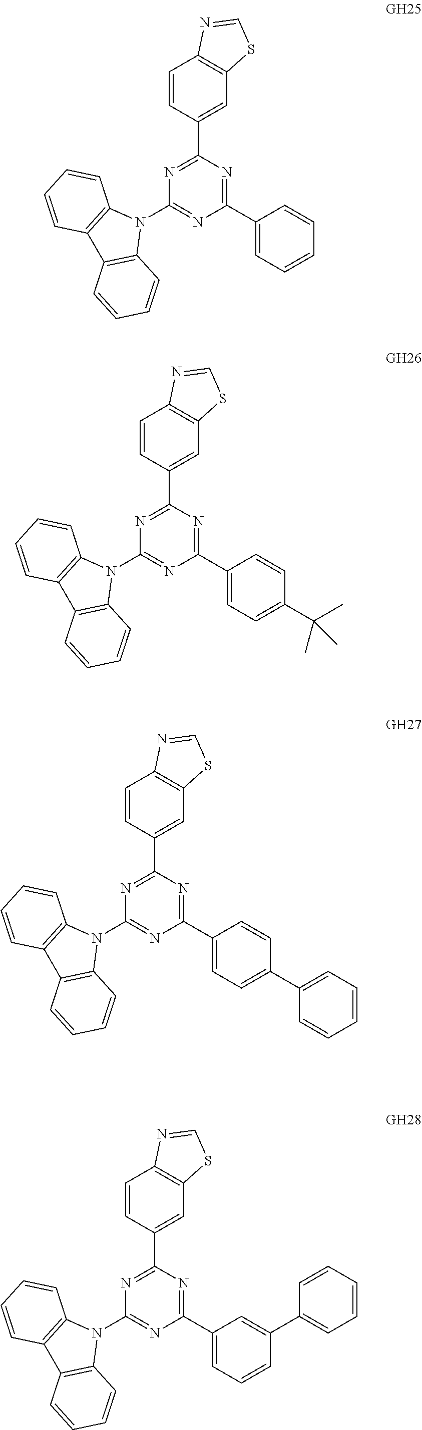 [SMILES: c1ccc(-c2nc(-c3ccc4ncsc4c3)nc(-n3c4ccccc4c4ccccc43)n2)cc1 ; CC(C)(C)c1ccc(-c2nc(-c3ccc4ncsc4c3)nc(-n3c4ccccc4c4ccccc43)n2)cc1 ; c1ccc(-c2ccc(-c3nc(-c4ccc5ncsc5c4)nc(-n4c5ccccc5c5ccccc54)n3)cc2)cc1 ; c1ccc(-c2cccc(-c3nc(-c4ccc5ncsc5c4)nc(-n4c5ccccc5c5ccccc54)n3)c2)cc1]

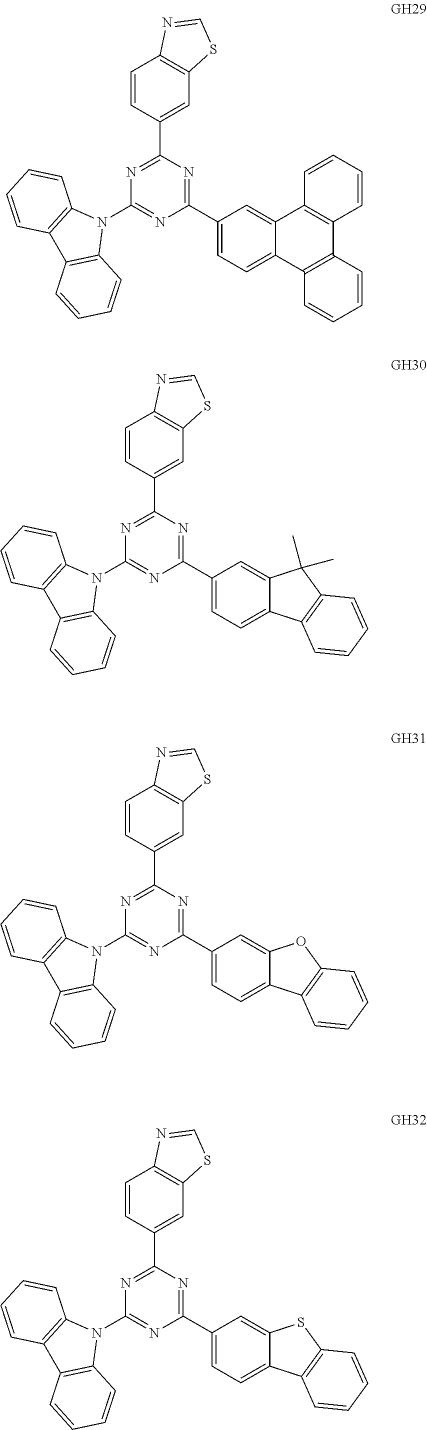 [SMILES: c1ccc2c(c1)c1ccccc1c1cc(-c3nc(-c4ccc5ncsc5c4)nc(-n4c5ccccc5c5ccccc54)n3)ccc21 ; CC1(C)c2ccccc2-c2ccc(-c3nc(-c4ccc5ncsc5c4)nc(-n4c5ccccc5c5ccccc54)n3)cc21 ; c1ccc2c(c1)oc1cc(-c3nc(-c4ccc5ncsc5c4)nc(-n4c5ccccc5c5ccccc54)n3)ccc12 ; c1ccc2c(c1)sc1cc(-c3nc(-c4ccc5ncsc5c4)nc(-n4c5ccccc5c5ccccc54)n3)ccc12]

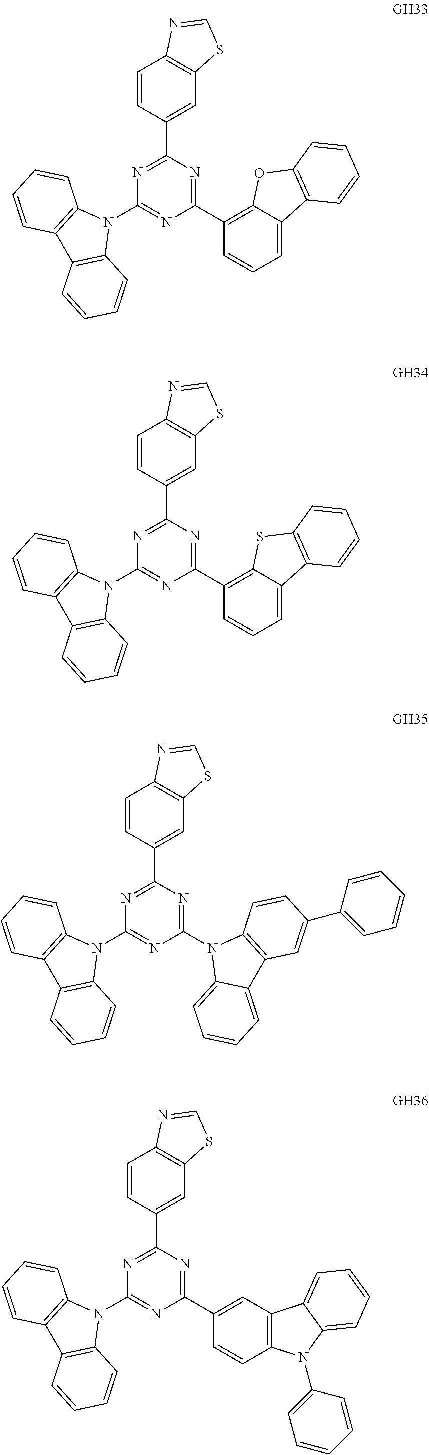 [SMILES: c1ccc2c(c1)oc1c(-c3nc(-c4ccc5ncsc5c4)nc(-n4c5ccccc5c5ccccc54)n3)cccc12 ; c1ccc2c(c1)sc1c(-c3nc(-c4ccc5ncsc5c4)nc(-n4c5ccccc5c5ccccc54)n3)cccc12 ; c1ccc(-c2ccc3c(c2)c2ccccc2n3-c2nc(-c3ccc4ncsc4c3)nc(-n3c4ccccc4c4ccccc43)n2)cc1 ; c1ccc(-n2c3ccccc3c3cc(-c4nc(-c5ccc6ncsc6c5)nc(-n5c6ccccc6c6ccccc65)n4)ccc32)cc1]

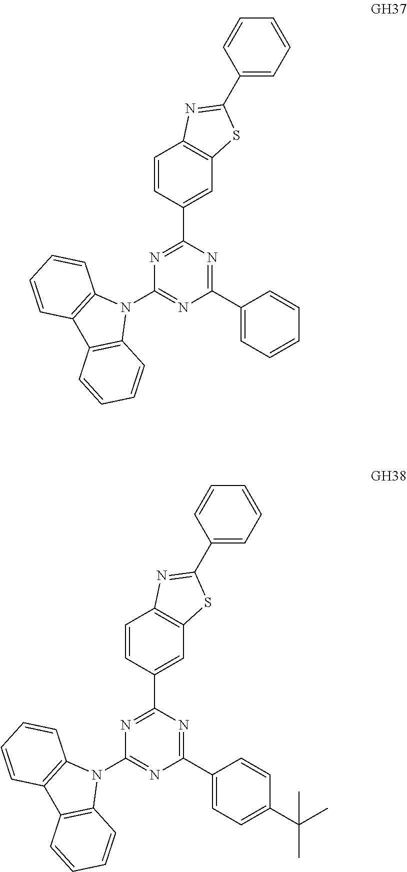 [SMILES: c1ccc(-c2nc(-c3ccc4nc(-c5ccccc5)sc4c3)nc(-n3c4ccccc4c4ccccc43)n2)cc1 ; CC(C)(C)c1ccc(-c2nc(-c3ccc4nc(-c5ccccc5)sc4c3)nc(-n3c4ccccc4c4ccccc43)n2)cc1]

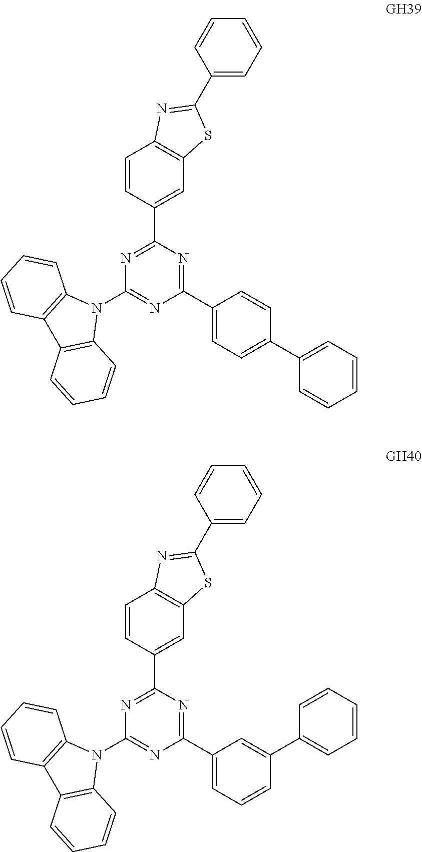 [SMILES: c1ccc(-c2ccc(-c3nc(-c4ccc5nc(-c6ccccc6)sc5c4)nc(-n4c5ccccc5c5ccccc54)n3)cc2)cc1 ; c1ccc(-c2cccc(-c3nc(-c4ccc5nc(-c6ccccc6)sc5c4)nc(-n4c5ccccc5c5ccccc54)n3)c2)cc1]

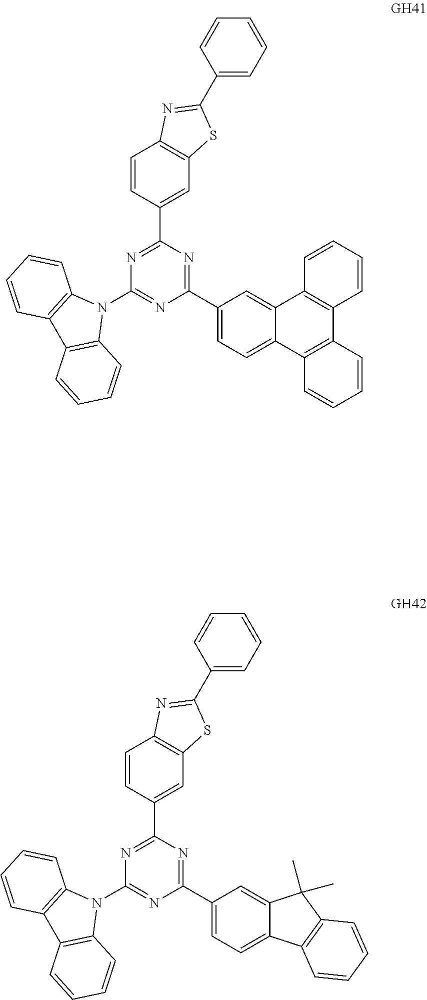 [SMILES: c1ccc(-c2nc3ccc(-c4nc(-c5ccc6c7ccccc7c7ccccc7c6c5)nc(-n5c6ccccc6c6ccccc65)n4)cc3s2)cc1 ; CC1(C)c2ccccc2-c2ccc(-c3nc(-c4ccc5nc(-c6ccccc6)sc5c4)nc(-n4c5ccccc5c5ccccc54)n3)cc21]

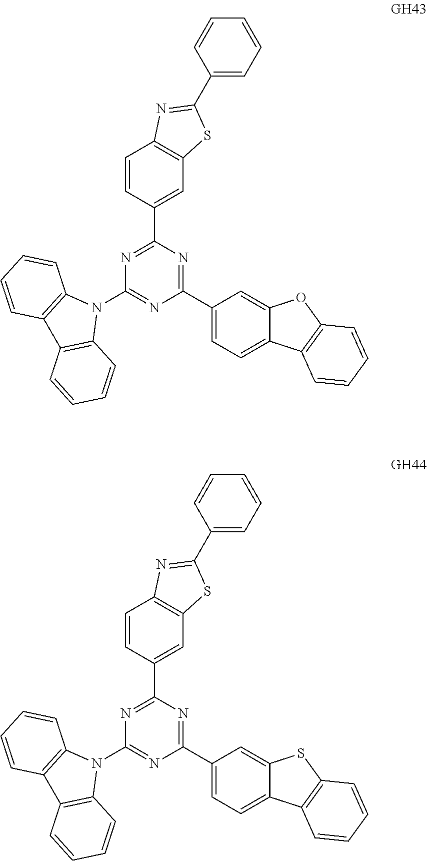 [SMILES: c1ccc(-c2nc3ccc(-c4nc(-c5ccc6c(c5)oc5ccccc56)nc(-n5c6ccccc6c6ccccc65)n4)cc3s2)cc1 ; c1ccc(-c2nc3ccc(-c4nc(-c5ccc6c(c5)sc5ccccc56)nc(-n5c6ccccc6c6ccccc65)n4)cc3s2)cc1]

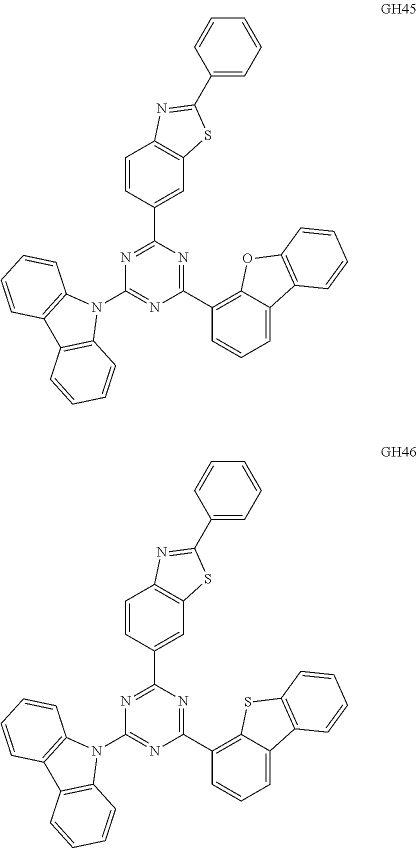 [SMILES: c1ccc(-c2nc3ccc(-c4nc(-c5cccc6c5oc5ccccc56)nc(-n5c6ccccc6c6ccccc65)n4)cc3s2)cc1 ; c1ccc(-c2nc3ccc(-c4nc(-c5cccc6c5sc5ccccc56)nc(-n5c6ccccc6c6ccccc65)n4)cc3s2)cc1]

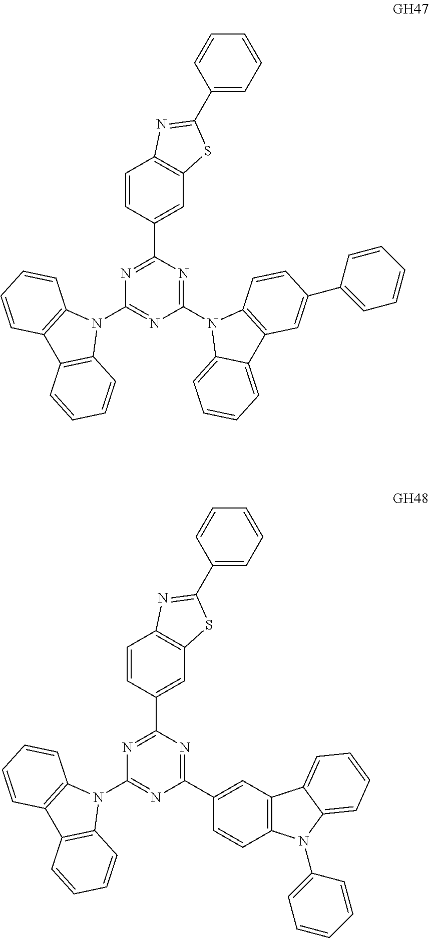 [SMILES: c1ccc(-c2ccc3c(c2)c2ccccc2n3-c2nc(-c3ccc4nc(-c5ccccc5)sc4c3)nc(-n3c4ccccc4c4ccccc43)n2)cc1 ; c1ccc(-c2nc3ccc(-c4nc(-c5ccc6c(c5)c5ccccc5n6-c5ccccc5)nc(-n5c6ccccc6c6ccccc65)n4)cc3s2)cc1]

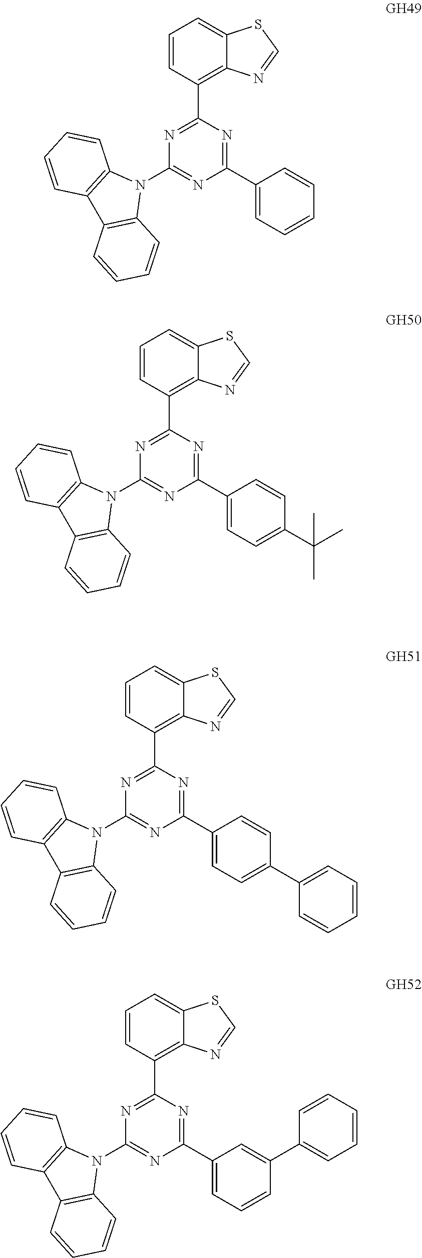 [SMILES: c1ccc(-c2nc(-c3cccc4scnc34)nc(-n3c4ccccc4c4ccccc43)n2)cc1 ; CC(C)(C)c1ccc(-c2nc(-c3cccc4scnc34)nc(-n3c4ccccc4c4ccccc43)n2)cc1 ; c1ccc(-c2ccc(-c3nc(-c4cccc5scnc45)nc(-n4c5ccccc5c5ccccc54)n3)cc2)cc1 ; c1ccc(-c2cccc(-c3nc(-c4cccc5scnc45)nc(-n4c5ccccc5c5ccccc54)n3)c2)cc1]

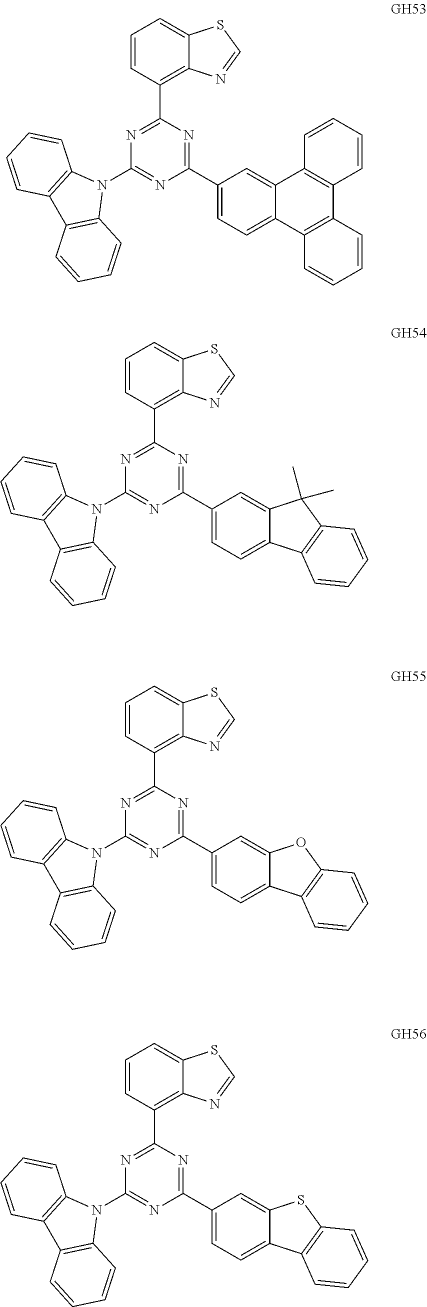 [SMILES: c1cc(-c2nc(-c3ccc4c5ccccc5c5ccccc5c4c3)nc(-n3c4ccccc4c4ccccc43)n2)c2ncsc2c1 ; CC1(C)c2ccccc2-c2ccc(-c3nc(-c4cccc5scnc45)nc(-n4c5ccccc5c5ccccc54)n3)cc21 ; c1ccc2c(c1)oc1cc(-c3nc(-c4cccc5scnc45)nc(-n4c5ccccc5c5ccccc54)n3)ccc12 ; c1cc(-c2nc(-c3ccc4c(c3)sc3ccccc34)nc(-n3c4ccccc4c4ccccc43)n2)c2ncsc2c1]

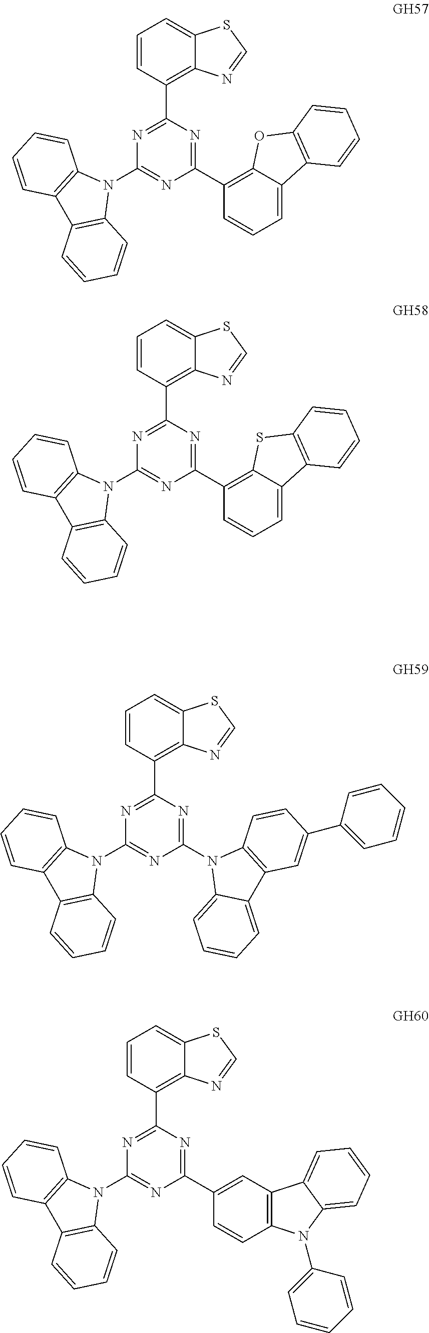 [SMILES: c1ccc2c(c1)oc1c(-c3nc(-c4cccc5scnc45)nc(-n4c5ccccc5c5ccccc54)n3)cccc12 ; c1cc(-c2nc(-c3cccc4c3sc3ccccc34)nc(-n3c4ccccc4c4ccccc43)n2)c2ncsc2c1 ; c1ccc(-c2ccc3c(c2)c2ccccc2n3-c2nc(-c3cccc4scnc34)nc(-n3c4ccccc4c4ccccc43)n2)cc1 ; c1ccc(-n2c3ccccc3c3cc(-c4nc(-c5cccc6scnc56)nc(-n5c6ccccc6c6ccccc65)n4)ccc32)cc1]

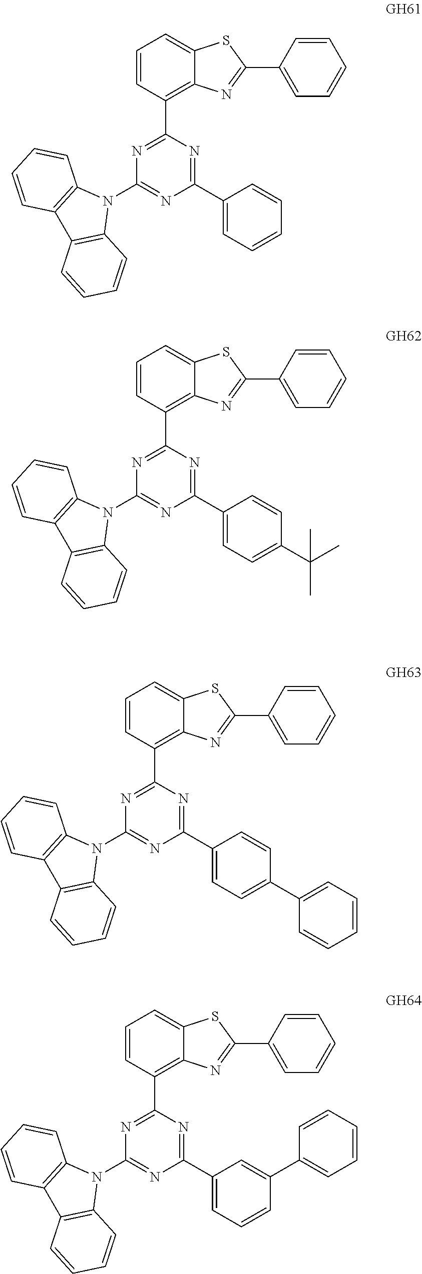 [SMILES: c1ccc(-c2nc(-c3cccc4sc(-c5ccccc5)nc34)nc(-n3c4ccccc4c4ccccc43)n2)cc1 ; CC(C)(C)c1ccc(-c2nc(-c3cccc4sc(-c5ccccc5)nc34)nc(-n3c4ccccc4c4ccccc43)n2)cc1 ; c1ccc(-c2ccc(-c3nc(-c4cccc5sc(-c6ccccc6)nc45)nc(-n4c5ccccc5c5ccccc54)n3)cc2)cc1 ; c1ccc(-c2cccc(-c3nc(-c4cccc5sc(-c6ccccc6)nc45)nc(-n4c5ccccc5c5ccccc54)n3)c2)cc1]

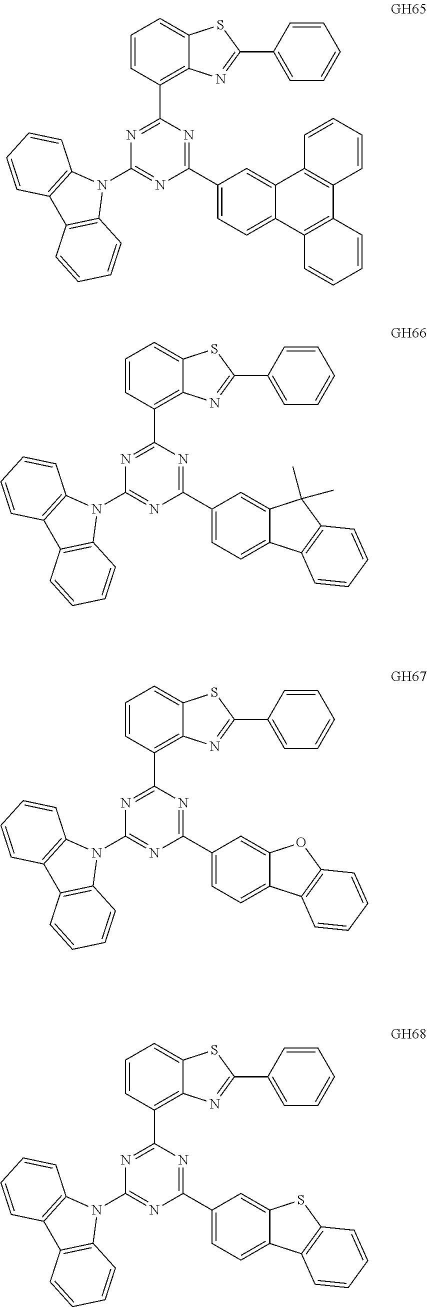 [SMILES: c1ccc(-c2nc3c(-c4nc(-c5ccc6c7ccccc7c7ccccc7c6c5)nc(-n5c6ccccc6c6ccccc65)n4)cccc3s2)cc1 ; CC1(C)c2ccccc2-c2ccc(-c3nc(-c4cccc5sc(-c6ccccc6)nc45)nc(-n4c5ccccc5c5ccccc54)n3)cc21 ; c1ccc(-c2nc3c(-c4nc(-c5ccc6c(c5)oc5ccccc56)nc(-n5c6ccccc6c6ccccc65)n4)cccc3s2)cc1 ; c1ccc(-c2nc3c(-c4nc(-c5ccc6c(c5)sc5ccccc56)nc(-n5c6ccccc6c6ccccc65)n4)cccc3s2)cc1]

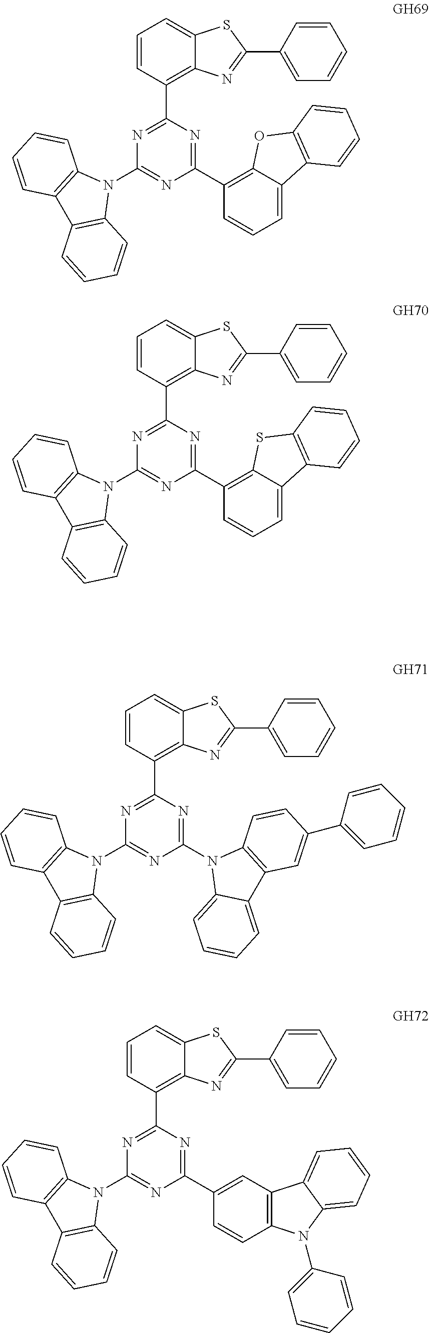 [SMILES: c1ccc(-c2nc3c(-c4nc(-c5cccc6c5oc5ccccc56)nc(-n5c6ccccc6c6ccccc65)n4)cccc3s2)cc1 ; c1ccc(-c2nc3c(-c4nc(-c5cccc6c5sc5ccccc56)nc(-n5c6ccccc6c6ccccc65)n4)cccc3s2)cc1 ; c1ccc(-c2ccc3c(c2)c2ccccc2n3-c2nc(-c3cccc4sc(-c5ccccc5)nc34)nc(-n3c4ccccc4c4ccccc43)n2)cc1 ; c1ccc(-c2nc3c(-c4nc(-c5ccc6c(c5)c5ccccc5n6-c5ccccc5)nc(-n5c6ccccc6c6ccccc65)n4)cccc3s2)cc1]

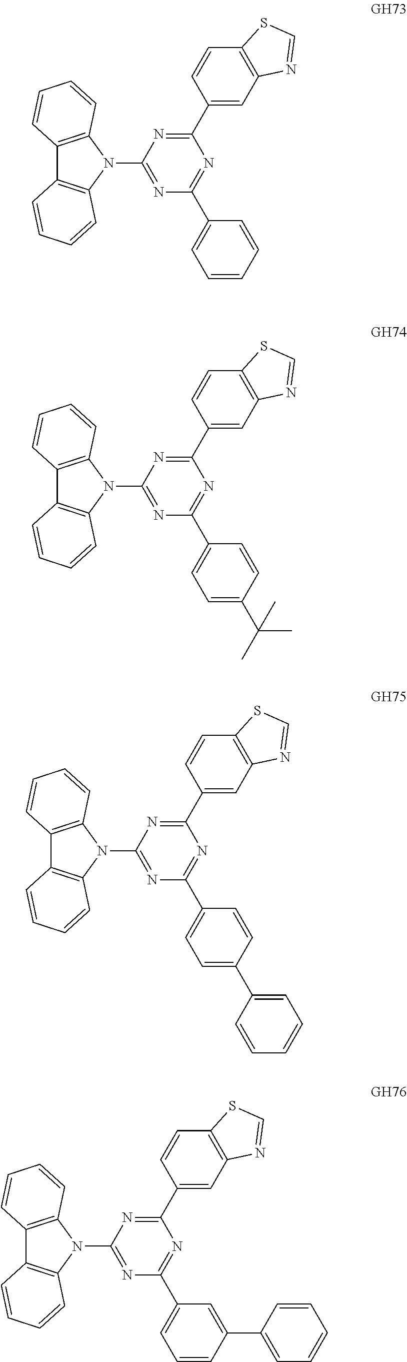 [SMILES: c1ccc(-c2nc(-c3ccc4scnc4c3)nc(-n3c4ccccc4c4ccccc43)n2)cc1 ; CC(C)(C)c1ccc(-c2nc(-c3ccc4scnc4c3)nc(-n3c4ccccc4c4ccccc43)n2)cc1 ; c1ccc(-c2ccc(-c3nc(-c4ccc5scnc5c4)nc(-n4c5ccccc5c5ccccc54)n3)cc2)cc1 ; c1ccc(-c2cccc(-c3nc(-c4ccc5scnc5c4)nc(-n4c5ccccc5c5ccccc54)n3)c2)cc1]

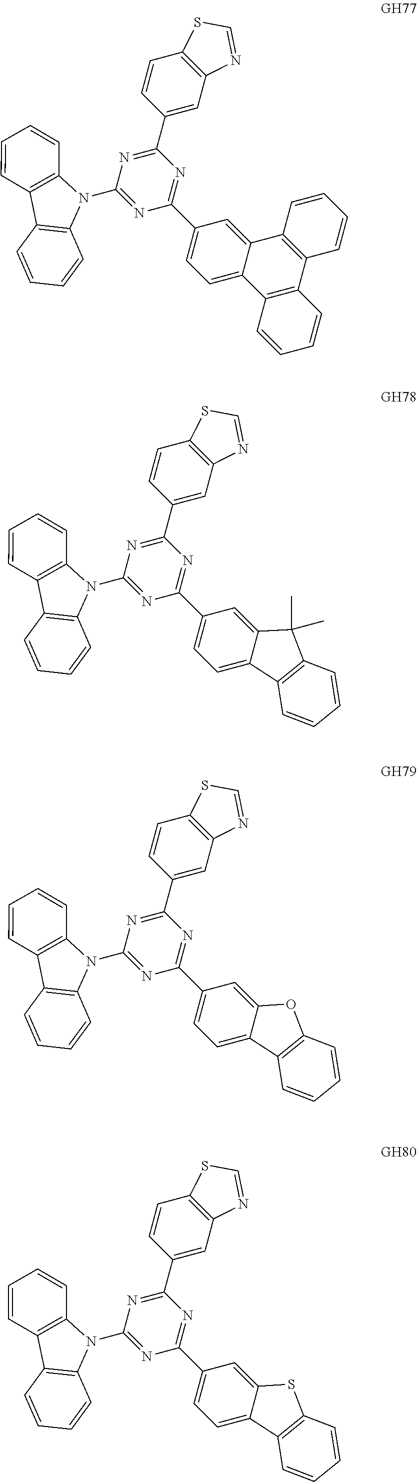 [SMILES: c1ccc2c(c1)c1ccccc1c1cc(-c3nc(-c4ccc5scnc5c4)nc(-n4c5ccccc5c5ccccc54)n3)ccc21 ; CC1(C)c2ccccc2-c2ccc(-c3nc(-c4ccc5scnc5c4)nc(-n4c5ccccc5c5ccccc54)n3)cc21 ; c1ccc2c(c1)oc1cc(-c3nc(-c4ccc5scnc5c4)nc(-n4c5ccccc5c5ccccc54)n3)ccc12 ; c1ccc2c(c1)sc1cc(-c3nc(-c4ccc5scnc5c4)nc(-n4c5ccccc5c5ccccc54)n3)ccc12]

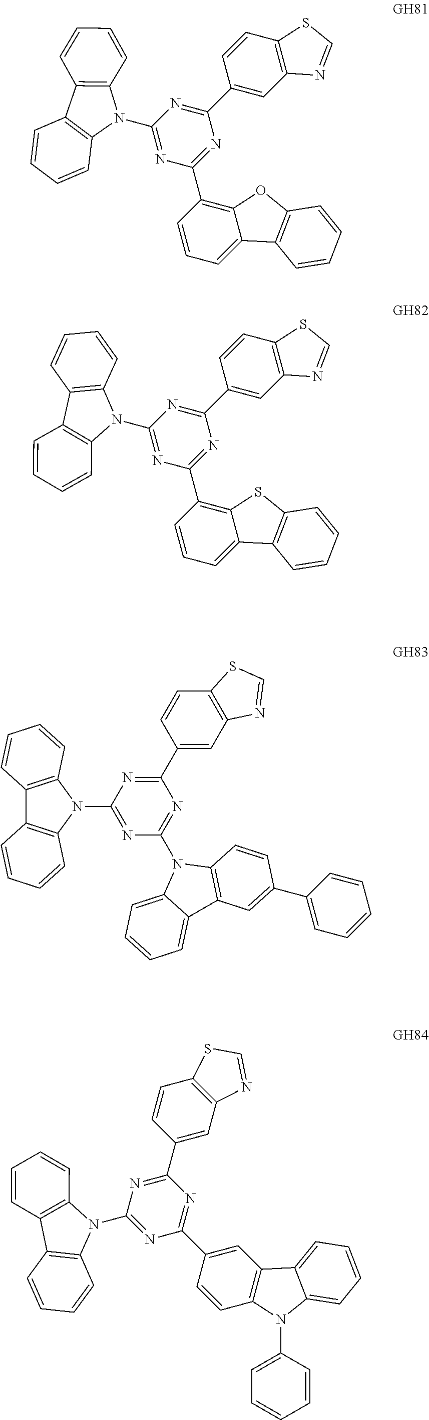 [SMILES: c1ccc2c(c1)oc1c(-c3nc(-c4ccc5scnc5c4)nc(-n4c5ccccc5c5ccccc54)n3)cccc12 ; c1ccc2c(c1)sc1c(-c3nc(-c4ccc5scnc5c4)nc(-n4c5ccccc5c5ccccc54)n3)cccc12 ; c1ccc(-c2ccc3c(c2)c2ccccc2n3-c2nc(-c3ccc4scnc4c3)nc(-n3c4ccccc4c4ccccc43)n2)cc1 ; c1ccc(-n2c3ccccc3c3cc(-c4nc(-c5ccc6scnc6c5)nc(-n5c6ccccc6c6ccccc65)n4)ccc32)cc1]

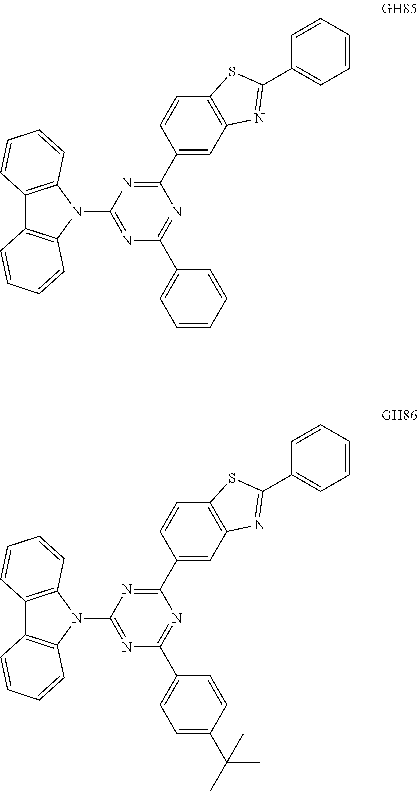 [SMILES: c1ccc(-c2nc(-c3ccc4sc(-c5ccccc5)nc4c3)nc(-n3c4ccccc4c4ccccc43)n2)cc1 ; CC(C)(C)c1ccc(-c2nc(-c3ccc4sc(-c5ccccc5)nc4c3)nc(-n3c4ccccc4c4ccccc43)n2)cc1]

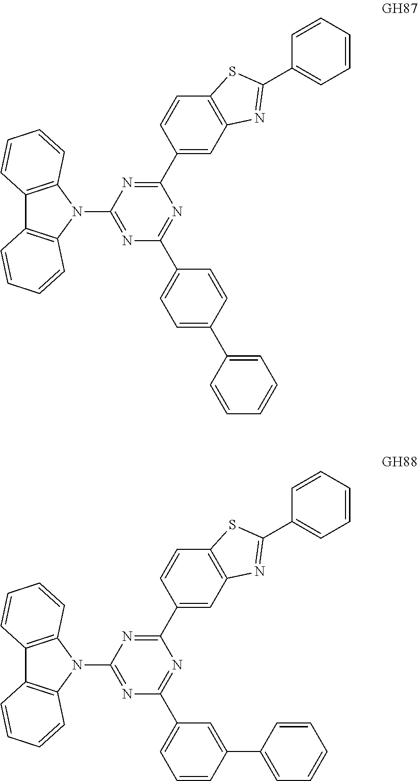 [SMILES: c1ccc(-c2ccc(-c3nc(-c4ccc5sc(-c6ccccc6)nc5c4)nc(-n4c5ccccc5c5ccccc54)n3)cc2)cc1 ; c1ccc(-c2cccc(-c3nc(-c4ccc5sc(-c6ccccc6)nc5c4)nc(-n4c5ccccc5c5ccccc54)n3)c2)cc1]

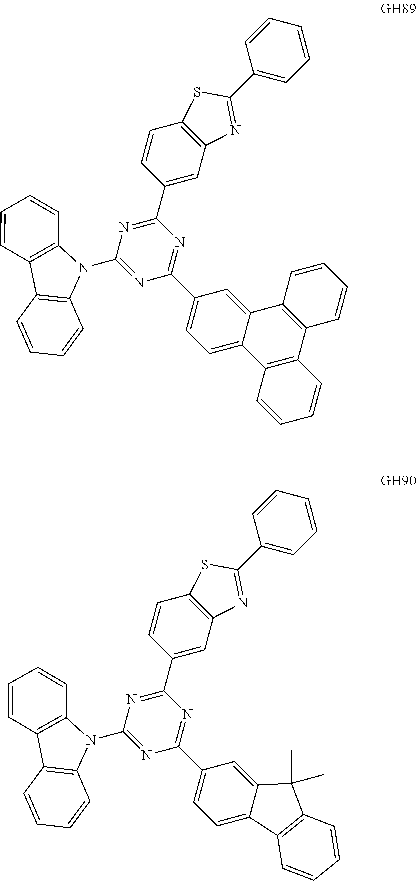 [SMILES: c1ccc(-c2nc3cc(-c4nc(-c5ccc6c7ccccc7c7ccccc7c6c5)nc(-n5c6ccccc6c6ccccc65)n4)ccc3s2)cc1 ; CC1(C)c2ccccc2-c2ccc(-c3nc(-c4ccc5sc(-c6ccccc6)nc5c4)nc(-n4c5ccccc5c5ccccc54)n3)cc21]

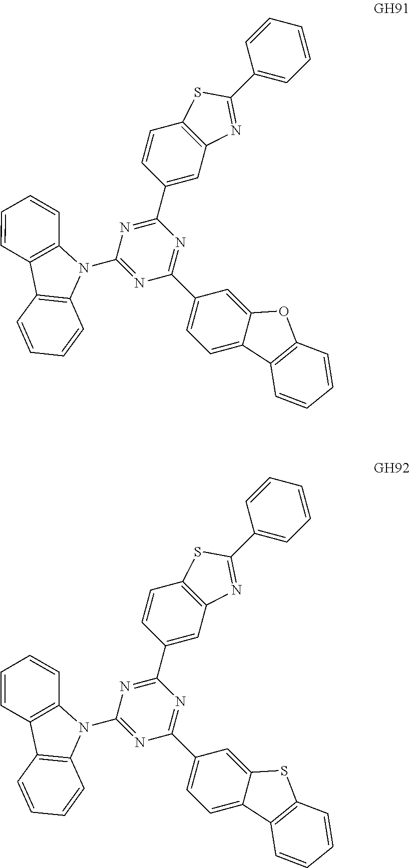 [SMILES: c1ccc(-c2nc3cc(-c4nc(-c5ccc6c(c5)oc5ccccc56)nc(-n5c6ccccc6c6ccccc65)n4)ccc3s2)cc1 ; c1ccc(-c2nc3cc(-c4nc(-c5ccc6c(c5)sc5ccccc56)nc(-n5c6ccccc6c6ccccc65)n4)ccc3s2)cc1]

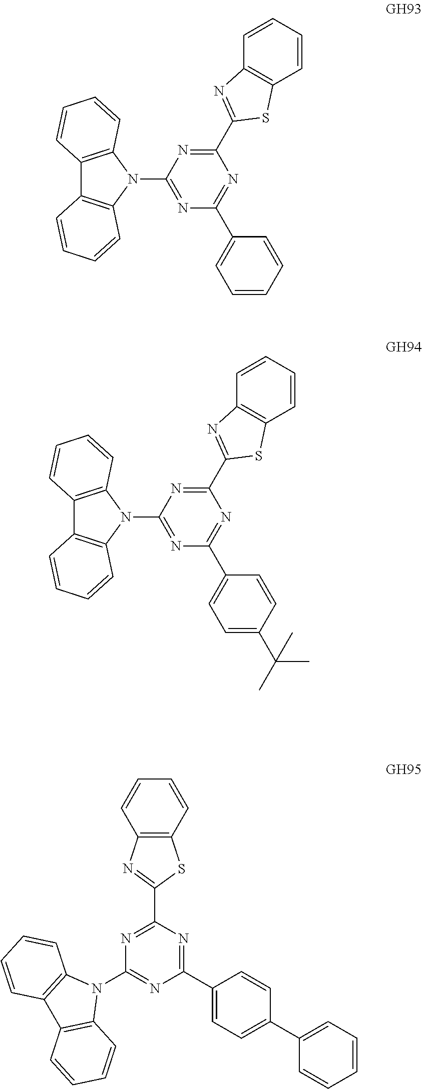 [SMILES: c1ccc(-c2nc(-c3nc4ccccc4s3)nc(-n3c4ccccc4c4ccccc43)n2)cc1 ; CC(C)(C)c1ccc(-c2nc(-c3nc4ccccc4s3)nc(-n3c4ccccc4c4ccccc43)n2)cc1 ; c1ccc(-c2ccc(-c3nc(-c4nc5ccccc5s4)nc(-n4c5ccccc5c5ccccc54)n3)cc2)cc1]

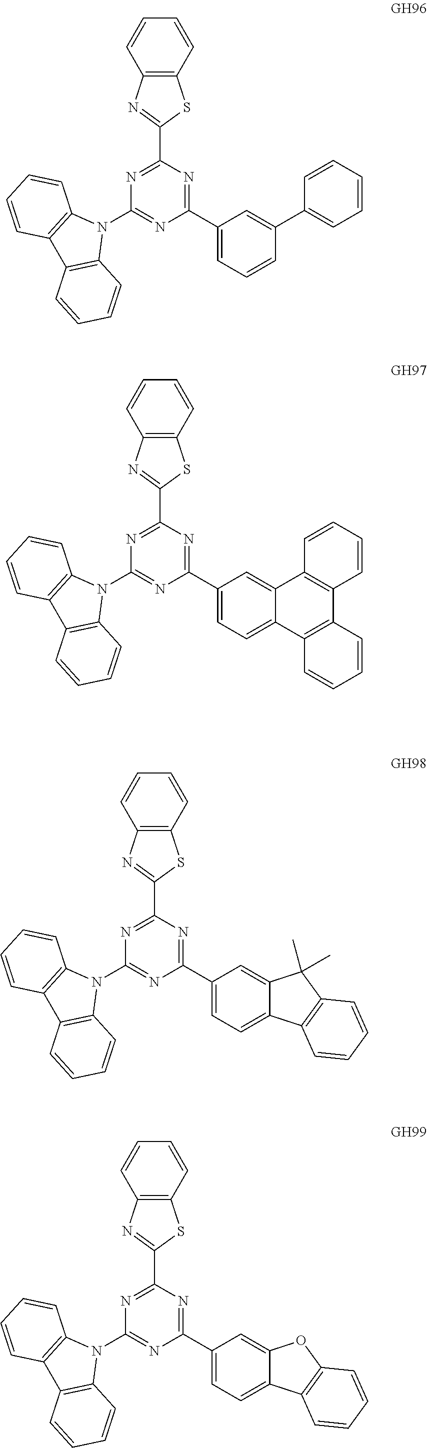 [SMILES: c1ccc(-c2cccc(-c3nc(-c4nc5ccccc5s4)nc(-n4c5ccccc5c5ccccc54)n3)c2)cc1 ; c1ccc2sc(-c3nc(-c4ccc5c6ccccc6c6ccccc6c5c4)nc(-n4c5ccccc5c5ccccc54)n3)nc2c1 ; CC1(C)c2ccccc2-c2ccc(-c3nc(-c4nc5ccccc5s4)nc(-n4c5ccccc5c5ccccc54)n3)cc21 ; c1ccc2sc(-c3nc(-c4ccc5c(c4)oc4ccccc45)nc(-n4c5ccccc5c5ccccc54)n3)nc2c1]

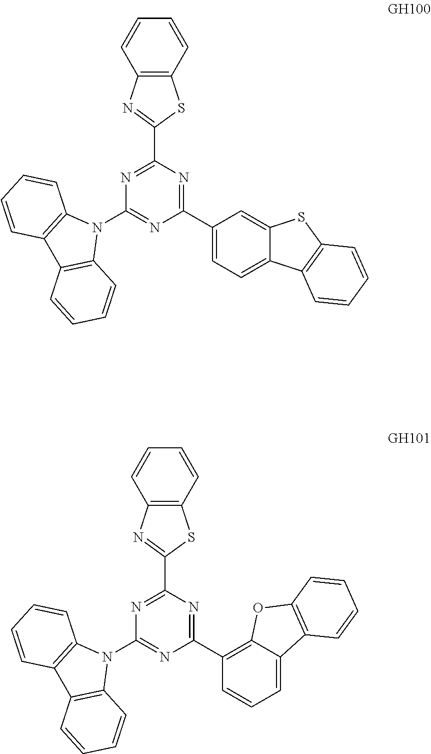 [SMILES: c1ccc2sc(-c3nc(-c4ccc5c(c4)sc4ccccc45)nc(-n4c5ccccc5c5ccccc54)n3)nc2c1 ; c1ccc2sc(-c3nc(-c4cccc5c4oc4ccccc45)nc(-n4c5ccccc5c5ccccc54)n3)nc2c1]

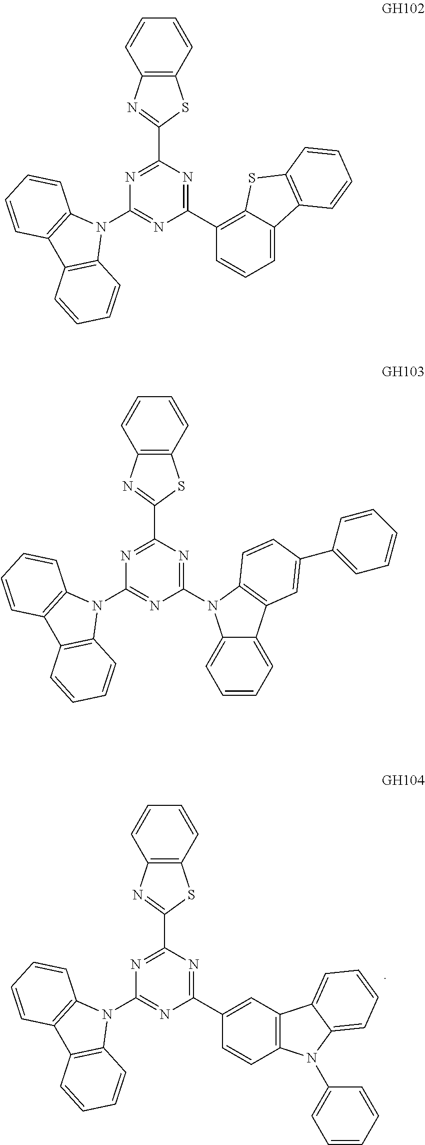 [SMILES: c1ccc2sc(-c3nc(-c4cccc5c4sc4ccccc45)nc(-n4c5ccccc5c5ccccc54)n3)nc2c1 ; c1ccc(-c2ccc3c(c2)c2ccccc2n3-c2nc(-c3nc4ccccc4s3)nc(-n3c4ccccc4c4ccccc43)n2)cc1 ; c1ccc(-n2c3ccccc3c3cc(-c4nc(-c5nc6ccccc6s5)nc(-n5c6ccccc6c6ccccc65)n4)ccc32)cc1]

20. The organic light emitting device according to claim 13, wherein a weight % of the first host to the second host is 1:9 to 9:1, 2:8 to 8:2 or 7:3 to 3:7, or wherein the weight % of the first host and the weight % of the second host may be the same.

21. The organic light emitting device according to claim 6, wherein the organic light emitting diode further includes:
a second emitting part including a first blue emitting material layer, a second electron transporting layer and a second hole blocking layer and positioned between the first electrode and the first emitting part.

22. The organic light emitting device according to claim 21, wherein at least one of the second electron transporting layer and the second hole blocking layer includes an organic compound represented by Formula 1, wherein the organic compound is the same as or different to the first compound.

23. The organic light emitting device according to claim 21, wherein the first emitting part further includes a red emitting material layer between the second emitting part and the first emitting material layer.

24. The organic light emitting device according to claim 22, wherein the first emitting part further includes a yellow-green emitting material layer between the first emitting material layer and the red emitting material layer.

25. The organic light emitting device according to claim 6, further comprising:
a color filter layer corresponding to a red pixel region, a green pixel region and a blue pixel region.
